(19) United States
(12) Patent Application Publication
(10) Pub. No.: US 2026/0263503 A1
(43) Pub. Date: Sep. 10, 2026

(54) COMPOUNDS AND METHODS FOR MODULATING ANGIOTENSINOGEN EXPRESSION

(71) Applicant: Ionis Pharmaceuticals, Inc., Carlsbad, CA (US)

(72) Inventors: Adam MULLICK, Carlsbad, CA (US); Mark J. GRAHAM, San Clemente, CA (US); Punit P. SETH, Carlsbad, CA (US); Susan M. FREIER, San Diego, CA (US)

(73) Assignee: Ionis Pharmaceuticals, Inc., Carlsbad, CA (US)

(21) Appl. No.: 19/455,543

(22) Filed: Jan. 21, 2026

Related U.S. Application Data
(63) Continuation of application No. 17/101,299, filed on Nov. 23, 2020, now abandoned.
Application No. 17/101,299 is a continuation of application No. 15/763,942, filed on Oct. 7, 2016, now Pat. No. 10,912,792.
(60) Provisional application No. 62/238,831, filed on Oct. 8, 2015.

(51) Int. Cl. A61K 31/712; A61P 9/12; C07H 21/00; C07H 21/02; C07H 21/04; C12N 15/113

(52) U.S. Cl. CPC A61K 31/712; A61P 9/12; C07H 21/00; C07H 21/02; C07H 21/04; C12N 15/113; C12N 2310/11

(57) ABSTRACT

Disclosed herein are compositions and compounds comprising modified oligonucleotides for modulating AGT and modulating a RAAS pathway related disease, disorder and/or condition in an individual in need thereof. A RAAS pathway related disease, disorder and/or condition in an individual such as hypertension can be treated, ameliorated, delayed or prevented with the administration of antisense compounds targeted to AGT.

### CROSS REFERENCE TO RELATED APPLICATIONS

This application is a continuation of U.S. patent application Ser. No. 17/101,299, filed on Nov. 23, 2020; which is a continuation of U.S. patent application Ser. No. 15/763,942, filed Mar. 28, 2018, which issued on Feb. 9, 2021, as U.S. Pat. No. 10,912,792 and is a PCT International Application under 35 U.S.C. § 371 of PCT application PCT/US2016/056068, filed Oct. 7, 2016; which claims the benefit of priority to U.S. Provisional Application No. 62/238,831, filed Oct. 8, 2015; each of which is incorporated herein by reference in its entirety.

### SEQUENCE LISTING

The present application is being filed along with a Sequence Listing in electronic format. The Sequence Listing is provided as a file entitled 277023-580545_Sequence_Listing.xml created May 18, 2026, which is 1,846,766 bytes in size. The information in the electronic format of the sequence listing is incorporated herein by reference in its entirety.

### FIELD OF THE INVENTION

The present invention provides compounds, compositions and methods for modulating angiotensinogen (AGT) expression for the purpose of modulating a RAAS pathway related disease, disorder or condition in an animal. The present invention also provides compounds, compositions and methods for reducing hypertension and organ damage by administering an AGT inhibitor to an animal

### BACKGROUND OF THE INVENTION

Angiotensinogen (AGT), also known as SERPINA8 or ANHU, is a member of the serpin family and is a component of the renin-angiotensin-aldosterone system (RAAS). It is primarily produced in the liver and is released into the circulation where renin converts it into angiotensin I. Angiotensin I is subsequently converted into angiotensin II by angiotension converting enzyme (ACE). Angiotensin II is a peptide hormone which causes vasoconstriction which, in turn, can increase blood pressure. Angiotensin II also stimulates secretion of the hormone aldosterone from the adrenal cortex. Aldosterone causes the kidneys to increase reabsorption of sodium and water leading to an increase of the fluid volume in a body which, in turn, can increase blood pressure. Over stimulation or activity of the RAAS pathway can lead to high blood pressure. Chronic high blood pressure is known as hypertension. The high blood pressure in a hypertensive subject requires the heart to work harder to circulate blood through the blood vessels.

The World Health Organization (WHO) has identified hypertension as a leading cause of cardiovascular morbidity. Hypertension is a major risk factor for various disease, disorders and conditions such as shortened life expectancy, chronic kidney disease, stroke, myocardial infarction, heart failure, aneurysms of the blood vessels (e.g. aortic aneurysm), peripheral artery disease, heart damage (e.g., heart enlargement or hypertrophy) and other cardiovascular related diseases, disorders and/or conditions.

The prevelance of resistant hypertension (RHTN), hypertension resistant to drug treatment, has steadily increased in number likely due to an ageing population and an ever increasing incidence of obesity. The current projection of approximately 10 million RHTN adults in the United States is expected to continue to rise.

Anti-hypertensive drugs, renal denervation, baroreceptor activation therapy, diet changes and lifestyle changes may reduce hypertension and reduce the diseases, disorders and/or conditions associated with hypertension (Paulis et al., Nat Rev Cardiol, 2012, 9:276-285). However, there are limitations to the therapies currently approved for treating hypertension as a significant subset of all hypertensive patients do not achieve adequate blood pressure control. For example, drugs such as ACE inhibitors and angiotensin receptor blockers (ARBs) that target parts of the renin-angiotensin system (RAS) pathway are limited in their ability to inhibit the RAAS pathway (Nobakht et al., Nat Rev Nephrol, 2011, 7:356-359). Additionally, certain anti-hypertensive drugs such as ACE inhibitors are contra-indicated in hypertensive patients with renal disease due to their potential to compromise renal function in patients.

Accordingly, there is a need to find alternative treatments to inhibit the RAAS pathway and treat hypertension. Antisense technology is emerging as an effective means for reducing the expression of certain gene products. However, early antisense oligonucleotides targeting AGT provided limited benefit (WO 1997/33623) or targeted non-human AGT (WO 2014/018930). The compounds and compositions herein provide novel, highly potent and tolerable compounds to inhibit human AGT and are suitable for use in human subjects. Additionally, compounds disclosed herein, by using a conjugate strategy that delivers antisense compounds to the liver and limits their renal distribution and activity, are predicted to mitigate the tolerability issues of traditional RAS blockers in patients at risk for hyperkalemia and/or renal disease.

All documents, or portions of documents, cited in this application, including, but not limited to, patents, patent applications, articles, books, and treatises, are hereby expressly incorporated-by-reference for the portions of the document discussed herein, as well as in their entirety.

### SUMMARY OF THE INVENTION

Provided herein are compositions, compounds and methods for lowering the levels of AGT mRNA and/or protein in an animal.

Certain embodiments disclosed herein provide a compound comprising a modified oligonucleotide targeting a nucleic acid sequence encoding AGT. In certain embodiments, the compound targets an AGT sequence as shown in the nucleobase sequences of any of SEQ ID NOs: 1-6.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2250 to 2337 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2281 to 2300 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides and having a nucleobase sequence comprising at least 8 contiguous nucleobases of any of the nucleobase sequences of SEQ ID NOs: 46, 53-54, 61, 68, 76, 83, 85, 93, 96-97, 109, 127, 129-130, 132, 134-15, 137-39, 142, 163-172, 180-184, 186, 189, 234, 236, 238-239, 267, 313, 411, 452, 463-470, 475-478, 480, 500-503, 512, 517-518, 524-526, 654, 689, 702, 725-726, 728, 738, 779, 786-787, 800, 808, 810-811, 825, 865, 868, 889, 894, 903, 905, 909, 954, 966, 1011, 1015, 1021, 1024, 1080, 1085, 1258-1259, 1261-1262, 1293-1294, 1299, 1325, 1470, 1472-1473, 1522, 1542, 1604, 1623-1624, 1667, 1670, 1682-1683, 1687, 1700, 1703-1704, 1708, 1714, 1716, 1719-1720, 1724-1726, 1729-1730, 1827, 1936, 1843-1844, 1846, 1886, 1893-1894, 1914, 1923, 1925, 1932, 1979, 1986, 1988, 1990, 2003, 2015, 2018, 2020, 2027-2028, 2035, 2037, 2039, 2044.

Certain embodiments disclosed herein provide a compound comprising a modified oligonucleotide according to the following formula: mCes Aes mCes Aes Aes Ads mCds Ads Ads Gds mCds Tds Gds Gds Tds mCes Ges Ges Tes Te (SEQ ID NO: 1914); wherein, A is an adenine, mC is a 5′-methylcytosine, G is a guanine, T is a thymine, e is a 2′-O-methoxyethyl modified nucleoside, d is a 2′-deoxynucleoside, and s is a phosphorothioate internucleoside linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

Certain embodiments disclosed herein provide a compound comprising a modified oligonucleotide with the following formula:

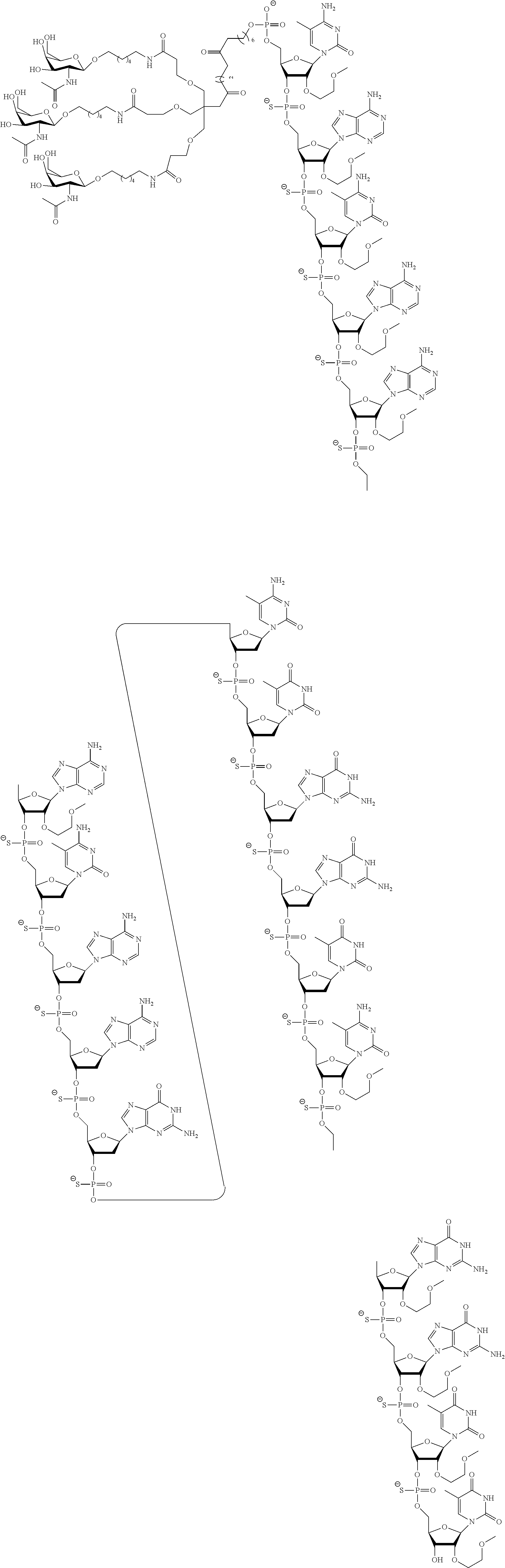

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“About” means within ±10% of a value. For example, if it is stated, “a marker may be increased by about 50%”, it is implied that the marker may be increased between 45%-55%.

“ACE escape”, also known as angiotensin II reactivation, refers to the inability of currently available ACE inhibitor treatment to reliably suppress plasma angiotensin II levels. The increase in plasma angiotensin II levels during ACE inhibition occurs via other enzymes converting angiotensin I to angiotensin. This incomplete blockage of angiotensin II levels prevents the ACE inhibitors from effectively treating some hypertensive subjects. Angiotensin Receptor Blockers (ARBs) may also be susceptible to ACE escape as other receptors besides the ATI receptor engage angiotensin metabolites.

“Active pharmaceutical agent” or “Pharmaceutical agent” means the substance or substances in a pharmaceutical composition that provide a therapeutic benefit when administered to an individual. For example, in certain embodiments, an antisense oligonucleotide targeted to AGT is an active pharmaceutical agent.

“Active target region” or “target region” means a region to which one or more active antisense compounds is targeted.

“Active antisense compounds” means antisense compounds that reduce target nucleic acid levels or protein levels.

“Administered concomitantly” refers to the co-administration of two agents in any manner in which the pharmacological effects of both are manifest in the patient time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Aldosterone escape” or “aldosterone breakthrough” refers to the inability of currently available ACE inhibitor Angiotensin Receptor Blocker (ARB) and/or Direct Renin Inhibitor (DRI) treatment to reliably suppress aldosterone release in some treated subjects. This incomplete blockage of aldosterone prevents the ACE inhibitors, DRIs and ARBs from effectively treating some hypertensive subjects.

“Agent” means an active substance that can provide a therapeutic benefit when administered to an animal. “First Agent” means a therapeutic compound provided herein. For example, a first agent is an antisense oligonucleotide targeting AGT. “Second agent” means a second therapeutic compound described herein. For example, a second agent can be a second antisense oligonucleotide targeting AGT or a non-AGT target. Alternatively, a second agent can be a compound other than an antisense oligonucleotide.

“Amelioration” or “ameliorate” refers to a lessening of at least one indicator, marker, sign, or symptom of an associated disease, disorder and/or condition. In certain embodiments, amelioration includes a delay or slowing in the progression of one or more indicators of a condition, disorder and/or disease. The severity of indicators may be determined by subjective or objective measures, which are known to those skilled in the art.

“Angiotensinogen” and “AGT” is used interchangeably herein. Angiotensinogen is also known as SERPINA8 and ANHU.

“Angiotensinogen nucleic acid” or “AGT nucleic acid” means any nucleic acid encoding AGT. For example, in certain embodiments, an AGT nucleic acid includes a DNA sequence encoding AGT, an RNA sequence transcribed from DNA encoding AGT (including genomic DNA comprising introns and exons), and an mRNA sequence encoding AGT. “AGT mRNA” means an mRNA encoding an AGT protein.

“AGT specific inhibitor” refers to any agent capable of specifically inhibiting the expression of AGT mRNA and/or AGT protein. For example, AGT specific inhibitors include nucleic acids (including antisense compounds such as RNasH, siRNA and blockmer antisense compounds), peptides, antibodies, small molecules, and other agents capable of specifically inhibiting the expression of AGT mRNA and/or AGT protein. In certain embodiments, by specifically modulating AGT mRNA level and/or AGT protein expression, AGT specific inhibitors can affect components of the renin-angiotensin-aldosterone system (RAAS) pathway. In certain embodiments, by specifically modulating AGT mRNA level and/or AGT protein expression, AGT specific inhibitors can affect RAAS pathway related diseases, disorders and/or conditions such as blood pressure. Similarly, in certain embodiments, AGT specific inhibitors can affect other molecular processes in an animal.

“Animal” refers to a human or non-human animal, including, but not limited to, mice, rats, rabbits, dogs, cats, pigs, and non-human primates, including, but not limited to, monkeys and chimpanzees.

“Anti-hypertensive drug” refers to a drug capable of lowering blood pressure. Examples of such drugs include, but are not limited to, RAAS inhibitors, diuretics, calcium channel blockers, adrenergic receptor antagonists, adrenergic agonists and vasodilators. In one example, the anti-hypertensive drug captopril can be used in combination with the AGT compound described herein to treat an animal having or at risk of having a RAAS pathway related disease, disorder and/or condition.

“Anti-hypertensive procedure” refers to a medical procedure performed on a subject to reduce hypertension. Examples of such procedures include renal denervation and baroreceptor activation therapy. “Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Blood pressure” refers to the pressure of the blood in the circulatory system against the walls of the blood vessel. The blood pressure is due mainly to the beating of the heart in an animal. During each heartbeat, the blood pressure varies between a maximum (systolic) blood pressure (SBP) and minimum (diastolic) blood pressure (DBP). The mean arterial pressure (MAP) is the average arterial pressure during a heartbeat cycle. Blood pressure can be measure by a blood pressure meter (i.e., a sphygmomanometer). Normal blood pressure at rest is within the range of 100-140 mmHg systolic and 60-90 mmHg diastolic and is commonly expressed as the systolic pressure (top reading)/diastolic pressure (bottom reading) mmHg.

“Cap structure” or “terminal cap moiety” means chemical modifications, which have been incorporated at either terminus of an antisense compound.

“cEt” or “constrained ethyl” means a bicyclic sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses concomitant, parallel or sequential administration.

“Complementarity” means the capacity for pairing between nucleobases of a first nucleic acid and a second nucleic acid. In certain embodiments, the first nucleic acid is an antisense compound and the second nucleic acid is a target nucleic acid.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Diluent” means an ingredient in a composition that lacks pharmacological activity, but is pharmaceutically necessary or desirable. For example, the diluent in an injected composition may be a liquid, e.g. phosphate buffered saline (PBS) or water.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors. In an example, an effective amount of an AGT antisense oligonucleotide decreases blood pressure and/or ameliorates organ damage due to hypertension.

“Fully complementary” or “100% complementary” means that each nucleobase of a nucleobase sequence of a first nucleic acid has a complementary nucleobase in a second nucleobase sequence of a second nucleic acid. In certain embodiments, the first nucleic acid is an antisense compound and the second nucleic acid is a target nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap segment” and the external regions may be referred to as “wing segments.”

“Gap-widened” means a chimeric antisense compound having a gap segment of 12 or more contiguous 2′-deoxynucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Hypertension” or “HTN” refers to a chronic medical condition where the blood pressure in an animal is elevated. The elevated blood pressure requires the heart to work harder to circulate blood through the blood vessels. High blood pressure is said to be present if it is persistently at or above 140/90 mmHg.

Hypertension is classified as primary (essential) or secondary. Primary hypertension has no clear cause and is thought to be linked to genetics, diet, lack of exercise and obesity. Secondary hypertension is caused by another medical condition. Hypertension is a major risk factor for shortened life expectancy, chronic kidney disease, stroke, myocardial infarction, heart failure, aneurysms of the blood vessels (e.g. aortic aneurysm), peripheral artery disease, organ damage (e.g., heart enlargement or hypertrophy) and other cardiovascular diseases, disorders and/or conditions or symptoms thereof. Anti-hypertensive drugs, diet changes and lifestyle changes may reduce hypertension and reduce the diseases, disorders and/or conditions associated with hypertension. Hypertension can be nonresistant to drug intervention (i.e., controllable by commercially available drug therapies) or resistant to drug intervention.

“Identifying an animal having, or at risk for, a RAAS related disease, disorder and/or condition” means identifying an animal having been diagnosed with a RAAS related disease, disorder and/or condition or identifying an animal predisposed to develop a RAAS related disease, disorder and/or condition. Individuals predisposed to develop a RAAS related disease, disorder and/or condition include, for example, individuals with a familial history a RAAS related disease such as hypertension. Such identification may be accomplished by any method including evaluating an individual's medical history and standard clinical tests or assessments.

“Immediately adjacent” means that there are no intervening elements between the immediately adjacent elements.

“Individual” or “subject” or “animal” means a human or non-human animal selected for treatment or therapy.

“Inhibiting the expression or activity” refers to a reduction or blockade of the expression or activity of a RNA or protein and does not necessarily indicate a total elimination of expression or activity.

“Internucleoside linkage” refers to the chemical bond between nucleosides.

“Intravenous administration” means administration into a vein.

“Linked nucleosides” means adjacent nucleosides which are bonded together.

“Marker” or “biomarker” is any measurable and quantifiable biological parameter that serves as an index for health- or physiology-related assessments. For example, an increase in blood pressure, or a decrease in organ damage (e.g., fibrosis) can be considered markers of an RAAS related disease, disorder and/or condition.

“Mismatch” or “non-complementary nucleobase” or “MM” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. For example, a modified nucleobase can be 5′-methylcytosine. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, and/or modified nucleobase.

“Modified oligonucleotide” means an oligonucleotide comprising a modified internucleoside linkage, a modified sugar, and/or a modified nucleobase.

“Modified sugar” refers to a substitution or change from a natural sugar. For example, a modified sugar can be 2′-MOE.

“Modulating” refers to changing or adjusting a feature in a cell, tissue, organ or organism. For example, modulating AGT mRNA can mean to increase or decrease the level of AGT mRNA and/or AGT protein in a cell, tissue, organ or organism. Modulating AGT mRNA and/or protein can lead to an increase or decrease in a RAAS related disease, disorder and/or condition in a cell, tissue, organ or organism. A “modulator” effects the change in the cell, tissue, organ or organism. For example, an AGT antisense compound can be a modulator that increases or decreases the amount of AGT mRNA and/or AGT protein in a cell, tissue, organ or organism.

“Monomer” refers to a single unit of an oligomer. Monomers include, but are not limited to, nucleosides and nucleotides, whether naturally occurring or modified.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nonresistant hypertension”, “nonrefractory hypertension” or “controlled hypertension” is defined as hypertension that responds to treatment resulting in, for example, blood pressure <140 mmHg SBP or <90 mmHg DBP with concurrent use of up to 3 anti-hypertensive agents.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound; such as, for example, nucleoside mimetics having morpholino, cyclohexenyl, cyclohexyl, tetrahydropyranyl, bicyclo or tricyclo sugar mimetics e.g. non furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound; such as, for example, peptide nucleic acids or morpholinos (morpholinos linked by —N(H)—C(═O)—O— or other non-phosphodiester linkage).

“Organ damage” or “end organ damage” refers to damage occurring in major organs fed by the circulatory system such as the heart (e.g., heart muscle hypertrophy, reduced heart function and/or heart failure), kidney (e.g., albuminurea, proteinurea, reduced renal function and/or renal failure), eyes (e.g., hypertensive retinopathy), brain (e.g., stroke) and the like. The organs can be damaged by hypertension in an animal. In certain embodiments, the heart damage is fibrosis, heart cell and/or muscle hypertrophy leading to heart enlargement.

“Oligomeric compound” or “oligomer” refers to a polymeric structure comprising two or more sub-structures (monomers) and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antisense oligonucleotides. In certain embodiments, oligomeric compounds are chimeric oligonucleotides.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intra-arterial administration, intraperitoneal administration, or intracranial administration, e.g., intrathecal or intracerebroventricular administration. Administration can be continuous, or chronic, or short or intermittent.

“Peptide” refers to a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable carrier” means a medium or diluent that does not interfere with the structure of the oligonucleotide. Certain of such carriers enable pharmaceutical compositions to be formulated as, for example, tablets, pills, dragees, capsules, liquids, gels, syrups, slurries, suspension and lozenges for the oral ingestion by a subject. For example, a pharmaceutically acceptable carrier can be a sterile aqueous solution, such as sterile water or PBS.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset, development, or progression of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Renin-angiotensin-aldosterone system”, “Renin-angiotensin-aldosterone system pathway”, “RAAS pathway” or “RAAS” refer to a multi-component enzymatic pathway where a precursor component (angiotensinogen) is converted by various enzymes such as renin and enzyme angiotensin-converting-enzyme (ACE) into downstream components such as angiotensin I and angiotensin II. Angiotensin I stimulates secretion of the steroid aldosterone in the pathway. The RAAS pathway regulates blood pressure and fluid balance in a body.

“Renin-angiotensin System”, or “RAS” or “RAS pathway” refer to a portion of the RAAS pathway. Various components of this pathway have been targeted by agonists or antagonists to block the production of the components. For example renin inhibitors, ACE inhibitors, angiotensin-receptor blockers (ARBs) and the like have been developed to inhibit or block the RAS pathway. However, commercially available therapies targeting various RAS pathway components have been ineffective in completely inhibiting or blocking the RAS pathway due to various mechanisms (Nobakht et al., Nat Rev Nephrol, 2011, 7:356-359).

“RAAS related disease, disorder and/or condition” or “RAAS pathway related disease, disorder and/or condition” refers to any disease, disorder or condition related to RAAS in an animal. Examples of RAAS related diseases, disorders and/or conditions include shortened life expectancy, hypertension (e.g. nonresistant hypertension, resistant hypertension), kidney disease (e.g., chronic kidney disease, polycystic kidney disease), stroke, heart disease (e.g., myocardial infarction, heart failure, valvular heart disease), aneurysms of the blood vessels (e.g. aortic aneurysm), peripheral artery disease, organ damage (e.g., heart damage or hypertrophy), tissue fibrosis and other cardiovascular diseases, disorders and/or conditions or symptoms thereof. In certain embodiments, RAAS related disease, disorder and/or condition does not include hypertension.

“Resistant hypertension” or “RHTN” is defined as (1) blood pressure ≥140 mmHg SBP or ≥90 mmHg DBP despite concurrent use of 3 anti-hypertensive agents from different drug classes or (2) use of ≥4 anti-hypertensive drugs regardless of blood pressure.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, e.g., under physiological conditions in the case of in vivo assays and therapeutic treatments. In an example, an antisense compound is specifically hybridizable to a target when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired.

“Subcutaneous administration” means administration just below the skin.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an animal.

“Treat” refers to administering a pharmaceutical composition to an animal in order to effect an alteration or improvement of a disease, disorder, or condition in the animal. In certain embodiments, one or more pharmaceutical compositions can be administered to the animal.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleotide) or a DNA nucleotide (i.e. β-D-deoxyribonucleotide).

### CERTAIN EMBODIMENTS

Certain embodiments provide compounds specifically modulating AGT. In certain embodiments, the AGT specific modulators are AGT specific inhibitors, for use in treating, preventing, or ameliorating a RAAS related disease, disorder and/or condition. In certain embodiments, AGT specific inhibitors are nucleic acid compounds capable of inhibiting the expression of AGT mRNA and/or AGT protein. In certain embodiments, the nucleic acid compounds are oligomeric compounds. In certain embodiments, the oligomeric compounds are antisense oligonucleotides. In certain embodiments, the antisense oligonucleotides are modified antisense oligonucleotides. In certain embodiments, the modified antisense oligonucleotides are chimeric antisense oligonucleotides.

In certain embodiments, the compounds target an AGT nucleic acid. In certain embodiments, the AGT nucleic acid is any of the human sequences set forth in GENBANK Accession No. NM 000029.3 (incorporated herein as SEQ ID NO: 1), the complement of the nucleotides 24354000 to 24370100 of GENBANK Accession No. NT_167186.1 (incorporated herein as SEQ ID NO: 2), GENBANK Accession No. AK307978.1 (incorporated herein as SEQ ID NO: 3), GENBANK Accession No. AK303755.1 (incorporated herein as SEQ ID NO: 4), GENBANK Accession No. AK293507.1 (incorporated herein as SEQ ID NO: 5), and GENBANK Accession No. CR606672.1 (incorporated herein as SEQ ID NO: 6).

Certain embodiments disclosed herein provide a compound comprising a modified oligonucleotide targeting a nucleic acid sequence encoding AGT. In certain embodiments, the compound targets an AGT sequence as shown in the nucleobase sequences of any of SEQ ID NOs: 1-6.

Certain embodiments disclosed herein provide a compound comprising a modified oligonucleotide consisting of 12 to 30 linked nucleosides having a nucleobase sequence comprising at least 8, least 9, least 10, least 11, at least 12, least 13, at least 14, at least 15, at least 16, least 17, least 18, least 19, or 20 contiguous nucleobases complementary to an equal length portion of SEQ ID NOs: 1-6.

In certain embodiments, the nucleobase sequence of the modified oligonucleotide is at least 70%, at least 75%, at least 80%, at least 85%, at least 90%, at least 95%, at least 96%, at least 97%, at least 98%, or at least 99% complementary to an equal length portion of any of SEQ ID NOs: 1-6. In certain embodiments, the modified oligonucleotide comprises a nucleobase sequence 100% complementary to an equal length portion of any of SEQ ID NOs: 1-6.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2027-2068 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 2027 to 2068 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2250 to 2337 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 2250 to 2337 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2266 to 2337 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 2266 to 2337 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2281 to 2300 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 2281 to 2300 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8 contiguous nucleobases complementary to an equal length portion of nucleobases 2324 to 2346 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 2324 to 2346 of SEQ ID NO: 1, wherein the nucleobase sequence of the modified oligonucleotide is at least 80% complementary to SEQ ID NO: 1.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 14-2051.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 40, 42, 46, 47, 49, 53 to 55, 61, 62, 68, 71, 76, 82, 84, 85, 89, 93, 96 to 98, 102, 109, 114, 119, 127, 129, 130 to 135, 137 to 140, 142, 143, 160, 162 to 207, 209, 210, 223, 225 to 227, 230 to 243, 252 to 254, 257, 258, 262 to 273, 276, 278, 279, 281, 284, 452, 463, 464, 466, 467, 470, 477, 480, 500, 502, 512, 517, 525, 526, 726, 728, 868, 905, 906, 954, 961, 962, 963, 965, 966, 971, 973, 986, 987, 989, 990, 991, 994, 997, 998, 1000, 1001, 1011, 1015, 1021, 1024, 1035, 1080, 1085, 1150, 1258, 1259 to 1262, 1293, 1294, 1299, 1325, 1326, 1354, 1355 to 1357, 1370, 1384, 1391, 1393 to 1395, 1406 to 1408, 1431, 1467, 1468, 1470, 1472 to 1474, 1476, 1488, 1489, 1500, 1503, 1504, 1522, 1524, 1526, 1528, 1535, 1536, 1539, 1542, 1543, 1545, 1585, 1592, 1594, 1595, 1599, 1604, 1610 to 1612, 1615, 1618, 1619 to 1624, 1626, 1628, 1629, 1631, 1632, 1635 to 1637, 1640, 1658, 1662, 1665 to 1671, 1673, 1676 to 1679, 1681 to 1683, 1686, 1687, 1699 to 1710, 1712, 1714 to 1721, 1724 to 1726, 1728 to 1731, 1735, 1736, 1739 to 1741, 1751, 1755, 1771, 1778, 1781 to 1783, 1827, 1834, 1836, 1843 to 1846, 1872, 1874, 1875 to 1888, 1890 to 1895, 1897, 1898, 1900, 1904 to 1927, 1931 to 1933, 1937, 1939, 1940, 1943, 1950, 1951, 1953, 1955 to 1959, 1962, 1964 to 1967, 1969 to 1971, 1973, 1977 to 1981, 1984 to 1991, 1993 to 1996, 2000 to 2005, 2007 to 2012, 2014 to 2025, 2027, 2028, 2030, 2032 to 2037, 2039-2045, 2047, 2051.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 46, 53, 54, 68, 76, 85, 96, 97, 114, 127, 129 to 132, 134, 135, 137 to 139, 142, 162 to 207, 225, 226, 230 to 243, 252, 264, 266 to 270, 284, 464, 467, 962, 963, 965, 966, 973, 990, 991, 997, 1000, 1001, 1011, 1261, 1299, 1355, 1356, 1470, 1472, 1473, 1503, 1504, 1522, 1526, 1535, 1536, 1542, 1543, 1545, 1595, 1599, 1604, 1620, 1623, 1624, 1626, 1640, 1662, 1666, 1667, 1669, 1670, 1673, 1682, 1683, 1687, 1699 to 1706, 1708, 1712, 1714 to 1716, 1719 to 1721, 1724 to 1726, 1729, 1730, 1736, 1778, 1783, 1836, 1843, 1875 to 1888, 1893 to 1895, 1897, 1900, 1904 to 1908, 1911, 1914 to 1918, 1920, 1922, 1923, 1925, 1926, 1931 to 1933, 1937, 1939, 1955, 1958, 1959, 1962, 1966, 1967, 1970, 1971, 1973, 1977, 1978 to 1981, 1985, 1986, 1987, 1988, 1990, 1991, 1994, 1996, 2000, 2002 to 2005, 2010, 2011, 2014 to 2025, 2027, 2028, 2035 to 2037, 2039, 2041 to 2045.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 96, 127, 129 to 132, 139, 162 to 169, 171 to 189, 191 to 193, 195, 196, 198 to 206, 234, 236, 238 to 240, 267 to 270, 966, 1000, 1522, 1542, 1623, 1624, 1667, 1682, 1683, 1700, 1703, 1704, 1708, 1714, 1719, 1720, 1724 to 1726, 1729, 1875, 1876, 1878, 1884 to 1886, 1893, 1894, 1906, 1908, 1914, 1917, 1918, 1922, 1923, 1925, 1926, 1932, 1933, 1967, 1970, 1978 to 1981, 1985, 1986, 1988, 1990, 1991, 2003, 2010, 2015, 2016, 2018, 2020, 2021, 2024, 2025, 2027, 2028, 2035, 2037, 2039, 2044.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 129, 130, 132, 163 to 168, 171, 172, 175 to 186, 188, 189, 192, 193, 195, 198 to 206, 238, 239, 966, 1703, 1720, 1726, 1923, 1925, 2003, 2015.

Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 46, 53-54, 61, 68, 76, 83, 85, 93, 96-97, 109, 127, 129-130, 132, 134-15, 137-39, 142, 163-172, 180-184, 186, 189, 234, 236, 238-239, 267, 313, 411, 452, 463-470, 475-478, 480, 500-503, 512, 517-518, 524-526, 654, 689, 702, 725-726, 728, 738, 779, 786-787, 800, 808, 810-811, 825, 865, 868, 889, 894, 903, 905, 909, 954, 966, 1011, 1015, 1021, 1024, 1080, 1085, 1258-1259, 1261-1262, 1293-1294, 1299, 1325, 1470, 1472-1473, 1522, 1542, 1604, 1623-1624, 1667, 1670, 1682-1683, 1687, 1700, 1703-1704, 1708, 1714, 1716, 1719-1720, 1724-1726, 1729-1730, 1827, 1936, 1843-1844, 1846, 1886, 1893-1894, 1914, 1923, 1925, 1932, 1979, 1986, 1988, 1990, 2003, 2015, 2018, 2020, 2027-2028, 2035, 2037, 2039, 2044. Certain embodiments disclosed herein provide a compound comprising a modified oligonucleotide consisting of 12 to 30 linked nucleosides having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 238, 1714, 1719, 1893-1894, 1914, 1923, 1925, 2003.

In certain embodiments, the compound comprises a modified oligonucleotide consisting of 8 to 80, 20 to 80, 10 to 50, 20 to 35, 10 to 30, 12 to 30, 15 to 30, 16 to 30, 20 to 30, 20 to 29, 20 to 28, 20 to 27, 20 to 26, 20 to 25, 20 to 24, 20 to 23, 20 to 22, 20 to 21, 15 to 25, 16 to 25, 15 to 24, 16 to 24, 17 to 24, 18 to 24, 19 to 24, 19 to 22, 16 to 21, 18 to 21 or 16 to 20 linked nucleobases. In certain embodiments, the compound comprises a modified oligonucleotide consisting of 16 linked nucleosides. In certain embodiments, the compound comprises a modified oligonucleotide consisting of 20 linked nucleosides.

In certain embodiments, the compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values.

In certain embodiments, the modified oligonucleotide is single-stranded.

In certain embodiments, the modified oligonucleotide comprises at least one modified internucleoside linkage. In certain embodiments, the modified internucleoside linkage is a phosphorothioate internucleoside linkage. In certain embodiments, at least one modified internucleoside linkage is a phosphorothioate internucleoside linkage. In certain embodiments, each modified internucleoside linkage is a phosphorothioate internucleoside linkage.

In certain embodiments, the modified oligonucleotide comprises at least one nucleoside comprising a modified sugar. In certain embodiments, at least one modified sugar comprises a bicyclic sugar. In certain embodiments, at least one modified sugar comprises a 2′-O-methoxyethyl, a constrained ethyl, a 3′-fluoro-HNA or a 4′-(CH₂)ₙ—O-2′ bridge, wherein n is 1 or 2.

In certain embodiments, the modified oligonucleotide comprises at least one nucleoside comprising a modified nucleobase. In certain embodiments, the modified nucleobase is a 5-methylcytosine.

In certain embodiments, the modified oligonucleotide comprises a conjugate group. In certain embodiments, the conjugate is a carbohydrate moiety. In certain embodiments, the conjugate is a GalNAc moiety. In certain embodiments, the GalNAc is 5′-Trishexylamino-(THA)-C6 GalNAc₃. In certain embodiments, the 5′-Trishexylamino-(THA)-C6 GalNAc₃ conjugate has the formula

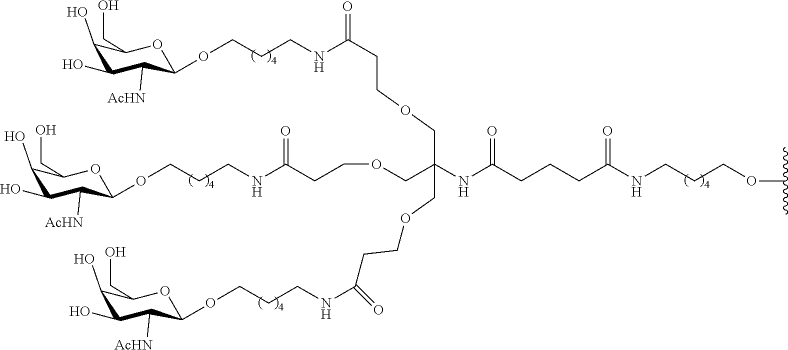

In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprises a modified oligonucleotide consisting of 12 to 30 linked nucleosides and targeted to or complementary to an equal length portion of region 2250 to 2337 of SEQ ID NO: 1, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment and wherein each nucleoside of each wing segment comprises a modified sugar. In certain embodiments, the modified oligonucleotide further comprises at least one phosphorothioate internucleoside linkage. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprises a modified oligonucleotide consisting of 12 to 30 linked nucleosides and targeted to or complementary to an equal length portion of region 2266 to 2337 of SEQ ID NO: 1, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment and wherein each nucleoside of each wing segment comprises a modified sugar. In certain embodiments, the modified oligonucleotide further comprises at least one phosphorothioate internucleoside linkage. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprises a modified oligonucleotide consisting of 12 to 30 linked nucleosides and targeted to or complementary to an equal length portion of region 2281 to 2300 of SEQ ID NO: 1, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment and wherein each nucleoside of each wing segment comprises a modified sugar. In certain embodiments, the modified oligonucleotide further comprises at least one phosphorothioate internucleoside linkage. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprises a modified oligonucleotide consisting of 20 linked nucleosides and targeted to or complementary to an equal length portion of region 2027 to 2068 of SEQ ID NO: 1, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a 2′-O-methoxyethyl sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 14-2051, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group. In certain embodiments, the compound comprising a modified oligonucleotide consisting of 16 to 20 linked nucleosides and having a nucleobase sequence comprising at least 8 contiguous nucleobases of SEQ ID NOs: 40, 42, 46, 47, 49, 53 to 55, 61, 62, 68, 71, 76, 82, 84, 85, 89, 93, 96 to 98, 102, 109, 114, 119, 127, 129, 130 to 135, 137 to 140, 142, 143, 160, 162 to 207, 209, 210, 223, 225 to 227, 230 to 243, 252 to 254, 257, 258, 262 to 273, 276, 278, 279, 281, 284, 452, 463, 464, 466, 467, 470, 477, 480, 500, 502, 512, 517, 525, 526, 726, 728, 868, 905, 906, 954, 961, 962, 963, 965, 966, 971, 973, 986, 987, 989, 990, 991, 994, 997, 998, 1000, 1001, 1011, 1015, 1021, 1024, 1035, 1080, 1085, 1150, 1258, 1259 to 1262, 1293, 1294, 1299, 1325, 1326, 1354, 1355 to 1357, 1370, 1384, 1391, 1393 to 1395, 1406 to 1408, 1431, 1467, 1468, 1470, 1472 to 1474, 1476, 1488, 1489, 1500, 1503, 1504, 1522, 1524, 1526, 1528, 1535, 1536, 1539, 1542, 1543, 1545, 1585, 1592, 1594, 1595, 1599, 1604, 1610 to 1612, 1615, 1618, 1619 to 1624, 1626, 1628, 1629, 1631, 1632, 1635 to 1637, 1640, 1658, 1662, 1665 to 1671, 1673, 1676 to 1679, 1681 to 1683, 1686, 1687, 1699 to 1710, 1712, 1714 to 1721, 1724 to 1726, 1728 to 1731, 1735, 1736, 1739 to 1741, 1751, 1755, 1771, 1778, 1781 to 1783, 1827, 1834, 1836, 1843 to 1846, 1872, 1874, 1875 to 1888, 1890 to 1895, 1897, 1898, 1900, 1904 to 1927, 1931 to 1933, 1937, 1939, 1940, 1943, 1950, 1951, 1953, 1955 to 1959, 1962, 1964 to 1967, 1969 to 1971, 1973, 1977 to 1981, 1984 to 1991, 1993 to 1996, 2000 to 2005, 2007 to 2012, 2014 to 2025, 2027, 2028, 2030, 2032 to 2037, 2039-2045, 2047, 2051, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 46, 53, 54, 68, 76, 85, 96, 97, 114, 127, 129 to 132, 134, 135, 137 to 139, 142, 162 to 207, 225, 226, 230 to 243, 252, 264, 266 to 270, 284, 464, 467, 962, 963, 965, 966, 973, 990, 991, 997, 1000, 1001, 1011, 1261, 1299, 1355, 1356, 1470, 1472, 1473, 1503, 1504, 1522, 1526, 1535, 1536, 1542, 1543, 1545, 1595, 1599, 1604, 1620, 1623, 1624, 1626, 1640, 1662, 1666, 1667, 1669, 1670, 1673, 1682, 1683, 1687, 1699 to 1706, 1708, 1712, 1714 to 1716, 1719 to 1721, 1724 to 1726, 1729, 1730, 1736, 1778, 1783, 1836, 1843, 1875 to 1888, 1893 to 1895, 1897, 1900, 1904 to 1908, 1911, 1914 to 1918, 1920, 1922, 1923, 1925, 1926, 1931 to 1933, 1937, 1939, 1955, 1958, 1959, 1962, 1966, 1967, 1970, 1971, 1973, 1977, 1978 to 1981, 1985, 1986, 1987, 1988, 1990, 1991, 1994, 1996, 2000, 2002 to 2005, 2010, 2011, 2014 to 2025, 2027, 2028, 2035 to 2037, 2039, 2041 to 2045, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 96, 127, 129 to 132, 139, 162 to 169, 171 to 189, 191 to 193, 195, 196, 198 to 206, 234, 236, 238 to 240, 267 to 270, 966, 1000, 1522, 1542, 1623, 1624, 1667, 1682, 1683, 1700, 1703, 1704, 1708, 1714, 1719, 1720, 1724 to 1726, 1729, 1875, 1876, 1878, 1884 to 1886, 1893, 1894, 1906, 1908, 1914, 1917, 1918, 1922, 1923, 1925, 1926, 1932, 1933, 1967, 1970, 1978 to 1981, 1985, 1986, 1988, 1990, 1991, 2003, 2010, 2015, 2016, 2018, 2020, 2021, 2024, 2025, 2027, 2028, 2035, 2037, 2039, 2044, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 129, 130, 132, 163 to 168, 171, 172, 175 to 186, 188, 189, 192, 193, 195, 198 to 206, 238, 239, 966, 1703, 1720, 1726, 1923, 1925, 2003, 2015, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 46, 53-54, 61, 68, 76, 83, 85, 93, 96-97, 109, 127, 129-130, 132, 134-15, 137-39, 142, 163-172, 180-184, 186, 189, 234, 236, 238-239, 267, 313, 411, 452, 463-470, 475-478, 480, 500-503, 512, 517-518, 524-526, 654, 689, 702, 725-726, 728, 738, 779, 786-787, 800, 808, 810-811, 825, 865, 868, 889, 894, 903, 905, 909, 954, 966, 1011, 1015, 1021, 1024, 1080, 1085, 1258-1259, 1261-1262, 1293-1294, 1299, 1325, 1470, 1472-1473, 1522, 1542, 1604, 1623-1624, 1667, 1670, 1682-1683, 1687, 1700, 1703-1704, 1708, 1714, 1716, 1719-1720, 1724-1726, 1729-1730, 1827, 1936, 1843-1844, 1846, 1886, 1893-1894, 1914, 1923, 1925, 1932, 1979, 1986, 1988, 1990, 2003, 2015, 2018, 2020, 2027-2028, 2035, 2037, 2039, 2044, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 16 to 20 linked nucleosides and having a nucleobase sequence comprising at least 8 contiguous nucleobases of SEQ ID NOs: 238, 1714, 1719, 1893-1894, 1914, 1923, 1925, 2003, wherein the modified oligonucleotide comprises: (a) a gap segment consisting of linked deoxynucleosides; (b) a 5′ wing segment consisting of linked nucleosides; and (c) a 3′ wing segment consisting of linked nucleosides; wherein the gap segment is positioned immediately adjacent to and between the 5′ wing segment and the 3′ wing segment, wherein each nucleoside of each wing segment comprises a modified sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

In certain embodiments, the compound comprising a modified oligonucleotide consisting of 20 linked nucleosides and having a nucleobase sequence comprising at least 8 contiguous nucleobases of SEQ ID NO: 1914, wherein the modified oligonucleotide comprises: (a) a gap segment consisting of ten linked deoxynucleosides; (b) a 5′ wing segment consisting of five linked nucleosides; and (c) a 3′ wing segment consisting of five linked nucleosides; wherein the gap segment is positioned immediately adjacent to and between the 5′ wing segment and the 3′ wing segment, wherein each nucleoside of each wing segment comprises a 2′-O-methoxyethyl sugar, wherein at least one internucleoside linkage is a phosphorothioate linkage and wherein each cytosine residue is a 5-methylcytosine. In certain embodiments, each internucleoside linkage is a phosphorothioate linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

Certain embodiments disclosed herein provide a compound comprising a modified oligonucleotide according to the following formula: mCes Aes mCes Aes Aes Ads mCds Ads Ads Gds mCds Tds Gds Gds Tds mCes Ges Ges Tes Te (SEQ ID NO: 1914); wherein, A is an adenine, mC is a 5′-methylcytosine, G is a guanine, T is a thymine, e is a 2′-O-methoxyethyl modified nucleoside, d is a 2′-deoxynucleoside, and s is a phosphorothioate internucleoside linkage. In certain embodiments, the modified oligonucleotide further comprises a GalNAc conjugate. In certain embodiments, the conjugate is a 5′-Trishexylamino-(THA)-C6 GalNAc₃ conjugate. In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

Certain embodiments disclosed herein provide a compound comprising a modified oligonucleotide with the following formula:

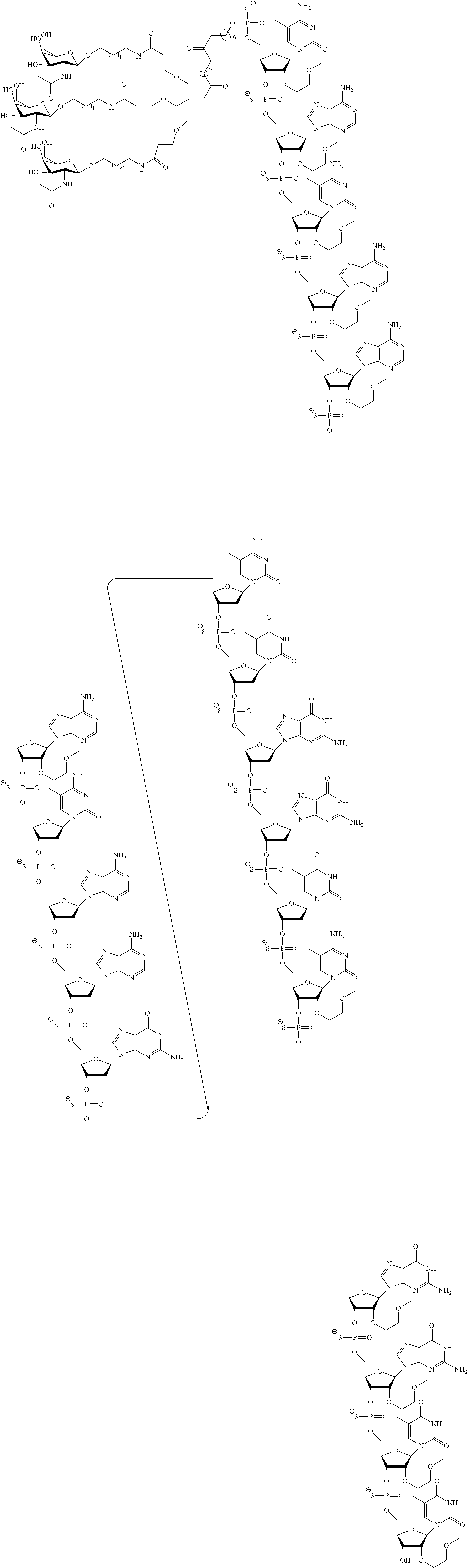

In certain embodiments, the compounds or compositions disclosed herein comprise a salt of the modified oligonucleotide.

In certain embodiments, the compounds or compositions disclosed herein further comprise a pharmaceutically acceptable carrier or diluent.

In certain embodiments, the animal is a human.

Certain embodiments provide a composition or compound comprising a modified oligonucleotide as described herein, wherein the viscosity level is less than 40 cP. In certain embodiments, the composition has a viscosity level less than 15 cP. In certain embodiments, the composition has a viscosity level less than 12 cP. In certain embodiments, the composition has a viscosity level less than 10 cP.

Certain embodiments disclosed herein provide compounds and compositions comprising a modified oligonucleotide targeting AGT for use in reducing AGT in a cell, tissue, organ or animal. In certain embodiments, reducing AGT treats, prevents, slows the progression, delays the onset of, and/or reduces a RAAS pathway related disease, disorder and/or condition, or symptom thereof. In certain embodiments, reducing AGT decreases hypertension. In certain embodiments, reducing AGT decreases or prevents fibrosis. In certain embodiments, reducing AGT modulates a symptom or marker of a RAAS pathway related disease, disorder and/or condition. In certain embodiments, the marker can be selected from one or more of shortened life expectancy, hypertension, chronic kidney disease, stroke, myocardial infarction, heart failure, valvular heart disease, aneurysms of the blood vessels, peripheral artery disease, organ damage and other cardiovascular diseases, disorders and/or conditions or symptoms thereof.

In certain embodiments, provided are compounds and compositions comprising a modified oligonucleotide targeting AGT for use in therapy. In certain embodiments, the compounds and compositions comprising a modified oligonucleotide targeting AGT are administered to an animal in a therapeutically effective amount.

In certain embodiments, provided are compounds and compositions comprising a modified oligonucleotide targeting AGT for use in the preparation of a medicament. In certain embodiments, the medicament is used for treating, preventing, slowing the progression, delaying the onset of, and/or reducing a RAAS pathway related disease, disorder and/or condition, or symptom thereof.

In certain embodiments, provided is a kit for treating, preventing, or ameliorating a RAAS pathway related disease and/or condition, disease, disorder or condition, wherein the kit comprises: (i) an AGT specific inhibitor as described herein; and optionally (ii) an additional agent or therapy as described herein. A kit of the present invention may further include instructions for using the kit to treat, prevent, or ameliorate a RAAS pathway related disease, disorder or condition as described herein.

In certain embodiments, the RAAS pathway related disease, disorder or condition is shortened life expectancy, hypertension, kidney disease (e.g., chronic kidney disease), stroke, cardiac disease (e.g., myocardial infarction, heart failure, valvular heart disease), aneurysms of the blood vessels, peripheral artery disease, organ damage and other RAAS related diseases, disorders and/or conditions or symptoms thereof. In certain embodiments, the hypertension is nonresistant hypertension or resistant hypertension. In certain embodiments, the aneurysm of the blood vessels is aortic aneurysm. In certain embodiments, the organ damage is heart muscle hypertrophy or fibrosis in an organ or tissue. In certain embodiments, the organ is heart, liver or kidney and the tissue is derived from the heart, liver or kidney.

The compound can be used in combination therapy with one or more additional agent or therapy as described herein. Agents or therapies can be administered concomitantly or sequentially to an animal. In certain embodiments, the composition or compound comprising a modified oligonucleotide targeting AGT is co-administered with one or more second agent(s). In certain embodiments the second agent includes procedures to reduce hypertension, diet changes, lifestyle changes, anti-fibrotic drugs and anti-hypertensive drugs such as RAS or RAAS inhibitors, diuretics, calcium channel blockers, adrenergic receptor antagonists, adrenergic agonists and vasodilators. In certain embodiments, the second agent is a second antisense compound. In further embodiments, the second antisense compound targets AGT. In other embodiments, the second antisense compound targets a non-AGT compound.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to AGT nucleic acid is 10 to 30 nucleotides in length. In other words, antisense compounds are from 10 to 30 linked nucleobases. In other embodiments, the antisense compound comprises a modified oligonucleotide consisting of 8 to 80, 10 to 80, 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In some embodiments, the antisense compound is an antisense oligonucleotide.

In certain embodiments, the antisense compound comprises a shortened or truncated modified oligonucleotide. The shortened or truncated modified oligonucleotide can have a single nucleoside deleted from the 5′ end (5′ truncation), the central portion or alternatively from the 3′ end (3′ truncation). A shortened or truncated oligonucleotide can have two or more nucleosides deleted from the 5′ end, two or more nucleosides deleted from the central portion or alternatively can have two or more nucleosides deleted from the 3′ end. Alternatively, the deleted nucleosides can be dispersed throughout the modified oligonucleotide, for example, in an antisense compound having one or more nucleoside deleted from the 5′ end, one or more nucleoside deleted from the central portion and/or one or more nucleoside deleted from the 3′ end.

When a single additional nucleoside is present in a lengthened oligonucleotide, the additional nucleoside can be located at the 5′ end, 3′ end or central portion of the oligonucleotide. When two or more additional nucleosides are present, the added nucleosides can be adjacent to each other, for example, in an oligonucleotide having two nucleosides added to the 5′ end (5′ addition), to the 3′ end (3′ addition) or the central portion, of the oligonucleotide. Alternatively, the added nucleoside can be dispersed throughout the antisense compound, for example, in an oligonucleotide having one or more nucleoside added to the 5′ end, one or more nucleoside added to the 3′ end, and/or one or more nucleoside added to the central portion.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Certain Antisense Compound Motifs and Mechanisms

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the RISC pathway and include, without limitation, siRNA, ssRNA and microRNA mechanisms; and occupancy based mechanisms. Certain antisense compounds may act through more than one such mechanism and/or through additional mechanisms.

### RNase H-Mediated Antisense

In certain embodiments, antisense activity results at least in part from degradation of target RNA by RNase H. RNase His a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE and 2′-O—CH₃, among others), and bicyclic sugar modified nucleosides (such bicyclic sugar modified nucleosides may include those having a constrained ethyl). In certain embodiments, nucleosides in the wings may include several modified sugar moieties, including, for example 2′-MOE and bicyclic sugar moieties such as constrained ethyl (cEt) or LNA. In certain embodiments, wings may include several modified and unmodified sugar moieties. In certain embodiments, wings may include various combinations of 2′-MOE nucleosides, bicyclic sugar moieties such as constrained ethyl nucleosides or LNA nucleosides, and 2′-deoxynucleosides.

Each distinct region may comprise uniform sugar moieties, variant, or alternating sugar moieties. The wing-gap-wing motif is frequently described as “X—Y—Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X—Y—Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, the antisense compound targeted to an AGT nucleic acid has a gapmer motif in which the gap consists of 6, 7, 8, 9, 10, 11, 12, 13, 14, 15, or 16 linked nucleosides.

In certain embodiments, the antisense oligonucleotide has a sugar motif described by Formula A as follows: (J)ₘ-(B)ₙ-(J)ₚ-(B)ᵣ-(A)ₜ-(D)_{g}-(A)ᵥ-(B)_{w}-(J)ₓ-(B)_{y}-(J)_{z}
wherein:
each A is independently a 2′-substituted nucleoside;
each B is independently a bicyclic nucleoside;
each J is independently either a 2′-substituted nucleoside or a 2′-deoxynucleoside;
each D is a 2′-deoxynucleoside;
m is 0-4; n is 0-2; p is 0-2; r is 0-2; t is 0-2; v is 0-2; w is 0-4; x is 0-2; y is 0-2; z is 0-4; g is 6-14;
   provided that:
at least one of m, n, and r is other than 0;
at least one of w and y is other than 0;
the sum of m, n, p, r, and t is from 2 to 5; and
the sum of v, w, x, y, and z is from 2 to 5.

### RNAi Compounds

In certain embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). In certain embodiments, antisense compounds comprise modifications that make them particularly suited for such mechanisms.

i. SSRNA Compounds

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO 2011/139702.

In certain embodiments, the 5′-nucleoside of an ssRNA compound has Formula IIc:

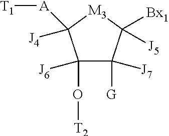

wherein:
T₁ is an optionally protected phosphorus moiety;
T₂ is an internucleoside linking group linking the compound of Formula IIc to the oligomeric compound;
A has one of the formulas:

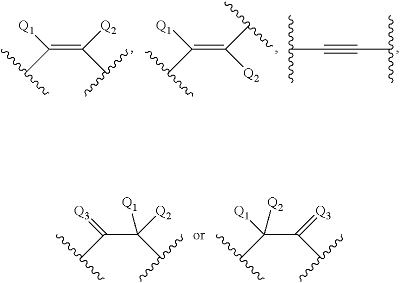

Q₁ and Q₂ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl or N(R₃)(R₄);
Q₃ is O, S, N(R₅) or C(R₆)(R₇);
each R₃, R₄ R₅, R₆ and R₇ is, independently, H, C₁-C₆ alkyl, substituted C₁-C₆ alkyl or C₁-C₆ alkoxy;
M₃ is O, S, NR₁₄, C(R₁₅)(R₁₆), C(R₁₅)(R₁₆) C(R₁₇)(R₁₈), C(R₁₅)═C(R₁₇), OC(R₁₅)(R₁₆) or OC(R₁₅)(Bx₂);
R₁₄ is H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
R₁₅, R₁₆, R₁₇ and R₁₈ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
Bx₁ is a heterocyclic base moiety;
or if Bx₂ is present then Bx₂ is a heterocyclic base moiety and Bx₁ is H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
J₄, J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
or J₄ forms a bridge with one of J₅ or J₇ wherein said bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
each R₁₉, R₂₀ and R₂₁ is, independently, H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
G is H, OH, halogen or O—[C(R₈)(R₉)]ₙ—[(C═O)ₘ—X₁]ⱼ—Z;
each R₈ and R₉ is, independently, H, halogen, C₁-C₆ alkyl or substituted C₁-C₆ alkyl;
X₁ is O, S or N(E₁);
Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to about 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(═X₂)N(J₁)(J₂);
X₂ is O, S or NJ₃;
each J₁, J₂ and J₃ is, independently, H or C₁-C₆ alkyl;
when j is 1 then Z is other than halogen or N(E₂)(E₃); and
wherein said oligomeric compound comprises from 8 to 40 monomeric subunits and is hybridizable to at least a portion of a target nucleic acid.

In certain embodiments, M₃ is O, CH═CH, OCH₂ or OC(H)(Bx₂). In certain embodiments, M₃ is O.

In certain embodiments, J₄, J₅, J₆ and J₇ are each H. In certain embodiments, J₄ forms a bridge with one of J₅ or J₇.

In certain embodiments, A has one of the formulas:

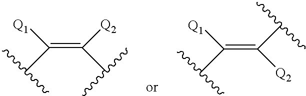

wherein:

Q₁ and Q₂ are each, independently, H, halogen, C₁-C₆ alkyl, substituted C₁-C₆ alkyl, C₁-C₆ alkoxy or substituted C₁-C₆ alkoxy. In certain embodiments, Q₁ and Q₂ are each H. In certain embodiments, Q₁ and Q₂ are each, independently, H or halogen. In certain embodiments, Q₁ and Q₂ is H and the other of Q₁ and Q₂ is F, CH₃ or OCH₃.

In certain embodiments, T₁ has the formula:

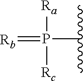

wherein:
Rₐ and R_{c} are each, independently, protected hydroxyl, protected thiol, C₁-C₆ alkyl, substituted C₁-C₆ alkyl, C₁-C₆ alkoxy, substituted C₁-C₆ alkoxy, protected amino or substituted amino; and
R_{b} is O or S. In certain embodiments, R_{b} is O and Rₐ and R_{c} are each, independently, OCH₃, OCH₂CH₃ or CH(CH₃)₂.

In certain embodiments, G is halogen, OCH₃, OCH₂F, OCHF₂, OCF₃, OCH₂CH₃, O(CH₂)₂F, OCH₂CHF₂, OCH₂CF₃, OCH₂—CH═H₂, O(CH₂)₂—OCH₃, O(CH₂)₂—SCH₃, O(CH₂)₂—OCF₃, O(CH₂)₃—N(R₁₀)(R₁₁), O(CH₂)₂—ON(R₁₀)(R₁₁), O(CH₂)₂—O(CH₂)₂—N(R₁₀)(R₁₁), OCH₂C(═O)—N(R₁₀)(R₁₁), OCH₂C(═O)—N(R₁₂)—(CH₂)₂—N(R₁₀)(R₁₁) or O(CH₂)₂—N(R₁₂)—C(═NR₁₃) [N(R₁₀)(R₁₁)] wherein R₁₀, R₁₁, R₁₂ and R₁₃ are each, independently, H or C₁-C₆ alkyl. In certain embodiments, G is halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H) CH₃, OCH₂C(═O)—N(H)—(CH₂)₂—N(CH₃)₂ or OCH₂—N(H)—C(═NH)NH₂. In certain embodiments, G is F, OCH₃ or O(CH₂)₂—OCH₃. In certain embodiments, G is O(CH₂)₂—OCH₃.

In certain embodiments, the 5′-terminal nucleoside has Formula Ile:

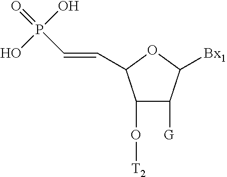

In certain embodiments, antisense compounds, including those particularly suitable for ssRNA comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having uniform sugar modifications. In certain such embodiments, each nucleoside of the region comprises the same RNA-like sugar modification. In certain embodiments, each nucleoside of the region is a 2′-F nucleoside. In certain embodiments, each nucleoside of the region is a 2′-OMe nucleoside. In certain embodiments, each nucleoside of the region is a 2′-MOE nucleoside. In certain embodiments, each nucleoside of the region is a cEt nucleoside. In certain embodiments, each nucleoside of the region is an LNA nucleoside. In certain embodiments, the uniform region constitutes all or essentially all of the oligonucleotide. In certain embodiments, the region constitutes the entire oligonucleotide except for 1-4 terminal nucleosides. In certain embodiments, oligonucleotides comprise one or more regions of alternating sugar modifications, wherein the nucleosides alternate between nucleotides having a sugar modification of a first type and nucleotides having a sugar modification of a second type. In certain embodiments, nucleosides of both types are RNA-like nucleosides. In certain embodiments the alternating nucleosides are selected from: 2′-OMe, 2′-F, 2′-MOE, LNA, and cEt. In certain embodiments, the alternating modifications are 2′-F and 2′-OMe. Such regions may be contiguous or may be interrupted by differently modified nucleosides or conjugated nucleosides.

In certain embodiments, the alternating region of alternating modifications each consist of a single nucleoside (i.e., the pattern is (AB)ₓA_{y} wherein A is a nucleoside having a sugar modification of a first type and B is a nucleoside having a sugar modification of a second type; x is 1-20 and y is 0 or 1). In certain embodiments, one or more alternating regions in an alternating motif includes more than a single nucleoside of a type. For example, oligonucleotides may include one or more regions of any of the following nucleoside motifs:

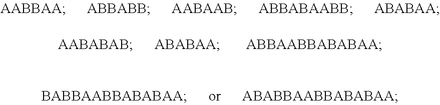

wherein A is a nucleoside of a first type and B is a nucleoside of a second type. In certain embodiments, A and B are each selected from 2′-F, 2′-OMe, BNA, and MOE.

In certain embodiments, oligonucleotides having such an alternating motif also comprise a modified 5′ terminal nucleoside, such as those of formula IIc or Ile.

In certain embodiments, oligonucleotides comprise a region having a 2-2-3 motif. Such regions comprises the following motif:

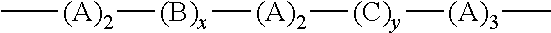

wherein: A is a first type of modified nucleoside;
B and C, are nucleosides that are differently modified than A, however, B and C may have the same or different modifications as one another;
x and y are from 1 to 15.

In certain embodiments, A is a 2′-OMe modified nucleoside. In certain embodiments, B and C are both 2′-F modified nucleosides. In certain embodiments, A is a 2′-OMe modified nucleoside and B and C are both 2′-F modified nucleosides.

In certain embodiments, oligonucleosides have the following sugar motif:

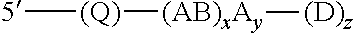

wherein:
Q is a nucleoside comprising a stabilized phosphate moiety. In certain embodiments, Q is a nucleoside having Formula IIc or Ile;
A is a first type of modified nucleoside;
B is a second type of modified nucleoside;
D is a modified nucleoside comprising a modification different from the nucleoside adjacent to it. Thus, if y is 0, then D must be differently modified than B and if y is 1, then D must be differently modified than A. In certain embodiments, D differs from both A and B.
X is 5-15;
Y is 0 or 1;
Z is 0-4.

In certain embodiments, oligonucleosides have the following sugar motif: 5′-(Q)-(A)ₓ-(D)_{z}

wherein:
Q is a nucleoside comprising a stabilized phosphate moiety. In certain embodiments, Q is a nucleoside having Formula IIc or Ile;
A is a first type of modified nucleoside;
D is a modified nucleoside comprising a modification different from A.
X is 11-30;
Z is 0-4.

In certain embodiments A, B, C, and D in the above motifs are selected from: 2′-OMe, 2′-F, 2′-MOE, LNA, and cEt. In certain embodiments, D represents terminal nucleosides. In certain embodiments, such terminal nucleosides are not designed to hybridize to the target nucleic acid (though one or more might hybridize by chance). In certain embodiments, the nucleobase of each D nucleoside is adenine, regardless of the identity of the nucleobase at the corresponding position of the target nucleic acid. In certain embodiments the nucleobase of each D nucleoside is thymine.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Oligonucleotides having any of the various sugar motifs described herein, may have any linkage motif. For example, the oligonucleotides, including but not limited to those described above, may have a linkage motif selected from non-limiting the table below:

| 5′ most linkage | Central region | 3′-region |
|---|---|---|
| PS | Alternating PO/PS | 6 PS |
| PS | Alternating PO/PS | 7 PS |
| PS | Alternating PO/PS | 8 PS |

ii. siRNA Compounds

In certain embodiments, antisense compounds are double-stranded RNAi compounds (siRNA). In such embodiments, one or both strands may comprise any modification motif described above for ssRNA. In certain embodiments, ssRNA compounds may be unmodified RNA. In certain embodiments, siRNA compounds may comprise unmodified RNA nucleosides, but modified internucleoside linkages.

Several embodiments relate to double-stranded compositions wherein each strand comprises a motif defined by the location of one or more modified or unmodified nucleosides. In certain embodiments, compositions are provided comprising a first and a second oligomeric compound that are fully or at least partially hybridized to form a duplex region and further comprising a region that is complementary to and hybridizes to a nucleic acid target. It is suitable that such a composition comprise a first oligomeric compound that is an antisense strand having full or partial complementarity to a nucleic acid target and a second oligomeric compound that is a sense strand having one or more regions of complementarity to and forming at least one duplex region with the first oligomeric compound.

The compositions of several embodiments modulate gene expression by hybridizing to a nucleic acid target resulting in loss of its normal function. In some embodiments, the target nucleic acid is AGT. In certain embodiment, the degradation of the targeted AGT is facilitated by an activated RISC complex that is formed with compositions of the invention.

Several embodiments are directed to double-stranded compositions wherein one of the strands is useful in, for example, influencing the preferential loading of the opposite strand into the RISC (or cleavage) complex. The compositions are useful for targeting selected nucleic acid molecules and modulating the expression of one or more genes. In some embodiments, the compositions of the present invention hybridize to a portion of a target RNA resulting in loss of normal function of the target RNA.

Certain embodiments are drawn to double-stranded compositions wherein both the strands comprises a hemimer motif, a fully modified motif, a positionally modified motif or an alternating motif. Each strand of the compositions of the present invention can be modified to fulfil a particular role in for example the siRNA pathway. Using a different motif in each strand or the same motif with different chemical modifications in each strand permits targeting the antisense strand for the RISC complex while inhibiting the incorporation of the sense strand. Within this model, each strand can be independently modified such that it is enhanced for its particular role. The antisense strand can be modified at the 5′-end to enhance its role in one region of the RISC while the 3′-end can be modified differentially to enhance its role in a different region of the RISC.

The double-stranded oligonucleotide molecules can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double-stranded structure, for example wherein the double-stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the double-stranded oligonucleotide molecule are complementary to the target nucleic acid or a portion thereof). Alternatively, the double-stranded oligonucleotide is assembled from a single oligonucleotide, where the self-complementary sense and antisense regions of the siRNA are linked by means of a nucleic acid based or non-nucleic acid-based linker(s).

The double-stranded oligonucleotide can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi.

In certain embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the double-stranded oligonucleotide comprises nucleotide sequence that is complementary to nucleotide sequence of a target gene. In another embodiment, the double-stranded oligonucleotide interacts with nucleotide sequence of a target gene in a manner that causes inhibition of expression of the target gene.

As used herein, double-stranded oligonucleotides need not be limited to those molecules containing only RNA, but further encompasses chemically modified nucleotides and non-nucleotides. In certain embodiments, the short interfering nucleic acid molecules lack 2′-hydroxy (2′-OH) containing nucleotides. In certain embodiments short interfering nucleic acids optionally do not include any ribonucleotides (e.g., nucleotides having a 2′-OH group). Such double-stranded oligonucleotides that do not require the presence of ribonucleotides within the molecule to support RNAi can however have an attached linker or linkers or other attached or associated groups, moieties, or chains containing one or more nucleotides with 2′-OH groups. Optionally, double-stranded oligonucleotides can comprise ribonucleotides at about 5, 10, 20, 30, 40, or 50% of the nucleotide positions. As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of several embodiments provided herein can target AGT by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments, RNA/DNA hybrids include a DNA strand or region that is an antisense strand or region (e.g, has at least 70, 80, 90, 95, 98, or 100% complementarity to a target nucleic acid) and an RNA strand or region that is a sense strand or region (e.g, has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999.) Exemplary circular nucleic acids include lariat structures in which the free 5′ phosphoryl group of a nucleotide becomes linked to the 2′ hydroxyl group of another nucleotide in a loop back fashion.

In other embodiments, the dsRNA includes one or more modified nucleotides in which the 2′ position in the sugar contains a halogen (such as fluorine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. The dsRNAs may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000, or U.S. Ser. No. 60/130,377, filed Apr. 21, 1999.

In other embodiments, the dsRNA can be any of the at least partially dsRNA molecules disclosed in WO 00/63364, as well as any of the dsRNA molecules described in U.S. Provisional Application 60/399,998; and U.S. Provisional Application 60/419,532, and PCT/US2003/033466, the teaching of which is hereby incorporated by reference. Any of the dsRNAs may be expressed in vitro or in vivo using the methods described herein or standard methods, such as those described in WO 00/63364.

### Occupancy

In certain embodiments, antisense compounds are not expected to result in cleavage or the target nucleic acid via RNase H or to result in cleavage or sequestration through the RISC pathway. In certain such embodiments, antisense activity may result from occupancy, wherein the presence of the hybridized antisense compound disrupts the activity of the target nucleic acid. In certain such embodiments, the antisense compound may be uniformly modified or may comprise a mix of modifications and/or modified and unmodified nucleosides.

### Target Nucleic Acids, Target Regions and Nucleotide Sequences

Nucleotide sequences that encode AGT include, without limitation, the following: GENBANK Accession No. NM_000029.3 (incorporated herein as SEQ ID NO: 1), the complement of the nucleotides 24354000 to 24370100 of GENBANK Accession No. NT_167186.1 (incorporated herein as SEQ ID NO: 2), GENBANK Accession No. AK307978.1 (incorporated herein as SEQ ID NO: 3), GENBANK Accession No. AK303755.1 (incorporated herein as SEQ ID NO: 4), GENBANK Accession No. AK293507.1 (incorporated herein as SEQ ID NO: 5), and GENBANK Accession No. CR606672.1 (incorporated herein as SEQ ID NO: 6). In certain embodiments, an antisense compound described herein targets a nucleic acid sequence encoding AGT. In certain embodiments, an antisense compound described herein targets the sequence of any of SEQ ID NOs: 1-6.

It is understood that the sequence set forth in each SEQ ID NO in the examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for AGT can be obtained by accession number from sequence databases such as NCBI and such information is incorporated herein by reference. In certain embodiments, a target region may encompass the sequence from a 5′ target site of one target segment within the target region to a 3′ target site of another target segment within the target region. In certain embodiments, a target region may encompass at least 8 consecutive nucleobases selected from within an antisense compound at least 8 consecutive nucleobases from the 5′-terminus of the antisense compound (the remaining nucleobases being a consecutive stretch the beginning immediately upstream of the 5′-terminus of the antisense compound which is specifically hybridizable to the target nucleic acid and continuing until the region contains about 8 to about 80 nucleobases). In certain embodiments, a target region may encompass at least 8 consecutive nucleobases selected from within an antisense compound at least 8 consecutive nucleobases from the 3′-terminus of the antisense compound (the remaining nucleobases being a consecutive stretch beginning immediately downstream of the 3′-terminus of the antisense compound which is specifically hybridizable to the target nucleic acid and continuing until the region contains about 8 to about 80 nucleobases). In certain embodiments, the target region comprises at least 8 consecutive nucleobases selected from any of SEQ ID NOs: 14-2051 and continues up to 80 nucleobases 5′ or 3′ of the 8 consecutive nucleobase sequence.

In certain embodiments, a “target segment” is a smaller, sub-portion of a target region within a nucleic acid. For example, a target segment can be the sequence of nucleotides of a target nucleic acid to which one or more antisense compound is targeted. “5′ target site” refers to the 5′-most nucleotide of a target segment. “3′ target site” refers to the 3′-most nucleotide of a target segment.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in AGT mRNA levels are indicative of inhibition of AGT expression. Reductions in levels of an AGT protein are also indicative of inhibition of AGT expression. Further, phenotypic changes are indicative of inhibition of AGT expression. For example, a decrease in fibrosis in tissues can be indicative of inhibition of AGT expression. In another example, an decrease in hypertension can be indicative of inhibition of AGT expression.

### Hybridization

In some embodiments, hybridization occurs between an antisense compound disclosed herein and an AGT nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrook and Russell, Molecular Cloning: A Laboratory Manual, 3^{rd} Ed., 2001). In certain embodiments, the antisense compounds provided herein are specifically hybridizable with an AGT nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as an AGT nucleic acid).

Non-complementary nucleobases between an antisense compound and an AGT nucleic acid may be tolerated provided that the antisense compound remains able to specifically hybridize to the AGT nucleic acid. Moreover, an antisense compound may hybridize over one or more segments of an AGT nucleic acid such that intervening or adjacent segments are not involved in the hybridization event (e.g., a loop structure, mismatch or hairpin structure).

In certain embodiments, the antisense compounds provided herein, or a specified portion thereof, are, or are at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to an AGT nucleic acid, a target region, target segment, or specified portion thereof. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention.

Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound may be fully complementary to an AGT nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as an AGT nucleic acid, or specified portion thereof.

In certain embodiments, antisense compounds that are, or are up to, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as an AGT nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, at least a 10, at least an 11, at least a 12, at least a 13, at least a 14, at least a 15, at least a 16, at least a 17, at least an 18, at least a 19, at least a 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, at least 75%, at least 80%, at least 85%, at least 90%, at least 95%, at least 96%, at least 97%, at least 98%, at least 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to an AGT nucleic acid comprise one or more modified internucleoside linkages. In certain embodiments, at least one of the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂)(R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Zhou et al., J. Org. Chem., 2009, 74, 118-134); and 4′-CH₂—C(═CH₂)-2′ (and analogs thereof see PCT/US2008/066154 published as WO 2008/154401, published on Dec. 8, 2008).

Further bicyclic nucleosides have been reported in published literature (see for example: Srivastava et al., i J. Am. Chem. Soc., 2007, 129 (26) 8362-8379; Frieden et al., Nucleic Acids Research, 2003, 21, 6365-6372; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wahlestedt et al., Proc. Natl. Acad. Sci. U.S.A., 2000, 97, 5633-5638; Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; U.S. Pat. Nos. 7,399,845; 7,053,207; 7,034,133; 6,794,499; 6,770,748; 6,670,461; 6,525,191; 6,268,490; U.S. Patent Publication Nos.: US2008-0039618; US2007-0287831; US2004-0171570; U.S. patent application Ser. Nos. 12/129,154; 61/099,844; 61/097,787; 61/086,231; 61/056,564; 61/026,998; 61/026,995; 60/989,574; International applications WO 2007/134181; WO 2005/021570; WO 2004/106356; WO 99/14226; and PCT International Applications Nos.: PCT/US2008/068922; PCT/US-2008/066154; and PCT/US2008/064591). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

As used herein, “monocyclic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

In certain embodiments, bicyclic sugar moieties of BNA nucleosides include, but are not limited to, compounds having at least one bridge between the 4′ and the 2′ carbon atoms of the pentofuranosyl sugar moiety including without limitation, bridges comprising 1 or from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-(CH₂)—O-2′ bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include those having a 4′ to 2′ bridge wherein such bridges include without limitation, α-L-4′-(CH₂)—O-2′, β-D-4′-CH₂—O-2′, 4′-(CH₂)₂—O-2′, 4′-CH₂—O—N(R)-2′, 4′-CH₂—N(R)—O-2′, 4′-CH(CH₃)—O-2′, 4′-CH₂—S-2′, 4′-CH₂—N(R)-2′, 4′-CH₂—CH(CH₃)-2′, and 4′-(CH₂)₃-2′, wherein R is H, a protecting group or C₁-C₁₂ alkyl.

In certain embodiment, bicyclic nucleosides have the formula:

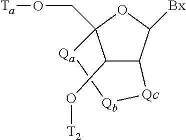

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides have the formula:

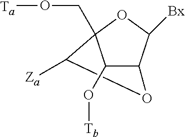

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thiol.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides have the formula:

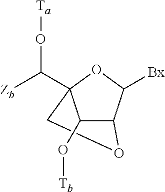

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides have the formula:

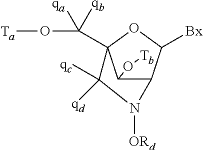

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides have the formula:

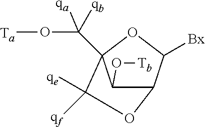

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of adenine, cytosine, guanine, 5-methyl-cytosine, thymine and uracil bicyclic nucleosides having a 4′-CH₂—O-2′ bridge, along with their oligomerization, and nucleic acid recognition properties have been described (Koshkin et al., Tetrahedron, 1998, 54, 3607-3630). The synthesis of bicyclic nucleosides has also been described in WO 98/39352 and WO 99/14226.

Analogs of various bicyclic nucleosides that have 4′ to 2′ bridging groups such as 4′-CH₂—O-2′ and 4′-CH₂—S-2′, have also been prepared (Kumar et al., Bioorg. Med. Chem. Lett., 1998, 8, 2219-2222). Preparation of oligodeoxyribonucleotide duplexes comprising bicyclic nucleosides for use as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides have the formula:

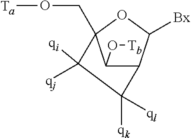

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O) NJⱼJₖ, C(═O)Jⱼ, O—C(═O)NJⱼJₖ, N(H)C(═NH) NJⱼJₖ, N(H)C(═O)NJⱼJₖ or N(H)C(═S)NJⱼJₖ; and
qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Frier et al., Nucleic Acids Research, 1997, 25 (22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129 (26), 8362-8379).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, (F) methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) vinyl BNA as depicted below.

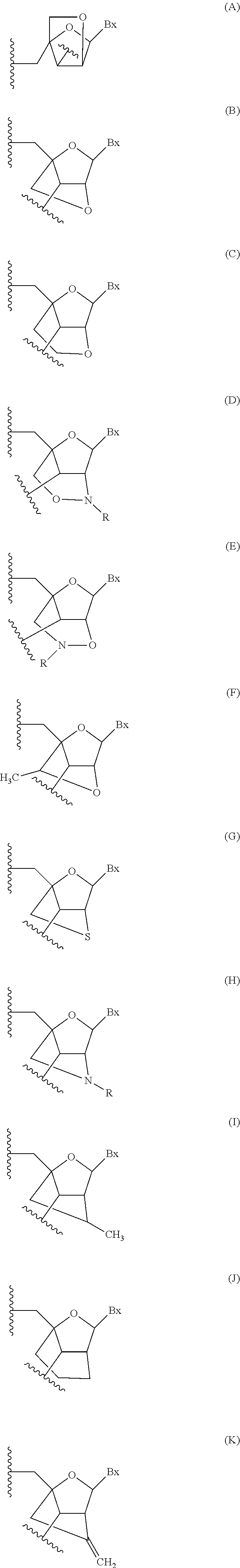

wherein Bx is the base moiety and R is, independently, H, a protecting group, C₁-C₆ alkyl or C₁-C₆ alkoxy.

As used herein, the term “modified tetrahydropyran nucleoside” or “modified THP nucleoside” means a nucleoside having a six-membered tetrahydropyran “sugar” substituted for the pentofuranosyl residue in normal nucleosides and can be referred to as a sugar surrogate. Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F-HNA) having a tetrahydropyranyl ring system as illustrated below.

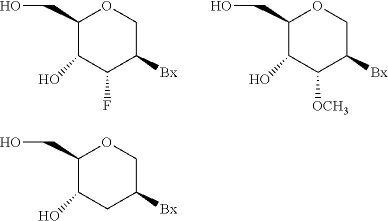

In certain embodiment, sugar surrogates are selected having the formula:

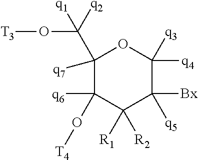

wherein:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to an oligomeric compound or oligonucleotide and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and
one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides are provided wherein one of R₁ and R₂ is F. In certain embodiments, R₁ is fluoro and R₂ is H; R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

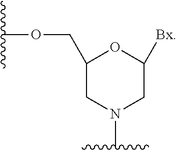

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129 (26), 8362-8379).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130 (6), 1979-1984; Horváth et al., Tetrahedron Letters, 2007, 48, 3621-3623; Nauwelaerts et al., J. Am. Chem. Soc., 2007, 129 (30), 9340-9348; Gu et al., Nucleosides, Nucleotides & Nucleic Acids, 2005, 24 (5-7), 993-998; Nauwelaerts et al., Nucleic Acids Research, 2005, 33 (8), 2452-2463; Robeyns et al., Acta Crystallographica, Section F: Structural Biology and Crystallization Communications, 2005, F61 (6), 585-586; Gu et al., Tetrahedron, 2004, 60 (9), 2111-2123; Gu et al., Oligonucleotides, 2003, 13 (6), 479-489; Wang et al., J. Org. Chem., 2003, 68, 4499-4505; Verbeure et al., Nucleic Acids Research, 2001, 29 (24), 4941-4947; Wang et al., J. Org. Chem., 2001, 66, 8478-82; Wang et al., Nucleosides, Nucleotides & Nucleic Acids, 2001, 20 (4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have Formula X.

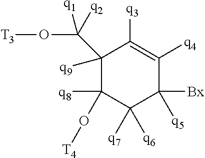

wherein independently for each of said at least one cyclohexenyl nucleoside analog of Formula X:
Bx is a heterocyclic base moiety;
T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and
q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

Many other monocyclic, bicyclic and tricyclic ring systems are known in the art and are suitable as sugar surrogates that can be used to modify nucleosides for incorporation into oligomeric compounds as provided herein (see for example review article: Leumann, Christian J. Bioorg. & Med. Chem., 2002, 10, 841-854). Such ring systems can undergo various additional substitutions to further enhance their activity. As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, “2′-modified” or “2′-substituted” refers to a nucleoside comprising a sugar comprising a substituent at the 2′ position other than H or OH. 2′-modified nucleosides, include, but are not limited to, nucleosides with non-bridging 2′-substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl, 2′-modified nucleosides may further comprise other modifications, for example at other positions of the sugar and/or at the nucleobase.

As used herein, “2′-F” refers to a nucleoside comprising a sugar comprising a fluoro group at the 2′ position of the sugar ring.

As used herein, “2′-OMe” or “2′-OCH₃”, “2′-O-methyl” or “2′-methoxy” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring. Methods for the preparations of modified sugars are well known to those skilled in the art. Some representative U.S. patents that teach the preparation of such modified sugars include without limitation, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to an AGT nucleic acid comprise one or more modified nucleobases. In certain embodiments, gap-widened antisense oligonucleotides targeted to an AGT nucleic acid comprise one or more modified nucleobases. In certain embodiments, at least one of the modified nucleobases is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to an AGT nucleic acid can be utilized in pharmaceutical compositions by combining the antisense compound with a suitable pharmaceutically acceptable diluent or carrier. A pharmaceutically acceptable diluent includes water e.g., water-for-injection (WFI). A pharmaceutically acceptable diluent includes saline e.g., phosphate-buffered saline (PBS). Water or saline is a diluent suitable for use in compositions to be delivered parenterally. Accordingly, in one embodiment, employed in the methods described herein is a pharmaceutical composition comprising an antisense compound targeted to an AGT nucleic acid and a pharmaceutically acceptable diluent. In certain embodiments, the pharmaceutically acceptable diluent is water or saline. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure herein is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

Pharmaceutically acceptable salts of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Sodium salts of antisense oligonucleotides are useful and are well accepted for therapeutic administration to humans. Accordingly, in one embodiment the compounds described herein are in the form of a sodium salt.

A prodrug can include the incorporation of additional nucleosides at one or both ends of an antisense compound which are cleaved by endogenous nucleases within the body, to form the active antisense compound.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of AGT or the prevention, reduction, amelioration or slowing the progression of a disease, disorder or condition associated with AGT.

In certain embodiments, the variables of the dosing regimen are adjusted to result in a desired concentration of pharmaceutical composition in a subject. “Concentration of pharmaceutical composition” as used with regard to dose regimen can refer to the compound, oligonucleotide, or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from 0.01 μg to 100 mg per kg of body weight, or within a range of 0.001 mg to 1000 mg dosing, and may be given once or more daily, weekly, biweekly, monthly, quarterly, semi-annually or yearly, or even once every 2 to 20 years. Following successful treatment, it may be desirable to have the patient undergo maintenance therapy to prevent the recurrence of the disease state, wherein the oligonucleotide is administered in maintenance doses, ranging from 0.01 μg to 100 mg per kg of body weight, once or more daily, to once every 20 years or ranging from 0.001 mg to 1000 mg dosing.

### Administration

The compounds or pharmaceutical compositions of the present invention can be administered in a number of ways depending upon whether local or systemic treatment is desired and upon the area to be treated. Administration can be inhaled (i.e., pulmonary), enteral (i.e., enteric), parenteral or topical.

In certain embodiments, the compounds and compositions as described herein are administered parenterally. Parenteral administration includes, but is not limited to, intravenous, intra-arterial, subcutaneous, intraperitoneal, intraocular, intramuscular, intracranial, intrathecal, intramedullary, intraventricular or intratumoral injection or infusion. Parenteral administration also includes intranasal administration.

In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

In certain embodiments, formulations for parenteral administration can include sterile aqueous solutions which can also contain buffers, diluents and other suitable additives such as, but not limited to, penetration enhancers, carrier compounds and other pharmaceutically acceptable carriers or excipients.

In certain embodiments, the compounds and compositions as described herein are administered enterally. Enteric administration includes, but is not limited to, oral, transmucosal, intestinal or rectal (e.g., suppository, enema). In certain embodiments, formulations for enteral administration of the compounds or compositions can include, but is not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, enteral formulations are those in which compounds provided herein are administered in conjunction with one or more penetration enhancers, surfactants and chelators.

In certain embodiments, administration includes pulmonary administration. In certain embodiments, pulmonary administration comprises delivery of aerosolized oligonucleotide to the lung of a subject by inhalation. Following inhalation by a subject of aerosolized oligonucleotide, oligonucleotide distributes to cells of both normal and inflamed lung tissue, including alveolar macrophages, eosinophils, epithelium, blood vessel endothelium, and bronchiolar epithelium. A suitable device for the delivery of a pharmaceutical composition comprising a modified oligonucleotide includes, but is not limited to, a standard nebulizer device. Additional suitable devices include dry powder inhalers or metered dose inhalers.

In certain embodiments, pharmaceutical compositions are administered to achieve local rather than systemic exposures. For example, pulmonary administration delivers a pharmaceutical composition to the lung, with minimal systemic exposure.

### Conjugated Antisense Compounds

In certain embodiments, the oligonucleotides or oligomeric compounds as provided herein are modified by covalent attachment of one or more conjugate groups. In general, conjugate groups modify one or more properties of the attached oligonucleotide or oligomeric compound including but not limited to pharmacodynamics, pharmacokinetics, stability, binding, absorption, cellular distribution, cellular uptake, charge and clearance. As used herein, “conjugate group” means a radical group comprising a group of atoms that are attached to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties. Conjugate groups are routinely used in the chemical arts and can include a conjugate linker that covalently links the conjugate group to an oligonucleotide or oligomeric compound. In certain embodiments, conjugate groups include a cleavable moiety that covalently links the conjugate group to an oligonucleotide or oligomeric compound. In certain embodiments, conjugate groups include a conjugate linker and a cleavable moiety to covalently link the conjugate group to an oligonucleotide or oligomeric compound. In certain embodiments, a conjugate group has the general formula:

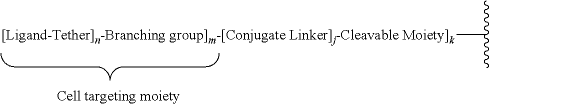

wherein n is from 1 to about 3, m is 0 when n is 1 or m is 1 when n is 2 or 3, j is 1 or 0, k is 1 or 0 and the sum of j and k is at least one.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

Conjugate groups are shown herein as radicals, providing a bond for forming covalent attachment to an oligomeric compound such as an oligonucleotide. In certain embodiments, the point of attachment on the oligomeric compound is at the 3′-terminal nucleoside or modified nucleoside. In certain embodiments, the point of attachment on the oligomeric compound is the 3′-oxygen atom of the 3′-hydroxyl group of the 3′ terminal nucleoside or modified nucleoside. In certain embodiments, the point of attachment on the oligomeric compound is at the 5′-terminal nucleoside or modified nucleoside. In certain embodiments the point of attachment on the oligomeric compound is the 5′-oxygen atom of the 5′-hydroxyl group of the 5′-terminal nucleoside or modified nucleoside. In certain embodiments, the point of attachment on the oligomeric compound is at any reactive site on a nucleoside, a modified nucleoside or an internucleoside linkage.

As used herein, “cleavable moiety” and “cleavable bond” mean a cleavable bond or group of atoms that is capable of being split or cleaved under certain physiological conditions. In certain embodiments, a cleavable moiety is a cleavable bond. In certain embodiments, a cleavable moiety comprises a cleavable bond. In certain embodiments, a cleavable moiety is a group of atoms. In certain embodiments, a cleavable moiety is selectively cleaved inside a cell or sub-cellular compartment, such as a lysosome. In certain embodiments, a cleavable moiety is selectively cleaved by endogenous enzymes, such as nucleases. In certain embodiments, a cleavable moiety comprises a group of atoms having one, two, three, four, or more than four cleavable bonds.

In certain embodiments, conjugate groups comprise a cleavable moiety. In certain such embodiments, the cleavable moiety covalently attaches the oligomeric compound to the conjugate linker. In certain such embodiments, the cleavable moiety covalently attaches the oligomeric compound to the cell-targeting moiety.

In certain embodiments, a cleavable bond is selected from among: an amide, a polyamide, an ester, an ether, one or both esters of a phosphodiester, a phosphate ester, a carbamate, a di-sulfide, or a peptide. In certain embodiments, a cleavable bond is one of the esters of a phosphodiester. In certain embodiments, a cleavable bond is one or both esters of a phosphodiester. In certain embodiments, the cleavable moiety is a phosphodiester linkage between an oligomeric compound and the remainder of the conjugate group. In certain embodiments, the cleavable moiety comprises a phosphodiester linkage that is located between an oligomeric compound and the remainder of the conjugate group. In certain embodiments, the cleavable moiety comprises a phosphate or phosphodiester. In certain embodiments, the cleavable moiety is attached to the conjugate linker by either a phosphodiester or a phosphorothioate linkage. In certain embodiments, the cleavable moiety is attached to the conjugate linker by a phosphodiester linkage. In certain embodiments, the conjugate group does not include a cleavable moiety.

In certain embodiments, the cleavable moiety is a cleavable nucleoside or a modified nucleoside. In certain embodiments, the nucleoside or modified nucleoside comprises an optionally protected heterocyclic base selected from a purine, substituted purine, pyrimidine or substituted pyrimidine. In certain embodiments, the cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine.

In certain embodiments, the cleavable moiety is 2′-deoxy nucleoside that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to the 3′-oxygen atom of the 3′-hydroxyl group of the 3′-terminal nucleoside or modified nucleoside by a phosphodiester linkage. In certain embodiments, the cleavable moiety is 2′-deoxy adenosine that is attached to the 5′-oxygen atom of the 5′-hydroxyl group of the 5′-terminal nucleoside or modified nucleoside by a phosphodiester linkage. In certain embodiments, the cleavable moiety is attached to a 2′-position of a nucleoside or modified nucleoside of an oligomeric compound.

As used herein, “conjugate linker” in the context of a conjugate group means a portion of a conjugate group comprising any atom or group of atoms that covalently link the cell-targeting moiety to the oligomeric compound either directly or through the cleavable moiety. In certain embodiments, the conjugate linker comprises groups selected from alkyl, amino, oxo, amide, disulfide, polyethylene glycol, ether, thioether (—S—) and hydroxylamino (—O—N(H)—). In certain embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus linking group. In certain embodiments, the conjugate linker comprises at least one phosphodiester group. In certain embodiments, the conjugate linker includes at least one neutral linking group.

In certain embodiments, the conjugate linker is covalently attached to the oligomeric compound. In certain embodiments, the conjugate linker is covalently attached to the oligomeric compound and the branching group. In certain embodiments, the conjugate linker is covalently attached to the oligomeric compound and a tethered ligand. In certain embodiments, the conjugate linker is covalently attached to the cleavable moiety. In certain embodiments, the conjugate linker is covalently attached to the cleavable moiety and the branching group. In certain embodiments, the conjugate linker is covalently attached to the cleavable moiety and a tethered ligand. In certain embodiments, the conjugate linker includes one or more cleavable bonds. In certain embodiments, the conjugate group does not include a conjugate linker.

As used herein, “branching group” means a group of atoms having at least 3 positions that are capable of forming covalent linkages to two or more tether-ligands and the remainder of the conjugate group. In general a branching group provides a plurality of reactive sites for connecting tethered ligands to the oligomeric compound through the conjugate linker and/or the cleavable moiety. In certain embodiments, the branching group comprises groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, the branching group is covalently attached to the conjugate linker. In certain embodiments, the branching group is covalently attached to the cleavable moiety. In certain embodiments, the branching group is covalently attached to the conjugate linker and each of the tethered ligands. In certain embodiments, the branching group comprises one or more cleavable bond. In certain embodiments, the conjugate group does not include a branching group.

In certain embodiments, conjugate groups as provided herein include a cell-targeting moiety that has at least one tethered ligand. In certain embodiments, the cell-targeting moiety comprises two tethered ligands covalently attached to a branching group. In certain embodiments, the cell-targeting moiety comprises three tethered ligands covalently attached to a branching group.

As used herein, “tether” means a group of atoms that connect a ligand to the remainder of the conjugate group. In certain embodiments, each tether is a linear aliphatic group comprising one or more groups selected from alkyl, substituted alkyl, ether, thioether, disulfide, amino, oxo, amide, phosphodiester and polyethylene glycol groups in any combination. In certain embodiments, each tether is a linear aliphatic group comprising one or more groups selected from alkyl, ether, thioether, disulfide, amino, oxo, amide and polyethylene glycol groups in any combination. In certain embodiments, each tether is a linear aliphatic group comprising one or more groups selected from alkyl, substituted alkyl, phosphodiester, ether and amino, oxo, amide groups in any combination. In certain embodiments, each tether is a linear aliphatic group comprising one or more groups selected from alkyl, ether and amino, oxo, amide groups in any combination.

In certain embodiments, each tether is a linear aliphatic group comprising one or more groups selected from alkyl, amino and oxo groups in any combination. In certain embodiments, each tether is a linear aliphatic group comprising one or more groups selected from alkyl and oxo groups in any combination. In certain embodiments, each tether is a linear aliphatic group comprising one or more groups selected from alkyl and phosphodiester in any combination. In certain embodiments, each tether comprises at least one phosphorus linking group or neutral linking group.

In certain embodiments, tethers include one or more cleavable bond. In certain embodiments, each tethered ligand is attached to a branching group. In certain embodiments, each tethered ligand is attached to a branching group through an amide group. In certain embodiments, each tethered ligand is attached to a branching group through an ether group. In certain embodiments, each tethered ligand is attached to a branching group through a phosphorus linking group or neutral linking group. In certain embodiments, each tethered ligand is attached to a branching group through a phosphodiester group. In certain embodiments, each tether is attached to a ligand through either an amide or an ether group. In certain embodiments, each tether is attached to a ligand through an ether group.

In certain embodiments, each tether comprises from about 8 to about 20 atoms in chain length between the ligand and the branching group. In certain embodiments, each tether comprises from about 10 to about 18 atoms in chain length between the ligand and the branching group. In certain embodiments, each tether comprises about 13 atoms in chain length.

In certain embodiments, the present disclosure provides ligands wherein each ligand is covalently attached to the remainder of the conjugate group through a tether. In certain embodiments, each ligand is selected to have an affinity for at least one type of receptor on a target cell. In certain embodiments, ligands are selected that have an affinity for at least one type of receptor on the surface of a mammalian liver cell. In certain embodiments, ligands are selected that have an affinity for the hepatic asialoglycoprotein receptor (ASGP-R). In certain embodiments, each ligand is a carbohydrate. In certain embodiments, each ligand is, independently selected from galactose, N-acetyl galactoseamine, mannose, glucose, glucosamone and fucose.

In certain embodiments, each ligand is N-acetyl galactoseamine (GalNAc). In certain embodiments, the targeting moiety comprises 1 to 3 ligands. In certain embodiments, the targeting moiety comprises 3 ligands.

In certain embodiments, the targeting moiety comprises 2 ligands. In certain embodiments, the targeting moiety comprises 1 ligand. In certain embodiments, the targeting moiety comprises 3 N-acetyl galactoseamine ligands. In certain embodiments, the targeting moiety comprises 2 N-acetyl galactoseamine ligands. In certain embodiments, the targeting moiety comprises 1 N-acetyl galactoseamine ligand.

In certain embodiments, each ligand is a carbohydrate, carbohydrate derivative, modified carbohydrate, multivalent carbohydrate cluster, polysaccharide, modified polysaccharide, or polysaccharide derivative. In certain embodiments, each ligand is an amino sugar or a thio sugar. For example, amino sugars may be selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O—[(R)-1-carboxyethyl]-2-deoxy-β-D-glucopyranose (β-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose and N-sulfo-D-glucosamine, and N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, and ethyl 3,4,6,7-tetra-O-acetyl-2-deoxy-1,5-dithio-α-D-gluco-heptopyranoside.

In certain embodiments, conjugate groups as provided herein comprise a carbohydrate cluster. As used herein, “carbohydrate cluster” means a portion of a conjugate group wherein two or more carbohydrate residues are attached to a branching group through tether groups. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808, for examples of carbohydrate conjugate clusters).

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative.

In certain embodiments, conjugate groups are provided wherein the cell-targeting moiety has the formula:

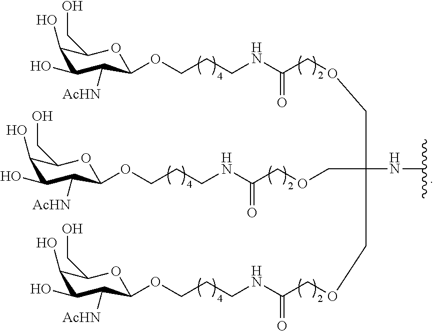

In certain embodiments, conjugate groups are provided wherein the cell-targeting moiety has the formula:

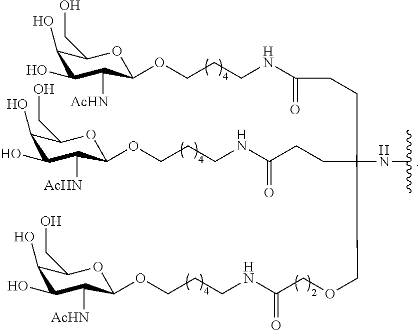

In certain embodiments, conjugate groups are provided wherein the cell-targeting moiety has the formula:

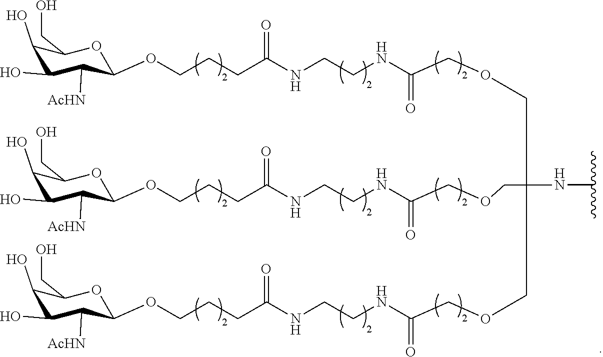

In certain embodiments, conjugate groups have the formula:

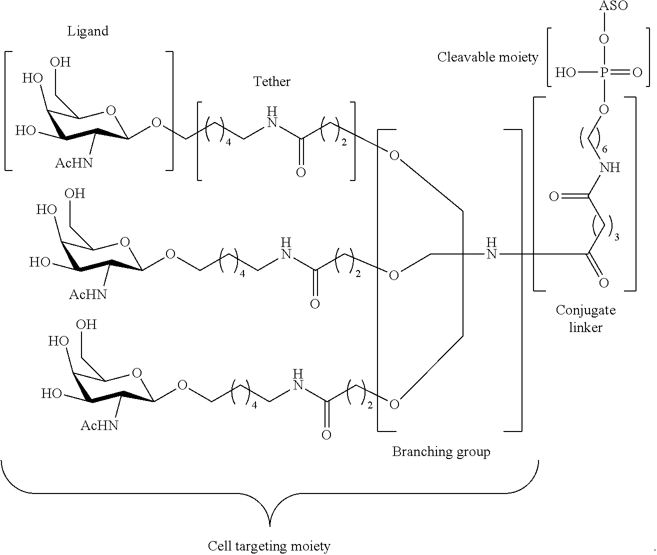

In certain embodiments, an antisense oligonucleotide linked to the conjugate group shown in the formula above has the nucleobase sequence of SEQ ID NO: 1914.

Representative United States patents, United States patent application publications, and international patent application publications that teach the preparation of certain of the above noted conjugate groups, conjugated oligomeric compounds such as antisense compounds comprising a conjugate group, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230, WO 2014/179620 and WO 2012/037254, each of which is incorporated by reference herein in its entirety.

Representative publications that teach the preparation of certain of the above noted conjugate groups, conjugated oligomeric compounds such as antisense compounds comprising a conjugate group, tethers, conjugate linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent glyco-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40): 37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, and Valentijn et al., “Solid-phase synthesis of lysine-based cluster galactosides with high affinity for the Asialoglycoprotein Receptor” Tetrahedron, 1997, 53 (2), 759-770, each of which is incorporated by reference herein in its entirety.

In certain embodiments, conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Some nonlimiting examples of conjugate linkers include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′ end of an oligomeric compound, but before one or more terminal group nucleosides. In certain embodiments, conjugate groups are placed within a terminal group.

In certain embodiments, conjugate groups are at the 5′-end of an oligonucleotide of an oligomeric compound. In certain embodiments, conjugate groups are near the 5′-end.

In certain embodiments, a modified oligonucleotide targeting AGT described herein further comprises a GalNAc conjugate group. In certain embodiments, the GalNAc conjugate group is 5′-Trishexylamino-(THA)-C6 GalNAc₃. In certain embodiments, the 5′-Trishexylamino-(THA)-C6 GalNAc₃ conjugate has the formula

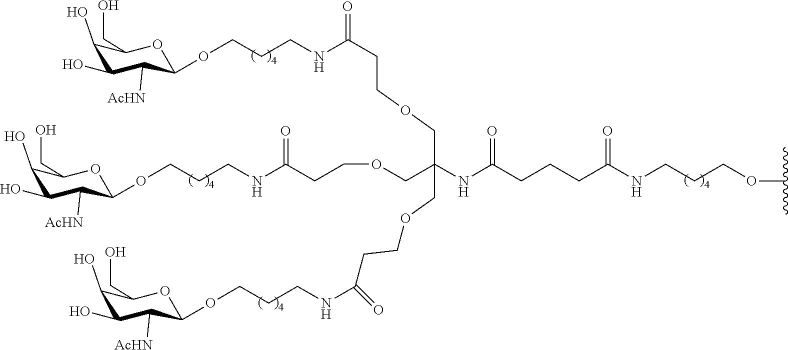

In certain embodiments, the modified oligonucleotide is linked to the 5′-Trishexylamino-(THA)-C6 GalNAc₃ conjugate by a cleavable moiety. In certain embodiments, the cleavable moiety is a phosphate group.

### Cell Culture and Antisense Compounds Treatment

The effects of antisense compounds on the level, activity or expression of AGT nucleic acids can be tested in vitro in a variety of cell types. Cell types used for such analyses are available from commercial vendors (e.g., American Type Culture Collection, Manassas, VA; Zen-Bio, Inc., Research Triangle Park, NC; Clonetics Corporation, Walkersville, MD) and cells are cultured according to the vendor's instructions using commercially available reagents (e.g., Invitrogen Life Technologies, Carlsbad, CA). Illustrative cell types include, but are not limited to, HepG2 cells, Hep3B cells, Huh7 (hepatocellular carcinoma) cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, CA). Antisense oligonucleotides are mixed with LIPOFECTIN® in OPTI-MEM® 1 (Invitrogen, Carlsbad, CA) to achieve the desired final concentration of antisense oligonucleotide and a LIPOFECTIN® concentration that typically ranges 2 to 12 μg/mL per 100 nM antisense oligonucleotide.

Another reagent used to introduce antisense oligonucleotides into cultured cells includes LIPOFECTAMINE 2000® (Invitrogen, Carlsbad, CA). Antisense oligonucleotide is mixed with LIPOFECTAMINE 2000® in OPTI-MEM® 1 reduced serum medium (Invitrogen, Carlsbad, CA) to achieve the desired concentration of antisense oligonucleotide and a LIPOFECTAMINE® concentration that typically ranges 2 to 12 μg/mL per 100 nM antisense oligonucleotide.

Another reagent used to introduce antisense oligonucleotides into cultured cells includes Cytofectin® (Invitrogen, Carlsbad, CA). Antisense oligonucleotide is mixed with Cytofectin® in OPTI-MEM® 1 reduced serum medium (Invitrogen, Carlsbad, CA) to achieve the desired concentration of antisense oligonucleotide and a Cytofectin® concentration that typically ranges 2 to 12 μg/mL per 100 nM antisense oligonucleotide.

Another reagent used to introduce antisense oligonucleotides into cultured cells includes Oligofectamine™ (Invitrogen Life Technologies, Carlsbad, CA). Antisense oligonucleotide is mixed with Oligofectamine™ in Opti-MEM™-1 reduced serum medium (Invitrogen Life Technologies, Carlsbad, CA) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, IN). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM.

Another technique used to introduce antisense oligonucleotides into cultured cells includes electroporation (Sambrook and Russell in Molecular Cloning. A Laboratory Manual. Third Edition. Cold Spring Harbor laboratory Press, Cold Spring Harbor, New York. 2001).

Cells are treated with antisense oligonucleotides by routine methods. Cells are typically harvested 16-24 hours after antisense oligonucleotide treatment, at which time RNA or protein levels of target nucleic acids are measured by methods known in the art and described herein (Sambrook and Russell in Molecular Cloning. A Laboratory Manual. Third Edition. Cold Spring Harbor laboratory Press, Cold Spring Harbor, New York. 2001). In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art (Sambrook and Russell in Molecular Cloning. A Laboratory Manual. Third Edition. Cold Spring Harbor laboratory Press, Cold Spring Harbor, New York. 2001). Antisense oligonucleotides are typically used at concentrations ranging from 1 nM to 300 nM when transfected with LIPOFECTAMINE2000®, Lipofectin or Cytofectin. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art (Sambrook and Russell, Molecular Cloning: A Laboratory Manual, 3^{rd} Ed., 2001). RNA is prepared using methods well known in the art, for example, using the TRIZOL® Reagent (Invitrogen, Carlsbad, CA) according to the manufacturer's recommended protocols.

### Analysis of Inhibition of Target Levels or Expression

Inhibition of levels or expression of an AGT nucleic acid can be assayed in a variety of ways known in the art (Sambrook and Russell, Molecular Cloning: A Laboratory Manual, 3^{rd} Ed., 2001). For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, CA and used according to manufacturer's instructions.

Quantitative Real-Time PCR Analysis of Target RNA Levels Quantitation of target RNA levels may be accomplished by quantitative real-time PCR using the ABI PRISM® 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, CA). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, CA). Cyclophilin A expression is quantified by real time PCR, by being run simultaneously with the target, multiplexing, or separately. Total RNA is quantified using RIBOGREEN® RNA quantification reagent (Invitrogen, Inc. Eugene, OR). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to an AGT nucleic acid. Methods for designing real-time PCR probes and primers are well known in the art, and may include the use of software such as PRIMER EXPRESS® Software (Applied Biosystems, Foster City, CA).

### Analysis of Protein Levels

Antisense inhibition of AGT nucleic acids can be assessed by measuring AGT protein levels. Protein levels of AGT can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS) (Sambrook and Russell, Molecular Cloning: A Laboratory Manual, 3^{rd} Ed., 2001). Antibodies directed to a target can be identified and obtained from a variety of commercially available sources, or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of AGT and produce phenotypic changes, such as, reduced hypertension in the body. Testing can be performed in normal animals, or in experimental disease models. For administration to animals, antisense oligonucleotides are formulated in a pharmaceutically acceptable diluent, such as sterile water-for-injection or phosphate-buffered saline. Administration includes parenteral routes of administration, such as intraperitoneal, intravenous, and subcutaneous. Calculation of antisense oligonucleotide dosage and dosing frequency depends upon factors such as route of administration and animal body weight. In one embodiment, following a period of treatment with antisense oligonucleotides, RNA is isolated from liver tissue and changes in AGT nucleic acid expression are measured. Changes in AGT protein levels can be directly measured. Changes in AGT expression can also be measured by determining the level of inhibition of the RAAS pathway. RAAS pathway related diseases, disorders and/or conditions may be used as markers for determining the level of AGT inhibition.

### Certain Indications

Certain embodiments of the invention provide compounds, compositions and methods of using the compounds and compositions to reduce AGT levels. In certain embodiments, the invention provides compounds, compositions and methods of using the compounds and compositions to treat a subject comprising administering a therapeutically effective amount of the compounds or compositions to the subject. In certain embodiments, the subject has, or is at risk for, a RAAS pathway related disease, disorder or condition. In certain embodiments, the compound or composition comprises and antisense compound.

In certain embodiments, administration of a therapeutically effective amount of an antisense compound targeted to an AGT nucleic acid is accompanied by monitoring of AGT levels in the serum or tissue of a subject to determine a subject's response to the antisense compound. A subject's response to administration of the antisense compound is used by a physician to determine the amount and duration of therapeutic intervention.

In certain embodiments, administration of an antisense compound targeted to an AGT nucleic acid results in reduction of AGT expression by at least 15, 20, 25, 30, 35, 40, 45, 50, 55, 60, 65, 70, 75, 80, 85, 90, 95, 99% or 100% or a range defined by any two of these values. In certain embodiments, administration of an antisense compound targeted to an AGT nucleic acid results in inhibition of the RAAS pathway by at least 15, 20, 25, 30, 35, 40, 45, 50, 55, 60, 65, 70, 75, 80, 85, 90, 95, 99% or 100% or a range defined by any two of these values. In certain embodiments, administration of an antisense compound targeted to an AGT nucleic acid results in a change the RAAS pathway related disease, disorder, condition, symptom or marker (e.g., hypertension or organ damage). In certain embodiments, administration of an AGT antisense compound increases or decreases the RAAS related disease, disorder, condition, symptom or marker by at least 15, 20, 25, 30, 35, 40, 45, 50, 55, 60, 65, 70, 75, 80, 85, 90, 95, 99% or 100% or a range defined by any two of these values.

In certain embodiments, pharmaceutical compositions comprising an antisense compound targeted to AGT are used in the preparation of a medicament for reducing AGT levels. In certain embodiments, pharmaceutical compositions comprising an antisense compound targeted to AGT are used in the preparation of a medicament for treating a subject suffering from, or susceptible to, a RAAS related disease, disorder or condition.

In certain embodiments, reducing AGT levels in a subject treats, ameliorates, prevents, slows the progression, or delays the onset of a disease, condition or disorder. In certain embodiments, the disease, condition or disorder is shortened life expectancy, hypertension, hypertensive emergency (i.e. malignant hypertension), kidney disease (e.g., chronic kidney disease, polycystic kidney disease), pre-eclampsia, Marfan Syndrome, stroke, cardiac disease (e.g., myocardial infarction, heart failure, congestive heart failure, valvular heart disease), aneurysms of the blood vessels, abdominal aneurysm, peripheral artery disease, organ damage, pulmonary arterial hypertension, obesity, metabolic syndrome, non-alcoholic steatohepatitis (NASH), non-alcoholic fatty liver disease (NAFLD) and RAAS related diseases, disorders and/or conditions or symptoms thereof. In certain embodiments, the hypertension is nonresistant hypertension or resistant hypertension. In certain embodiments, the aneurysm of the blood vessels is aortic aneurysm. In certain embodiments, the organ damage is heart muscle hypertrophy or fibrosis in an organ or tissue. In certain embodiments, the organ is heart, liver or kidney and the tissue is derived from the heart, liver or kidney.

In certain embodiments, reducing AGT levels in a subject treats, ameliorates, prevents, slows the progression, or delays the onset of a RAAS pathway related disease, disorder or condition. In certain embodiments, the RAAS pathway related disease, disorder or condition is shortened life expectancy, hypertension, hypertensive emergency (i.e. malignant hypertension), kidney disease (e.g., chronic kidney disease, polycystic kidney disease), pre-eclampsia, Marfan Syndrome, stroke, cardiac disease (e.g., myocardial infarction, heart failure, congestive heart failure, valvular heart disease), aneurysms of the blood vessels, abdominal aneurysm, peripheral artery disease, organ damage, pulmonary arterial hypertension, obesity, metabolic syndrome, NASH, NAFLD and other RAAS related diseases, disorders and/or conditions or symptoms thereof. In certain embodiments, the hypertension is nonresistant hypertension or resistant hypertension. In certain embodiments, the aneurysm of the blood vessels is aortic aneurysm. In certain embodiments, the organ damage is heart muscle hypertrophy or fibrosis in an organ or tissue. In certain embodiments, the organ is heart, liver or kidney and the tissue is derived from the heart, liver or kidney.

In certain embodiments, provided are compounds, compositions and methods for modulating a symptom or marker of a disease, disorder and/or condition. In certain embodiments, the marker can be selected from one or more of shortened life expectancy, hypertension, hypertensive emergency (i.e. malignant hypertension), kidney disease (e.g., chronic kidney disease, polycystic kidney disease), pre-eclampsia, Marfan Syndrome, stroke, cardiac disease (e.g., myocardial infarction, heart failure, congestive heart failure, valvular heart disease), aneurysms of the blood vessels, abdominal aneurysm, peripheral artery disease, organ damage and other RAAS related diseases, disorders and/or conditions or symptoms thereof.

### Certain Combination Therapies

In certain embodiments, a first agent comprising an antisense compound provided herein is co-administered with one or more secondary agents. In certain embodiments, the antisense compound is an antisense oligonucleotide. In certain embodiments, the antisense oligonucleotide is a modified oligonucleotide.

In certain embodiments, such second agents are designed to treat the same RAAS pathway related disease, disorder or condition as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, such first agents are designed to treat an undesired side effect of a second agent. In certain embodiments, second agents are co-administered with the first agent to treat an undesired effect of the first agent. In certain embodiments, second agents are co-administered with the first agent to produce a combinational or additive effect. In certain embodiments, second agents are co-administered with the first agent to produce a synergistic effect.

In certain embodiments, the co-administration of the first and second agents permits use of lower dosages than would be required to achieve a therapeutic or prophylactic effect if the agents were administered as independent therapy. In certain embodiments the dose of a co-administered second agent is the same as the dose that would be administered if the second agent was administered alone. In certain embodiments the dose of a co-administered second agent is greater than the dose that would be administered if the second agent was administered alone.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

In certain embodiments, second agents include, but are not limited to, certain procedures to reduce hypertension, diet changes, lifestyle changes, anti-fibrotic drugs and anti-hypertensive drugs such as RAAS inhibitors, endothelin receptor antagonists, neprilysin inhibitors, diuretics, calcium channel blockers, adrenergic receptor antagonists, adrenergic agonists and vasodilators.

Examples of procedures that can reduce hypertension include, but are not limited to, renal denervation and baroreceptor activation therapy.

Examples of RAS or RAAS inhibitors include, but are not limited to ACE inhibitors (e.g., captopril, enalapril, fosinopril, lisinopril, perindopril, quinapril, ramipril, trandolapril and benazepril), angiotensin II receptor antagonists (e.g., candesartan, eprosartan, irbesartan, losartan, olmesartan, telmisartan and valsartan), renin inhibitors (e.g., aliskiren), aldosterone receptor antagonists (e.g., eplerenone, spironolactone and finerenone).

Examples of endothelin receptor antagonists include ambrisentan, sitaxentan, atrasentan, BQ-123, zibotentan, bosentan, macitentan and tezosentan.

Examples of neprilysin inhibitors include sacubitril and omapatrilat.

Examples of diuretics include loop diuretics (e.g., bumetanide, ethacrynic acid, furosemide, torsemide), thiazide diuretics (e.g., epitizide, hydrochlorothiazide, chlorothiazide and bendroflumethiazide), thiazide-like diuretics (e.g., indapamide, chlorthalidone and metolazone) and potassium-sparing diuretics (e.g., amiloride, triamterene and spironolactone).

Examples of calcium channel blockers include dihydropyridines (e.g., amlodipine, felodipine, isradipine, lercanidipine, nicardipine, nifedipine, nimodipine and nitrendipine) and non-dihydropyridines (e.g., diltiazem and verapamil).

Examples of adrenergic receptor antagonists include Beta blockers (e.g., atenolol, metoprolol, nadolol, oxprenolol, pindolol, propranolol and timolol), Alpha blockers (e.g., doxazosin, phentolamine, indoramin, phenoxybenzamine, prazosin, terazosin and tolazoline) and mixed Alpha+Beta blockers (e.g., bucindolol, carvedilol and labetalol).

Examples of vasodilators include sodium nitroprusside and hydralazine and its derivatives.

Examples of adrenergic agonists include alpha-2 agonists (e.g., clonidine, guanabenz, methyldopa and moxonidine).

Additional examples of anti-hypertensive drugs include guanethidine, reserpine and the like.

The second agents can be used in combination with the therapeutic compounds described herein to decrease a disease, disorder and/or condition such as hypertension, organ damage and the like.

### Certain Compounds

Preferred antisense compounds with beneficial properties that enhance their use as therapeutic treatments in humans are demonstrated in the examples herein. For brevity, only the studies that contributed to the selection of the preferred antisense compounds are described. A non-exhaustive summary of the examples is provided below for ease of reference.

Over 2000 antisense compounds with a MOE containing and/or a cEt containing gapmer motif targeting human AGT were designed. Example 1 shows representative single dose inhibition data for the over 2000 potent antisense compounds tested in HepG2 cells for their effect on human AGT mRNA.

Of the over 2000 antisense compounds tested with a single dose in vitro, over 160 antisense compounds were chosen for testing in dose-dependent inhibition studies to determine their half maximal inhibitory concentration (IC₅₀) in HepG2 cells (Example 2).

Base on the in vitro dose response studies, over 50 antisense compounds were selected for single dose potency and tolerability testing in human AGT transgenic (huAGT tg) mice as described in the exemplary studies in Example 3. Of the over 50 antisense compounds, about 14 antisense compounds were further selected for dose response and tolerability studies in huAGT tg mice (Example 4).

Nine antisense compounds exhibiting significant potency and tolerability in huAGT mice were chosen for further studies: in a viscosity assay (Example 5); in CD1 mice (Example 6) and Sprague-Dawlay rats (Example 7) to assess tolerability of the antisense compounds; in monkey hepatocytes to test cross-species potency in inhibiting monkey AGT (Example 8); and in cynomolgus monkeys to assess potency and tolerability (Example 9). Although the antisense compounds in the studies described in Example 9 were tested in cynomolgus monkeys, the cynomolgus monkey AGT sequence was not available for comparison to the sequences of the antisense compounds, therefore the sequences of the antisense compounds were compared to that of the closely related rhesus monkey (Example 8).

Based on the extensive capacitation of the 9 antisense compounds, the sequence of antisense compound ISIS 654472 (parent compound) was selected for further study (Example 10). Six antisense compounds were designed with the sequence of parent compound ISIS 654472 but with different chemical modifications and a GalNAc conjugate. The 6 newly designed compounds were administered to CD1 mice (Example 10) and Sprague-Dawley rats (Example 11) to test their tolerability in these animal models. Of the 6 GalNAc conjugated antisense compounds, compound ISIS 757456 was selected to test in huAGT mice compared to the parent antisense compound ISIS 654472. ISIS 757456 showed an 8× improvement in potency compared to unconjugated compound ISIS 654472.

Accordingly, provided herein are antisense compounds with any one or more characteristics that are beneficial for their use as a therapeutic agent. In certain embodiments, provided herein are antisense compounds comprising a modified oligonucleotide as described herein targeted to, or specifically hybridizable with, a region of nucleotides selected from any of SEQ ID NOs: 1-6.

In certain embodiments, certain antisense compounds as described herein are efficacious by virtue of their potency in inhibiting AGT expression. In certain embodiments, the compounds or compositions inhibit AGT by at least 40%, at least 45%, at least 50%, at least 55%, at least 60%, at least 65%, at least 70%, at least 75%, at least 80%, at least 85%, at least 90% or at least 95%.

In certain embodiments, certain antisense compounds as described herein are efficacious by virtue of an in vitro IC₅₀ of less than 20 μM, less than 10 μM, less than 8 μM, less than 5 μM, less than 2 μM, less than 1 μM, less than 0.9 μM, less than 0.8 μM, less than 0.7 μM, less than 0.6 μM, or less than 0.5 μM when tested in human cells, for example, in the Hep3B cell line (as described in Example 2).

In certain embodiments, certain antisense compounds as described herein are efficacious by virtue of a median effective dose (ED₅₀) of ≤10 mpk/wk, ≤9 mpk/wk, ≤8 mpk/wk, ≤7 mpk/wk, ≤6 mpk/wk, ≤5 mpk/wk, ≤4 mpk/wk, ≤3 mpk/wk, ≤2 mpk/wk, or ≤1 mpk/wk in vivo as shown in Example 4. In certain embodiments, a preferred antisense compound such as antisense compound ISIS 757456 has an ED₅₀≤3 mpk/wk as shown in Example 12.

In certain embodiments, certain antisense compounds as described herein are efficacious by virtue of having a viscosity of less than 40 cP, less than 35 cP, less than 30 cP, less than 25 cP, less than 20 cP, less than 15 cP, or less than 12 cP as described in Example 5. Oligonucleotides having a viscosity greater than 40 cP would have less than optimal viscosity.

In certain embodiments, certain antisense compounds as described herein are highly tolerable, as demonstrated by the in vivo tolerability measurements described in the examples. In certain embodiments, the certain antisense compounds as described herein are highly tolerable, as demonstrated by having an increase in ALT and/or AST value of no more than 3 fold, 2 fold or 1.5 fold over saline treated animals.

In certain embodiments, certain antisense compounds as described herein are efficacious by virtue of having one or more of an inhibition potency of greater than 50%, an ED₅₀≤5 mpk/wk, a viscosity of less than 40 cP, and no more than a 3 fold increase in ALT and/or AST in transgenic mice.

In certain embodiments, ISIS 757456 (SEQ ID NO: 1914) is preferred. This compound was found to be a potent inhibitor in AGT transgenic mice and a very tolerable antisense compound in CD-1 mice. In mice it had less than a 3 fold increase in ALT and/or AST levels over saline treated animals. It had an ED₅₀≤3 mpk/wk in huAGT transgenic mice.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human Angiotensinogen (AGT) in HepG2 Cells

Over 2000 antisense oligonucleotides were designed targeting human AGT nucleic acid and were tested for their effects on AGT mRNA in vitro in a series of experiments that had similar culture conditions. The results for representative antisense oligonucleotides are presented in tables shown below.

The newly designed chimeric antisense oligonucleotides in the Tables below were designed as MOE and/or cEt containing gapmers. The MOE containing oligonucleotides have a central gap segment comprising 2′-deoxynucleosides which is flanked by wing segments on the 5′ direction and the 3′ direction. At least one nucleoside in the 5′ wing segment and/or one nucleoside in the 3′ wing segment has a 2′-MOE sugar modification. The cEt containing oligonucleotides have a central gap segment comprising 2′-deoxynucleosides which is flanked by wing segments on the 5′ direction and the 3′ direction. At least one nucleoside in the 5′ wing segment and/or one nucleoside in the 3′ wing segment has a cEt sugar modification. In some instances oligonucleotides were designed to contain both a MOE and a cEt. The MOE and cEt containing oligonucleotides have a central gap segment comprising 2′-deoxynucleosides which is flanked by wing segments on the 5′ direction and the 3′ direction. At least one nucleoside in the 5′ wing segment and/or one nucleoside in the 3′ wing segment has a MOE and/or cEt sugar modification.

The “Chemistry” column describes the sugar modifications of each oligonucleotide. “k” indicates an cEt sugar modification; “d” indicates deoxyribose; and “e” indicates a MOE modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human AGT mRNA, designated herein as SEQ ID NO: 1 (GENBANK Accession NM_000029.3) and/or the human AGT genomic sequence, designated herein as SEQ ID NO: 2 (GENBANK Accession NT_167186.1 truncated from nucleotides 24354000 to 24370100).

Table 1 shows inhibition of AGT mRNA in HepG2 cells cultured at a density of 20,000 cells per well which were transfected using electroporation with 4500 nM antisense oligonucleotide. After a treatment period of approximately 24 hours, RNA was isolated from the cells and AGT mRNA levels were measured by quantitative real-time PCR. Human primer probe set RTS3721 (forward sequence CCCTGATGGGAGCCAGTGT, designated herein as SEQ ID NO: 8; reverse sequence AGCAGGGAGAAGCCCTTCA, designated herein as SEQ ID NO: 9; and probe sequence CCCTGGCTTTCAACACCTACGTCCACTX, where X is a fluorescent label, designated herein as SEQ ID NO: 10) was used to measure mRNA levels. AGT mRNA levels were adjusted according to total RNA content, as measured by RIBOGREEN®. Results are presented as percent inhibition of AGT, relative to untreated control cells.

TABLE 1
Inhibition of AGT mRNA by MOE and/or cEt containing gapmers targeting SEQ ID NO: 1 and/or 2

| | | SEQ | | | | | | |
|---|---|---|---|---|---|---|---|---|
| | SEQ | ID: | | | | SEQ | SEQ | |
| | ID: 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568518 | 1 | 16 | TGCCCGCTCATGGGAT | eekddddddddddkke | 26 | 1986 | 2001 | 14 |
| 568519 | 20 | 35 | GGGCCACTTCTGACCC | eekddddddddddkke | 34 | 2005 | 2020 | 15 |
| 568520 | 35 | 50 | GCTTAGGCAACACGGG | eekddddddddddkke | 20 | 2020 | 2035 | 16 |
| 568521 | 45 | 60 | GGAGAGTCTTGCTTAG | eekddddddddddkke | 26 | 2030 | 2045 | 17 |
| 568522 | 80 | 95 | CATGCAGGCCGGAGGT | eekddddddddddkke | 25 | 2065 | 2080 | 18 |
| 568523 | 90 | 105 | GCCACAGGGACATGCA | eekddddddddddkke | 30 | 2075 | 2090 | 19 |
| 568524 | 122 | 137 | TGACCCAGCCCCGGGA | eekddddddddddkke | 40 | 2107 | 2122 | 20 |
| 568525 | 155 | 170 | TGTGACAGCCTGAGGC | eekddddddddddkke | 25 | 2140 | 2155 | 21 |
| 568526 | 165 | 180 | TCCCTAGGTGTGTGAC | eekddddddddddkke | 34 | 2150 | 2165 | 22 |
| 568527 | 179 | 194 | GAAACGGGAGCATCTC | eekddddddddddkke | 9 | 2164 | 2179 | 23 |
| 568528 | 189 | 204 | AAGGTTCCCAGAAACG | eekddddddddddkke | 19 | 2174 | 2189 | 24 |
| 568529 | 209 | 224 | AGTTTGCAGGAGTCGG | eekddddddddddkke | 26 | 2194 | 2209 | 25 |
| 568530 | 229 | 244 | TCGAGTTACACATTTA | eekddddddddddkke | 24 | 2214 | 2229 | 26 |
| 568531 | 248 | 263 | AGAGTGAGCCGGTGCA | eekddddddddddkke | 21 | 2233 | 2248 | 27 |
| 568532 | 258 | 273 | ACTGCTGAACAGAGTG | eekddddddddddkke | 19 | 2243 | 2258 | 28 |
| 568533 | 268 | 283 | GCAGAGTTTCACTGCT | eekddddddddddkke | 16 | 2253 | 2268 | 29 |
| 568534 | 278 | 293 | AGTGATCGATGCAGAG | eekddddddddddkke | 32 | 2263 | 2278 | 30 |
| 568535 | 288 | 303 | AGGAAGTCTTAGTGAT | eekddddddddddkke | 15 | 2273 | 2288 | 31 |
| 568536 | 301 | 316 | TGGGACCTCTTCCAGG | eekddddddddddkke | 31 | 2286 | 2301 | 32 |
| 568537 | 353 | 368 | GGCCAGACCACAGGCT | eekddddddddddkke | 16 | 2338 | 2353 | 33 |
| 568538 | 363 | 378 | TACATCACTTGGCCAG | eekddddddddddkke | 30 | 2348 | 2363 | 34 |
| 568539 | 373 | 388 | AGAGGAGGGTTACATC | eekddddddddddkke | 24 | 2358 | 2373 | 35 |
| 568540 | 386 | 401 | GTGCACAGGCTGGAGA | eekddddddddddkke | 43 | 2371 | 2386 | 36 |
| 568541 | 431 | 446 | TATTTATAGCTGAGGG | eekddddddddddkke | 29 | 2416 | 2431 | 37 |
| 568542 | 441 | 456 | CACGATGCCCTATTTA | eekddddddddddkke | 34 | 2426 | 2441 | 38 |
| 568543 | 478 | 493 | TACCCAGAACAACGGC | eekddddddddddkke | 28 | 2463 | 2478 | 39 |
| 568544 | 525 | 540 | GCCATCTCAGACTGGG | eekddddddddddkke | 69 | 5742 | 5757 | 40 |
| 568545 | 535 | 550 | ACCGGCAGGAGCCATC | eekddddddddddkke | 42 | 5752 | 5767 | 41 |
| 568546 | 545 | 560 | TCAGGCTCACACCGGC | eekddddddddddkke | 67 | 5762 | 5777 | 42 |
| 568547 | 555 | 570 | ATGGTGGCCCTCAGGC | eekddddddddddkke | 39 | 5772 | 5787 | 43 |
| 568548 | 596 | 611 | GGTCACCTGCAGCCAG | eekddddddddddkke | 59 | 5813 | 5828 | 44 |
| 568549 | 606 | 621 | ATGTACACCCGGTCAC | eekddddddddddkke | 25 | 5823 | 5838 | 45 |
| 568550 | 643 | 658 | GGTACTCTCATTGTGG | eekddddddddddkke | 76 | 5860 | 5875 | 46 |
| 568551 | 654 | 669 | AGCTGCTCACAGGTAC | eekddddddddddkke | 60 | 5871 | 5886 | 47 |
| 568552 | 676 | 691 | CTTCCCGGCATTGGCC | eekddddddddddkke | 50 | 5893 | 5908 | 48 |
| 568553 | 703 | 718 | AGCAGGTATGAAGGTG | eekddddddddddkke | 62 | 5920 | 5935 | 49 |
| 568554 | 713 | 728 | CCTGAATTGGAGCAGG | eekddddddddddkke | 43 | 5930 | 5945 | 50 |
| 568555 | 723 | 738 | GATGTCTTGGCCTGAA | eekddddddddddkke | 21 | 5940 | 5955 | 51 |
| 568556 | 739 | 754 | CTTTTCATCCACAGGG | eekddddddddddkke | 39 | 5956 | 5971 | 52 |
| 568557 | 762 | 777 | AGCACCAGCTGGTCCT | eekddddddddddkke | 71 | 5979 | 5994 | 53 |
| 568558 | 772 | 787 | TGCAGCGACTAGCACC | eekddddddddddkke | 71 | 5989 | 6004 | 54 |
| 568559 | 782 | 797 | TGTCAAGTTTTGCAGC | eekddddddddddkke | 61 | 5999 | 6014 | 55 |
| 568560 | 803 | 818 | CGGCCCTCAACTTGTC | eekddddddddddkke | 45 | 6020 | 6035 | 56 |
| 568561 | 815 | 830 | TCCCGACCATTGCGGC | eekddddddddddkke | 25 | 6032 | 6047 | 57 |
| 568562 | 825 | 840 | TTGGCCAGCATCCCGA | eekddddddddddkke | 51 | 6042 | 6057 | 58 |
| 568563 | 835 | 850 | GCCCAAGAAGTTGGCC | eekddddddddddkke | 13 | 6052 | 6067 | 59 |
| 568564 | 845 | 860 | ATATACGGAAGCCCAA | eekddddddddddkke | 52 | 6062 | 6077 | 60 |
| 568565 | 855 | 870 | TGCATGCCATATATAC | eekddddddddddkke | 64 | 6072 | 6087 | 61 |
| 568566 | 871 | 886 | GCCCCATAGCTCACTG | eekddddddddddkke | 64 | 6088 | 6103 | 62 |
| 568567 | 886 | 901 | GGCCCCATGGACCACG | eekddddddddddkke | 38 | 6103 | 6118 | 63 |
| 568568 | 913 | 928 | AAAGACAGCCGTTGGG | eekddddddddddkke | 58 | 6130 | 6145 | 64 |
| 568569 | 923 | 938 | CCAGGGTGCCAAAGAC | eekddddddddddkke | 36 | 6140 | 6155 | 65 |
| 568570 | 937 | 952 | CAGATAGAGAGAGGCC | eekddddddddddkke | 59 | 6154 | 6169 | 66 |
| 568571 | 954 | 969 | GTGTGGTCCAAGGCTC | eekddddddddddkke | 37 | 6171 | 6186 | 67 |
| 568572 | 983 | 998 | CACCCAGGATTGCCTG | eekddddddddddkke | 72 | 6200 | 6215 | 68 |
| 568573 | 993 | 1008 | TTCCAAGGAACACCCA | eekddddddddddkke | 35 | 6210 | 6225 | 69 |
| 568574 | 1017 | 1032 | AGCCGGGAGGTGCAGT | eekddddddddddkke | 53 | 6234 | 6249 | 70 |
| 568575 | 1020 | 1035 | TCCAGCCGGGAGGTGC | eekddddddddddkke | 62 | 6237 | 6252 | 71 |
| 568576 | 1053 | 1068 | ACAGCCTGCAGGGCAG | eekddddddddddkke | 47 | 6270 | 6285 | 72 |
| 568577 | 1070 | 1085 | CCACTAGCAGGCCCTG | eekddddddddddkke | 33 | 6287 | 6302 | 73 |
| 568578 | 1088 | 1103 | TATCAGCCCTGCCCTG | eekddddddddddkke | 37 | 6305 | 6320 | 74 |
| 568579 | 1098 | 1113 | TGGGCCTGGCTATCAG | eekddddddddddkke | 42 | 6315 | 6330 | 75 |
| 568580 | 1114 | 1129 | CGTGGACAGCAGCAGC | eekddddddddddkke | 70 | 6331 | 6346 | 76 |
| 568581 | 1131 | 1146 | GTGAACACGCCCACCA | eekddddddddddkke | 48 | 6348 | 6363 | 77 |
| 568582 | 1151 | 1166 | TCAGGTGCAGGCCTGG | eekddddddddddkke | 36 | 6368 | 6383 | 78 |
| 568583 | 1171 | 1186 | GCCCTGCACAAACGGC | eekddddddddddkke | 16 | 6388 | 6403 | 79 |
| 568584 | 1182 | 1197 | TAGAGAGCCAGGCCCT | eekddddddddddkke | 52 | 6399 | 6414 | 80 |
| 568585 | 1203 | 1218 | CGTGGGAGGACCACAG | eekddddddddddkke | 47 | 6420 | 6435 | 81 |
| 568586 | 1217 | 1232 | TGAAGTCCAGAGAGCG | eekddddddddddkke | 60 | 6434 | 6449 | 82 |
| 568587 | 1233 | 1248 | GCAACATCCAGTTCTG | eekddddddddddkke | 50 | 6450 | 6465 | 83 |
| 568588 | 1244 | 1259 | TCTTCTCAGCAGCAAC | eekddddddddddkke | 54 | 6461 | 6476 | 84 |
| 568589 | 1272 | 1287 | CCTGTCACAGCCTGCA | eekddddddddddkke | 77 | 6489 | 6504 | 85 |
| 568590 | 1278 | 1293 | TTCCATCCTGTCACAG | eekddddddddddkke | 51 | 6495 | 6510 | 86 |
| 568595 | 1403 | 1418 | TGTCCACCCAGAACTC | eekddddddddddkke | 33 | 10414 | 10429 | 87 |
| 568596 | 1406 | 1421 | TGTTGTCCACCCAGAA | eekddddddddddkke | 59 | 10417 | 10432 | 88 |
| 568597 | 1409 | 1424 | TGCTGTTGTCCACCCA | eekddddddddddkke | 60 | 10420 | 10435 | 89 |
| 568598 | 1412 | 1427 | AGGTGCTGTTGTCCAC | eekddddddddddkke | 57 | 10423 | 10438 | 90 |
| 568599 | 1415 | 1430 | CTGAGGTGCTGTTGTC | eekddddddddddkke | 56 | 10426 | 10441 | 91 |
| 568600 | 1418 | 1433 | ACACTGAGGTGCTGTT | eekddddddddddkke | 28 | 10429 | 10444 | 92 |
| 568601 | 1421 | 1436 | CAGACACTGAGGTGCT | eekddddddddddkke | 67 | 10432 | 10447 | 93 |
| 568602 | 1431 | 1446 | AGCATGGGAACAGACA | eekddddddddddkke | 27 | 10442 | 10457 | 94 |
| 568603 | 1443 | 1458 | CCCATGCCAGAGAGCA | eekddddddddddkke | 30 | 10454 | 10469 | 95 |
| 568604 | 1462 | 1477 | ACTCCAGTGCTGGAAG | eekddddddddddkke | 41 | 10473 | 10488 | 96 |
| 568605 | 1465 | 1480 | GTCACTCCAGTGCTGG | eekddddddddddkke | 73 | 10476 | 10491 | 97 |
| 568606 | 1474 | 1489 | GTCCTGGATGTCACTC | eekddddddddddkke | 68 | 10485 | 10500 | 98 |
| 568607 | 1484 | 1499 | CCGAGAAGTTGTCCTG | eekddddddddddkke | 47 | 10495 | 10510 | 99 |
| 568608 | 1494 | 1509 | ACTTGAGTCACCGAGA | eekddddddddddkke | 39 | 10505 | 10520 | 100 |
| 568609 | 1504 | 1519 | AGTGAAGGGCACTTGA | eekddddddddddkke | 28 | 10515 | 10530 | 101 |
| 568610 | 1531 | 1546 | CTGGATCAGCAGCAGG | eekddddddddddkke | 69 | 10542 | 10557 | 102 |
| 568611 | 1550 | 1565 | GGTCAGAGGCATAGTG | eekddddddddddkke | 43 | 10561 | 10576 | 103 |
| 568612 | 1578 | 1593 | TGGAAAGTGAGACCCT | eekddddddddddkke | 45 | 10589 | 10604 | 104 |
| 568613 | 1588 | 1603 | GGAGTTTTGCTGGAAA | eekddddddddddkke | 53 | 10599 | 10614 | 105 |
| 568614 | 1598 | 1613 | TCCAGTTGAGGGAGTT | eekddddddddddkke | 38 | 10609 | 10624 | 106 |
| 568615 | 1614 | 1629 | GGAGATAGTTTCTTCA | eekddddddddddkke | 24 | 10625 | 10640 | 107 |
| 568616 | 1631 | 1646 | TCAGGTGGATGGTCCG | eekddddddddddkke | 34 | N/A | N/A | 108 |
| 568617 | 1653 | 1668 | TGCAGCACCAGTTGGG | eekddddddddddkke | 65 | 12259 | 12274 | 109 |
| 568618 | 1663 | 1678 | ATAAGATCCTTGCAGC | eekddddddddddkke | 21 | 12269 | 12284 | 110 |
| 568619 | 1680 | 1695 | AGCAGGTCCTGCAGGT | eekddddddddddkke | 50 | 12286 | 12301 | 111 |
| 568620 | 1700 | 1715 | CGGGCAGCTCAGCCTG | eekddddddddddkke | 39 | 12306 | 12321 | 112 |
| 568621 | 1710 | 1725 | TGCAGAATGGCGGGCA | eekddddddddddkke | 57 | 12316 | 12331 | 113 |
| 568622 | 1720 | 1735 | CAGCTCGGTGTGCAGA | eekddddddddddkke | 70 | 12326 | 12341 | 114 |
| 568623 | 1730 | 1745 | TTTGCAGGTTCAGCTC | eekddddddddddkke | 44 | 12336 | 12351 | 115 |
| 568624 | 1745 | 1760 | GGTCATTGCTCAATTT | eekddddddddddkke | 45 | 12351 | 12366 | 116 |
| 568625 | 1755 | 1770 | ACCCTGATGCGGTCAT | eekddddddddddkke | 43 | 12361 | 12376 | 117 |
| 568626 | 1794 | 1809 | GCTTCAAGCTCAAAAA | eekddddddddddkke | 56 | 13263 | 13278 | 118 |
| 568627 | 1827 | 1842 | TGTTGGGTAGACTCTG | eekddddddddddkke | 61 | 13296 | 13311 | 119 |
| 568628 | 1841 | 1856 | CAGGCTTGTTAAGCTG | eekddddddddddkke | 53 | 13310 | 13325 | 120 |
| 568629 | 1851 | 1866 | TCCAAGACCTCAGGCT | eekddddddddddkke | 46 | 13320 | 13335 | 121 |
| 568630 | 1875 | 1890 | AGGAATGGGCGGTTCA | eekddddddddddkke | 58 | 13344 | 13359 | 122 |
| 568631 | 1923 | 1938 | CGGCCCAGGAAGTGCA | eekddddddddddkke | 30 | 13392 | 13407 | 123 |
| 568632 | 1933 | 1948 | GTTGGCCACGCGGCCC | eekddddddddddkke | 11 | 13402 | 13417 | 124 |
| 568633 | 1943 | 1958 | TGCTCAGCGGGTTGGC | eekddddddddddkke | 49 | 13412 | 13427 | 125 |
| 568634 | 1961 | 1976 | GGCCCTGGCCTCATGC | eekddddddddddkke | 44 | 13430 | 13445 | 126 |
| 568635 | 1986 | 2001 | GGCCTTGCCAGGCACT | eekddddddddddkke | 86 | 13455 | 13470 | 127 |
| 568636 | 2007 | 2022 | GCCTCAAAGGCCAGGG | eekddddddddddkke | 49 | 13476 | 13491 | 128 |
| 568637 | 2046 | 2061 | CGCTGATTTGTCCGGG | eekddddddddddkke | 92 | 13515 | 13530 | 129 |
| 568638 | 2056 | 2071 | GGTGACACATCGCTGA | eekddddddddddkke | 86 | 13525 | 13540 | 130 |
| 568639 | 2075 | 2090 | GAAAAGGTGGGAGACT | eekddddddddddkke | 39 | 13544 | 13559 | 131 |
| 568640 | 2088 | 2103 | CGACTCATTAGAAGAA | eekddddddddddkke | 87 | 13557 | 13572 | 132 |
| 568641 | 2111 | 2126 | ACGGCTGCTTTCCAGC | eekddddddddddkke | 64 | 13580 | 13595 | 133 |
| 568642 | 2121 | 2136 | CCAAGGAGAAACGGCT | eekddddddddddkke | 79 | 13590 | 13605 | 134 |
| 568643 | 2131 | 2146 | CACACTTAGACCAAGG | eekddddddddddkke | 78 | 13600 | 13615 | 135 |
| 568644 | 2166 | 2181 | TGCCGCTGCAGGCTTC | eekddddddddddkke | 57 | 13635 | 13650 | 136 |
| 568645 | 2176 | 2191 | GGTGCATTTGTGCCGC | eekddddddddddkke | 75 | 13645 | 13660 | 137 |
| 568646 | 2274 | 2289 | TGGTCGGTTGGAATTC | eekddddddddddkke | 77 | 13743 | 13758 | 138 |
| 568647 | 2284 | 2299 | ACAAACAAGCTGGTCG | eekddddddddddkke | 84 | 13753 | 13768 | 139 |
| 568648 | 2311 | 2326 | CTTGAAAAGGGAACAC | eekddddddddddkke | 62 | 13780 | 13795 | 140 |
| 568649 | 2331 | 2346 | AACCCAATTTTTGTTC | eekddddddddddkke | 56 | 13800 | 13815 | 141 |
| 568650 | 2362 | 2377 | GGCAATGCAAAAATGT | eekddddddddddkke | 78 | 13831 | 13846 | 142 |
| 568651 | 2391 | 2406 | TACATTCAAGACACTA | eekddddddddddkke | 60 | 13860 | 13875 | 143 |
| 568652 | 2402 | 2417 | GGTCATGTTCTTACAT | eekddddddddddkke | 55 | 13871 | 13886 | 144 |
| 568653 | 2412 | 2427 | ACTACACGGAGGTCAT | eekddddddddddkke | 55 | 13881 | 13896 | 145 |
| 568654 | 2422 | 2437 | TATTACAGACACTACA | eekddddddddddkke | 35 | 13891 | 13906 | 146 |
| 568655 | 2482 | 2497 | GGTGCTTGCATCTTTC | eekddddddddddkke | 58 | 13951 | 13966 | 147 |
| 568656 | 2492 | 2507 | CAGAAATTCAGGTGCT | eekddddddddddkke | 47 | 13961 | 13976 | 148 |
| 568657 | 2503 | 2518 | CCGCATTCAAACAGAA | eekddddddddddkke | 38 | 13972 | 13987 | 149 |
| 568658 | 2513 | 2528 | AGCTATGGTTCCGCAT | eekddddddddddkke | 55 | 13982 | 13997 | 150 |
| 568659 | 2537 | 2552 | TACTAACACAAGGGAG | eekddddddddddkke | 37 | 14006 | 14021 | 151 |
| 568660 | 2558 | 2573 | TTATTGTGGCAAGACG | eekddddddddddkke | 48 | 14027 | 14042 | 152 |
| 568661 | N/A | N/A | TTACTAATACAGCCCA | eekddddddddddkke | 31 | 3322 | 3337 | 153 |
| 568662 | N/A | N/A | GGTTTCCCTGATGCAG | eekddddddddddkke | 34 | 3516 | 3531 | 154 |
| 568663 | N/A | N/A | TGATAGTTGGATTCCT | eekddddddddddkke | 21 | 4783 | 4798 | 155 |
| 568664 | N/A | N/A | TGTGGTCCCAACATGC | eekddddddddddkke | 41 | 4944 | 4959 | 156 |
| 568665 | N/A | N/A | TTGAAGTCCTCAACCC | eekddddddddddkke | 26 | 5460 | 5475 | 157 |
| 568670 | N/A | N/A | CTCTTGGATGTCACAG | eekddddddddddkke | 56 | 10997 | 11012 | 158 |
| 568671 | N/A | N/A | GATGGCAAATTTTGTT | eekddddddddddkke | 23 | 11321 | 11336 | 159 |
| 568672 | N/A | N/A | TGTGTTACTTGGGTAA | eekddddddddddkke | 68 | 11933 | 11948 | 160 |
| 568673 | N/A | N/A | GCCACACAGTGAGGGC | eekddddddddddkke | 22 | 12189 | 12204 | 161 |

Table 2 shows the percent inhibition of AGT mRNA by additional gapmer oligonucleotides. Cultured HepG2 cells at a density of about 20,000 cells per well were transfected using electroporation with 4,000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 2
Inhibition of AGT mRNA by MOE and/or cEt
containing gapmers targeting
SEQ ID NO: 1 and/or 2

| | SEQ | SEQ | | | | SEQ | SEQ | |
|---|---|---|---|---|---|---|---|---|
| | ID: | ID: | | | % | ID: | ID | |
| | 1 | 1 | | | Inhi- | 2 | 2: | SEQ |
| ISIS | Start | Stop | Se- | Chem- | bi- | Start | Stop | ID |
| NO | Site | Site | quence | istry | tion | Site | Site | NO |
| 568637 | 2046 | 2061 | CGCT | eekd | 92 | 13515 | 13530 | 129 |
| | | | GATT | dddd | | | | |
| | | | TGTC | dddd | | | | |
| | | | CGGG | dkke | | | | |
| 568637 | 2046 | 2061 | CGCT | eekd | 91 | 13515 | 13530 | 129 |
| | | | GATT | dddd | | | | |
| | | | TGTC | dddd | | | | |
| | | | CGGG | dkke | | | | |
| 568637 | 2046 | 2061 | CGCT | eekd | 97 | 13515 | 13530 | 129 |
| | | | GATT | dddd | | | | |
| | | | TGTC | dddd | | | | |
| | | | CGGG | dkke | | | | |
| 568637 | 2046 | 2061 | CGCT | eekd | 94 | 13515 | 13530 | 129 |
| | | | GATT | dddd | | | | |
| | | | TGTC | dddd | | | | |
| | | | CGGG | dkke | | | | |
| 568637 | 2046 | 2061 | CGCT | eekd | 92 | 13515 | 13530 | 129 |
| | | | GATT | dddd | | | | |
| | | | TGTC | dddd | | | | |
| | | | CGGG | dkke | | | | |
| 568638 | 2056 | 2071 | GGTG | eekd | 82 | 13525 | 13540 | 130 |
| | | | ACAC | dddd | | | | |
| | | | ATCG | dddd | | | | |
| | | | CTGA | dkke | | | | |
| 594621 | 2022 | 2037 | CTGC | kkkd | 87 | 13491 | 13506 | 162 |
| | | | TGCT | dddd | | | | |
| | | | GGCC | dddd | | | | |
| | | | TTTG | dkkk | | | | |
| 594622 | 2027 | 2042 | GTTA | kkkd | 97 | 13496 | 13511 | 163 |
| | | | TCTG | dddd | | | | |
| | | | CTGC | dddd | | | | |
| | | | TGGC | dkkk | | | | |
| 594622 | 2027 | 2042 | GTTA | kkkd | 99 | 13496 | 13511 | 163 |
| | | | TCTG | dddd | | | | |
| | | | CTGC | dddd | | | | |
| | | | TGGC | dkkk | | | | |
| 594623 | 2032 | 2047 | GGGT | kkkd | 90 | 13501 | 13516 | 164 |
| | | | TGTT | dddd | | | | |
| | | | ATCT | dddd | | | | |
| | | | GCTG | dkkk | | | | |
| 594624 | 2046 | 2061 | CGCT | kkkd | 94 | 13515 | 13530 | 129 |
| | | | GATT | dddd | | | | |
| | | | TGTC | dddd | | | | |
| | | | CGGG | dkkk | | | | |
| 594625 | 2047 | 2062 | TCGC | kkkd | 91 | 13516 | 13531 | 165 |
| | | | TGAT | dddd | | | | |
| | | | TTGT | dddd | | | | |
| | | | CCGG | dkkk | | | | |
| 594625 | 2047 | 2062 | TCGC | kkkd | 97 | 13516 | 13531 | 165 |
| | | | TGAT | dddd | | | | |
| | | | TTGT | dddd | | | | |
| | | | CCGG | dkkk | | | | |
| 594626 | 2049 | 2064 | CATC | kkkd | 0 | 13518 | 13533 | 166 |
| | | | GCTG | dddd | | | | |
| | | | ATTT | dddd | | | | |
| | | | GTCC | dkkk | | | | |
| 594627 | 2053 | 2068 | GACA | kkkd | 92 | 13522 | 13537 | 167 |
| | | | CATC | dddd | | | | |
| | | | GCTG | dddd | | | | |
| | | | ATTT | dkkk | | | | |
| 594628 | 2073 | 2088 | AAAG | kkkd | 81 | 13542 | 13557 | 168 |
| | | | GTGG | dddd | | | | |
| | | | GAGA | dddd | | | | |
| | | | CTGG | dkkk | | | | |
| 594629 | 2082 | 2097 | ATTA | kkkd | 84 | 13551 | 13566 | 169 |
| | | | GAAG | dddd | | | | |
| | | | AAAA | dddd | | | | |
| | | | GGTG | dkkk | | | | |
| 594630 | 2090 | 2105 | GTCG | kkkd | 79 | 13559 | 13574 | 170 |
| | | | ACTC | dddd | | | | |
| | | | ATTA | dddd | | | | |
| | | | GAAG | dkkk | | | | |
| 594631 | 2095 | 2110 | TCAA | kkkd | 91 | 13564 | 13579 | 171 |
| | | | AGTC | dddd | | | | |
| | | | GACT | dddd | | | | |
| | | | CATT | dkkk | | | | |
| 594632 | 2099 | 2114 | CAGC | kkkd | 96 | 13568 | 13583 | 172 |
| | | | TCAA | dddd | | | | |
| | | | AGTC | dddd | | | | |
| | | | GACT | dkkk | | | | |
| 594641 | 2022 | 2037 | CTGC | eekd | 61 | 13491 | 13506 | 162 |
| | | | TGCT | dddd | | | | |
| | | | GGCC | dddd | | | | |
| | | | TTTG | dkke | | | | |
| 594642 | 2027 | 2042 | GTTA | eekd | 91 | 13496 | 13511 | 163 |
| | | | TCTG | dddd | | | | |
| | | | CTGC | dddd | | | | |
| | | | TGGC | dkke | | | | |
| 594643 | 2032 | 2047 | GGGT | eekd | 91 | 13501 | 13516 | 164 |
| | | | TGTT | dddd | | | | |
| | | | ATCT | dddd | | | | |
| | | | GCTG | dkke | | | | |
| 594644 | 2047 | 2062 | TCGC | eekd | 87 | 13516 | 13531 | 165 |
| | | | TGAT | dddd | | | | |
| | | | TTGT | dddd | | | | |
| | | | CCGG | dkke | | | | |
| 594645 | 2049 | 2064 | CATC | eekd | 79 | 13518 | 13533 | 166 |
| | | | GCTG | dddd | | | | |
| | | | ATTT | dddd | | | | |
| | | | GTCC | dkke | | | | |
| 594646 | 2053 | 2068 | GACA | eekd | 80 | 13522 | 13537 | 167 |
| | | | CATC | dddd | | | | |
| | | | GCTG | dddd | | | | |
| | | | ATTT | dkke | | | | |
| 594647 | 2073 | 2088 | AAAG | eekd | 62 | 13542 | 13557 | 168 |
| | | | GTGG | dddd | | | | |
| | | | GAGA | dddd | | | | |
| | | | CTGG | dkke | | | | |
| 609078 | 2020 | 2035 | GCTG | kkkd | 73 | 13489 | 13504 | 173 |
| | | | CTGG | dddd | | | | |
| | | | CCTT | dddd | | | | |
| | | | TGCC | dkkk | | | | |
| 609079 | 2021 | 2036 | TGCT | kkkd | 69 | 13490 | 13505 | 174 |
| | | | GCTG | dddd | | | | |
| | | | GCCT | dddd | | | | |
| | | | TTGC | dkkk | | | | |
| 609080 | 2023 | 2038 | TCTG | kkkd | 91 | 13492 | 13507 | 175 |
| | | | CTGC | dddd | | | | |
| | | | TGGC | dddd | | | | |
| | | | CTTT | dkkk | | | | |
| 609081 | 2024 | 2039 | ATCT | kkkd | 90 | 13493 | 13508 | 176 |
| | | | GCTG | dddd | | | | |
| | | | CTGG | dddd | | | | |
| | | | CCTT | dkkk | | | | |
| 609082 | 2025 | 2040 | TATC | kkkd | 84 | 13494 | 13509 | 177 |
| | | | TGCT | dddd | | | | |
| | | | GCTG | dddd | | | | |
| | | | GCCT | dkkk | | | | |
| 609083 | 2026 | 2041 | TTAT | kkkd | 91 | 13495 | 13510 | 178 |
| | | | CTGC | dddd | | | | |
| | | | TGCT | dddd | | | | |
| | | | GGCC | dkkk | | | | |
| 609084 | 2028 | 2043 | TGTT | kkkd | 89 | 13497 | 13512 | 179 |
| | | | ATCT | dddd | | | | |
| | | | GCTG | dddd | | | | |
| | | | CTGG | dkkk | | | | |
| 609085 | 2029 | 2044 | TTGT | kkkd | 91 | 13498 | 13513 | 180 |
| | | | TATC | dddd | | | | |
| | | | TGCT | dddd | | | | |
| | | | GCTG | dkkk | | | | |
| 609086 | 2030 | 2045 | GTTG | kkkd | 98 | 13499 | 13514 | 181 |
| | | | TTAT | dddd | | | | |
| | | | CTGC | dddd | | | | |
| | | | TGCT | dkkk | | | | |
| 609087 | 2031 | 2046 | GGTT | kkkd | 97 | 13500 | 13515 | 182 |
| | | | GTTA | dddd | | | | |
| | | | TCTG | dddd | | | | |
| | | | CTGC | dkkk | | | | |
| 609088 | 2048 | 2063 | ATCG | kkkd | 98 | 13517 | 13532 | 183 |
| | | | CTGA | dddd | | | | |
| | | | TTTG | dddd | | | | |
| | | | TCCG | dkkk | | | | |
| 609089 | 2050 | 2065 | ACAT | kkkd | 92 | 13519 | 13534 | 184 |
| | | | CGCT | dddd | | | | |
| | | | GATT | dddd | | | | |
| | | | TGTC | dkkk | | | | |
| 609090 | 2051 | 2066 | CACA | kkkd | 91 | 13520 | 13535 | 185 |
| | | | TCGC | dddd | | | | |
| | | | TGAT | dddd | | | | |
| | | | TTGT | dkkk | | | | |
| 609091 | 2052 | 2067 | ACAC | kkkd | 96 | 13521 | 13536 | 186 |
| | | | ATCG | dddd | | | | |
| | | | CTGA | dddd | | | | |
| | | | TTTG | dkkk | | | | |
| 609092 | 2054 | 2069 | TGAC | kkkd | 34 | 13523 | 13538 | 187 |
| | | | ACAT | dddd | | | | |
| | | | CGCT | dddd | | | | |
| | | | GATT | dkkk | | | | |
| 609093 | 2055 | 2070 | GTGA | kkkd | 78 | 13524 | 13539 | 188 |
| | | | CACA | dddd | | | | |
| | | | TCGC | dddd | | | | |
| | | | TGAT | dkkk | | | | |
| 609094 | 2056 | 2071 | GGTG | kkkd | 93 | 13525 | 13540 | 130 |
| | | | ACAC | dddd | | | | |
| | | | ATCG | dddd | | | | |
| | | | CTGA | dkkk | | | | |
| 609095 | 2057 | 2072 | GGGT | kkkd | 96 | 13526 | 13541 | 189 |
| | | | GACA | dddd | | | | |
| | | | CATC | dddd | | | | |
| | | | GCTG | dkkk | | | | |
| 609096 | 2074 | 2089 | AAAA | kkkd | 70 | 13543 | 13558 | 190 |
| | | | GGTG | dddd | | | | |
| | | | GGAG | dddd | | | | |
| | | | ACTG | dkkk | | | | |
| 609097 | 2075 | 2090 | GAAA | kkkd | 80 | 13544 | 13559 | 131 |
| | | | AGGT | dddd | | | | |
| | | | GGGA | dddd | | | | |
| | | | GACT | dkkk | | | | |
| 609098 | 2076 | 2091 | AGAA | kkkd | 85 | 13545 | 13560 | 191 |
| | | | AAGG | dddd | | | | |
| | | | TGGG | dddd | | | | |
| | | | AGAC | dkkk | | | | |
| 609099 | 2080 | 2095 | TAGA | kkkd | 90 | 13549 | 13564 | 192 |
| | | | AGAA | dddd | | | | |
| | | | AAGG | dddd | | | | |
| | | | TGGG | dkkk | | | | |
| 609100 | 2081 | 2096 | TTAG | kkkd | 95 | 13550 | 13565 | 193 |
| | | | AAGA | dddd | | | | |
| | | | AAAG | dddd | | | | |
| | | | GTGG | dkkk | | | | |
| 609101 | 2083 | 2098 | CATT | kkkd | 76 | 13552 | 13567 | 194 |
| | | | AGAA | dddd | | | | |
| | | | GAAA | dddd | | | | |
| | | | AGGT | dkkk | | | | |
| 609102 | 2084 | 2099 | TCAT | kkkd | 97 | 13553 | 13568 | 195 |
| | | | TAGA | dddd | | | | |
| | | | AGAA | dddd | | | | |
| | | | AAGG | dkkk | | | | |
| 609103 | 2085 | 2100 | CTCA | kkkd | 87 | 13554 | 13569 | 196 |
| | | | TTAG | dddd | | | | |
| | | | AAGA | dddd | | | | |
| | | | AAAG | dkkk | | | | |
| 609104 | 2086 | 2101 | ACTC | kkkd | 70 | 13555 | 13570 | 197 |
| | | | ATTA | dddd | | | | |
| | | | GAAG | dddd | | | | |
| | | | AAAA | dkkk | | | | |
| 609105 | 2087 | 2102 | GACT | kkkd | 93 | 13556 | 13571 | 198 |
| | | | CATT | dddd | | | | |
| | | | AGAA | dddd | | | | |
| | | | GAAA | dkkk | | | | |
| 609106 | 2088 | 2103 | CGAC | kkkd | 98 | 13557 | 13572 | 132 |
| | | | TCAT | dddd | | | | |
| | | | TAGA | dddd | | | | |
| | | | AGAA | dkkk | | | | |
| 609107 | 2089 | 2104 | TCGA | kkkd | 97 | 13558 | 13573 | 199 |
| | | | CTCA | dddd | | | | |
| | | | TTAG | dddd | | | | |
| | | | AAGA | dkkk | | | | |
| 609108 | 2091 | 2106 | AGTC | kkkd | 97 | 13560 | 13575 | 200 |
| | | | GACT | dddd | | | | |
| | | | CATT | dddd | | | | |
| | | | AGAA | dkkk | | | | |
| 609109 | 2092 | 2107 | AAGT | kkkd | 96 | 13561 | 13576 | 201 |
| | | | CGAC | dddd | | | | |
| | | | TCAT | dddd | | | | |
| | | | TAGA | dkkk | | | | |
| 609110 | 2093 | 2108 | AAAG | kkkd | 96 | 13562 | 13577 | 202 |
| | | | TCGA | dddd | | | | |
| | | | CTCA | dddd | | | | |
| | | | TTAG | dkkk | | | | |
| 609111 | 2094 | 2109 | CAAA | kkkd | 92 | 13563 | 13578 | 203 |
| | | | GTCG | dddd | | | | |
| | | | ACTC | dddd | | | | |
| | | | ATTA | dkkk | | | | |
| 609112 | 2096 | 2111 | CTCA | kkkd | 93 | 13565 | 13580 | 204 |
| | | | AAGT | dddd | | | | |
| | | | CGAC | dddd | | | | |
| | | | TCAT | dkkk | | | | |
| 609113 | 2097 | 2112 | GCTC | kkkd | 97 | 13566 | 13581 | 205 |
| | | | AAAG | dddd | | | | |
| | | | TCGA | dddd | | | | |
| | | | CTCA | dkkk | | | | |
| 609114 | 2098 | 2113 | AGCT | kkkd | 95 | 13567 | 13582 | 206 |
| | | | CAAA | dddd | | | | |
| | | | GTCG | dddd | | | | |
| | | | ACTC | dkkk | | | | |
| 609115 | 2020 | 2035 | GCTG | eekd | 71 | 13489 | 13504 | 173 |
| | | | CTGG | dddd | | | | |
| | | | CCTT | dddd | | | | |
| | | | TGCC | dkke | | | | |
| 609116 | 2021 | 2036 | TGCT | eekd | 47 | 13490 | 13505 | 174 |
| | | | GCTG | dddd | | | | |
| | | | GCCT | dddd | | | | |
| | | | TTGC | dkke | | | | |
| 609117 | 2023 | 2038 | TCTG | eekd | 74 | 13492 | 13507 | 175 |
| | | | CTGC | dddd | | | | |
| | | | TGGC | dddd | | | | |
| | | | CTTT | dkke | | | | |
| 609118 | 2024 | 2039 | ATCT | eekd | 81 | 13493 | 13508 | 176 |
| | | | GCTG | dddd | | | | |
| | | | CTGG | dddd | | | | |
| | | | CCTT | dkke | | | | |
| 609119 | 2025 | 2040 | TATC | eekd | 76 | 13494 | 13509 | 177 |
| | | | TGCT | dddd | | | | |
| | | | GCTG | dddd | | | | |
| | | | GCCT | dkke | | | | |
| 609120 | 2026 | 2041 | TTAT | eekd | 56 | 13495 | 13510 | 178 |
| | | | CTGC | dddd | | | | |
| | | | TGCT | dddd | | | | |
| | | | GGCC | dkke | | | | |
| 609121 | 2028 | 2043 | TGTT | eekd | 73 | 13497 | 13512 | 179 |
| | | | ATCT | dddd | | | | |
| | | | GCTG | dddd | | | | |
| | | | CTGG | dkke | | | | |
| 609122 | 2029 | 2044 | TTGT | eekd | 87 | 13498 | 13513 | 180 |
| | | | TATC | dddd | | | | |
| | | | TGCT | dddd | | | | |
| | | | GCTG | dkke | | | | |
| 609123 | 2030 | 2045 | GTTG | eekd | 92 | 13499 | 13514 | 181 |
| | | | TTAT | dddd | | | | |
| | | | CTGC | dddd | | | | |
| | | | TGCT | dkke | | | | |
| 609124 | 2031 | 2046 | GGTT | eekd | 90 | 13500 | 13515 | 182 |
| | | | GTTA | dddd | | | | |
| | | | TCTG | dddd | | | | |
| | | | CTGC | dkke | | | | |
| 609125 | 2048 | 2063 | ATCG | eekd | 91 | 13517 | 13532 | 183 |
| | | | CTGA | dddd | | | | |
| | | | TTTG | dddd | | | | |
| | | | TCCG | dkke | | | | |
| 609126 | 2050 | 2065 | ACAT | eekd | 66 | 13519 | 13534 | 184 |
| | | | CGCT | dddd | | | | |
| | | | GATT | dddd | | | | |
| | | | TGTC | dkke | | | | |
| 609127 | 2051 | 2066 | CACA | eekd | 79 | 13520 | 13535 | 185 |
| | | | TCGC | dddd | | | | |
| | | | TGAT | dddd | | | | |
| | | | TTGT | dkke | | | | |
| 609128 | 2052 | 2067 | ACAC | eekd | 72 | 13521 | 13536 | 186 |
| | | | ATCG | dddd | | | | |
| | | | CTGA | dddd | | | | |
| | | | TTTG | dkke | | | | |
| 609129 | 2054 | 2069 | TGAC | eekd | 60 | 13523 | 13538 | 187 |
| | | | ACAT | dddd | | | | |
| | | | CGCT | dddd | | | | |
| | | | GATT | dkke | | | | |
| 609130 | 2055 | 2070 | GTGA | eekd | 77 | 13524 | 13539 | 188 |
| | | | CACA | dddd | | | | |
| | | | TCGC | dddd | | | | |
| | | | TGAT | dkke | | | | |
| 609131 | 2057 | 2072 | GGGT | eekd | 85 | 13526 | 13541 | 189 |
| | | | GACA | dddd | | | | |
| | | | CATC | dddd | | | | |
| | | | GCTG | dkke | | | | |
| 609132 | 2020 | 2035 | GCTG | eekk | 47 | 13489 | 13504 | 173 |
| | | | CTGG | dddd | | | | |
| | | | CCTT | dddk | | | | |
| | | | TGCC | keee | | | | |
| 609133 | 2021 | 2036 | TGCT | eekk | 44 | 13490 | 13505 | 174 |
| | | | GCTG | dddd | | | | |
| | | | GCCT | dddk | | | | |
| | | | TTGC | keee | | | | |
| 609134 | 2022 | 2037 | CTGC | eekk | 62 | 13491 | 13506 | 162 |
| | | | TGCT | dddd | | | | |
| | | | GGCC | dddk | | | | |
| | | | TTTG | keee | | | | |
| 609135 | 2023 | 2038 | TCTG | eekk | 59 | 13492 | 13507 | 175 |
| | | | CTGC | dddd | | | | |
| | | | TGGC | dddk | | | | |
| | | | CTTT | keee | | | | |
| 609136 | 2024 | 2039 | ATCT | eekk | 70 | 13493 | 13508 | 176 |
| | | | GCTG | dddd | | | | |
| | | | CTGG | dddk | | | | |
| | | | CCTT | keee | | | | |
| 609137 | 2025 | 2040 | TATC | eekk | 59 | 13494 | 13509 | 177 |
| | | | TGCT | dddd | | | | |
| | | | GCTG | dddk | | | | |
| | | | GCCT | keee | | | | |
| 609138 | 2026 | 2041 | TTAT | eekk | 78 | 13495 | 13510 | 178 |
| | | | CTGC | dddd | | | | |
| | | | TGCT | dddk | | | | |
| | | | GGCC | keee | | | | |
| 609139 | 2027 | 2042 | GTTA | eekk | 79 | 13496 | 13511 | 163 |
| | | | TCTG | dddd | | | | |
| | | | CTGC | dddk | | | | |
| | | | TGGC | keee | | | | |
| 609140 | 2028 | 2043 | TGTT | eekk | 83 | 13497 | 13512 | 179 |
| | | | ATCT | dddd | | | | |
| | | | GCTG | dddk | | | | |
| | | | CTGG | keee | | | | |
| 609141 | 2029 | 2044 | TTGT | eekk | 67 | 13498 | 13513 | 180 |
| | | | TATC | dddd | | | | |
| | | | TGCT | dddk | | | | |
| | | | GCTG | keee | | | | |
| 609142 | 2030 | 2045 | GTTG | eekk | 68 | 13499 | 13514 | 181 |
| | | | TTAT | dddd | | | | |
| | | | CTGC | dddk | | | | |
| | | | TGCT | keee | | | | |
| 609143 | 2031 | 2046 | GGTT | eekk | 81 | 13500 | 13515 | 182 |
| | | | GTTA | dddd | | | | |
| | | | TCTG | dddk | | | | |
| | | | CTGC | keee | | | | |
| 609144 | 2032 | 2047 | GGGT | eekk | 81 | 13501 | 13516 | 164 |
| | | | TGTT | dddd | | | | |
| | | | ATCT | dddk | | | | |
| | | | GCTG | keee | | | | |
| 609145 | 2046 | 2061 | CGCT | eekk | 53 | 13515 | 13530 | 129 |
| | | | GATT | dddd | | | | |
| | | | TGTC | dddk | | | | |
| | | | CGGG | keee | | | | |
| 609146 | 2047 | 2062 | TCGC | eekk | 80 | 13516 | 13531 | 165 |
| | | | TGAT | dddd | | | | |
| | | | TTGT | dddk | | | | |
| | | | CCGG | keee | | | | |
| 609147 | 2048 | 2063 | ATCG | eekk | 88 | 13517 | 13532 | 183 |
| | | | CTGA | dddd | | | | |
| | | | TTTG | dddk | | | | |
| | | | TCCG | keee | | | | |
| 609148 | 2049 | 2064 | CATC | eekk | 75 | 13518 | 13533 | 166 |
| | | | GCTG | dddd | | | | |
| | | | ATTT | dddk | | | | |
| | | | GTCC | keee | | | | |
| 609149 | 2050 | 2065 | ACAT | eekk | 64 | 13519 | 13534 | 184 |
| | | | CGCT | dddd | | | | |
| | | | GATT | dddk | | | | |
| | | | TGTC | keee | | | | |
| 609150 | 2051 | 2066 | CACA | eekk | 77 | 13520 | 13535 | 185 |
| | | | TCGC | dddd | | | | |
| | | | TGAT | dddk | | | | |
| | | | TTGT | keee | | | | |
| 609151 | 2052 | 2067 | ACAC | eekk | 57 | 13521 | 13536 | 186 |
| | | | ATCG | dddd | | | | |
| | | | CTGA | dddk | | | | |
| | | | TTTG | keee | | | | |
| 609152 | 2053 | 2068 | GACA | eekk | 52 | 13522 | 13537 | 167 |
| | | | CATC | dddd | | | | |
| | | | GCTG | dddk | | | | |
| | | | ATTT | keee | | | | |
| 609153 | 2054 | 2069 | TGAC | eekk | 37 | 13523 | 13538 | 187 |
| | | | ACAT | dddd | | | | |
| | | | CGCT | dddk | | | | |
| | | | GATT | keee | | | | |
| 609154 | 2055 | 2070 | GTGA | eekk | 50 | 13524 | 13539 | 188 |
| | | | CACA | dddd | | | | |
| | | | TCGC | dddk | | | | |
| | | | TGAT | keee | | | | |
| 609155 | 2056 | 2071 | GGTG | eekk | 60 | 13525 | 13540 | 130 |
| | | | ACAC | dddd | | | | |
| | | | ATCG | dddk | | | | |
| | | | CTGA | keee | | | | |
| 609156 | 2057 | 2072 | GGGT | eekk | 54 | 13526 | 13541 | 189 |
| | | | GACA | dddd | | | | |
| | | | CATC | dddk | | | | |
| | | | GCTG | keee | | | | |
| 609157 | 2073 | 2088 | AAAG | eekk | 40 | 13542 | 13557 | 168 |
| | | | GTGG | dddd | | | | |
| | | | GAGA | dddk | | | | |
| | | | CTGG | keee | | | | |
| 609158 | 2020 | 2035 | GCTG | eekk | 77 | 13489 | 13504 | 173 |
| | | | CTGG | dddd | | | | |
| | | | CCTT | dddd | | | | |
| | | | TGCC | kkee | | | | |
| 609159 | 2021 | 2036 | TGCT | eekk | 85 | 13490 | 13505 | 174 |
| | | | GCTG | dddd | | | | |
| | | | GCCT | dddd | | | | |
| | | | TTGC | kkee | | | | |
| 609160 | 2022 | 2037 | CTGC | eekk | 81 | 13491 | 13506 | 162 |
| | | | TGCT | dddd | | | | |
| | | | GGCC | dddd | | | | |
| | | | TTTG | kkee | | | | |
| 609161 | 2023 | 2038 | TCTG | eekk | 91 | 13492 | 13507 | 175 |
| | | | CTGC | dddd | | | | |
| | | | TGGC | dddd | | | | |
| | | | CTTT | kkee | | | | |
| 609162 | 2024 | 2039 | ATCT | eekk | 92 | 13493 | 13508 | 176 |
| | | | GCTG | dddd | | | | |
| | | | CTGG | dddd | | | | |
| | | | CCTT | kkee | | | | |
| 609163 | 2025 | 2040 | TATC | eekk | 83 | 13494 | 13509 | 177 |
| | | | TGCT | dddd | | | | |
| | | | GCTG | dddd | | | | |
| | | | GCCT | kkee | | | | |
| 609164 | 2026 | 2041 | TTAT | eekk | 93 | 13495 | 13510 | 178 |
| | | | CTGC | dddd | | | | |
| | | | TGCT | dddd | | | | |
| | | | GGCC | kkee | | | | |
| 609165 | 2027 | 2042 | GTTA | eekk | 93 | 13496 | 13511 | 163 |
| | | | TCTG | dddd | | | | |
| | | | CTGC | dddd | | | | |
| | | | TGGC | kkee | | | | |
| 609166 | 2028 | 2043 | TGTT | eekk | 98 | 13497 | 13512 | 179 |
| | | | ATCT | dddd | | | | |
| | | | GCTG | dddd | | | | |
| | | | CTGG | kkee | | | | |
| 609167 | 2029 | 2044 | TTGT | eekk | 95 | 13498 | 13513 | 180 |
| | | | TATC | dddd | | | | |
| | | | TGCT | dddd | | | | |
| | | | GCTG | kkee | | | | |
| 609168 | 2030 | 2045 | GTTG | eekk | 95 | 13499 | 13514 | 181 |
| | | | TTAT | dddd | | | | |
| | | | CTGC | dddd | | | | |
| | | | TGCT | kkee | | | | |
| 609169 | 2031 | 2046 | GGTT | eekk | 95 | 13500 | 13515 | 182 |
| | | | GTTA | dddd | | | | |
| | | | TCTG | dddd | | | | |
| | | | CTGC | kkee | | | | |
| 609170 | 2032 | 2047 | GGGT | eekk | 96 | 13501 | 13516 | 164 |
| | | | TGTT | dddd | | | | |
| | | | ATCT | dddd | | | | |
| | | | GCTG | kkee | | | | |
| 609171 | 2046 | 2061 | CGCT | eekk | 90 | 13515 | 13530 | 129 |
| | | | GATT | dddd | | | | |
| | | | TGTC | dddd | | | | |
| | | | CGGG | kkee | | | | |
| 609172 | 2047 | 2062 | TCGC | eekk | 92 | 13516 | 13531 | 165 |
| | | | TGAT | dddd | | | | |
| | | | TTGT | dddd | | | | |
| | | | CCGG | kkee | | | | |
| 609173 | 2048 | 2063 | ATCG | eekk | 94 | 13517 | 13532 | 183 |
| | | | CTGA | dddd | | | | |
| | | | TTTG | dddd | | | | |
| | | | TCCG | kkee | | | | |
| 609174 | 2049 | 2064 | CATC | eekk | 96 | 13518 | 13533 | 166 |
| | | | GCTG | dddd | | | | |
| | | | ATTT | dddd | | | | |
| | | | GTCC | kkee | | | | |
| 609175 | 2050 | 2065 | ACAT | eekk | 91 | 13519 | 13534 | 184 |
| | | | CGCT | dddd | | | | |
| | | | GATT | dddd | | | | |
| | | | TGTC | kkee | | | | |
| 609176 | 2051 | 2066 | CACA | eekk | 94 | 13520 | 13535 | 185 |
| | | | TCGC | dddd | | | | |
| | | | TGAT | dddd | | | | |
| | | | TTGT | kkee | | | | |
| 609177 | 2052 | 2067 | ACAC | eekk | 96 | 13521 | 13536 | 186 |
| | | | ATCG | dddd | | | | |
| | | | CTGA | dddd | | | | |
| | | | TTTG | kkee | | | | |
| 609178 | 2053 | 2068 | GACA | eekk | 88 | 13522 | 13537 | 167 |
| | | | CATC | dddd | | | | |
| | | | GCTG | dddd | | | | |
| | | | ATTT | kkee | | | | |
| 609179 | 2054 | 2069 | TGAC | eekk | 84 | 13523 | 13538 | 187 |
| | | | ACAT | dddd | | | | |
| | | | CGCT | dddd | | | | |
| | | | GATT | kkee | | | | |
| 609180 | 2055 | 2070 | GTGA | eekk | 83 | 13524 | 13539 | 188 |
| | | | CACA | dddd | | | | |
| | | | TCGC | dddd | | | | |
| | | | TGAT | kkee | | | | |
| 609181 | 2056 | 2071 | GGTG | eekk | 87 | 13525 | 13540 | 130 |
| | | | ACAC | dddd | | | | |
| | | | ATCG | dddd | | | | |
| | | | CTGA | kkee | | | | |
| 609182 | 2057 | 2072 | GGGT | eekk | 90 | 13526 | 13541 | 189 |
| | | | GACA | dddd | | | | |
| | | | CATC | dddd | | | | |
| | | | GCTG | kkee | | | | |
| 609183 | 2073 | 2088 | AAAG | eekk | 82 | 13542 | 13557 | 168 |
| | | | GTGG | dddd | | | | |
| | | | GAGA | dddd | | | | |
| | | | CTGG | kkee | | | | |
| 609184 | 2020 | 2035 | GCTG | ekkd | 84 | 13489 | 13504 | 173 |
| | | | CTGG | dddd | | | | |
| | | | CCTT | dddd | | | | |
| | | | TGCC | kkee | | | | |
| 609185 | 2021 | 2036 | TGCT | ekkd | 88 | 13490 | 13505 | 174 |
| | | | GCTG | dddd | | | | |
| | | | GCCT | dddd | | | | |
| | | | TTGC | kkee | | | | |
| 609186 | 2022 | 2037 | CTGC | ekkd | 88 | 13491 | 13506 | 162 |
| | | | TGCT | dddd | | | | |
| | | | GGCC | dddd | | | | |
| | | | TTTG | kkee | | | | |
| 609187 | 2023 | 2038 | TCTG | ekkd | 74 | 13492 | 13507 | 175 |
| | | | CTGC | dddd | | | | |
| | | | TGGC | dddd | | | | |
| | | | CTTT | kkee | | | | |
| 609188 | 2024 | 2039 | ATCT | ekkd | 90 | 13493 | 13508 | 176 |
| | | | GCTG | dddd | | | | |
| | | | CTGG | dddd | | | | |
| | | | CCTT | kkee | | | | |
| 609189 | 2025 | 2040 | TATC | ekkd | 91 | 13494 | 13509 | 177 |
| | | | TGCT | dddd | | | | |
| | | | GCTG | dddd | | | | |
| | | | GCCT | kkee | | | | |
| 609190 | 2026 | 2041 | TTAT | ekkd | 87 | 13495 | 13510 | 178 |
| | | | CTGC | dddd | | | | |
| | | | TGCT | dddd | | | | |
| | | | GGCC | kkee | | | | |
| 609191 | 2027 | 2042 | GTTA | ekkd | 97 | 13496 | 13511 | 163 |
| | | | TCTG | dddd | | | | |
| | | | CTGC | dddd | | | | |
| | | | TGGC | kkee | | | | |
| 609192 | 2028 | 2043 | TGTT | ekkd | 95 | 13497 | 13512 | 179 |
| | | | ATCT | dddd | | | | |
| | | | GCTG | dddd | | | | |
| | | | CTGG | kkee | | | | |
| 609193 | 2029 | 2044 | TTGT | ekkd | 96 | 13498 | 13513 | 180 |
| | | | TATC | dddd | | | | |
| | | | TGCT | dddd | | | | |
| | | | GCTG | kkee | | | | |
| 609194 | 2030 | 2045 | GTTG | ekkd | 97 | 13499 | 13514 | 181 |
| | | | TTAT | dddd | | | | |
| | | | CTGC | dddd | | | | |
| | | | TGCT | kkee | | | | |
| 609195 | 2031 | 2046 | GGTT | ekkd | 97 | 13500 | 13515 | 182 |
| | | | GTTA | dddd | | | | |
| | | | TCTG | dddd | | | | |
| | | | CTGC | kkee | | | | |
| 609196 | 2032 | 2047 | GGGT | ekkd | 98 | 13501 | 13516 | 164 |
| | | | TGTT | dddd | | | | |
| | | | ATCT | dddd | | | | |
| | | | GCTG | kkee | | | | |
| 609197 | 2046 | 2061 | CGCT | ekkd | 96 | 13515 | 13530 | 129 |
| | | | GATT | dddd | | | | |
| | | | TGTC | dddd | | | | |
| | | | CGGG | kkee | | | | |
| 609198 | 2047 | 2062 | TCGC | ekkd | 95 | 13516 | 13531 | 165 |
| | | | TGAT | dddd | | | | |
| | | | TTGT | dddd | | | | |
| | | | CCGG | kkee | | | | |
| 609199 | 2048 | 2063 | ATCG | ekkd | 96 | 13517 | 13532 | 183 |
| | | | CTGA | dddd | | | | |
| | | | TTTG | dddd | | | | |
| | | | TCCG | kkee | | | | |
| 609200 | 2049 | 2064 | CATC | ekkd | 94 | 13518 | 13533 | 166 |
| | | | GCTG | dddd | | | | |
| | | | ATTT | dddd | | | | |
| | | | GTCC | kkee | | | | |
| 609201 | 2050 | 2065 | ACAT | ekkd | 94 | 13519 | 13534 | 184 |
| | | | CGCT | dddd | | | | |
| | | | GATT | dddd | | | | |
| | | | TGTC | kkee | | | | |
| 609202 | 2051 | 2066 | CACA | ekkd | 94 | 13520 | 13535 | 185 |
| | | | TCGC | dddd | | | | |
| | | | TGAT | dddd | | | | |
| | | | TTGT | kkee | | | | |
| 609203 | 2052 | 2067 | ACAC | ekkd | 91 | 13521 | 13536 | 186 |
| | | | ATCG | dddd | | | | |
| | | | CTGA | dddd | | | | |
| | | | TTTG | kkee | | | | |
| 609204 | 2053 | 2068 | GACA | ekkd | 94 | 13522 | 13537 | 167 |
| | | | CATC | dddd | | | | |
| | | | GCTG | dddd | | | | |
| | | | ATTT | kkee | | | | |
| 609205 | 2054 | 2069 | TGAC | ekkd | 87 | 13523 | 13538 | 187 |
| | | | ACAT | dddd | | | | |
| | | | CGCT | dddd | | | | |
| | | | GATT | kkee | | | | |
| 609206 | 2055 | 2070 | GTGA | ekkd | 91 | 13524 | 13539 | 188 |
| | | | CACA | dddd | | | | |
| | | | TCGC | dddd | | | | |
| | | | TGAT | kkee | | | | |
| 609207 | 2056 | 2071 | GGTG | ekkd | 93 | 13525 | 13540 | 130 |
| | | | ACAC | dddd | | | | |
| | | | ATCG | dddd | | | | |
| | | | CTGA | kkee | | | | |
| 609208 | 2057 | 2072 | GGGT | ekkd | 97 | 13526 | 13541 | 189 |
| | | | GACA | dddd | | | | |
| | | | CATC | dddd | | | | |
| | | | GCTG | kkee | | | | |
| 609209 | 2073 | 2088 | AAAG | ekkd | 95 | 13542 | 13557 | 168 |
| | | | GTGG | dddd | | | | |
| | | | GAGA | dddd | | | | |
| | | | CTGG | kkee | | | | |
| 609983 | 1983 | 2002 | AGGC | eeee | 75 | 13452 | 13471 | 207 |
| | | | CTTG | eddd | | | | |
| | | | CCAG | dddd | | | | |
| | | | GCAC | ddde | | | | |
| | | | TGTG | eeee | | | | |
| 609984 | 1984 | 2003 | GAGG | eeee | 54 | 13453 | 13472 | 208 |
| | | | CCTT | eddd | | | | |
| | | | GCCA | dddd | | | | |
| | | | GGCA | ddde | | | | |
| | | | CTGT | eeee | | | | |
| 609985 | 1985 | 2004 | AGAG | eeee | 63 | 13454 | 13473 | 209 |
| | | | GCCT | eddd | | | | |
| | | | TGCC | dddd | | | | |
| | | | AGGC | ddde | | | | |
| | | | ACTG | eeee | | | | |
| 609986 | 1986 | 2005 | CAGA | eeee | 63 | 13455 | 13474 | 210 |
| | | | GGCC | eddd | | | | |
| | | | TTGC | dddd | | | | |
| | | | CAGG | ddde | | | | |
| | | | CACT | eeee | | | | |
| 609987 | 1987 | 2006 | GCAG | eeee | 36 | 13456 | 13475 | 211 |
| | | | AGGC | eddd | | | | |
| | | | CTTG | dddd | | | | |
| | | | CCAG | ddde | | | | |
| | | | GCAC | eeee | | | | |
| 609988 | 1988 | 2007 | GGCA | eeee | 48 | 13457 | 13476 | 212 |
| | | | GAGG | eddd | | | | |
| | | | CCTT | dddd | | | | |
| | | | GCCA | ddde | | | | |
| | | | GGCA | eeee | | | | |
| 609989 | 1989 | 2008 | GGGC | eeee | 55 | 13458 | 13477 | 213 |
| | | | AGAG | eddd | | | | |
| | | | GCCT | dddd | | | | |
| | | | TGCC | ddde | | | | |
| | | | AGGC | eeee | | | | |
| 609990 | 2007 | 2026 | CTTT | eeee | 38 | 13476 | 13495 | 214 |
| | | | GCCT | eddd | | | | |
| | | | CAAA | dddd | | | | |
| | | | GGCC | ddde | | | | |
| | | | AGGG | eeee | | | | |
| 609991 | 2008 | 2027 | CCTT | eeee | 12 | 13477 | 13496 | 215 |
| | | | TGCC | eddd | | | | |
| | | | TCAA | dddd | | | | |
| | | | AGGC | ddde | | | | |
| | | | CAGG | eeee | | | | |
| 609992 | 2009 | 2028 | GCCT | eeee | 11 | 13478 | 13497 | 216 |
| | | | TTGC | eddd | | | | |
| | | | CTCA | dddd | | | | |
| | | | AAGG | ddde | | | | |
| | | | CCAG | eeee | | | | |
| 609993 | 2010 | 2029 | GGCC | eeee | 16 | 13479 | 13498 | 217 |
| | | | TTTG | eddd | | | | |
| | | | CCTC | dddd | | | | |
| | | | AAAG | ddde | | | | |
| | | | GCCA | eeee | | | | |
| 609994 | 2011 | 2030 | TGGC | eeee | 13 | 13480 | 13499 | 218 |
| | | | CTTT | eddd | | | | |
| | | | GCCT | dddd | | | | |
| | | | CAAA | ddde | | | | |
| | | | GGCC | eeee | | | | |
| 609995 | 2012 | 2031 | CTGG | eeee | 13 | 13481 | 13500 | 219 |
| | | | CCTT | eddd | | | | |
| | | | TGCC | dddd | | | | |
| | | | TCAA | ddde | | | | |
| | | | AGGC | eeee | | | | |
| 609996 | 2013 | 2032 | GCTG | eeee | 35 | 13482 | 13501 | 220 |
| | | | GCCT | eddd | | | | |
| | | | TTGC | dddd | | | | |
| | | | CTCA | ddde | | | | |
| | | | AAGG | eeee | | | | |
| 609997 | 2014 | 2033 | TGCT | eeee | 20 | 13483 | 13502 | 221 |
| | | | GGCC | eddd | | | | |
| | | | TTTG | dddd | | | | |
| | | | CCTC | ddde | | | | |
| | | | AAAG | eeee | | | | |
| 609998 | 2015 | 2034 | CTGC | eeee | 33 | 13484 | 13503 | 222 |
| | | | TGGC | eddd | | | | |
| | | | CTTT | dddd | | | | |
| | | | GCCT | ddde | | | | |
| | | | CAAA | eeee | | | | |
| 609999 | 2016 | 2035 | GCTG | eeee | 69 | 13485 | 13504 | 223 |
| | | | CTGG | eddd | | | | |
| | | | CCTT | dddd | | | | |
| | | | TGCC | ddde | | | | |
| | | | TCAA | eeee | | | | |
| 610000 | 2017 | 2036 | TGCT | eeee | 55 | 13486 | 13505 | 224 |
| | | | GCTG | eddd | | | | |
| | | | GCCT | dddd | | | | |
| | | | TTGC | ddde | | | | |
| | | | CTCA | eeee | | | | |
| 610001 | 2018 | 2037 | CTGC | eeee | 73 | 13487 | 13506 | 225 |
| | | | TGCT | eddd | | | | |
| | | | GGCC | dddd | | | | |
| | | | TTTG | ddde | | | | |
| | | | CCTC | eeee | | | | |
| 610002 | 2019 | 2038 | TCTG | eeee | 72 | 13488 | 13507 | 226 |
| | | | CTGC | eddd | | | | |
| | | | TGGC | dddd | | | | |
| | | | CTTT | ddde | | | | |
| | | | GCCT | eeee | | | | |
| 610003 | 2020 | 2039 | ATCT | eeee | 69 | 13489 | 13508 | 227 |
| | | | GCTG | eddd | | | | |
| | | | CTGG | dddd | | | | |
| | | | CCTT | ddde | | | | |
| | | | TGCC | eeee | | | | |
| 610004 | 2021 | 2040 | TATC | eeee | 56 | 13490 | 13509 | 228 |
| | | | TGCT | eddd | | | | |
| | | | GCTG | dddd | | | | |
| | | | GCCT | ddde | | | | |
| | | | TTGC | eeee | | | | |
| 610005 | 2022 | 2041 | TTAT | eeee | 29 | 13491 | 13510 | 229 |
| | | | CTGC | eddd | | | | |
| | | | TGCT | dddd | | | | |
| | | | GGCC | ddde | | | | |
| | | | TTTG | eeee | | | | |
| 610006 | 2023 | 2042 | GTTA | eeee | 74 | 13492 | 13511 | 230 |
| | | | TCTG | eddd | | | | |
| | | | CTGC | dddd | | | | |
| | | | TGGC | ddde | | | | |
| | | | CTTT | eeee | | | | |
| 610007 | 2024 | 2043 | TGTT | eeee | 74 | 13493 | 13512 | 231 |
| | | | ATCT | eddd | | | | |
| | | | GCTG | dddd | | | | |
| | | | CTGG | ddde | | | | |
| | | | CCTT | eeee | | | | |
| 610008 | 2025 | 2044 | TTGT | eeee | 72 | 13494 | 13513 | 232 |
| | | | TATC | eddd | | | | |
| | | | TGCT | dddd | | | | |
| | | | GCTG | ddde | | | | |
| | | | GCCT | eeee | | | | |
| 610009 | 2026 | 2045 | GTTG | eeee | 73 | 13495 | 13514 | 233 |
| | | | TTAT | eddd | | | | |
| | | | CTGC | dddd | | | | |
| | | | TGCT | ddde | | | | |
| | | | GGCC | eeee | | | | |
| 610010 | 2027 | 2046 | GGTT | eeee | 83 | 13496 | 13515 | 234 |
| | | | GTTA | eddd | | | | |
| | | | TCTG | dddd | | | | |
| | | | CTGC | ddde | | | | |
| | | | TGGC | eeee | | | | |
| 610011 | 2028 | 2047 | GGGT | eeee | 76 | 13497 | 13516 | 235 |
| | | | TGTT | eddd | | | | |
| | | | ATCT | dddd | | | | |
| | | | GCTG | ddde | | | | |
| | | | CTGG | eeee | | | | |
| 610012 | 2046 | 2065 | ACAT | eeee | 79 | 13515 | 13534 | 236 |
| | | | CGCT | eddd | | | | |
| | | | GATT | dddd | | | | |
| | | | TGTC | ddde | | | | |
| | | | CGGG | eeee | | | | |
| 610013 | 2047 | 2066 | CACA | eeee | 79 | 13516 | 13535 | 237 |
| | | | TCGC | eddd | | | | |
| | | | TGAT | dddd | | | | |
| | | | TTGT | ddde | | | | |
| | | | CCGG | eeee | | | | |
| 610014 | 2048 | 2067 | ACAC | eeee | 77 | 13517 | 13536 | 238 |
| | | | ATCG | eddd | | | | |
| | | | CTGA | dddd | | | | |
| | | | TTTG | ddde | | | | |
| | | | TCCG | eeee | | | | |
| 610015 | 2049 | 2068 | GACA | eeee | 89 | 13518 | 13537 | 239 |
| | | | CATC | eddd | | | | |
| | | | GCTG | dddd | | | | |
| | | | ATTT | ddde | | | | |
| | | | GTCC | eeee | | | | |
| 610016 | 2050 | 2069 | TGAC | eeee | 83 | 13519 | 13538 | 240 |
| | | | ACAT | eddd | | | | |
| | | | CGCT | dddd | | | | |
| | | | GATT | ddde | | | | |
| | | | TGTC | eeee | | | | |
| 610017 | 2051 | 2070 | GTGA | eeee | 74 | 13520 | 13539 | 241 |
| | | | CACA | eddd | | | | |
| | | | TCGC | dddd | | | | |
| | | | TGAT | ddde | | | | |
| | | | TTGT | eeee | | | | |
| 610018 | 2052 | 2071 | GGTG | eeee | 74 | 13521 | 13540 | 242 |
| | | | ACAC | eddd | | | | |
| | | | ATCG | dddd | | | | |
| | | | CTGA | ddde | | | | |
| | | | TTTG | eeee | | | | |
| 610019 | 2053 | 2072 | GGGT | eeee | 76 | 13522 | 13541 | 243 |
| | | | GACA | eddd | | | | |
| | | | CATC | dddd | | | | |
| | | | GCTG | ddde | | | | |
| | | | ATTT | eeee | | | | |
| 610020 | 2073 | 2092 | AAGA | eeee | 24 | 13542 | 13561 | 244 |
| | | | AAAG | eddd | | | | |
| | | | GTGG | dddd | | | | |
| | | | GAGA | ddde | | | | |
| | | | CTGG | eeee | | | | |
| 610021 | 2074 | 2093 | GAAG | eeee | 23 | 13543 | 13562 | 245 |
| | | | AAAA | eddd | | | | |
| | | | GGTG | dddd | | | | |
| | | | GGAG | ddde | | | | |
| | | | ACTG | eeee | | | | |
| 610022 | 2075 | 2094 | AGAA | eeee | 26 | 13544 | 13563 | 246 |
| | | | GAAA | eddd | | | | |
| | | | AGGT | dddd | | | | |
| | | | GGGA | ddde | | | | |
| | | | GACT | eeee | | | | |
| 610023 | 2076 | 2095 | TAGA | eeee | 24 | 13545 | 13564 | 247 |
| | | | AGAA | eddd | | | | |
| | | | AAGG | dddd | | | | |
| | | | TGGG | ddde | | | | |
| | | | AGAC | eeee | | | | |
| 610024 | 2077 | 2096 | TTAG | eeee | 19 | 13546 | 13565 | 248 |
| | | | AAGA | eddd | | | | |
| | | | AAAG | dddd | | | | |
| | | | GTGG | ddde | | | | |
| | | | GAGA | eeee | | | | |
| 610025 | 2078 | 2097 | ATTA | eeee | 30 | 13547 | 13566 | 249 |
| | | | GAAG | eddd | | | | |
| | | | AAAA | dddd | | | | |
| | | | GGTG | ddde | | | | |
| | | | GGAG | eeee | | | | |
| 610026 | 2079 | 2098 | CATT | eeee | 40 | 13548 | 13567 | 250 |
| | | | AGAA | eddd | | | | |
| | | | GAAA | dddd | | | | |
| | | | AGGT | ddde | | | | |
| | | | GGGA | eeee | | | | |
| 610027 | 2080 | 2099 | TCAT | eeee | 56 | 13549 | 13568 | 251 |
| | | | TAGA | eddd | | | | |
| | | | AGAA | dddd | | | | |
| | | | AAGG | ddde | | | | |
| | | | TGGG | eeee | | | | |
| 610028 | 2081 | 2100 | CTCA | eeee | 74 | 13550 | 13569 | 252 |
| | | | TTAG | eddd | | | | |
| | | | AAGA | dddd | | | | |
| | | | AAAG | ddde | | | | |
| | | | GTGG | eeee | | | | |
| 610029 | 2082 | 2101 | ACTC | eeee | 62 | 13551 | 13570 | 253 |
| | | | ATTA | eddd | | | | |
| | | | GAAG | dddd | | | | |
| | | | AAAA | ddde | | | | |
| | | | GGTG | eeee | | | | |
| 610030 | 2083 | 2102 | GACT | eeee | 69 | 13552 | 13571 | 254 |
| | | | CATT | eddd | | | | |
| | | | AGAA | dddd | | | | |
| | | | GAAA | ddde | | | | |
| | | | AGGT | eeee | | | | |
| 610031 | 2084 | 2103 | CGAC | eeee | 59 | 13553 | 13572 | 255 |
| | | | TCAT | eddd | | | | |
| | | | TAGA | dddd | | | | |
| | | | AGAA | ddde | | | | |
| | | | AAGG | eeee | | | | |
| 610032 | 2085 | 2104 | TCGA | eeee | 50 | 13554 | 13573 | 256 |
| | | | CTCA | eddd | | | | |
| | | | TTAG | dddd | | | | |
| | | | AAGA | ddde | | | | |
| | | | AAAG | eeee | | | | |
| 610033 | 2086 | 2105 | GTCG | eeee | 67 | 13555 | 13574 | 257 |
| | | | ACTC | eddd | | | | |
| | | | ATTA | dddd | | | | |
| | | | GAAG | ddde | | | | |
| | | | AAAA | eeee | | | | |
| 610034 | 2087 | 2106 | AGTC | eeee | 62 | 13556 | 13575 | 258 |
| | | | GACT | eddd | | | | |
| | | | CATT | dddd | | | | |
| | | | AGAA | ddde | | | | |
| | | | GAAA | eeee | | | | |
| 610035 | 2088 | 2107 | AAGT | eeee | 45 | 13557 | 13576 | 259 |
| | | | CGAC | eddd | | | | |
| | | | TCAT | dddd | | | | |
| | | | TAGA | ddde | | | | |
| | | | AGAA | eeee | | | | |
| 610036 | 2089 | 2108 | AAAG | eeee | 43 | 13558 | 13577 | 260 |
| | | | TCGA | eddd | | | | |
| | | | CTCA | dddd | | | | |
| | | | TTAG | ddde | | | | |
| | | | AAGA | eeee | | | | |
| 610037 | 2090 | 2109 | CAAA | eeee | 46 | 13559 | 13578 | 261 |
| | | | GTCG | eddd | | | | |
| | | | ACTC | dddd | | | | |
| | | | ATTA | ddde | | | | |
| | | | GAAG | eeee | | | | |
| 610038 | 2091 | 2110 | TCAA | eeee | 67 | 13560 | 13579 | 262 |
| | | | AGTC | eddd | | | | |
| | | | GACT | dddd | | | | |
| | | | CATT | ddde | | | | |
| | | | AGAA | eeee | | | | |
| 610039 | 2092 | 2111 | CTCA | eeee | 65 | 13561 | 13580 | 263 |
| | | | AAGT | eddd | | | | |
| | | | CGAC | dddd | | | | |
| | | | TCAT | ddde | | | | |
| | | | TAGA | eeee | | | | |
| 610040 | 2093 | 2112 | GCTC | eeee | 74 | 13562 | 13581 | 264 |
| | | | AAAG | eddd | | | | |
| | | | TCGA | dddd | | | | |
| | | | CTCA | ddde | | | | |
| | | | TTAG | eeee | | | | |
| 610041 | 2094 | 2113 | AGCT | eeee | 61 | 13563 | 13582 | 265 |
| | | | CAAA | eddd | | | | |
| | | | GTCG | dddd | | | | |
| | | | ACTC | ddde | | | | |
| | | | ATTA | eeee | | | | |
| 610042 | 2095 | 2114 | CAGC | eeee | 71 | 13564 | 13583 | 266 |
| | | | TCAA | eddd | | | | |
| | | | AGTC | dddd | | | | |
| | | | GACT | ddde | | | | |
| | | | CATT | eeee | | | | |
| 610043 | 2096 | 2115 | CCAG | eeee | 77 | 13565 | 13584 | 267 |
| | | | CTCA | eddd | | | | |
| | | | AAGT | dddd | | | | |
| | | | CGAC | ddde | | | | |
| | | | TCAT | eeee | | | | |
| 610044 | 2097 | 2116 | TCCA | eeee | 82 | 13566 | 13585 | 268 |
| | | | GCTC | eddd | | | | |
| | | | AAAG | dddd | | | | |
| | | | TCGA | ddde | | | | |
| | | | CTCA | eeee | | | | |
| 610045 | 2098 | 2117 | TTCC | eeee | 80 | 13567 | 13586 | 269 |
| | | | AGCT | eddd | | | | |
| | | | CAAA | dddd | | | | |
| | | | GTCG | ddde | | | | |
| | | | ACTC | eeee | | | | |
| 610046 | 2099 | 2118 | TTTC | eeee | 84 | 13568 | 13587 | 270 |
| | | | CAGC | eddd | | | | |
| | | | TCAA | dddd | | | | |
| | | | AGTC | ddde | | | | |
| | | | GACT | eeee | | | | |
| 610047 | 2100 | 2119 | CTTT | eeee | 65 | 13569 | 13588 | 271 |
| | | | CCAG | eddd | | | | |
| | | | CTCA | dddd | | | | |
| | | | AAGT | ddde | | | | |
| | | | CGAC | eeee | | | | |
| 610048 | 2101 | 2120 | GCTT | eeee | 61 | 13570 | 13589 | 272 |
| | | | TCCA | eddd | | | | |
| | | | GCTC | dddd | | | | |
| | | | AAAG | ddde | | | | |
| | | | TCGA | eeee | | | | |
| 610049 | 2102 | 2121 | TGCT | eeee | 69 | 13571 | 13590 | 273 |
| | | | TTCC | eddd | | | | |
| | | | AGCT | dddd | | | | |
| | | | CAAA | ddde | | | | |
| | | | GTCG | eeee | | | | |
| 610050 | 2103 | 2122 | CTGC | eeee | 54 | 13572 | 13591 | 274 |
| | | | TTTC | eddd | | | | |
| | | | CAGC | dddd | | | | |
| | | | TCAA | ddde | | | | |
| | | | AGTC | eeee | | | | |
| 610051 | 2104 | 2123 | GCTG | eeee | 57 | 13573 | 13592 | 275 |
| | | | CTTT | eddd | | | | |
| | | | CCAG | dddd | | | | |
| | | | CTCA | ddde | | | | |
| | | | AAGT | eeee | | | | |
| 610052 | 2105 | 2124 | GGCT | eeee | 63 | 13574 | 13593 | 276 |
| | | | GCTT | eddd | | | | |
| | | | TCCA | dddd | | | | |
| | | | GCTC | ddde | | | | |
| | | | AAAG | eeee | | | | |
| 610053 | 2106 | 2125 | CGGC | eeee | 40 | 13575 | 13594 | 277 |
| | | | TGCT | eddd | | | | |
| | | | TTCC | dddd | | | | |
| | | | AGCT | ddde | | | | |
| | | | CAAA | eeee | | | | |
| 610054 | 2107 | 2126 | ACGG | eeee | 62 | 13576 | 13595 | 278 |
| | | | CTGC | eddd | | | | |
| | | | TTTC | dddd | | | | |
| | | | CAGC | ddde | | | | |
| | | | TCAA | eeee | | | | |
| 610055 | 2108 | 2127 | AACG | eeee | 69 | 13577 | 13596 | 279 |
| | | | GCTG | eddd | | | | |
| | | | CTTT | dddd | | | | |
| | | | CCAG | ddde | | | | |
| | | | CTCA | eeee | | | | |
| 610056 | 2109 | 2128 | AAAC | eeee | 54 | 13578 | 13597 | 280 |
| | | | GGCT | eddd | | | | |
| | | | GCTT | dddd | | | | |
| | | | TCCA | ddde | | | | |
| | | | GCTC | eeee | | | | |
| 610057 | 2110 | 2129 | GAAA | eeee | 64 | 13579 | 13598 | 281 |
| | | | CGGC | eddd | | | | |
| | | | TGCT | dddd | | | | |
| | | | TTCC | ddde | | | | |
| | | | AGCT | eeee | | | | |
| 610058 | 2111 | 2130 | AGAA | eeee | 57 | 13580 | 13599 | 282 |
| | | | ACGG | eddd | | | | |
| | | | CTGC | dddd | | | | |
| | | | TTTC | ddde | | | | |
| | | | CAGC | eeee | | | | |
| 610059 | 2112 | 2131 | GAGA | eeee | 56 | 13581 | 13600 | 283 |
| | | | AACG | eddd | | | | |
| | | | GCTG | dddd | | | | |
| | | | CTTT | ddde | | | | |
| | | | CCAG | eeee | | | | |
| 610060 | 2113 | 2132 | GGAG | eeee | 73 | 13582 | 13601 | 284 |
| | | | AAAC | eddd | | | | |
| | | | GGCT | dddd | | | | |
| | | | GCTT | ddde | | | | |
| | | | TCCA | eeee | | | | |

Table 3 shows inhibition of AGT mRNA in HepG2 cells cultured at a density of 20,000 cells per well which were transfected using electroporation with 500 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 3
Inhibition of AGT mRNA by MOE and/or cEt
containing gapmers targeting
SEQ ID NO: 1 and/or 2

| | SEQ | SEQ | | | | SEQ | SEQ | |
|---|---|---|---|---|---|---|---|---|
| | ID: | ID: | | | % | ID: | ID | |
| | 1 | 1 | | | Inhi- | 2 | 2: | SEQ |
| ISIS | Start | Stop | | | bi- | Start | Stop | ID |
| NO | Site | Site | Sequence | Chemistry | tion | Site | Site | NO |
| 568637 | 2046 | 2061 | CGCTGATTTGTCCGGG | eekddddddddddkke | 65 | 13515 | 13530 | 129 |
| 568637 | 2046 | 2061 | CGCTGATTTGTCCGGG | eekddddddddddkke | 75 | 13515 | 13530 | 129 |
| 568637 | 2046 | 2061 | CGCTGATTTGTCCGGG | eekddddddddddkke | 0 | 13515 | 13530 | 129 |
| 568637 | 2046 | 2061 | CGCTGATTTGTCCGGG | eekddddddddddkke | 72 | 13515 | 13530 | 129 |
| 568637 | 2046 | 2061 | CGCTGATTTGTCCGGG | eekddddddddddkke | 65 | 13515 | 13530 | 129 |
| 594622 | 2027 | 2042 | GTTATCTGCTGCTGGC | kkkddddddddddkkk | 80 | 13496 | 13511 | 163 |
| 594622 | 2027 | 2042 | GTTATCTGCTGCTGGC | kkkddddddddddkkk | 81 | 13496 | 13511 | 163 |
| 594622 | 2027 | 2042 | GTTATCTGCTGCTGGC | kkkddddddddddkkk | 81 | 13496 | 13511 | 163 |
| 594622 | 2027 | 2042 | GTTATCTGCTGCTGGC | kkkddddddddddkkk | 76 | 13496 | 13511 | 163 |
| 594622 | 2027 | 2042 | GTTATCTGCTGCTGGC | kkkddddddddddkkk | 88 | 13496 | 13511 | 163 |
| 594625 | 2047 | 2062 | TCGCTGATTTGTCCGG | kkkddddddddddkkk | 68 | 13516 | 13531 | 165 |
| 594625 | 2047 | 2062 | TCGCTGATTTGTCCGG | kkkddddddddddkkk | 75 | 13516 | 13531 | 165 |
| 594625 | 2047 | 2062 | TCGCTGATTTGTCCGG | kkkddddddddddkkk | 62 | 13516 | 13531 | 165 |
| 594625 | 2047 | 2062 | TCGCTGATTTGTCCGG | kkkddddddddddkkk | 54 | 13516 | 13531 | 165 |
| 594625 | 2047 | 2062 | TCGCTGATTTGTCCGG | kkkddddddddddkkk | 81 | 13516 | 13531 | 165 |
| 611901 | 1 | 16 | TGCCCGCTCATGGGAT | ekkddddddddddkke | 13 | 1986 | 2001 | 14 |
| 611902 | 6 | 21 | CCTGCTGCCCGCTCAT | ekkddddddddddkke | 19 | 1991 | 2006 | 285 |
| 611903 | 11 | 26 | CTGACCCTGCTGCCCG | ekkddddddddddkke | 19 | 1996 | 2011 | 286 |
| 611904 | 16 | 31 | CACTTCTGACCCTGCT | ekkddddddddddkke | 0 | 2001 | 2016 | 287 |
| 611905 | 35 | 50 | GCTTAGGCAACACGGG | ekkddddddddddkke | 0 | 2020 | 2035 | 16 |
| 611906 | 40 | 55 | GTCTTGCTTAGGCAAC | ekkddddddddddkke | 27 | 2025 | 2040 | 288 |
| 611907 | 45 | 60 | GGAGAGTCTTGCTTAG | ekkddddddddddkke | 0 | 2030 | 2045 | 17 |
| 611908 | 67 | 82 | GGTGCAGAGGGCAGAG | ekkddddddddddkke | 0 | 2052 | 2067 | 289 |
| 611909 | 72 | 87 | CCGGAGGTGCAGAGGG | ekkddddddddddkke | 17 | 2057 | 2072 | 290 |
| 611910 | 77 | 92 | GCAGGCCGGAGGTGCA | ekkddddddddddkke | 10 | 2062 | 2077 | 291 |
| 611911 | 82 | 97 | GACATGCAGGCCGGAG | ekkddddddddddkke | 16 | 2067 | 2082 | 292 |
| 611912 | 87 | 102 | ACAGGGACATGCAGGC | ekkddddddddddkke | 20 | 2072 | 2087 | 293 |
| 611913 | 92 | 107 | AGGCCACAGGGACATG | ekkddddddddddkke | 5 | 2077 | 2092 | 294 |
| 611914 | 97 | 112 | CCAAGAGGCCACAGGG | ekkddddddddddkke | 0 | 2082 | 2097 | 295 |
| 611915 | 102 | 117 | TACCCCCAAGAGGCCA | ekkddddddddddkke | 0 | 2087 | 2102 | 296 |
| 611916 | 107 | 122 | AGATGTACCCCCAAGA | ekkddddddddddkke | 8 | 2092 | 2107 | 297 |
| 611917 | 112 | 127 | CCGGGAGATGTACCCC | ekkddddddddddkke | 16 | 2097 | 2112 | 298 |
| 611918 | 117 | 132 | CAGCCCCGGGAGATGT | ekkddddddddddkke | 15 | 2102 | 2117 | 299 |
| 611919 | 122 | 137 | TGACCCAGCCCCGGGA | ekkddddddddddkke | 4 | 2107 | 2122 | 20 |
| 611920 | 127 | 142 | CCTTCTGACCCAGCCC | ekkddddddddddkke | 23 | 2112 | 2127 | 300 |
| 611921 | 132 | 147 | CCAGGCCTTCTGACCC | ekkddddddddddkke | 20 | 2117 | 2132 | 301 |
| 611922 | 137 | 152 | ACCACCCAGGCCTTCT | ekkddddddddddkke | 15 | 2122 | 2137 | 302 |
| 611923 | 142 | 157 | GGCCAACCACCCAGGC | ekkddddddddddkke | 11 | 2127 | 2142 | 303 |
| 611924 | 147 | 162 | CCTGAGGCCAACCACC | ekkddddddddddkke | 0 | 2132 | 2147 | 304 |
| 611925 | 152 | 167 | GACAGCCTGAGGCCAA | ekkddddddddddkke | 18 | 2137 | 2152 | 305 |
| 611926 | 157 | 172 | TGTGTGACAGCCTGAG | ekkddddddddddkke | 12 | 2142 | 2157 | 306 |
| 611927 | 162 | 177 | CTAGGTGTGTGACAGC | ekkddddddddddkke | 23 | 2147 | 2162 | 307 |
| 611928 | 167 | 182 | TCTCCCTAGGTGTGTG | ekkddddddddddkke | 9 | 2152 | 2167 | 308 |
| 611929 | 172 | 187 | GAGCATCTCCCTAGGT | ekkddddddddddkke | 12 | 2157 | 2172 | 309 |
| 611930 | 177 | 192 | AACGGGAGCATCTCCC | ekkddddddddddkke | 8 | 2162 | 2177 | 310 |
| 611931 | 182 | 197 | CCAGAAACGGGAGCAT | ekkddddddddddkke | 9 | 2167 | 2182 | 311 |
| 611932 | 187 | 202 | GGTTCCCAGAAACGGG | ekkddddddddddkke | 13 | 2172 | 2187 | 312 |
| 611933 | 192 | 207 | GCCAAGGTTCCCAGAA | ekkddddddddddkke | 33 | 2177 | 2192 | 313 |
| 611934 | 208 | 223 | GTTTGCAGGAGTCGGG | ekkddddddddddkke | 17 | 2193 | 2208 | 314 |
| 611935 | 213 | 228 | CCGAAGTTTGCAGGAG | ekkddddddddddkke | 27 | 2198 | 2213 | 315 |
| 611936 | 218 | 233 | ATTTACCGAAGTTTGC | ekkddddddddddkke | 7 | 2203 | 2218 | 316 |
| 611937 | 223 | 238 | TACACATTTACCGAAG | ekkddddddddddkke | 14 | 2208 | 2223 | 317 |
| 611938 | 228 | 243 | CGAGTTACACATTTAC | ekkddddddddddkke | 12 | 2213 | 2228 | 318 |
| 611939 | 233 | 248 | AGGGTCGAGTTACACA | ekkddddddddddkke | 9 | 2218 | 2233 | 319 |
| 611940 | 238 | 253 | GGTGCAGGGTCGAGTT | ekkddddddddddkke | 28 | 2223 | 2238 | 320 |
| 611941 | 243 | 258 | GAGCCGGTGCAGGGTC | ekkddddddddddkke | 26 | 2228 | 2243 | 321 |
| 611942 | 248 | 263 | AGAGTGAGCCGGTGCA | ekkddddddddddkke | 8 | 2233 | 2248 | 27 |
| 611943 | 253 | 268 | TGAACAGAGTGAGCCG | ekkddddddddddkke | 16 | 2238 | 2253 | 322 |
| 611944 | 258 | 273 | ACTGCTGAACAGAGTG | ekkddddddddddkke | 17 | 2243 | 2258 | 28 |
| 611945 | 263 | 278 | GTTTCACTGCTGAACA | ekkddddddddddkke | 17 | 2248 | 2263 | 323 |
| 611946 | 268 | 283 | GCAGAGTTTCACTGCT | ekkddddddddddkke | 0 | 2253 | 2268 | 29 |
| 611947 | 273 | 288 | TCGATGCAGAGTTTCA | ekkddddddddddkke | 2 | 2258 | 2273 | 324 |
| 611948 | 278 | 293 | AGTGATCGATGCAGAG | ekkddddddddddkke | 12 | 2263 | 2278 | 30 |
| 611949 | 283 | 298 | GTCTTAGTGATCGATG | ekkddddddddddkke | 0 | 2268 | 2283 | 325 |
| 611950 | 288 | 303 | AGGAAGTCTTAGTGAT | ekkddddddddddkke | 3 | 2273 | 2288 | 31 |
| 611951 | 293 | 308 | CTTCCAGGAAGTCTTA | ekkddddddddddkke | 10 | 2278 | 2293 | 326 |
| 611952 | 299 | 314 | GGACCTCTTCCAGGAA | ekkddddddddddkke | 21 | 2284 | 2299 | 327 |
| 611953 | 304 | 319 | CGCTGGGACCTCTTCC | ekkddddddddddkke | 20 | 2289 | 2304 | 328 |
| 611954 | 309 | 324 | ACTCACGCTGGGACCT | ekkddddddddddkke | 0 | 2294 | 2309 | 329 |
| 611955 | 314 | 329 | GCGACACTCACGCTGG | ekkddddddddddkke | 21 | 2299 | 2314 | 330 |
| 611956 | 319 | 334 | CAGAAGCGACACTCAC | ekkddddddddddkke | 18 | 2304 | 2319 | 331 |
| 611957 | 324 | 339 | GATGCCAGAAGCGACA | ekkddddddddddkke | 1 | 2309 | 2324 | 332 |
| 611958 | 329 | 344 | GGACAGATGCCAGAAG | ekkddddddddddkke | 16 | 2314 | 2329 | 333 |
| 611959 | 334 | 349 | CAGAAGGACAGATGCC | ekkddddddddddkke | 0 | 2319 | 2334 | 334 |
| 611960 | 339 | 354 | CTGGCCAGAAGGACAG | ekkddddddddddkke | 13 | 2324 | 2339 | 335 |
| 611961 | 344 | 359 | ACAGGCTGGCCAGAAG | ekkddddddddddkke | 15 | 2329 | 2344 | 336 |
| 611962 | 349 | 364 | AGACCACAGGCTGGCC | ekkddddddddddkke | 17 | 2334 | 2349 | 337 |
| 611963 | 354 | 369 | TGGCCAGACCACAGGC | ekkddddddddddkke | 21 | 2339 | 2354 | 338 |
| 611964 | 359 | 374 | TCACTTGGCCAGACCA | ekkddddddddddkke | 7 | 2344 | 2359 | 339 |
| 611965 | 364 | 379 | TTACATCACTTGGCCA | ekkddddddddddkke | 21 | 2349 | 2364 | 340 |
| 611966 | 369 | 384 | GAGGGTTACATCACTT | ekkddddddddddkke | 20 | 2354 | 2369 | 341 |
| 611967 | 374 | 389 | GAGAGGAGGGTTACAT | ekkddddddddddkke | 18 | 2359 | 2374 | 342 |
| 611968 | 386 | 401 | GTGCACAGGCTGGAGA | ekkddddddddddkke | 4 | 2371 | 2386 | 36 |
| 611969 | 391 | 406 | TGCCTGTGCACAGGCT | ekkddddddddddkke | 10 | 2376 | 2391 | 343 |
| 611970 | 396 | 411 | CAGGCTGCCTGTGCAC | ekkddddddddddkke | 26 | 2381 | 2396 | 344 |
| 611971 | 401 | 416 | GTTCCCAGGCTGCCTG | ekkddddddddddkke | 30 | 2386 | 2401 | 345 |
| 611972 | 406 | 421 | GAGCTGTTCCCAGGCT | ekkddddddddddkke | 15 | 2391 | 2406 | 346 |
| 611973 | 411 | 426 | GGATGGAGCTGTTCCC | ekkddddddddddkke | 19 | 2396 | 2411 | 347 |
| 611974 | 431 | 446 | TATTTATAGCTGAGGG | ekkddddddddddkke | 11 | 2416 | 2431 | 37 |
| 611975 | 436 | 451 | TGCCCTATTTATAGCT | ekkddddddddddkke | 20 | 2421 | 2436 | 348 |
| 611976 | 441 | 456 | CACGATGCCCTATTTA | ekkddddddddddkke | 11 | 2426 | 2441 | 38 |
| 612381 | 1852 | 1867 | CTCCAAGACCTCAGGC | ekkddddddddddkke | 4 | 13321 | 13336 | 349 |
| 612382 | 1855 | 1870 | CACCTCCAAGACCTCA | ekkddddddddddkke | 18 | 13324 | 13339 | 350 |
| 612383 | 1858 | 1873 | GGTCACCTCCAAGACC | ekkddddddddddkke | 0 | 13327 | 13342 | 351 |
| 612384 | 1861 | 1876 | CAGGGTCACCTCCAAG | ekkddddddddddkke | 16 | 13330 | 13345 | 352 |
| 612385 | 1864 | 1879 | GTTCAGGGTCACCTCC | ekkddddddddddkke | 28 | 13333 | 13348 | 353 |
| 612386 | 1867 | 1882 | GCGGTTCAGGGTCACC | ekkddddddddddkke | 18 | 13336 | 13351 | 354 |
| 612387 | 1873 | 1888 | GAATGGGCGGTTCAGG | ekkddddddddddkke | 6 | 13342 | 13357 | 355 |
| 612388 | 1876 | 1891 | CAGGAATGGGCGGTTC | ekkddddddddddkke | 13 | 13345 | 13360 | 356 |
| 612389 | 1879 | 1894 | AAACAGGAATGGGCGG | ekkddddddddddkke | 16 | 13348 | 13363 | 357 |
| 612390 | 1883 | 1898 | CAGCAAACAGGAATGG | ekkddddddddddkke | 11 | 13352 | 13367 | 358 |
| 612391 | 1887 | 1902 | TACACAGCAAACAGGA | ekkddddddddddkke | 8 | 13356 | 13371 | 359 |
| 612392 | 1892 | 1907 | GATCATACACAGCAAA | ekkddddddddddkke | 6 | 13361 | 13376 | 360 |
| 612393 | 1895 | 1910 | TTTGATCATACACAGC | ekkddddddddddkke | 15 | 13364 | 13379 | 361 |
| 612394 | 1898 | 1913 | CGCTTTGATCATACAC | ekkddddddddddkke | 16 | 13367 | 13382 | 362 |
| 612395 | 1916 | 1931 | GGAAGTGCAGGGCAGT | ekkddddddddddkke | 8 | 13385 | 13400 | 363 |
| 612396 | 1923 | 1938 | CGGCCCAGGAAGTGCA | ekkddddddddddkke | 0 | 13392 | 13407 | 123 |
| 612397 | 1926 | 1941 | ACGCGGCCCAGGAAGT | ekkddddddddddkke | 1 | 13395 | 13410 | 364 |
| 612398 | 1929 | 1944 | GCCACGCGGCCCAGGA | ekkddddddddddkke | 6 | 13398 | 13413 | 365 |
| 612399 | 1932 | 1947 | TTGGCCACGCGGCCCA | ekkddddddddddkke | 7 | 13401 | 13416 | 366 |
| 612400 | 1935 | 1950 | GGGTTGGCCACGCGGC | ekkddddddddddkke | 29 | 13404 | 13419 | 367 |
| 612401 | 1938 | 1953 | AGCGGGTTGGCCACGC | ekkddddddddddkke | 13 | 13407 | 13422 | 368 |
| 612402 | 1941 | 1956 | CTCAGCGGGTTGGCCA | ekkddddddddddkke | 0 | 13410 | 13425 | 369 |
| 612403 | 1944 | 1959 | GTGCTCAGCGGGTTGG | ekkddddddddddkke | 13 | 13413 | 13428 | 370 |
| 612404 | 1947 | 1962 | GCTGTGCTCAGCGGGT | ekkddddddddddkke | 39 | 13416 | 13431 | 371 |
| 612405 | 1949 | 1964 | ATGCTGTGCTCAGCGG | ekkddddddddddkke | 13 | 13418 | 13433 | 372 |
| 612406 | 1950 | 1965 | CATGCTGTGCTCAGCG | ekkddddddddddkke | 20 | 13419 | 13434 | 373 |
| 612407 | 1951 | 1966 | TCATGCTGTGCTCAGC | ekkddddddddddkke | 23 | 13420 | 13435 | 374 |
| 612408 | 1952 | 1967 | CTCATGCTGTGCTCAG | ekkddddddddddkke | 29 | 13421 | 13436 | 375 |
| 612409 | 1954 | 1969 | GCCTCATGCTGTGCTC | ekkddddddddddkke | 36 | 13423 | 13438 | 376 |
| 612410 | 1956 | 1971 | TGGCCTCATGCTGTGC | ekkddddddddddkke | 0 | 13425 | 13440 | 377 |
| 612411 | 1957 | 1972 | CTGGCCTCATGCTGTG | ekkddddddddddkke | 2 | 13426 | 13441 | 378 |
| 612412 | 1959 | 1974 | CCCTGGCCTCATGCTG | ekkddddddddddkke | 5 | 13428 | 13443 | 379 |
| 612413 | 1960 | 1975 | GCCCTGGCCTCATGCT | ekkddddddddddkke | 6 | 13429 | 13444 | 380 |
| 612414 | 1961 | 1976 | GGCCCTGGCCTCATGC | ekkddddddddddkke | 0 | 13430 | 13445 | 126 |
| 612415 | 1976 | 1991 | GGCACTGTGTTCTGGG | ekkddddddddddkke | 45 | 13445 | 13460 | 381 |
| 612416 | 1987 | 2002 | AGGCCTTGCCAGGCAC | ekkddddddddddkke | 35 | 13456 | 13471 | 382 |
| 612417 | 1992 | 2007 | GGCAGAGGCCTTGCCA | ekkddddddddddkke | 14 | 13461 | 13476 | 383 |
| 612418 | 2007 | 2022 | GCCTCAAAGGCCAGGG | ekkddddddddddkke | 0 | 13476 | 13491 | 128 |
| 612419 | 2008 | 2023 | TGCCTCAAAGGCCAGG | ekkddddddddddkke | 10 | 13477 | 13492 | 384 |
| 612420 | 2009 | 2024 | TTGCCTCAAAGGCCAG | ekkddddddddddkke | 7 | 13478 | 13493 | 385 |
| 612421 | 2010 | 2025 | TTTGCCTCAAAGGCCA | ekkddddddddddkke | 13 | 13479 | 13494 | 386 |
| 612422 | 2011 | 2026 | CTTTGCCTCAAAGGCC | ekkddddddddddkke | 0 | 13480 | 13495 | 387 |
| 612423 | 2012 | 2027 | CCTTTGCCTCAAAGGC | ekkddddddddddkke | 14 | 13481 | 13496 | 388 |
| 612424 | 2013 | 2028 | GCCTTTGCCTCAAAGG | ekkddddddddddkke | 3 | 13482 | 13497 | 389 |
| 612425 | 2014 | 2029 | GGCCTTTGCCTCAAAG | ekkddddddddddkke | 15 | 13483 | 13498 | 390 |
| 612426 | 2015 | 2030 | TGGCCTTTGCCTCAAA | ekkddddddddddkke | 0 | 13484 | 13499 | 391 |
| 612427 | 2016 | 2031 | CTGGCCTTTGCCTCAA | ekkddddddddddkke | 0 | 13485 | 13500 | 392 |
| 612428 | 2017 | 2032 | GCTGGCCTTTGCCTCA | ekkddddddddddkke | 5 | 13486 | 13501 | 393 |
| 612429 | 2100 | 2115 | CCAGCTCAAAGTCGAC | ekkddddddddddkke | 46 | 13569 | 13584 | 394 |
| 612430 | 2101 | 2116 | TCCAGCTCAAAGTCGA | ekkddddddddddkke | 34 | 13570 | 13585 | 395 |
| 612431 | 2102 | 2117 | TTCCAGCTCAAAGTCG | ekkddddddddddkke | 16 | 13571 | 13586 | 396 |
| 612432 | 2103 | 2118 | TTTCCAGCTCAAAGTC | ekkddddddddddkke | 5 | 13572 | 13587 | 397 |
| 612433 | 2105 | 2120 | GCTTTCCAGCTCAAAG | ekkddddddddddkke | 9 | 13574 | 13589 | 398 |
| 612434 | 2110 | 2125 | CGGCTGCTTTCCAGCT | ekkddddddddddkke | 0 | 13579 | 13594 | 399 |
| 612435 | 2111 | 2126 | ACGGCTGCTTTCCAGC | ekkddddddddddkke | 24 | 13580 | 13595 | 133 |
| 612436 | 2112 | 2127 | AACGGCTGCTTTCCAG | ekkddddddddddkke | 14 | 13581 | 13596 | 400 |
| 612437 | 2113 | 2128 | AAACGGCTGCTTTCCA | ekkddddddddddkke | 14 | 13582 | 13597 | 401 |
| 612438 | 2114 | 2129 | GAAACGGCTGCTTTCC | ekkddddddddddkke | 13 | 13583 | 13598 | 402 |
| 612439 | 2115 | 2130 | AGAAACGGCTGCTTTC | ekkddddddddddkke | 15 | 13584 | 13599 | 403 |
| 612440 | 2116 | 2131 | GAGAAACGGCTGCTTT | ekkddddddddddkke | 33 | 13585 | 13600 | 404 |
| 612441 | 2117 | 2132 | GGAGAAACGGCTGCTT | ekkddddddddddkke | 26 | 13586 | 13601 | 405 |
| 612442 | 2118 | 2133 | AGGAGAAACGGCTGCT | ekkddddddddddkke | 50 | 13587 | 13602 | 406 |
| 612443 | 2119 | 2134 | AAGGAGAAACGGCTGC | ekkddddddddddkke | 21 | 13588 | 13603 | 407 |
| 612444 | 2120 | 2135 | CAAGGAGAAACGGCTG | ekkddddddddddkke | 30 | 13589 | 13604 | 408 |
| 612445 | 2121 | 2136 | CCAAGGAGAAACGGCT | ekkddddddddddkke | 43 | 13590 | 13605 | 134 |
| 612446 | 2122 | 2137 | ACCAAGGAGAAACGGC | ekkddddddddddkke | 32 | 13591 | 13606 | 409 |
| 612447 | 2123 | 2138 | GACCAAGGAGAAACGG | ekkddddddddddkke | 33 | 13592 | 13607 | 410 |
| 612448 | 2124 | 2139 | AGACCAAGGAGAAACG | ekkddddddddddkke | 55 | 13593 | 13608 | 411 |
| 612449 | 2125 | 2140 | TAGACCAAGGAGAAAC | ekkddddddddddkke | 15 | 13594 | 13609 | 412 |
| 612450 | 2126 | 2141 | TTAGACCAAGGAGAAA | ekkddddddddddkke | 17 | 13595 | 13610 | 413 |
| 612451 | 2128 | 2143 | ACTTAGACCAAGGAGA | ekkddddddddddkke | 32 | 13597 | 13612 | 414 |
| 612452 | 2129 | 2144 | CACTTAGACCAAGGAG | ekkddddddddddkke | 38 | 13598 | 13613 | 415 |
| 612453 | 2130 | 2145 | ACACTTAGACCAAGGA | ekkddddddddddkke | 48 | 13599 | 13614 | 416 |
| 612454 | 2133 | 2148 | AGCACACTTAGACCAA | ekkddddddddddkke | 29 | 13602 | 13617 | 417 |
| 612455 | 2134 | 2149 | CAGCACACTTAGACCA | ekkddddddddddkke | 31 | 13603 | 13618 | 418 |
| 612456 | 2135 | 2150 | GCAGCACACTTAGACC | ekkddddddddddkke | 13 | 13604 | 13619 | 419 |
| 612457 | 2136 | 2151 | TGCAGCACACTTAGAC | ekkddddddddddkke | 18 | 13605 | 13620 | 420 |
| 612458 | 2137 | 2152 | ATGCAGCACACTTAGA | ekkddddddddddkke | 0 | 13606 | 13621 | 421 |
| 612459 | 2138 | 2153 | CATGCAGCACACTTAG | ekkddddddddddkke | 0 | 13607 | 13622 | 422 |
| 612460 | 2139 | 2154 | CCATGCAGCACACTTA | ekkddddddddddkke | 0 | 13608 | 13623 | 423 |
| 612461 | 2140 | 2155 | TCCATGCAGCACACTT | ekkddddddddddkke | 24 | 13609 | 13624 | 424 |
| 612462 | 2141 | 2156 | CTCCATGCAGCACACT | ekkddddddddddkke | 40 | 13610 | 13625 | 425 |
| 612463 | 2142 | 2157 | ACTCCATGCAGCACAC | ekkddddddddddkke | 0 | 13611 | 13626 | 426 |
| 612464 | 2143 | 2158 | CACTCCATGCAGCACA | ekkddddddddddkke | 18 | 13612 | 13627 | 427 |
| 612465 | 2144 | 2159 | TCACTCCATGCAGCAC | ekkddddddddddkke | 14 | 13613 | 13628 | 428 |
| 612466 | 2162 | 2177 | GCTGCAGGCTTCTACT | ekkddddddddddkke | 12 | 13631 | 13646 | 429 |
| 612467 | 2163 | 2178 | CGCTGCAGGCTTCTAC | ekkddddddddddkke | 2 | 13632 | 13647 | 430 |
| 612468 | 2164 | 2179 | CCGCTGCAGGCTTCTA | ekkddddddddddkke | 2 | 13633 | 13648 | 431 |
| 612469 | 2165 | 2180 | GCCGCTGCAGGCTTCT | ekkddddddddddkke | 12 | 13634 | 13649 | 432 |
| 612470 | 2166 | 2181 | TGCCGCTGCAGGCTTC | ekkddddddddddkke | 1 | 13635 | 13650 | 136 |
| 612471 | 2167 | 2182 | GTGCCGCTGCAGGCTT | ekkddddddddddkke | 12 | 13636 | 13651 | 433 |
| 612472 | 2168 | 2183 | TGTGCCGCTGCAGGCT | ekkddddddddddkke | 31 | 13637 | 13652 | 434 |
| 612473 | 2169 | 2184 | TTGTGCCGCTGCAGGC | ekkddddddddddkke | 20 | 13638 | 13653 | 435 |
| 612474 | 2170 | 2185 | TTTGTGCCGCTGCAGG | ekkddddddddddkke | 27 | 13639 | 13654 | 436 |
| 612475 | 2171 | 2186 | ATTTGTGCCGCTGCAG | ekkddddddddddkke | 29 | 13640 | 13655 | 437 |
| 612476 | 2172 | 2187 | CATTTGTGCCGCTGCA | ekkddddddddddkke | 33 | 13641 | 13656 | 438 |
| 612477 | 2173 | 2188 | GCATTTGTGCCGCTGC | ekkddddddddddkke | 48 | 13642 | 13657 | 439 |
| 612478 | 2174 | 2189 | TGCATTTGTGCCGCTG | ekkddddddddddkke | 13 | 13643 | 13658 | 440 |
| 612479 | 2175 | 2190 | GTGCATTTGTGCCGCT | ekkddddddddddkke | 49 | 13644 | 13659 | 441 |
| 612480 | 2176 | 2191 | GGTGCATTTGTGCCGC | ekkddddddddddkke | 32 | 13645 | 13660 | 137 |
| 612481 | 2177 | 2192 | AGGTGCATTTGTGCCG | ekkddddddddddkke | 40 | 13646 | 13661 | 442 |
| 612482 | 2178 | 2193 | GAGGTGCATTTGTGCC | ekkddddddddddkke | 48 | 13647 | 13662 | 443 |
| 612483 | 2179 | 2194 | GGAGGTGCATTTGTGC | ekkddddddddddkke | 17 | 13648 | 13663 | 444 |
| 612484 | 2180 | 2195 | GGGAGGTGCATTTGTG | ekkddddddddddkke | 15 | 13649 | 13664 | 445 |
| 612485 | 2181 | 2196 | TGGGAGGTGCATTTGT | ekkddddddddddkke | 25 | 13650 | 13665 | 446 |
| 612486 | 2182 | 2197 | CTGGGAGGTGCATTTG | ekkddddddddddkke | 25 | 13651 | 13666 | 447 |
| 612487 | 2183 | 2198 | ACTGGGAGGTGCATTT | ekkddddddddddkke | 19 | 13652 | 13667 | 448 |
| 612488 | 2184 | 2199 | AACTGGGAGGTGCATT | ekkddddddddddkke | 0 | 13653 | 13668 | 449 |
| 612489 | 2185 | 2200 | AAACTGGGAGGTGCAT | ekkddddddddddkke | 14 | 13654 | 13669 | 450 |
| 612490 | 2186 | 2201 | CAAACTGGGAGGTGCA | ekkddddddddddkke | 53 | 13655 | 13670 | 451 |
| 612491 | 2187 | 2202 | GCAAACTGGGAGGTGC | ekkddddddddddkke | 63 | 13656 | 13671 | 452 |
| 612492 | 2188 | 2203 | AGCAAACTGGGAGGTG | ekkddddddddddkke | 26 | 13657 | 13672 | 453 |
| 612493 | 2192 | 2207 | ACCCAGCAAACTGGGA | ekkddddddddddkke | 0 | 13661 | 13676 | 454 |
| 612494 | 2193 | 2208 | AACCCAGCAAACTGGG | ekkddddddddddkke | 0 | 13662 | 13677 | 455 |
| 612495 | 2195 | 2210 | TAAACCCAGCAAACTG | ekkddddddddddkke | 8 | 13664 | 13679 | 456 |
| 612496 | 2196 | 2211 | ATAAACCCAGCAAACT | ekkddddddddddkke | 4 | 13665 | 13680 | 457 |
| 612497 | 2210 | 2225 | CCCCATTCTCTAAAAT | ekkddddddddddkke | 24 | 13679 | 13694 | 458 |
| 612498 | 2211 | 2226 | CCCCCATTCTCTAAAA | ekkddddddddddkke | 0 | 13680 | 13695 | 459 |
| 612499 | 2212 | 2227 | ACCCCCATTCTCTAAA | ekkddddddddddkke | 0 | 13681 | 13696 | 460 |
| 612500 | 2213 | 2228 | CACCCCCATTCTCTAA | ekkddddddddddkke | 6 | 13682 | 13697 | 461 |
| 612501 | 2214 | 2229 | CCACCCCCATTCTCTA | ekkddddddddddkke | 39 | 13683 | 13698 | 462 |
| 612502 | 2226 | 2241 | GTTCTTGCCTCCCCAC | ekkddddddddddkke | 61 | 13695 | 13710 | 463 |
| 612503 | 2227 | 2242 | GGTTCTTGCCTCCCCA | ekkddddddddddkke | 76 | 13696 | 13711 | 464 |
| 612504 | 2228 | 2243 | TGGTTCTTGCCTCCCC | ekkddddddddddkke | 59 | 13697 | 13712 | 465 |
| 612505 | 2229 | 2244 | CTGGTTCTTGCCTCCC | ekkddddddddddkke | 66 | 13698 | 13713 | 466 |
| 612506 | 2230 | 2245 | ACTGGTTCTTGCCTCC | ekkddddddddddkke | 70 | 13699 | 13714 | 467 |
| 612507 | 2231 | 2246 | CACTGGTTCTTGCCTC | ekkddddddddddkke | 57 | 13700 | 13715 | 468 |
| 612508 | 2232 | 2247 | ACACTGGTTCTTGCCT | ekkddddddddddkke | 45 | 13701 | 13716 | 469 |
| 612509 | 2233 | 2248 | AACACTGGTTCTTGCC | ekkddddddddddkke | 66 | 13702 | 13717 | 470 |
| 612510 | 2234 | 2249 | AAACACTGGTTCTTGC | ekkddddddddddkke | 52 | 13703 | 13718 | 471 |
| 612511 | 2235 | 2250 | TAAACACTGGTTCTTG | ekkddddddddddkke | 17 | 13704 | 13719 | 472 |
| 612512 | 2236 | 2251 | CTAAACACTGGTTCTT | ekkddddddddddkke | 35 | 13705 | 13720 | 473 |
| 612513 | 2237 | 2252 | GCTAAACACTGGTTCT | ekkddddddddddkke | 53 | 13706 | 13721 | 474 |
| 612514 | 2238 | 2253 | CGCTAAACACTGGTTC | ekkddddddddddkke | 56 | 13707 | 13722 | 475 |
| 612515 | 2239 | 2254 | GCGCTAAACACTGGTT | ekkddddddddddkke | 59 | 13708 | 13723 | 476 |
| 612516 | 2240 | 2255 | CGCGCTAAACACTGGT | ekkddddddddddkke | 66 | 13709 | 13724 | 477 |
| 612517 | 2241 | 2256 | CCGCGCTAAACACTGG | ekkddddddddddkke | 57 | 13710 | 13725 | 478 |
| 612518 | 2242 | 2257 | CCCGCGCTAAACACTG | ekkddddddddddkke | 35 | 13711 | 13726 | 479 |
| 612519 | 2243 | 2258 | TCCCGCGCTAAACACT | ekkddddddddddkke | 60 | 13712 | 13727 | 480 |
| 612520 | 2244 | 2259 | GTCCCGCGCTAAACAC | ekkddddddddddkke | 38 | 13713 | 13728 | 481 |
| 612521 | 2245 | 2260 | AGTCCCGCGCTAAACA | ekkddddddddddkke | 35 | 13714 | 13729 | 482 |
| 612522 | 2246 | 2261 | TAGTCCCGCGCTAAAC | ekkddddddddddkke | 1 | 13715 | 13730 | 483 |
| 612524 | 2248 | 2263 | AGTAGTCCCGCGCTAA | ekkddddddddddkke | 47 | 13717 | 13732 | 484 |
| 612525 | 2249 | 2264 | CAGTAGTCCCGCGCTA | ekkddddddddddkke | 13 | 13718 | 13733 | 485 |
| 612526 | 2250 | 2265 | ACAGTAGTCCCGCGCT | ekkddddddddddkke | 32 | 13719 | 13734 | 486 |
| 612527 | 2251 | 2266 | AACAGTAGTCCCGCGC | ekkddddddddddkke | 46 | 13720 | 13735 | 487 |
| 612528 | 2252 | 2267 | GAACAGTAGTCCCGCG | ekkddddddddddkke | 27 | 13721 | 13736 | 488 |
| 612529 | 2253 | 2268 | GGAACAGTAGTCCCGC | ekkddddddddddkke | 46 | 13722 | 13737 | 489 |
| 612530 | 2254 | 2269 | TGGAACAGTAGTCCCG | ekkddddddddddkke | 17 | 13723 | 13738 | 490 |
| 612531 | 2255 | 2270 | TTGGAACAGTAGTCCC | ekkddddddddddkke | 42 | 13724 | 13739 | 491 |
| 612532 | 2256 | 2271 | TTTGGAACAGTAGTCC | ekkddddddddddkke | 14 | 13725 | 13740 | 492 |
| 612533 | 2257 | 2272 | TTTTGGAACAGTAGTC | ekkddddddddddkke | 7 | 13726 | 13741 | 493 |
| 612534 | 2258 | 2273 | TTTTTGGAACAGTAGT | ekkddddddddddkke | 4 | 13727 | 13742 | 494 |
| 612535 | 2259 | 2274 | CTTTTTGGAACAGTAG | ekkddddddddddkke | 31 | 13728 | 13743 | 495 |
| 612536 | 2264 | 2279 | GAATTCTTTTTGGAAC | ekkddddddddddkke | 6 | 13733 | 13748 | 496 |
| 612537 | 2265 | 2280 | GGAATTCTTTTTGGAA | ekkddddddddddkke | 45 | 13734 | 13749 | 497 |
| 612538 | 2266 | 2281 | TGGAATTCTTTTTGGA | ekkddddddddddkke | 42 | 13735 | 13750 | 498 |
| 612539 | 2267 | 2282 | TTGGAATTCTTTTTGG | ekkddddddddddkke | 26 | 13736 | 13751 | 499 |
| 612540 | 2270 | 2285 | CGGTTGGAATTCTTTT | ekkddddddddddkke | 61 | 13739 | 13754 | 500 |
| 612541 | 2271 | 2286 | TCGGTTGGAATTCTTT | ekkddddddddddkke | 58 | 13740 | 13755 | 501 |
| 612542 | 2272 | 2287 | GTCGGTTGGAATTCTT | ekkddddddddddkke | 60 | 13741 | 13756 | 502 |
| 612543 | 2273 | 2288 | GGTCGGTTGGAATTCT | ekkddddddddddkke | 58 | 13742 | 13757 | 503 |
| 612544 | 2274 | 2289 | TGGTCGGTTGGAATTC | ekkddddddddddkke | 46 | 13743 | 13758 | 138 |
| 612545 | 2275 | 2290 | CTGGTCGGTTGGAATT | ekkddddddddddkke | 0 | 13744 | 13759 | 504 |
| 612546 | 2276 | 2291 | GCTGGTCGGTTGGAAT | ekkddddddddddkke | 27 | 13745 | 13760 | 505 |
| 612547 | 2277 | 2292 | AGCTGGTCGGTTGGAA | ekkddddddddddkke | 33 | 13746 | 13761 | 506 |
| 612548 | 2278 | 2293 | AAGCTGGTCGGTTGGA | ekkddddddddddkke | 51 | 13747 | 13762 | 507 |
| 612549 | 2279 | 2294 | CAAGCTGGTCGGTTGG | ekkddddddddddkke | 32 | 13748 | 13763 | 508 |
| 612550 | 2280 | 2295 | ACAAGCTGGTCGGTTG | ekkddddddddddkke | 19 | 13749 | 13764 | 509 |
| 612551 | 2281 | 2296 | AACAAGCTGGTCGGTT | ekkddddddddddkke | 39 | 13750 | 13765 | 510 |
| 612552 | 2282 | 2297 | AAACAAGCTGGTCGGT | ekkddddddddddkke | 49 | 13751 | 13766 | 511 |
| 612553 | 2283 | 2298 | CAAACAAGCTGGTCGG | ekkddddddddddkke | 63 | 13752 | 13767 | 512 |
| 612554 | 2284 | 2299 | ACAAACAAGCTGGTCG | ekkddddddddddkke | 48 | 13753 | 13768 | 139 |
| 612555 | 2285 | 2300 | CACAAACAAGCTGGTC | ekkddddddddddkke | 37 | 13754 | 13769 | 513 |
| 612556 | 2286 | 2301 | TCACAAACAAGCTGGT | ekkddddddddddkke | 28 | 13755 | 13770 | 514 |
| 612557 | 2287 | 2302 | TTCACAAACAAGCTGG | ekkddddddddddkke | 52 | 13756 | 13771 | 515 |
| 612558 | 2288 | 2303 | TTTCACAAACAAGCTG | ekkddddddddddkke | 14 | 13757 | 13772 | 516 |
| 612559 | 2289 | 2304 | GTTTCACAAACAAGCT | ekkddddddddddkke | 65 | 13758 | 13773 | 517 |
| 612560 | 2290 | 2305 | TGTTTCACAAACAAGC | ekkddddddddddkke | 58 | 13759 | 13774 | 518 |
| 612561 | 2291 | 2306 | TTGTTTCACAAACAAG | ekkddddddddddkke | 8 | 13760 | 13775 | 519 |
| 612562 | 2304 | 2319 | AGGGAACACTTTTTTG | ekkddddddddddkke | 26 | 13773 | 13788 | 520 |
| 612563 | 2311 | 2326 | CTTGAAAAGGGAACAC | ekkddddddddddkke | 29 | 13780 | 13795 | 140 |
| 612564 | 2312 | 2327 | ACTTGAAAAGGGAACA | ekkddddddddddkke | 19 | 13781 | 13796 | 521 |
| 612565 | 2313 | 2328 | AACTTGAAAAGGGAAC | ekkddddddddddkke | 2 | 13782 | 13797 | 522 |
| 612566 | 2316 | 2331 | CTCAACTTGAAAAGGG | ekkddddddddddkke | 49 | 13785 | 13800 | 523 |
| 612567 | 2321 | 2336 | TTGTTCTCAACTTGAA | ekkddddddddddkke | 58 | 13790 | 13805 | 524 |
| 612568 | 2322 | 2337 | TTTGTTCTCAACTTGA | ekkddddddddddkke | 63 | 13791 | 13806 | 525 |
| 612569 | 2329 | 2344 | CCCAATTTTTGTTCTC | ekkddddddddddkke | 65 | 13798 | 13813 | 526 |
| 612570 | 2330 | 2345 | ACCCAATTTTTGTTCT | ekkddddddddddkke | 37 | 13799 | 13814 | 527 |
| 612571 | 2331 | 2346 | AACCCAATTTTTGTTC | ekkddddddddddkke | 30 | 13800 | 13815 | 141 |
| 612572 | 2362 | 2377 | GGCAATGCAAAAATGT | ekkddddddddddkke | 53 | 13831 | 13846 | 142 |
| 612573 | 2366 | 2381 | CGAAGGCAATGCAAAA | ekkddddddddddkke | 7 | 13835 | 13850 | 528 |
| 612574 | 2367 | 2382 | CCGAAGGCAATGCAAA | ekkddddddddddkke | 25 | 13836 | 13851 | 529 |
| 612575 | 2368 | 2383 | ACCGAAGGCAATGCAA | ekkddddddddddkke | 36 | 13837 | 13852 | 530 |
| 612576 | 2369 | 2384 | AACCGAAGGCAATGCA | ekkddddddddddkke | 36 | 13838 | 13853 | 531 |
| 612577 | 2370 | 2385 | AAACCGAAGGCAATGC | ekkddddddddddkke | 29 | 13839 | 13854 | 532 |
| 612578 | 2371 | 2386 | CAAACCGAAGGCAATG | ekkddddddddddkke | 6 | 13840 | 13855 | 533 |
| 612579 | 2372 | 2387 | ACAAACCGAAGGCAAT | ekkddddddddddkke | 0 | 13841 | 13856 | 534 |
| 612580 | 2373 | 2388 | TACAAACCGAAGGCAA | ekkddddddddddkke | 27 | 13842 | 13857 | 535 |
| 612581 | 2374 | 2389 | ATACAAACCGAAGGCA | ekkddddddddddkke | 13 | 13843 | 13858 | 536 |
| 612582 | 2375 | 2390 | AATACAAACCGAAGGC | ekkddddddddddkke | 0 | 13844 | 13859 | 537 |
| 612583 | 2376 | 2391 | AAATACAAACCGAAGG | ekkddddddddddkke | 0 | 13845 | 13860 | 538 |
| 612584 | 2377 | 2392 | TAAATACAAACCGAAG | ekkddddddddddkke | 25 | 13846 | 13861 | 539 |
| 612585 | 2378 | 2393 | CTAAATACAAACCGAA | ekkddddddddddkke | 0 | 13847 | 13862 | 540 |
| 612586 | 2379 | 2394 | ACTAAATACAAACCGA | ekkddddddddddkke | 19 | 13848 | 13863 | 541 |
| 612587 | 2380 | 2395 | CACTAAATACAAACCG | ekkddddddddddkke | 15 | 13849 | 13864 | 542 |
| 612588 | 2382 | 2397 | GACACTAAATACAAAC | ekkddddddddddkke | 0 | 13851 | 13866 | 543 |
| 612589 | 2385 | 2400 | CAAGACACTAAATACA | ekkddddddddddkke | 9 | 13854 | 13869 | 544 |
| 612590 | 2386 | 2401 | TCAAGACACTAAATAC | ekkddddddddddkke | 19 | 13855 | 13870 | 545 |
| 612591 | 2387 | 2402 | TTCAAGACACTAAATA | ekkddddddddddkke | 0 | 13856 | 13871 | 546 |
| 612592 | 2388 | 2403 | ATTCAAGACACTAAAT | ekkddddddddddkke | 2 | 13857 | 13872 | 547 |
| 612593 | 2389 | 2404 | CATTCAAGACACTAAA | ekkddddddddddkke | 0 | 13858 | 13873 | 548 |
| 612594 | 2390 | 2405 | ACATTCAAGACACTAA | ekkddddddddddkke | 8 | 13859 | 13874 | 549 |
| 612595 | 2391 | 2406 | TACATTCAAGACACTA | ekkddddddddddkke | 1 | 13860 | 13875 | 143 |
| 612596 | 2392 | 2407 | TTACATTCAAGACACT | ekkddddddddddkke | 3 | 13861 | 13876 | 550 |
| 612597 | 2393 | 2408 | CTTACATTCAAGACAC | ekkddddddddddkke | 0 | 13862 | 13877 | 551 |
| 612598 | 2394 | 2409 | TCTTACATTCAAGACA | ekkddddddddddkke | 0 | 13863 | 13878 | 552 |
| 612599 | 2395 | 2410 | TTCTTACATTCAAGAC | ekkddddddddddkke | 0 | 13864 | 13879 | 553 |
| 612600 | 2398 | 2413 | ATGTTCTTACATTCAA | ekkddddddddddkke | 10 | 13867 | 13882 | 554 |
| 612601 | 2401 | 2416 | GTCATGTTCTTACATT | ekkddddddddddkke | 0 | 13870 | 13885 | 555 |
| 612602 | 2402 | 2417 | GGTCATGTTCTTACAT | ekkddddddddddkke | 34 | 13871 | 13886 | 144 |
| 612603 | 2403 | 2418 | AGGTCATGTTCTTACA | ekkddddddddddkke | 35 | 13872 | 13887 | 556 |
| 612604 | 2404 | 2419 | GAGGTCATGTTCTTAC | ekkddddddddddkke | 37 | 13873 | 13888 | 557 |
| 612605 | 2405 | 2420 | GGAGGTCATGTTCTTA | ekkddddddddddkke | 25 | 13874 | 13889 | 558 |
| 612606 | 2406 | 2421 | CGGAGGTCATGTTCTT | ekkddddddddddkke | 31 | 13875 | 13890 | 559 |
| 612607 | 2407 | 2422 | ACGGAGGTCATGTTCT | ekkddddddddddkke | 23 | 13876 | 13891 | 560 |
| 612608 | 2408 | 2423 | CACGGAGGTCATGTTC | ekkddddddddddkke | 24 | 13877 | 13892 | 561 |
| 612685 | 2565 | 2580 | TGGAGGCTTATTGTGG | ekkddddddddddkke | 25 | 14034 | 14049 | 562 |
| 612686 | 2566 | 2581 | TTGGAGGCTTATTGTG | ekkddddddddddkke | 30 | 14035 | 14050 | 563 |
| 612687 | 2567 | 2582 | TTTGGAGGCTTATTGT | ekkddddddddddkke | 20 | 14036 | 14051 | 564 |
| 612688 | N/A | N/A | CGGCTTACCTTCTGCT | ekkddddddddddkke | 30 | 2483 | 2498 | 565 |
| 612689 | N/A | N/A | CCTCCCGGCCTTTTCC | ekkddddddddddkke | 23 | 2562 | 2577 | 566 |
| 612690 | N/A | N/A | TAGGGTGACCACTCTG | ekkddddddddddkke | 26 | 2897 | 2912 | 567 |
| 612691 | N/A | N/A | AGCAAATCGAGGTTCA | ekkddddddddddkke | 25 | 2970 | 2985 | 568 |
| 612692 | N/A | N/A | TATTAGTTCTCTTCAG | ekkddddddddddkke | 9 | 3047 | 3062 | 569 |
| 612693 | N/A | N/A | CCTTTTAGCTTATCCC | ekkddddddddddkke | 24 | 3089 | 3104 | 570 |
| 612694 | N/A | N/A | AATCTGCCTTTTAGCT | ekkddddddddddkke | 20 | 3095 | 3110 | 571 |
| 612695 | N/A | N/A | CAATCTACGCTGCCCT | ekkddddddddddkke | 27 | 3124 | 3139 | 572 |
| 612696 | N/A | N/A | AGCACCAATCTACGCT | ekkddddddddddkke | 16 | 3129 | 3144 | 573 |
| 612697 | N/A | N/A | CATCCTGGAGAAGTAG | ekkddddddddddkke | 9 | 3276 | 3291 | 574 |
| 612698 | N/A | N/A | GCATCCTGGAGAAGTA | ekkddddddddddkke | 13 | 3277 | 3292 | 575 |
| 612699 | N/A | N/A | ATACAGCCCACATTCC | ekkddddddddddkke | 17 | 3316 | 3331 | 576 |
| 612700 | N/A | N/A | CTGTACCATGTAGTTA | ekkddddddddddkke | 32 | 3418 | 3433 | 577 |
| 612701 | N/A | N/A | CCACACCGGGCACTCT | ekkddddddddddkke | 12 | 3476 | 3491 | 578 |
| 612702 | N/A | N/A | CCCACCACACCGGGCA | ekkddddddddddkke | 22 | 3480 | 3495 | 579 |
| 612703 | N/A | N/A | TTCCCCACCACACCGG | ekkddddddddddkke | 19 | 3483 | 3498 | 580 |
| 612704 | N/A | N/A | TTCACCCTGCAGCTTT | ekkddddddddddkke | 13 | 3497 | 3512 | 581 |
| 612705 | N/A | N/A | CATAGTCCTCACCTTC | ekkddddddddddkke | 16 | 3537 | 3552 | 582 |
| 612706 | N/A | N/A | GTGAAGATGACGGCTC | ekkddddddddddkke | 24 | 3615 | 3630 | 583 |
| 612707 | N/A | N/A | TATGTCTCCCTACTTC | ekkddddddddddkke | 25 | 3651 | 3666 | 584 |
| 612708 | N/A | N/A | GGGAGTAATGGTGCTC | ekkddddddddddkke | 33 | 3755 | 3770 | 585 |
| 612709 | N/A | N/A | GTCCTGGGAGTAATGG | ekkddddddddddkke | 24 | 3760 | 3775 | 586 |
| 612710 | N/A | N/A | GGGAACCGACTGCTGG | ekkddddddddddkke | 24 | 3977 | 3992 | 587 |
| 612711 | N/A | N/A | CCTGTGGGAACCGACT | ekkddddddddddkke | 14 | 3982 | 3997 | 588 |
| 612712 | N/A | N/A | CCTAATCTAGACAGTC | ekkddddddddddkke | 5 | 4024 | 4039 | 589 |
| 612713 | N/A | N/A | CATCCGCTGTTCTCAG | ekkddddddddddkke | 2 | 4133 | 4148 | 590 |
| 612714 | N/A | N/A | CTCCATCCGCTGTTCT | ekkddddddddddkke | 28 | 4136 | 4151 | 591 |
| 612715 | N/A | N/A | GACTCCATCCGCTGTT | ekkddddddddddkke | 30 | 4138 | 4153 | 592 |
| 612716 | N/A | N/A | TGACTCCATCCGCTGT | ekkddddddddddkke | 25 | 4139 | 4154 | 593 |
| 612717 | N/A | N/A | GCTGAAGTACCTGGTG | ekkddddddddddkke | 34 | 4230 | 4245 | 594 |
| 612718 | N/A | N/A | GCCCTCAACACGGTGC | ekkddddddddddkke | 25 | 4250 | 4265 | 595 |
| 612719 | N/A | N/A | TGCCCTCAACACGGTG | ekkddddddddddkke | 20 | 4251 | 4266 | 596 |
| 612720 | N/A | N/A | GTCATTCTTCTTACAT | ekkddddddddddkke | 14 | 4307 | 4322 | 597 |
| 612721 | N/A | N/A | GCTTCCTTGGAGCTGT | ekkddddddddddkke | 5 | 4390 | 4405 | 598 |
| 612722 | N/A | N/A | GTGTACTGCAATATCG | ekkddddddddddkke | 39 | 4446 | 4461 | 599 |
| 612723 | N/A | N/A | CACTCATTTCTTGTGG | ekkddddddddddkke | 8 | 4468 | 4483 | 600 |
| 612724 | N/A | N/A | TTGTACCACATCTCAC | ekkddddddddddkke | 21 | 4481 | 4496 | 601 |
| 612725 | N/A | N/A | GTTCTCTCAAAGGCCT | ekkddddddddddkke | 32 | 4651 | 4666 | 602 |
| 612726 | N/A | N/A | GCAGGGTTTAGAACCC | ekkddddddddddkke | 18 | 4694 | 4709 | 603 |
| 612727 | N/A | N/A | TATGTAAGCAGGGTTT | ekkddddddddddkke | 11 | 4701 | 4716 | 604 |
| 612728 | N/A | N/A | AAACCAGCTCTCAACC | ekkddddddddddkke | 5 | 4864 | 4879 | 605 |
| 612729 | N/A | N/A | TAAGACATGCTCCTGC | ekkddddddddddkke | 12 | 5094 | 5109 | 606 |
| 612730 | N/A | N/A | ACTTATGGCAGCCCAA | ekkddddddddddkke | 20 | 5116 | 5131 | 607 |
| 612731 | N/A | N/A | TACTTATGGCAGCCCA | ekkddddddddddkke | 12 | 5117 | 5132 | 608 |
| 612732 | N/A | N/A | CCATTATTTGGAGACA | ekkddddddddddkke | 9 | 5426 | 5441 | 609 |
| 612733 | N/A | N/A | TGCCATCTAACCAGAT | ekkddddddddddkke | 15 | 5655 | 5670 | 610 |
| 612745 | N/A | N/A | GTTTTCAGTAATGCCC | ekkddddddddddkke | 21 | 7085 | 7100 | 611 |

Table 4 shows inhibition of AGT mRNA in HepG2 cells cultured at a density of 20,000 cells per well which were transfected using electroporation with 1000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 4
Inhibition of AGT mRNA by MOE and/or cEt
containing gapmers targeting
SEQ ID NO: 1 and/or 2

| | SEQ | SEQ | | | | SEQ | SEQ | |
|---|---|---|---|---|---|---|---|---|
| | ID: | ID: | | | % | ID: | ID | |
| | 1 | 1 | | | Inhi- | 2 | 2: | SEQ |
| ISIS | Start | Stop | Se- | Chem- | bi- | Start | Stop | ID |
| NO | Site | Site | quence | istry | tion | Site | Site | NO |
| 568637 | 2046 | 2061 | CGC | eek | 43 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 36 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 20 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 51 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 594622 | 2027 | 2042 | GTT | kkk | 92 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 91 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 92 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 90 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594625 | 2047 | 2062 | TCG | kkk | 79 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 88 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 80 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 73 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 611977 | 446 | 461 | CGG | ekk | 13 | 2431 | 2446 | 612 |
| | | | GTC | ddd | | | | |
| | | | ACG | ddd | | | | |
| | | | ATG | ddd | | | | |
| | | | CCC | dkk | | | | |
| | | | T | e | | | | |
| 611978 | 451 | 466 | CCG | ekk | 20 | 2436 | 2451 | 613 |
| | | | GCC | ddd | | | | |
| | | | GGG | ddd | | | | |
| | | | TCA | ddd | | | | |
| | | | CGA | dkk | | | | |
| | | | T | e | | | | |
| 611979 | 454 | 469 | CCC | ekk | 26 | 2439 | 2454 | 614 |
| | | | CCG | ddd | | | | |
| | | | GCC | ddd | | | | |
| | | | GGG | ddd | | | | |
| | | | TCA | dkk | | | | |
| | | | C | e | | | | |
| 611980 | 457 | 472 | CTT | ekk | 20 | 2442 | 2457 | 615 |
| | | | CCC | ddd | | | | |
| | | | CCG | ddd | | | | |
| | | | GCC | ddd | | | | |
| | | | GGG | dkk | | | | |
| | | | T | e | | | | |
| 611981 | 460 | 475 | CTT | ekk | 24 | 2445 | 2460 | 616 |
| | | | CTT | ddd | | | | |
| | | | CCC | ddd | | | | |
| | | | CCG | ddd | | | | |
| | | | GCC | dkk | | | | |
| | | | G | e | | | | |
| 611982 | 463 | 478 | CAG | ekk | 41 | 2448 | 2463 | 617 |
| | | | CTT | ddd | | | | |
| | | | CTT | ddd | | | | |
| | | | CCC | ddd | | | | |
| | | | CCG | dkk | | | | |
| | | | G | e | | | | |
| 611983 | 466 | 481 | CGG | ekk | 15 | 2451 | 2466 | 618 |
| | | | CAG | ddd | | | | |
| | | | CTT | ddd | | | | |
| | | | CTT | ddd | | | | |
| | | | CCC | dkk | | | | |
| | | | C | e | | | | |
| 611984 | 469 | 484 | CAA | ekk | 20 | 2454 | 2469 | 619 |
| | | | CGG | ddd | | | | |
| | | | CAG | ddd | | | | |
| | | | CTT | ddd | | | | |
| | | | CTT | dkk | | | | |
| | | | C | e | | | | |
| 611985 | 472 | 487 | GAA | ekk | 27 | 2457 | 2472 | 620 |
| | | | CAA | ddd | | | | |
| | | | CGG | ddd | | | | |
| | | | CAG | ddd | | | | |
| | | | CTT | dkk | | | | |
| | | | C | e | | | | |
| 611986 | 475 | 490 | CCA | ekk | 23 | 2460 | 2475 | 621 |
| | | | GAA | ddd | | | | |
| | | | CAA | ddd | | | | |
| | | | CGG | ddd | | | | |
| | | | CAG | dkk | | | | |
| | | | C | e | | | | |
| 611987 | 478 | 493 | TAC | ekk | 40 | 2463 | 2478 | 39 |
| | | | CCA | ddd | | | | |
| | | | GAA | ddd | | | | |
| | | | CAA | ddd | | | | |
| | | | CGG | dkk | | | | |
| | | | C | e | | | | |
| 611988 | 481 | 496 | TAG | ekk | 10 | 2466 | 2481 | 622 |
| | | | TAC | ddd | | | | |
| | | | CCA | ddd | | | | |
| | | | GAA | ddd | | | | |
| | | | CAA | dkk | | | | |
| | | | C | e | | | | |
| 611989 | 484 | 499 | CTG | ekk | 21 | 2469 | 2484 | 623 |
| | | | TAG | ddd | | | | |
| | | | TAC | ddd | | | | |
| | | | CCA | ddd | | | | |
| | | | GAA | dkk | | | | |
| | | | C | e | | | | |
| 611990 | 487 | 502 | CTG | ekk | 28 | 2472 | 2487 | 624 |
| | | | CTG | ddd | | | | |
| | | | TAG | ddd | | | | |
| | | | TAC | ddd | | | | |
| | | | CCA | dkk | | | | |
| | | | G | e | | | | |
| 611991 | 490 | 505 | CTT | ekk | 33 | 2475 | 2490 | 625 |
| | | | CTG | ddd | | | | |
| | | | CTG | ddd | | | | |
| | | | TAG | ddd | | | | |
| | | | TAC | dkk | | | | |
| | | | C | e | | | | |
| 611992 | 493 | 508 | ACC | ekk | 39 | N/A | N/A | 626 |
| | | | CTT | ddd | | | | |
| | | | CTG | ddd | | | | |
| | | | CTG | ddd | | | | |
| | | | TAG | dkk | | | | |
| | | | T | e | | | | |
| 611993 | 496 | 511 | CAT | ekk | 19 | N/A | N/A | 627 |
| | | | ACC | ddd | | | | |
| | | | CTT | ddd | | | | |
| | | | CTG | ddd | | | | |
| | | | CTG | dkk | | | | |
| | | | T | e | | | | |
| 611994 | 499 | 514 | CCG | ekk | 11 | N/A | N/A | 628 |
| | | | CAT | ddd | | | | |
| | | | ACC | ddd | | | | |
| | | | CTT | ddd | | | | |
| | | | CTG | dkk | | | | |
| | | | C | e | | | | |
| 611995 | 502 | 517 | CTT | ekk | 21 | N/A | N/A | 629 |
| | | | CCG | ddd | | | | |
| | | | CAT | ddd | | | | |
| | | | ACC | ddd | | | | |
| | | | CTT | dkk | | | | |
| | | | C | e | | | | |
| 611996 | 505 | 520 | TCG | ekk | 53 | 5722 | 5737 | 630 |
| | | | CTT | ddd | | | | |
| | | | CCG | ddd | | | | |
| | | | CAT | ddd | | | | |
| | | | ACC | dkk | | | | |
| | | | C | e | | | | |
| 611997 | 508 | 523 | TGC | ekk | 6 | 5725 | 5740 | 631 |
| | | | TCG | ddd | | | | |
| | | | CTT | ddd | | | | |
| | | | CCG | ddd | | | | |
| | | | CAT | dkk | | | | |
| | | | A | e | | | | |
| 611998 | 511 | 526 | GGG | ekk | 38 | 5728 | 5743 | 632 |
| | | | TGC | ddd | | | | |
| | | | TCG | ddd | | | | |
| | | | CTT | ddd | | | | |
| | | | CCG | dkk | | | | |
| | | | C | e | | | | |
| 611999 | 525 | 540 | GCC | ekk | 31 | 5742 | 5757 | 40 |
| | | | ATC | ddd | | | | |
| | | | TCA | ddd | | | | |
| | | | GAC | ddd | | | | |
| | | | TGG | dkk | | | | |
| | | | G | e | | | | |
| 612000 | 533 | 548 | CGG | ekk | 31 | 5750 | 5765 | 633 |
| | | | CAG | ddd | | | | |
| | | | GAG | ddd | | | | |
| | | | CCA | ddd | | | | |
| | | | TCT | dkk | | | | |
| | | | C | e | | | | |
| 612001 | 536 | 551 | CAC | ekk | 19 | 5753 | 5768 | 634 |
| | | | CGG | ddd | | | | |
| | | | CAG | ddd | | | | |
| | | | GAG | ddd | | | | |
| | | | CCA | dkk | | | | |
| | | | T | e | | | | |
| 612002 | 539 | 554 | TCA | ekk | 19 | 5756 | 5771 | 635 |
| | | | CAC | ddd | | | | |
| | | | CGG | ddd | | | | |
| | | | CAG | ddd | | | | |
| | | | GAG | dkk | | | | |
| | | | C | e | | | | |
| 612003 | 542 | 557 | GGC | ekk | 41 | 5759 | 5774 | 636 |
| | | | TCA | ddd | | | | |
| | | | CAC | ddd | | | | |
| | | | CGG | ddd | | | | |
| | | | CAG | dkk | | | | |
| | | | G | e | | | | |
| 612004 | 545 | 560 | TCA | ekk | 46 | 5762 | 5777 | 42 |
| | | | GGC | ddd | | | | |
| | | | TCA | ddd | | | | |
| | | | CAC | ddd | | | | |
| | | | CGG | dkk | | | | |
| | | | C | e | | | | |
| 612005 | 549 | 564 | GCC | ekk | 18 | 5766 | 5781 | 637 |
| | | | CTC | ddd | | | | |
| | | | AGG | ddd | | | | |
| | | | CTC | ddd | | | | |
| | | | ACA | dkk | | | | |
| | | | C | e | | | | |
| 612006 | 552 | 567 | GTG | ekk | 29 | 5769 | 5784 | 638 |
| | | | GCC | ddd | | | | |
| | | | CTC | ddd | | | | |
| | | | AGG | ddd | | | | |
| | | | CTC | dkk | | | | |
| | | | A | e | | | | |
| 612007 | 555 | 570 | ATG | ekk | 32 | 5772 | 5787 | 43 |
| | | | GTG | ddd | | | | |
| | | | GCC | ddd | | | | |
| | | | CTC | ddd | | | | |
| | | | AGG | dkk | | | | |
| | | | C | e | | | | |
| 612008 | 561 | 576 | CAG | ekk | 33 | 5778 | 5793 | 639 |
| | | | AGG | ddd | | | | |
| | | | ATG | ddd | | | | |
| | | | GTG | ddd | | | | |
| | | | GCC | dkk | | | | |
| | | | C | e | | | | |
| 612009 | 596 | 611 | GGT | ekk | 38 | 5813 | 5828 | 44 |
| | | | CAC | ddd | | | | |
| | | | CTG | ddd | | | | |
| | | | CAG | ddd | | | | |
| | | | CCA | dkk | | | | |
| | | | G | e | | | | |
| 612010 | 599 | 614 | CCC | ekk | 47 | 5816 | 5831 | 640 |
| | | | GGT | ddd | | | | |
| | | | CAC | ddd | | | | |
| | | | CTG | ddd | | | | |
| | | | CAG | dkk | | | | |
| | | | C | e | | | | |
| 612011 | 602 | 617 | ACA | ekk | 29 | 5819 | 5834 | 641 |
| | | | CCC | ddd | | | | |
| | | | GGT | ddd | | | | |
| | | | CAC | ddd | | | | |
| | | | CTG | dkk | | | | |
| | | | C | e | | | | |
| 612012 | 605 | 620 | TGT | ekk | 22 | 5822 | 5837 | 642 |
| | | | ACA | ddd | | | | |
| | | | CCC | ddd | | | | |
| | | | GGT | ddd | | | | |
| | | | CAC | dkk | | | | |
| | | | C | e | | | | |
| 612013 | 608 | 623 | GTA | ekk | 5 | 5825 | 5840 | 643 |
| | | | TGT | ddd | | | | |
| | | | ACA | ddd | | | | |
| | | | CCC | ddd | | | | |
| | | | GGT | dkk | | | | |
| | | | C | e | | | | |
| 612014 | 611 | 626 | GGT | ekk | 0 | 5828 | 5843 | 644 |
| | | | GTA | ddd | | | | |
| | | | TGT | ddd | | | | |
| | | | ACA | ddd | | | | |
| | | | CCC | dkk | | | | |
| | | | G | e | | | | |
| 612015 | 626 | 641 | TGA | ekk | 21 | 5843 | 5858 | 645 |
| | | | CGA | ddd | | | | |
| | | | GGT | ddd | | | | |
| | | | GGA | ddd | | | | |
| | | | AGG | dkk | | | | |
| | | | G | e | | | | |
| 612016 | 629 | 644 | GGA | ekk | 32 | 5846 | 5861 | 646 |
| | | | TGA | ddd | | | | |
| | | | CGA | ddd | | | | |
| | | | GGT | ddd | | | | |
| | | | GGA | dkk | | | | |
| | | | A | e | | | | |
| 612017 | 632 | 647 | TGT | ekk | 48 | 5849 | 5864 | 647 |
| | | | GGA | ddd | | | | |
| | | | TGA | ddd | | | | |
| | | | CGA | ddd | | | | |
| | | | GGT | dkk | | | | |
| | | | G | e | | | | |
| 612018 | 635 | 650 | CAT | ekk | 28 | 5852 | 5867 | 648 |
| | | | TGT | ddd | | | | |
| | | | GGA | ddd | | | | |
| | | | TGA | ddd | | | | |
| | | | CGA | dkk | | | | |
| | | | G | e | | | | |
| 612019 | 638 | 653 | TCT | ekk | 34 | 5855 | 5870 | 649 |
| | | | CAT | ddd | | | | |
| | | | TGT | ddd | | | | |
| | | | GGA | ddd | | | | |
| | | | TGA | dkk | | | | |
| | | | C | e | | | | |
| 612020 | 639 | 654 | CTC | ekk | 38 | 5856 | 5871 | 650 |
| | | | TCA | ddd | | | | |
| | | | TTG | ddd | | | | |
| | | | TGG | ddd | | | | |
| | | | ATG | dkk | | | | |
| | | | A | e | | | | |
| 612021 | 640 | 655 | ACT | ekk | 45 | 5857 | 5872 | 651 |
| | | | CTC | ddd | | | | |
| | | | ATT | ddd | | | | |
| | | | GTG | ddd | | | | |
| | | | GAT | dkk | | | | |
| | | | G | e | | | | |
| 612022 | 641 | 656 | TAC | ekk | 29 | 5858 | 5873 | 652 |
| | | | TCT | ddd | | | | |
| | | | CAT | ddd | | | | |
| | | | TGT | ddd | | | | |
| | | | GGA | dkk | | | | |
| | | | T | e | | | | |
| 612023 | 642 | 657 | GTA | ekk | 46 | 5859 | 5874 | 653 |
| | | | CTC | ddd | | | | |
| | | | TCA | ddd | | | | |
| | | | TTG | ddd | | | | |
| | | | TGG | dkk | | | | |
| | | | A | e | | | | |
| 612024 | 643 | 658 | GGT | ekk | 58 | 5860 | 5875 | 46 |
| | | | ACT | ddd | | | | |
| | | | CTC | ddd | | | | |
| | | | ATT | ddd | | | | |
| | | | GTG | dkk | | | | |
| | | | G | e | | | | |
| 612025 | 645 | 660 | CAG | ekk | 59 | 5862 | 5877 | 654 |
| | | | GTA | ddd | | | | |
| | | | CTC | ddd | | | | |
| | | | TCA | ddd | | | | |
| | | | TTG | dkk | | | | |
| | | | T | e | | | | |
| 612026 | 646 | 661 | ACA | ekk | 50 | 5863 | 5878 | 655 |
| | | | GGT | ddd | | | | |
| | | | ACT | ddd | | | | |
| | | | CTC | ddd | | | | |
| | | | ATT | dkk | | | | |
| | | | G | e | | | | |
| 612027 | 647 | 662 | CAC | ekk | 37 | 5864 | 5879 | 656 |
| | | | AGG | ddd | | | | |
| | | | TAC | ddd | | | | |
| | | | TCT | ddd | | | | |
| | | | CAT | dkk | | | | |
| | | | T | e | | | | |
| 612028 | 648 | 663 | TCA | ekk | 31 | 5865 | 5880 | 657 |
| | | | CAG | ddd | | | | |
| | | | GTA | ddd | | | | |
| | | | CTC | ddd | | | | |
| | | | TCA | dkk | | | | |
| | | | T | e | | | | |
| 612029 | 649 | 664 | CTC | ekk | 22 | 5866 | 5881 | 658 |
| | | | ACA | ddd | | | | |
| | | | GGT | ddd | | | | |
| | | | ACT | ddd | | | | |
| | | | CTC | dkk | | | | |
| | | | A | e | | | | |
| 612030 | 652 | 667 | CTG | ekk | 4 | 5869 | 5884 | 659 |
| | | | CTC | ddd | | | | |
| | | | ACA | ddd | | | | |
| | | | GGT | ddd | | | | |
| | | | ACT | dkk | | | | |
| | | | C | e | | | | |
| 612031 | 659 | 674 | TTG | ekk | 39 | 5876 | 5891 | 660 |
| | | | CCA | ddd | | | | |
| | | | GCT | ddd | | | | |
| | | | GCT | ddd | | | | |
| | | | CAC | dkk | | | | |
| | | | A | e | | | | |
| 612032 | 662 | 677 | CCT | ekk | 45 | 5879 | 5894 | 661 |
| | | | TTG | ddd | | | | |
| | | | CCA | ddd | | | | |
| | | | GCT | ddd | | | | |
| | | | GCT | dkk | | | | |
| | | | C | e | | | | |
| 612033 | 665 | 680 | TGG | ekk | 30 | 5882 | 5897 | 662 |
| | | | CCT | ddd | | | | |
| | | | TTG | ddd | | | | |
| | | | CCA | ddd | | | | |
| | | | GCT | dkk | | | | |
| | | | G | e | | | | |
| 612034 | 668 | 683 | CAT | ekk | 18 | 5885 | 5900 | 663 |
| | | | TGG | ddd | | | | |
| | | | CCT | ddd | | | | |
| | | | TTG | ddd | | | | |
| | | | CCA | dkk | | | | |
| | | | G | e | | | | |
| 612035 | 671 | 686 | CGG | ekk | 18 | 5888 | 5903 | 664 |
| | | | CAT | ddd | | | | |
| | | | TGG | ddd | | | | |
| | | | CCT | ddd | | | | |
| | | | TTG | dkk | | | | |
| | | | C | e | | | | |
| 612036 | 674 | 689 | TCC | ekk | 27 | 5891 | 5906 | 665 |
| | | | CGG | ddd | | | | |
| | | | CAT | ddd | | | | |
| | | | TGG | ddd | | | | |
| | | | CCT | dkk | | | | |
| | | | T | e | | | | |
| 612037 | 677 | 692 | GCT | ekk | 15 | 5894 | 5909 | 666 |
| | | | TCC | ddd | | | | |
| | | | CGG | ddd | | | | |
| | | | CAT | ddd | | | | |
| | | | TGG | dkk | | | | |
| | | | C | e | | | | |
| 612038 | 680 | 695 | TGG | ekk | 2 | 5897 | 5912 | 667 |
| | | | GCT | ddd | | | | |
| | | | TCC | ddd | | | | |
| | | | CGG | ddd | | | | |
| | | | CAT | dkk | | | | |
| | | | T | e | | | | |
| 612039 | 683 | 698 | CTT | ekk | 44 | 5900 | 5915 | 668 |
| | | | TGG | ddd | | | | |
| | | | GCT | ddd | | | | |
| | | | TCC | ddd | | | | |
| | | | CGG | dkk | | | | |
| | | | C | e | | | | |
| 612040 | 686 | 701 | GGT | ekk | 36 | 5903 | 5918 | 669 |
| | | | CTT | ddd | | | | |
| | | | TGG | ddd | | | | |
| | | | GCT | ddd | | | | |
| | | | TCC | dkk | | | | |
| | | | C | e | | | | |
| 612041 | 701 | 716 | CAG | ekk | 42 | 5918 | 5933 | 670 |
| | | | GTA | ddd | | | | |
| | | | TGA | ddd | | | | |
| | | | AGG | ddd | | | | |
| | | | TGG | dkk | | | | |
| | | | G | e | | | | |
| 612042 | 704 | 719 | GAG | ekk | 39 | 5921 | 5936 | 671 |
| | | | CAG | ddd | | | | |
| | | | GTA | ddd | | | | |
| | | | TGA | ddd | | | | |
| | | | AGG | dkk | | | | |
| | | | T | e | | | | |
| 612043 | 707 | 722 | TTG | ekk | 28 | 5924 | 5939 | 672 |
| | | | GAG | ddd | | | | |
| | | | CAG | ddd | | | | |
| | | | GTA | ddd | | | | |
| | | | TGA | dkk | | | | |
| | | | A | e | | | | |
| 612044 | 710 | 725 | GAA | ekk | 20 | 5927 | 5942 | 673 |
| | | | TTG | ddd | | | | |
| | | | GAG | ddd | | | | |
| | | | CAG | ddd | | | | |
| | | | GTA | dkk | | | | |
| | | | T | e | | | | |
| 612045 | 713 | 728 | CCT | ekk | 7 | 5930 | 5945 | 50 |
| | | | GAA | ddd | | | | |
| | | | TTG | ddd | | | | |
| | | | GAG | ddd | | | | |
| | | | CAG | dkk | | | | |
| | | | G | e | | | | |
| 612046 | 716 | 731 | TGG | ekk | 23 | 5933 | 5948 | 674 |
| | | | CCT | ddd | | | | |
| | | | GAA | ddd | | | | |
| | | | TTG | ddd | | | | |
| | | | GAG | dkk | | | | |
| | | | C | e | | | | |
| 612047 | 719 | 734 | TCT | ekk | 29 | 5936 | 5951 | 675 |
| | | | TGG | ddd | | | | |
| | | | CCT | ddd | | | | |
| | | | GAA | ddd | | | | |
| | | | TTG | dkk | | | | |
| | | | G | e | | | | |
| 612048 | 722 | 737 | ATG | ekk | 22 | 5939 | 5954 | 676 |
| | | | TCT | ddd | | | | |
| | | | TGG | ddd | | | | |
| | | | CCT | ddd | | | | |
| | | | GAA | dkk | | | | |
| | | | T | e | | | | |
| 612049 | 725 | 740 | GGG | ekk | 35 | 5942 | 5957 | 677 |
| | | | ATG | ddd | | | | |
| | | | TCT | ddd | | | | |
| | | | TGG | ddd | | | | |
| | | | CCT | dkk | | | | |
| | | | G | e | | | | |
| 612050 | 739 | 754 | CTT | ekk | 21 | 5956 | 5971 | 52 |
| | | | TTC | ddd | | | | |
| | | | ATC | ddd | | | | |
| | | | CAC | ddd | | | | |
| | | | AGG | dkk | | | | |
| | | | G | e | | | | |
| 612051 | 742 | 757 | GGC | ekk | 3 | 5959 | 5974 | 678 |
| | | | CTT | ddd | | | | |
| | | | TTC | ddd | | | | |
| | | | ATC | ddd | | | | |
| | | | CAC | dkk | | | | |
| | | | A | e | | | | |
| 612052 | 745 | 760 | TAG | ekk | 10 | 5962 | 5977 | 679 |
| | | | GGC | ddd | | | | |
| | | | CTT | ddd | | | | |
| | | | TTC | ddd | | | | |
| | | | ATC | dkk | | | | |
| | | | C | e | | | | |
| 612053 | 748 | 763 | CTG | ekk | 5 | 5965 | 5980 | 680 |
| | | | TAG | ddd | | | | |
| | | | GGC | ddd | | | | |
| | | | CTT | ddd | | | | |
| | | | TTC | dkk | | | | |
| | | | A | e | | | | |
| 612054 | 751 | 766 | GTC | ekk | 6 | 5968 | 5983 | 681 |
| | | | CTG | ddd | | | | |
| | | | TAG | ddd | | | | |
| | | | GGC | ddd | | | | |
| | | | CTT | dkk | | | | |
| | | | T | e | | | | |
| 612055 | 754 | 769 | CTG | ekk | 19 | 5971 | 5986 | 682 |
| | | | GTC | ddd | | | | |
| | | | CTG | ddd | | | | |
| | | | TAG | ddd | | | | |
| | | | GGC | dkk | | | | |
| | | | C | e | | | | |
| 612056 | 758 | 773 | CCA | ekk | 34 | 5975 | 5990 | 683 |
| | | | GCT | ddd | | | | |
| | | | GGT | ddd | | | | |
| | | | CCT | ddd | | | | |
| | | | GTA | dkk | | | | |
| | | | G | e | | | | |
| 612057 | 759 | 774 | ACC | ekk | 31 | 5976 | 5991 | 684 |
| | | | AGC | ddd | | | | |
| | | | TGG | ddd | | | | |
| | | | TCC | ddd | | | | |
| | | | TGT | dkk | | | | |
| | | | A | e | | | | |
| 612058 | 762 | 777 | AGC | ekk | 56 | 5979 | 5994 | 53 |
| | | | ACC | ddd | | | | |
| | | | AGC | ddd | | | | |
| | | | TGG | ddd | | | | |
| | | | TCC | dkk | | | | |
| | | | T | e | | | | |
| 612059 | 763 | 778 | TAG | ekk | 35 | 5980 | 5995 | 685 |
| | | | CAC | ddd | | | | |
| | | | CAG | ddd | | | | |
| | | | CTG | ddd | | | | |
| | | | GTC | dkk | | | | |
| | | | C | e | | | | |
| 612060 | 764 | 779 | CTA | ekk | 18 | 5981 | 5996 | 686 |
| | | | GCA | ddd | | | | |
| | | | CCA | ddd | | | | |
| | | | GCT | ddd | | | | |
| | | | GGT | dkk | | | | |
| | | | C | e | | | | |
| 612061 | 765 | 780 | ACT | ekk | 10 | 5982 | 5997 | 687 |
| | | | AGC | ddd | | | | |
| | | | ACC | ddd | | | | |
| | | | AGC | ddd | | | | |
| | | | TGG | dkk | | | | |
| | | | T | e | | | | |
| 612062 | 766 | 781 | GAC | ekk | 32 | 5983 | 5998 | 688 |
| | | | TAG | ddd | | | | |
| | | | CAC | ddd | | | | |
| | | | CAG | ddd | | | | |
| | | | CTG | dkk | | | | |
| | | | G | e | | | | |
| 612063 | 767 | 782 | CGA | ekk | 49 | 5984 | 5999 | 689 |
| | | | CTA | ddd | | | | |
| | | | GCA | ddd | | | | |
| | | | CCA | ddd | | | | |
| | | | GCT | dkk | | | | |
| | | | G | e | | | | |
| 612064 | 768 | 783 | GCG | ekk | 39 | 5985 | 6000 | 690 |
| | | | ACT | ddd | | | | |
| | | | AGC | ddd | | | | |
| | | | ACC | ddd | | | | |
| | | | AGC | dkk | | | | |
| | | | T | e | | | | |
| 612065 | 769 | 784 | AGC | ekk | 29 | 5986 | 6001 | 691 |
| | | | GAC | ddd | | | | |
| | | | TAG | ddd | | | | |
| | | | CAC | ddd | | | | |
| | | | CAG | dkk | | | | |
| | | | C | e | | | | |
| 612066 | 770 | 785 | CAG | ekk | 38 | 5987 | 6002 | 692 |
| | | | CGA | ddd | | | | |
| | | | CTA | ddd | | | | |
| | | | GCA | ddd | | | | |
| | | | CCA | dkk | | | | |
| | | | G | e | | | | |
| 612067 | 771 | 786 | GCA | ekk | 39 | 5988 | 6003 | 693 |
| | | | GCG | ddd | | | | |
| | | | ACT | ddd | | | | |
| | | | AGC | ddd | | | | |
| | | | ACC | dkk | | | | |
| | | | A | e | | | | |
| 612068 | 772 | 787 | TGC | ekk | 31 | 5989 | 6004 | 54 |
| | | | AGC | ddd | | | | |
| | | | GAC | ddd | | | | |
| | | | TAG | ddd | | | | |
| | | | CAC | dkk | | | | |
| | | | C | e | | | | |
| 612069 | 773 | 788 | TTG | ekk | 28 | 5990 | 6005 | 694 |
| | | | CAG | ddd | | | | |
| | | | CGA | ddd | | | | |
| | | | CTA | ddd | | | | |
| | | | GCA | dkk | | | | |
| | | | C | e | | | | |
| 612070 | 774 | 789 | TTT | ekk | 31 | 5991 | 6006 | 695 |
| | | | GCA | ddd | | | | |
| | | | GCG | ddd | | | | |
| | | | ACT | ddd | | | | |
| | | | AGC | dkk | | | | |
| | | | A | e | | | | |
| 612071 | 775 | 790 | TTT | ekk | 28 | 5992 | 6007 | 696 |
| | | | TGC | ddd | | | | |
| | | | AGC | ddd | | | | |
| | | | GAC | ddd | | | | |
| | | | TAG | dkk | | | | |
| | | | C | e | | | | |
| 612072 | 776 | 791 | GTT | ekk | 11 | 5993 | 6008 | 697 |
| | | | TTG | ddd | | | | |
| | | | CAG | ddd | | | | |
| | | | CGA | ddd | | | | |
| | | | CTA | dkk | | | | |
| | | | G | e | | | | |
| 612073 | 777 | 792 | AGT | ekk | 7 | 5994 | 6009 | 698 |
| | | | TTT | ddd | | | | |
| | | | GCA | ddd | | | | |
| | | | GCG | ddd | | | | |
| | | | ACT | dkk | | | | |
| | | | A | e | | | | |
| 612074 | 778 | 793 | AAG | ekk | 10 | 5995 | 6010 | 699 |
| | | | TTT | ddd | | | | |
| | | | TGC | ddd | | | | |
| | | | AGC | ddd | | | | |
| | | | GAC | dkk | | | | |
| | | | T | e | | | | |
| 612075 | 781 | 796 | GTC | ekk | 49 | 5998 | 6013 | 700 |
| | | | AAG | ddd | | | | |
| | | | TTT | ddd | | | | |
| | | | TGC | ddd | | | | |
| | | | AGC | dkk | | | | |
| | | | G | e | | | | |
| 612076 | 784 | 799 | GGT | ekk | 39 | 6001 | 6016 | 701 |
| | | | GTC | ddd | | | | |
| | | | AAG | ddd | | | | |
| | | | TTT | ddd | | | | |
| | | | TGC | dkk | | | | |
| | | | A | e | | | | |
| 612077 | 787 | 802 | TTC | ekk | 53 | 6004 | 6019 | 702 |
| | | | GGT | ddd | | | | |
| | | | GTC | ddd | | | | |
| | | | AAG | ddd | | | | |
| | | | TTT | dkk | | | | |
| | | | T | e | | | | |
| 612078 | 790 | 805 | GTC | ekk | 39 | 6007 | 6022 | 703 |
| | | | TTC | ddd | | | | |
| | | | GGT | ddd | | | | |
| | | | GTC | ddd | | | | |
| | | | AAG | dkk | | | | |
| | | | T | e | | | | |
| 612079 | 793 | 808 | CTT | ekk | 35 | 6010 | 6025 | 704 |
| | | | GTC | ddd | | | | |
| | | | TTC | ddd | | | | |
| | | | GGT | ddd | | | | |
| | | | GTC | dkk | | | | |
| | | | A | e | | | | |
| 612080 | 796 | 811 | CAA | ekk | 42 | 6013 | 6028 | 705 |
| | | | CTT | ddd | | | | |
| | | | GTC | ddd | | | | |
| | | | TTC | ddd | | | | |
| | | | GGT | dkk | | | | |
| | | | G | e | | | | |
| 612081 | 799 | 814 | CCT | ekk | 1 | 6016 | 6031 | 706 |
| | | | CAA | ddd | | | | |
| | | | CTT | ddd | | | | |
| | | | GTC | ddd | | | | |
| | | | TTC | dkk | | | | |
| | | | G | e | | | | |
| 612082 | 802 | 817 | GGC | ekk | 0 | 6019 | 6034 | 707 |
| | | | CCT | ddd | | | | |
| | | | CAA | ddd | | | | |
| | | | CTT | ddd | | | | |
| | | | GTC | dkk | | | | |
| | | | T | e | | | | |
| 612083 | 805 | 820 | TGC | ekk | 13 | 6022 | 6037 | 708 |
| | | | GGC | ddd | | | | |
| | | | CCT | ddd | | | | |
| | | | CAA | ddd | | | | |
| | | | CTT | dkk | | | | |
| | | | G | e | | | | |
| 612084 | 808 | 823 | CAT | ekk | 0 | 6025 | 6040 | 709 |
| | | | TGC | ddd | | | | |
| | | | GGC | ddd | | | | |
| | | | CCT | ddd | | | | |
| | | | CAA | dkk | | | | |
| | | | C | e | | | | |
| 612085 | 811 | 826 | GAC | ekk | 30 | 6028 | 6043 | 710 |
| | | | CAT | ddd | | | | |
| | | | TGC | ddd | | | | |
| | | | GGC | ddd | | | | |
| | | | CCT | dkk | | | | |
| | | | C | e | | | | |
| 612086 | 814 | 829 | CCC | ekk | 32 | 6031 | 6046 | 711 |
| | | | GAC | ddd | | | | |
| | | | CAT | ddd | | | | |
| | | | TGC | ddd | | | | |
| | | | GGC | dkk | | | | |
| | | | C | e | | | | |
| 612087 | 817 | 832 | CAT | ekk | 49 | 6034 | 6049 | 712 |
| | | | CCC | ddd | | | | |
| | | | GAC | ddd | | | | |
| | | | CAT | ddd | | | | |
| | | | TGC | dkk | | | | |
| | | | G | e | | | | |
| 612088 | 820 | 835 | CAG | ekk | 17 | 6037 | 6052 | 713 |
| | | | CAT | ddd | | | | |
| | | | CCC | ddd | | | | |
| | | | GAC | ddd | | | | |
| | | | CAT | dkk | | | | |
| | | | T | e | | | | |
| 612089 | 823 | 838 | GGC | ekk | 46 | 6040 | 6055 | 714 |
| | | | CAG | ddd | | | | |
| | | | CAT | ddd | | | | |
| | | | CCC | ddd | | | | |
| | | | GAC | dkk | | | | |
| | | | C | e | | | | |
| 612090 | 826 | 841 | GTT | ekk | 10 | 6043 | 6058 | 715 |
| | | | GGC | ddd | | | | |
| | | | CAG | ddd | | | | |
| | | | CAT | ddd | | | | |
| | | | CCC | dkk | | | | |
| | | | G | e | | | | |
| 612091 | 829 | 844 | GAA | ekk | 0 | 6046 | 6061 | 716 |
| | | | GTT | ddd | | | | |
| | | | GGC | ddd | | | | |
| | | | CAG | ddd | | | | |
| | | | CAT | dkk | | | | |
| | | | C | e | | | | |
| 612092 | 832 | 847 | CAA | ekk | 0 | 6049 | 6064 | 717 |
| | | | GAA | ddd | | | | |
| | | | GTT | ddd | | | | |
| | | | GGC | ddd | | | | |
| | | | CAG | dkk | | | | |
| | | | C | e | | | | |
| 612093 | 835 | 850 | GCC | ekk | 0 | 6052 | 6067 | 59 |
| | | | CAA | ddd | | | | |
| | | | GAA | ddd | | | | |
| | | | GTT | ddd | | | | |
| | | | GGC | dkk | | | | |
| | | | C | e | | | | |
| 612094 | 838 | 853 | GAA | ekk | 28 | 6055 | 6070 | 718 |
| | | | GCC | ddd | | | | |
| | | | CAA | ddd | | | | |
| | | | GAA | ddd | | | | |
| | | | GTT | dkk | | | | |
| | | | G | e | | | | |
| 612095 | 841 | 856 | ACG | ekk | 13 | 6058 | 6073 | 719 |
| | | | GAA | ddd | | | | |
| | | | GCC | ddd | | | | |
| | | | CAA | ddd | | | | |
| | | | GAA | dkk | | | | |
| | | | G | e | | | | |
| 612096 | 844 | 859 | TAT | ekk | 18 | 6061 | 6076 | 720 |
| | | | ACG | ddd | | | | |
| | | | GAA | ddd | | | | |
| | | | GCC | ddd | | | | |
| | | | CAA | dkk | | | | |
| | | | G | e | | | | |
| 612097 | 847 | 862 | ATA | ekk | 0 | 6064 | 6079 | 721 |
| | | | TAT | ddd | | | | |
| | | | ACG | ddd | | | | |
| | | | GAA | ddd | | | | |
| | | | GCC | dkk | | | | |
| | | | C | e | | | | |
| 612098 | 850 | 865 | GCC | ekk | 42 | 6067 | 6082 | 722 |
| | | | ATA | ddd | | | | |
| | | | TAT | ddd | | | | |
| | | | ACG | ddd | | | | |
| | | | GAA | dkk | | | | |
| | | | G | e | | | | |
| 612099 | 853 | 868 | CAT | ekk | 20 | 6070 | 6085 | 723 |
| | | | GCC | ddd | | | | |
| | | | ATA | ddd | | | | |
| | | | TAT | ddd | | | | |
| | | | ACG | dkk | | | | |
| | | | G | e | | | | |
| 612100 | 856 | 871 | GTG | ekk | 47 | 6073 | 6088 | 724 |
| | | | CAT | ddd | | | | |
| | | | GCC | ddd | | | | |
| | | | ATA | ddd | | | | |
| | | | TAT | dkk | | | | |
| | | | A | e | | | | |
| 612101 | 859 | 874 | ACT | ekk | 52 | 6076 | 6091 | 725 |
| | | | GTG | ddd | | | | |
| | | | CAT | ddd | | | | |
| | | | GCC | ddd | | | | |
| | | | ATA | dkk | | | | |
| | | | T | e | | | | |
| 612102 | 862 | 877 | CTC | ekk | 62 | 6079 | 6094 | 726 |
| | | | ACT | ddd | | | | |
| | | | GTG | ddd | | | | |
| | | | CAT | ddd | | | | |
| | | | GCC | dkk | | | | |
| | | | A | e | | | | |
| 612103 | 865 | 880 | TAG | ekk | 45 | 6082 | 6097 | 727 |
| | | | CTC | ddd | | | | |
| | | | ACT | ddd | | | | |
| | | | GTG | ddd | | | | |
| | | | CAT | dkk | | | | |
| | | | G | e | | | | |
| 612104 | 868 | 883 | CCA | ekk | 66 | 6085 | 6100 | 728 |
| | | | TAG | ddd | | | | |
| | | | CTC | ddd | | | | |
| | | | ACT | ddd | | | | |
| | | | GTG | dkk | | | | |
| | | | C | e | | | | |
| 612105 | 871 | 886 | GCC | ekk | 16 | 6088 | 6103 | 62 |
| | | | CCA | ddd | | | | |
| | | | TAG | ddd | | | | |
| | | | CTC | ddd | | | | |
| | | | ACT | dkk | | | | |
| | | | G | e | | | | |
| 612107 | 877 | 892 | GAC | ekk | 0 | 6094 | 6109 | 729 |
| | | | CAC | ddd | | | | |
| | | | GCC | ddd | | | | |
| | | | CCA | ddd | | | | |
| | | | TAG | dkk | | | | |
| | | | C | e | | | | |
| 612108 | 880 | 895 | ATG | ekk | 0 | 6097 | 6112 | 730 |
| | | | GAC | ddd | | | | |
| | | | CAC | ddd | | | | |
| | | | GCC | ddd | | | | |
| | | | CCA | dkk | | | | |
| | | | T | e | | | | |
| 612109 | 884 | 899 | CCC | ekk | 0 | 6101 | 6116 | 731 |
| | | | CAT | ddd | | | | |
| | | | GGA | ddd | | | | |
| | | | CCA | ddd | | | | |
| | | | CGC | dkk | | | | |
| | | | C | e | | | | |
| 612110 | 887 | 902 | TGG | ekk | 24 | 6104 | 6119 | 732 |
| | | | CCC | ddd | | | | |
| | | | CAT | ddd | | | | |
| | | | GGA | ddd | | | | |
| | | | CCA | dkk | | | | |
| | | | C | e | | | | |
| 612111 | 890 | 905 | CGG | ekk | 1 | 6107 | 6122 | 733 |
| | | | TGG | ddd | | | | |
| | | | CCC | ddd | | | | |
| | | | CAT | ddd | | | | |
| | | | GGA | dkk | | | | |
| | | | C | e | | | | |
| 612112 | 893 | 908 | GGA | ekk | 4 | 6110 | 6125 | 734 |
| | | | CGG | ddd | | | | |
| | | | TGG | ddd | | | | |
| | | | CCC | ddd | | | | |
| | | | CAT | dkk | | | | |
| | | | G | e | | | | |
| 612113 | 896 | 911 | AGA | ekk | 7 | 6113 | 6128 | 735 |
| | | | GGA | ddd | | | | |
| | | | CGG | ddd | | | | |
| | | | TGG | ddd | | | | |
| | | | CCC | dkk | | | | |
| | | | C | e | | | | |
| 612114 | 899 | 914 | GGG | ekk | 28 | 6116 | 6131 | 736 |
| | | | AGA | ddd | | | | |
| | | | GGA | ddd | | | | |
| | | | CGG | ddd | | | | |
| | | | TGG | dkk | | | | |
| | | | C | e | | | | |
| 612115 | 913 | 928 | AAA | ekk | 30 | 6130 | 6145 | 64 |
| | | | GAC | ddd | | | | |
| | | | AGC | ddd | | | | |
| | | | CGT | ddd | | | | |
| | | | TGG | dkk | | | | |
| | | | G | e | | | | |
| 612116 | 916 | 931 | GCC | ekk | 45 | 6133 | 6148 | 737 |
| | | | AAA | ddd | | | | |
| | | | GAC | ddd | | | | |
| | | | AGC | ddd | | | | |
| | | | CGT | dkk | | | | |
| | | | T | e | | | | |
| 612117 | 919 | 934 | GGT | ekk | 52 | 6136 | 6151 | 738 |
| | | | GCC | ddd | | | | |
| | | | AAA | ddd | | | | |
| | | | GAC | ddd | | | | |
| | | | AGC | dkk | | | | |
| | | | C | e | | | | |
| 612118 | 922 | 937 | CAG | ekk | 20 | 6139 | 6154 | 739 |
| | | | GGT | ddd | | | | |
| | | | GCC | ddd | | | | |
| | | | AAA | ddd | | | | |
| | | | GAC | dkk | | | | |
| | | | A | e | | | | |
| 612119 | 926 | 941 | AGG | ekk | 20 | 6143 | 6158 | 740 |
| | | | CCA | ddd | | | | |
| | | | GGG | ddd | | | | |
| | | | TGC | ddd | | | | |
| | | | CAA | dkk | | | | |
| | | | A | e | | | | |
| 612120 | 937 | 952 | CAG | ekk | 0 | 6154 | 6169 | 66 |
| | | | ATA | ddd | | | | |
| | | | GAG | ddd | | | | |
| | | | AGA | ddd | | | | |
| | | | GGC | dkk | | | | |
| | | | C | e | | | | |
| 612121 | 940 | 955 | TCC | ekk | 0 | 6157 | 6172 | 741 |
| | | | CAG | ddd | | | | |
| | | | ATA | ddd | | | | |
| | | | GAG | ddd | | | | |
| | | | AGA | dkk | | | | |
| | | | G | e | | | | |
| 612122 | 943 | 958 | GGC | ekk | 11 | 6160 | 6175 | 742 |
| | | | TCC | ddd | | | | |
| | | | CAG | ddd | | | | |
| | | | ATA | ddd | | | | |
| | | | GAG | dkk | | | | |
| | | | A | e | | | | |
| 612123 | 946 | 961 | CAA | ekk | 5 | 6163 | 6178 | 743 |
| | | | GGC | ddd | | | | |
| | | | TCC | ddd | | | | |
| | | | CAG | ddd | | | | |
| | | | ATA | dkk | | | | |
| | | | G | e | | | | |
| 612124 | 949 | 964 | GTC | ekk | 14 | 6166 | 6181 | 744 |
| | | | CAA | ddd | | | | |
| | | | GGC | ddd | | | | |
| | | | TCC | ddd | | | | |
| | | | CAG | dkk | | | | |
| | | | A | e | | | | |
| 612125 | 952 | 967 | GTG | ekk | 19 | 6169 | 6184 | 745 |
| | | | GTC | ddd | | | | |
| | | | CAA | ddd | | | | |
| | | | GGC | ddd | | | | |
| | | | TCC | dkk | | | | |
| | | | C | e | | | | |
| 612126 | 955 | 970 | TGT | ekk | 25 | 6172 | 6187 | 746 |
| | | | GTG | ddd | | | | |
| | | | GTC | ddd | | | | |
| | | | CAA | ddd | | | | |
| | | | GGC | dkk | | | | |
| | | | T | e | | | | |
| 612127 | 958 | 973 | AGC | ekk | 40 | 6175 | 6190 | 747 |
| | | | TGT | ddd | | | | |
| | | | GTG | ddd | | | | |
| | | | GTC | ddd | | | | |
| | | | CAA | dkk | | | | |
| | | | G | e | | | | |
| 612128 | 961 | 976 | GTC | ekk | 22 | 6178 | 6193 | 748 |
| | | | AGC | ddd | | | | |
| | | | TGT | ddd | | | | |
| | | | GTG | ddd | | | | |
| | | | GTC | dkk | | | | |
| | | | C | e | | | | |
| 612281 | 1547 | 1562 | CAG | ekk | 25 | 10558 | 10573 | 749 |
| | | | AGG | ddd | | | | |
| | | | CAT | ddd | | | | |
| | | | AGT | ddd | | | | |
| | | | GAG | dkk | | | | |
| | | | G | e | | | | |
| 612282 | 1550 | 1565 | GGT | ekk | 20 | 10561 | 10576 | 103 |
| | | | CAG | ddd | | | | |
| | | | AGG | ddd | | | | |
| | | | CAT | ddd | | | | |
| | | | AGT | dkk | | | | |
| | | | G | e | | | | |
| 612283 | 1553 | 1568 | CCA | ekk | 36 | 10564 | 10579 | 750 |
| | | | GGT | ddd | | | | |
| | | | CAG | ddd | | | | |
| | | | AGG | ddd | | | | |
| | | | CAT | dkk | | | | |
| | | | A | e | | | | |
| 612284 | 1557 | 1572 | TTG | ekk | 24 | 10568 | 10583 | 751 |
| | | | TCC | ddd | | | | |
| | | | AGG | ddd | | | | |
| | | | TCA | ddd | | | | |
| | | | GAG | dkk | | | | |
| | | | G | e | | | | |
| 612285 | 1560 | 1575 | ACC | ekk | 37 | 10571 | 10586 | 752 |
| | | | TTG | ddd | | | | |
| | | | TCC | ddd | | | | |
| | | | AGG | ddd | | | | |
| | | | TCA | dkk | | | | |
| | | | G | e | | | | |
| 612286 | 1566 | 1581 | CCC | ekk | 9 | 10577 | 10592 | 753 |
| | | | TCC | ddd | | | | |
| | | | ACC | ddd | | | | |
| | | | TTG | ddd | | | | |
| | | | TCC | dkk | | | | |
| | | | A | e | | | | |
| 612287 | 1570 | 1585 | GAG | ekk | 31 | 10581 | 10596 | 754 |
| | | | ACC | ddd | | | | |
| | | | CTC | ddd | | | | |
| | | | CAC | ddd | | | | |
| | | | CTT | dkk | | | | |
| | | | G | e | | | | |
| 612288 | 1574 | 1589 | AAG | ekk | 5 | 10585 | 10600 | 755 |
| | | | TGA | ddd | | | | |
| | | | GAC | ddd | | | | |
| | | | CCT | ddd | | | | |
| | | | CCA | dkk | | | | |
| | | | C | e | | | | |
| 612289 | 1578 | 1593 | TGG | ekk | 13 | 10589 | 10604 | 104 |
| | | | AAA | ddd | | | | |
| | | | GTG | ddd | | | | |
| | | | AGA | ddd | | | | |
| | | | CCC | dkk | | | | |
| | | | T | e | | | | |
| 612290 | 1581 | 1596 | TGC | ekk | 27 | 10592 | 10607 | 756 |
| | | | TGG | ddd | | | | |
| | | | AAA | ddd | | | | |
| | | | GTG | ddd | | | | |
| | | | AGA | dkk | | | | |
| | | | C | e | | | | |
| 612291 | 1584 | 1599 | TTT | ekk | 0 | 10595 | 10610 | 757 |
| | | | TGC | ddd | | | | |
| | | | TGG | ddd | | | | |
| | | | AAA | ddd | | | | |
| | | | GTG | dkk | | | | |
| | | | A | e | | | | |
| 612292 | 1587 | 1602 | GAG | ekk | 15 | 10598 | 10613 | 758 |
| | | | TTT | ddd | | | | |
| | | | TGC | ddd | | | | |
| | | | TGG | ddd | | | | |
| | | | AAA | dkk | | | | |
| | | | G | e | | | | |
| 612293 | 1590 | 1605 | AGG | ekk | 27 | 10601 | 10616 | 759 |
| | | | GAG | ddd | | | | |
| | | | TTT | ddd | | | | |
| | | | TGC | ddd | | | | |
| | | | TGG | dkk | | | | |
| | | | A | e | | | | |
| 612294 | 1594 | 1609 | GTT | ekk | 0 | 10605 | 10620 | 760 |
| | | | GAG | ddd | | | | |
| | | | GGA | ddd | | | | |
| | | | GTT | ddd | | | | |
| | | | TTG | dkk | | | | |
| | | | C | e | | | | |
| 612295 | 1597 | 1612 | CCA | ekk | 6 | 10608 | 10623 | 761 |
| | | | GTT | ddd | | | | |
| | | | GAG | ddd | | | | |
| | | | GGA | ddd | | | | |
| | | | GTT | dkk | | | | |
| | | | T | e | | | | |
| 612296 | 1600 | 1615 | CAT | ekk | 8 | 10611 | 10626 | 762 |
| | | | CCA | ddd | | | | |
| | | | GTT | ddd | | | | |
| | | | GAG | ddd | | | | |
| | | | GGA | dkk | | | | |
| | | | G | e | | | | |
| 612297 | 1603 | 1618 | CTT | ekk | 11 | 10614 | 10629 | 763 |
| | | | CAT | ddd | | | | |
| | | | CCA | ddd | | | | |
| | | | GTT | ddd | | | | |
| | | | GAG | dkk | | | | |
| | | | G | e | | | | |
| 612298 | 1612 | 1627 | AGA | ekk | 0 | 10623 | 10638 | 764 |
| | | | TAG | ddd | | | | |
| | | | TTT | ddd | | | | |
| | | | CTT | ddd | | | | |
| | | | CAT | dkk | | | | |
| | | | C | e | | | | |
| 612299 | 1629 | 1644 | AGG | ekk | 36 | N/A | N/A | 765 |
| | | | TGG | ddd | | | | |
| | | | ATG | ddd | | | | |
| | | | GTC | ddd | | | | |
| | | | CGG | dkk | | | | |
| | | | G | e | | | | |
| 612300 | 1632 | 1647 | GTC | ekk | 25 | 12238 | 12253 | 766 |
| | | | AGG | ddd | | | | |
| | | | TGG | ddd | | | | |
| | | | ATG | ddd | | | | |
| | | | GTC | dkk | | | | |
| | | | C | e | | | | |
| 612301 | 1636 | 1651 | CAT | ekk | 26 | 12242 | 12257 | 767 |
| | | | GGT | ddd | | | | |
| | | | CAG | ddd | | | | |
| | | | GTG | ddd | | | | |
| | | | GAT | dkk | | | | |
| | | | G | e | | | | |
| 612302 | 1639 | 1654 | GGG | ekk | 40 | 12245 | 12260 | 768 |
| | | | CAT | ddd | | | | |
| | | | GGT | ddd | | | | |
| | | | CAG | ddd | | | | |
| | | | GTG | dkk | | | | |
| | | | G | e | | | | |
| 612303 | 1653 | 1668 | TGC | ekk | 33 | 12259 | 12274 | 109 |
| | | | AGC | ddd | | | | |
| | | | ACC | ddd | | | | |
| | | | AGT | ddd | | | | |
| | | | TGG | dkk | | | | |
| | | | G | e | | | | |
| 612304 | 1656 | 1671 | CCT | ekk | 3 | 12262 | 12277 | 769 |
| | | | TGC | ddd | | | | |
| | | | AGC | ddd | | | | |
| | | | ACC | ddd | | | | |
| | | | AGT | dkk | | | | |
| | | | T | e | | | | |
| 612305 | 1659 | 1674 | GAT | ekk | 12 | 12265 | 12280 | 770 |
| | | | CCT | ddd | | | | |
| | | | TGC | ddd | | | | |
| | | | AGC | ddd | | | | |
| | | | ACC | dkk | | | | |
| | | | A | e | | | | |
| 612306 | 1662 | 1677 | TAA | ekk | 8 | 12268 | 12283 | 771 |
| | | | GAT | ddd | | | | |
| | | | CCT | ddd | | | | |
| | | | TGC | ddd | | | | |
| | | | AGC | dkk | | | | |
| | | | A | e | | | | |
| 612307 | 1665 | 1680 | TCA | ekk | 8 | 12271 | 12286 | 772 |
| | | | TAA | ddd | | | | |
| | | | GAT | ddd | | | | |
| | | | CCT | ddd | | | | |
| | | | TGC | dkk | | | | |
| | | | A | e | | | | |
| 612308 | 1669 | 1684 | CAG | ekk | 8 | 12275 | 12290 | 773 |
| | | | GTC | ddd | | | | |
| | | | ATA | ddd | | | | |
| | | | AGA | ddd | | | | |
| | | | TCC | dkk | | | | |
| | | | T | e | | | | |
| 612309 | 1672 | 1687 | CTG | ekk | 0 | 12278 | 12293 | 774 |
| | | | CAG | ddd | | | | |
| | | | GTC | ddd | | | | |
| | | | ATA | ddd | | | | |
| | | | AGA | dkk | | | | |
| | | | T | e | | | | |
| 612310 | 1675 | 1690 | GTC | ekk | 10 | 12281 | 12296 | 775 |
| | | | CTG | ddd | | | | |
| | | | CAG | ddd | | | | |
| | | | GTC | ddd | | | | |
| | | | ATA | dkk | | | | |
| | | | A | e | | | | |
| 612311 | 1682 | 1697 | CGA | ekk | 32 | 12288 | 12303 | 776 |
| | | | GCA | ddd | | | | |
| | | | GGT | ddd | | | | |
| | | | CCT | ddd | | | | |
| | | | GCA | dkk | | | | |
| | | | G | e | | | | |
| 612312 | 1685 | 1700 | GGG | ekk | 11 | 12291 | 12306 | 777 |
| | | | CGA | ddd | | | | |
| | | | GCA | ddd | | | | |
| | | | GGT | ddd | | | | |
| | | | CCT | dkk | | | | |
| | | | G | e | | | | |
| 612313 | 1688 | 1703 | CCT | ekk | 22 | 12294 | 12309 | 778 |
| | | | GGG | ddd | | | | |
| | | | CGA | ddd | | | | |
| | | | GCA | ddd | | | | |
| | | | GGT | dkk | | | | |
| | | | C | e | | | | |
| 612314 | 1700 | 1715 | CGG | ekk | 0 | 12306 | 12321 | 112 |
| | | | GCA | ddd | | | | |
| | | | GCT | ddd | | | | |
| | | | CAG | ddd | | | | |
| | | | CCT | dkk | | | | |
| | | | G | e | | | | |
| 612315 | 1703 | 1718 | TGG | ekk | 55 | 12309 | 12324 | 779 |
| | | | CGG | ddd | | | | |
| | | | GCA | ddd | | | | |
| | | | GCT | ddd | | | | |
| | | | CAG | dkk | | | | |
| | | | C | e | | | | |
| 612316 | 1706 | 1721 | GAA | ekk | 16 | 12312 | 12327 | 780 |
| | | | TGG | ddd | | | | |
| | | | CGG | ddd | | | | |
| | | | GCA | ddd | | | | |
| | | | GCT | dkk | | | | |
| | | | C | e | | | | |
| 612317 | 1709 | 1724 | GCA | ekk | 16 | 12315 | 12330 | 781 |
| | | | GAA | ddd | | | | |
| | | | TGG | ddd | | | | |
| | | | CGG | ddd | | | | |
| | | | GCA | dkk | | | | |
| | | | G | e | | | | |
| 612318 | 1712 | 1727 | TGT | ekk | 24 | 12318 | 12333 | 782 |
| | | | GCA | ddd | | | | |
| | | | GAA | ddd | | | | |
| | | | TGG | ddd | | | | |
| | | | CGG | dkk | | | | |
| | | | G | e | | | | |
| 612319 | 1715 | 1730 | CGG | ekk | 35 | 12321 | 12336 | 783 |
| | | | TGT | ddd | | | | |
| | | | GCA | ddd | | | | |
| | | | GAA | ddd | | | | |
| | | | TGG | dkk | | | | |
| | | | C | e | | | | |
| 612320 | 1718 | 1733 | GCT | ekk | 13 | 12324 | 12339 | 784 |
| | | | CGG | ddd | | | | |
| | | | TGT | ddd | | | | |
| | | | GCA | ddd | | | | |
| | | | GAA | dkk | | | | |
| | | | T | e | | | | |
| 612321 | 1721 | 1736 | TCA | ekk | 28 | 12327 | 12342 | 785 |
| | | | GCT | ddd | | | | |
| | | | CGG | ddd | | | | |
| | | | TGT | ddd | | | | |
| | | | GCA | dkk | | | | |
| | | | G | e | | | | |
| 612322 | 1724 | 1739 | GGT | ekk | 49 | 12330 | 12345 | 786 |
| | | | TCA | ddd | | | | |
| | | | GCT | ddd | | | | |
| | | | CGG | ddd | | | | |
| | | | TGT | dkk | | | | |
| | | | G | e | | | | |
| 612323 | 1727 | 1742 | GCA | ekk | 53 | 12333 | 12348 | 787 |
| | | | GGT | ddd | | | | |
| | | | TCA | ddd | | | | |
| | | | GCT | ddd | | | | |
| | | | CGG | dkk | | | | |
| | | | T | e | | | | |
| 612324 | 1732 | 1747 | TTT | ekk | 8 | 12338 | 12353 | 788 |
| | | | TTG | ddd | | | | |
| | | | CAG | ddd | | | | |
| | | | GTT | ddd | | | | |
| | | | CAG | dkk | | | | |
| | | | C | e | | | | |
| 612325 | 1735 | 1750 | CAA | ekk | 14 | 12341 | 12356 | 789 |
| | | | TTT | ddd | | | | |
| | | | TTG | ddd | | | | |
| | | | CAG | ddd | | | | |
| | | | GTT | dkk | | | | |
| | | | C | e | | | | |
| 612326 | 1738 | 1753 | GCT | ekk | 38 | 12344 | 12359 | 790 |
| | | | CAA | ddd | | | | |
| | | | TTT | ddd | | | | |
| | | | TTG | ddd | | | | |
| | | | CAG | dkk | | | | |
| | | | G | e | | | | |
| 612327 | 1741 | 1756 | ATT | ekk | 2 | 12347 | 12362 | 791 |
| | | | GCT | ddd | | | | |
| | | | CAA | ddd | | | | |
| | | | TTT | ddd | | | | |
| | | | TTG | dkk | | | | |
| | | | C | e | | | | |
| 612328 | 1744 | 1759 | GTC | ekk | 38 | 12350 | 12365 | 792 |
| | | | ATT | ddd | | | | |
| | | | GCT | ddd | | | | |
| | | | CAA | ddd | | | | |
| | | | TTT | dkk | | | | |
| | | | T | e | | | | |
| 612329 | 1747 | 1762 | GCG | ekk | 32 | 12353 | 12368 | 793 |
| | | | GTC | ddd | | | | |
| | | | ATT | ddd | | | | |
| | | | GCT | ddd | | | | |
| | | | CAA | dkk | | | | |
| | | | T | e | | | | |
| 612330 | 1750 | 1765 | GAT | ekk | 27 | 12356 | 12371 | 794 |
| | | | GCG | ddd | | | | |
| | | | GTC | ddd | | | | |
| | | | ATT | ddd | | | | |
| | | | GCT | dkk | | | | |
| | | | C | e | | | | |
| 612331 | 1753 | 1768 | CCT | ekk | 15 | 12359 | 12374 | 795 |
| | | | GAT | ddd | | | | |
| | | | GCG | ddd | | | | |
| | | | GTC | ddd | | | | |
| | | | ATT | dkk | | | | |
| | | | G | e | | | | |
| 612332 | 1756 | 1771 | CAC | ekk | 1 | 12362 | 12377 | 796 |
| | | | CCT | ddd | | | | |
| | | | GAT | ddd | | | | |
| | | | GCG | ddd | | | | |
| | | | GTC | dkk | | | | |
| | | | A | e | | | | |
| 612333 | 1759 | 1774 | CCC | ekk | 11 | 12365 | 12380 | 797 |
| | | | CAC | ddd | | | | |
| | | | CCT | ddd | | | | |
| | | | GAT | ddd | | | | |
| | | | GCG | dkk | | | | |
| | | | G | e | | | | |
| 612334 | 1762 | 1777 | CTC | ekk | 0 | 12368 | 12383 | 798 |
| | | | CCC | ddd | | | | |
| | | | CAC | ddd | | | | |
| | | | CCT | ddd | | | | |
| | | | GAT | dkk | | | | |
| | | | G | e | | | | |
| 612335 | 1771 | 1786 | GTT | ekk | 12 | N/A | N/A | 799 |
| | | | CAG | ddd | | | | |
| | | | CAC | ddd | | | | |
| | | | CTC | ddd | | | | |
| | | | CCC | dkk | | | | |
| | | | C | e | | | | |
| 612336 | 1774 | 1789 | GCT | ekk | 57 | N/A | N/A | 800 |
| | | | GTT | ddd | | | | |
| | | | CAG | ddd | | | | |
| | | | CAC | ddd | | | | |
| | | | CTC | dkk | | | | |
| | | | C | e | | | | |
| 612337 | 1777 | 1792 | AAT | ekk | 29 | 13246 | 13261 | 801 |
| | | | GCT | ddd | | | | |
| | | | GTT | ddd | | | | |
| | | | CAG | ddd | | | | |
| | | | CAC | dkk | | | | |
| | | | C | e | | | | |
| 612338 | 1780 | 1795 | AAA | ekk | 38 | 13249 | 13264 | 802 |
| | | | AAT | ddd | | | | |
| | | | GCT | ddd | | | | |
| | | | GTT | ddd | | | | |
| | | | CAG | dkk | | | | |
| | | | C | e | | | | |
| 612339 | 1793 | 1808 | CTT | ekk | 0 | 13262 | 13277 | 803 |
| | | | CAA | ddd | | | | |
| | | | GCT | ddd | | | | |
| | | | CAA | ddd | | | | |
| | | | AAA | dkk | | | | |
| | | | A | e | | | | |
| 612340 | 1796 | 1811 | CCG | ekk | 41 | 13265 | 13280 | 804 |
| | | | CTT | ddd | | | | |
| | | | CAA | ddd | | | | |
| | | | GCT | ddd | | | | |
| | | | CAA | dkk | | | | |
| | | | A | e | | | | |
| 612341 | 1799 | 1814 | CAT | ekk | 27 | 13268 | 13283 | 805 |
| | | | CCG | ddd | | | | |
| | | | CTT | ddd | | | | |
| | | | CAA | ddd | | | | |
| | | | GCT | dkk | | | | |
| | | | C | e | | | | |
| 612342 | 1802 | 1817 | TCT | ekk | 32 | 13271 | 13286 | 806 |
| | | | CAT | ddd | | | | |
| | | | CCG | ddd | | | | |
| | | | CTT | ddd | | | | |
| | | | CAA | dkk | | | | |
| | | | G | e | | | | |
| 612343 | 1805 | 1820 | CTC | ekk | 26 | 13274 | 13289 | 807 |
| | | | TCT | ddd | | | | |
| | | | CAT | ddd | | | | |
| | | | CCG | ddd | | | | |
| | | | CTT | dkk | | | | |
| | | | C | e | | | | |
| 612344 | 1808 | 1823 | GCT | ekk | 44 | 13277 | 13292 | 808 |
| | | | CTC | ddd | | | | |
| | | | TCT | ddd | | | | |
| | | | CAT | ddd | | | | |
| | | | CCG | dkk | | | | |
| | | | C | e | | | | |
| 612345 | 1812 | 1827 | GTG | ekk | 15 | 13281 | 13296 | 809 |
| | | | GGC | ddd | | | | |
| | | | TCT | ddd | | | | |
| | | | CTC | ddd | | | | |
| | | | TCA | dkk | | | | |
| | | | T | e | | | | |
| 612346 | 1817 | 1832 | ACT | ekk | 42 | 13286 | 13301 | 810 |
| | | | CTG | ddd | | | | |
| | | | TGG | ddd | | | | |
| | | | GCT | ddd | | | | |
| | | | CTC | dkk | | | | |
| | | | T | e | | | | |
| 612347 | 1820 | 1835 | TAG | ekk | 55 | 13289 | 13304 | 811 |
| | | | ACT | ddd | | | | |
| | | | CTG | ddd | | | | |
| | | | TGG | ddd | | | | |
| | | | GCT | dkk | | | | |
| | | | C | e | | | | |
| 612348 | 1824 | 1839 | TGG | ekk | 23 | 13293 | 13308 | 812 |
| | | | GTA | ddd | | | | |
| | | | GAC | ddd | | | | |
| | | | TCT | ddd | | | | |
| | | | GTG | dkk | | | | |
| | | | G | e | | | | |
| 612349 | 1827 | 1842 | TGT | ekk | 30 | 13296 | 13311 | 119 |
| | | | TGG | ddd | | | | |
| | | | GTA | ddd | | | | |
| | | | GAC | ddd | | | | |
| | | | TCT | dkk | | | | |
| | | | G | e | | | | |
| 612350 | 1830 | 1845 | AGC | ekk | 34 | 13299 | 13314 | 813 |
| | | | TGT | ddd | | | | |
| | | | TGG | ddd | | | | |
| | | | GTA | ddd | | | | |
| | | | GAC | dkk | | | | |
| | | | T | e | | | | |
| 612351 | 1833 | 1848 | TTA | ekk | 13 | 13302 | 13317 | 814 |
| | | | AGC | ddd | | | | |
| | | | TGT | ddd | | | | |
| | | | TGG | ddd | | | | |
| | | | GTA | dkk | | | | |
| | | | G | e | | | | |
| 612352 | 1836 | 1851 | TTG | ekk | 33 | 13305 | 13320 | 815 |
| | | | TTA | ddd | | | | |
| | | | AGC | ddd | | | | |
| | | | TGT | ddd | | | | |
| | | | TGG | dkk | | | | |
| | | | G | e | | | | |
| 612353 | 1839 | 1854 | GGC | ekk | 30 | 13308 | 13323 | 816 |
| | | | TTG | ddd | | | | |
| | | | TTA | ddd | | | | |
| | | | AGC | ddd | | | | |
| | | | TGT | dkk | | | | |
| | | | T | e | | | | |
| 612354 | 1842 | 1857 | TCA | ekk | 10 | 13311 | 13326 | 817 |
| | | | GGC | ddd | | | | |
| | | | TTG | ddd | | | | |
| | | | TTA | ddd | | | | |
| | | | AGC | dkk | | | | |
| | | | T | e | | | | |
| 612355 | 1845 | 1860 | ACC | ekk | 17 | 13314 | 13329 | 818 |
| | | | TCA | ddd | | | | |
| | | | GGC | ddd | | | | |
| | | | TTG | ddd | | | | |
| | | | TTA | dkk | | | | |
| | | | A | e | | | | |
| 612356 | 1848 | 1863 | AAG | ekk | 33 | 13317 | 13332 | 819 |
| | | | ACC | ddd | | | | |
| | | | TCA | ddd | | | | |
| | | | GGC | ddd | | | | |
| | | | TTG | dkk | | | | |
| | | | T | e | | | | |
| 612609 | 2409 | 2424 | ACA | ekk | 20 | 13878 | 13893 | 820 |
| | | | CGG | ddd | | | | |
| | | | AGG | ddd | | | | |
| | | | TCA | ddd | | | | |
| | | | TGT | dkk | | | | |
| | | | T | e | | | | |
| 612610 | 2410 | 2425 | TAC | ekk | 25 | 13879 | 13894 | 821 |
| | | | ACG | ddd | | | | |
| | | | GAG | ddd | | | | |
| | | | GTC | ddd | | | | |
| | | | ATG | dkk | | | | |
| | | | T | e | | | | |
| 612611 | 2411 | 2426 | CTA | ekk | 24 | 13880 | 13895 | 822 |
| | | | CAC | ddd | | | | |
| | | | GGA | ddd | | | | |
| | | | GGT | ddd | | | | |
| | | | CAT | dkk | | | | |
| | | | G | e | | | | |
| 612612 | 2412 | 2427 | ACT | ekk | 26 | 13881 | 13896 | 145 |
| | | | ACA | ddd | | | | |
| | | | CGG | ddd | | | | |
| | | | AGG | ddd | | | | |
| | | | TCA | dkk | | | | |
| | | | T | e | | | | |
| 612613 | 2413 | 2428 | CAC | ekk | 30 | 13882 | 13897 | 823 |
| | | | TAC | ddd | | | | |
| | | | ACG | ddd | | | | |
| | | | GAG | ddd | | | | |
| | | | GTC | dkk | | | | |
| | | | A | e | | | | |
| 612614 | 2414 | 2429 | ACA | ekk | 49 | 13883 | 13898 | 824 |
| | | | CTA | ddd | | | | |
| | | | CAC | ddd | | | | |
| | | | GGA | ddd | | | | |
| | | | GGT | dkk | | | | |
| | | | C | e | | | | |
| 612615 | 2415 | 2430 | GAC | ekk | 56 | 13884 | 13899 | 825 |
| | | | ACT | ddd | | | | |
| | | | ACA | ddd | | | | |
| | | | CGG | ddd | | | | |
| | | | AGG | dkk | | | | |
| | | | T | e | | | | |
| 612616 | 2416 | 2431 | AGA | ekk | 40 | 13885 | 13900 | 826 |
| | | | CAC | ddd | | | | |
| | | | TAC | ddd | | | | |
| | | | ACG | ddd | | | | |
| | | | GAG | dkk | | | | |
| | | | G | e | | | | |
| 612617 | 2417 | 2432 | CAG | ekk | 48 | 13886 | 13901 | 827 |
| | | | ACA | ddd | | | | |
| | | | CTA | ddd | | | | |
| | | | CAC | ddd | | | | |
| | | | GGA | dkk | | | | |
| | | | G | e | | | | |
| 612618 | 2418 | 2433 | ACA | ekk | 44 | 13887 | 13902 | 828 |
| | | | GAC | ddd | | | | |
| | | | ACT | ddd | | | | |
| | | | ACA | ddd | | | | |
| | | | CGG | dkk | | | | |
| | | | A | e | | | | |
| 612619 | 2419 | 2434 | TAC | ekk | 39 | 13888 | 13903 | 829 |
| | | | AGA | ddd | | | | |
| | | | CAC | ddd | | | | |
| | | | TAC | ddd | | | | |
| | | | ACG | dkk | | | | |
| | | | G | e | | | | |
| 612620 | 2420 | 2435 | TTA | ekk | 28 | 13889 | 13904 | 830 |
| | | | CAG | ddd | | | | |
| | | | ACA | ddd | | | | |
| | | | CTA | ddd | | | | |
| | | | CAC | dkk | | | | |
| | | | G | e | | | | |
| 612621 | 2421 | 2436 | ATT | ekk | 21 | 13890 | 13905 | 831 |
| | | | ACA | ddd | | | | |
| | | | GAC | ddd | | | | |
| | | | ACT | ddd | | | | |
| | | | ACA | dkk | | | | |
| | | | C | e | | | | |
| 612622 | 2422 | 2437 | TAT | ekk | 0 | 13891 | 13906 | 146 |
| | | | TAC | ddd | | | | |
| | | | AGA | ddd | | | | |
| | | | CAC | ddd | | | | |
| | | | TAC | dkk | | | | |
| | | | A | e | | | | |
| 612623 | 2423 | 2438 | GTA | ekk | 35 | 13892 | 13907 | 832 |
| | | | TTA | ddd | | | | |
| | | | CAG | ddd | | | | |
| | | | ACA | ddd | | | | |
| | | | CTA | dkk | | | | |
| | | | C | e | | | | |
| 612624 | 2428 | 2443 | CTA | ekk | 8 | 13897 | 13912 | 833 |
| | | | AGG | ddd | | | | |
| | | | TAT | ddd | | | | |
| | | | TAC | ddd | | | | |
| | | | AGA | dkk | | | | |
| | | | C | e | | | | |
| 612625 | 2429 | 2444 | ACT | ekk | 14 | 13898 | 13913 | 834 |
| | | | AAG | ddd | | | | |
| | | | GTA | ddd | | | | |
| | | | TTA | ddd | | | | |
| | | | CAG | dkk | | | | |
| | | | A | e | | | | |
| 612626 | 2430 | 2445 | AAC | ekk | 14 | 13899 | 13914 | 835 |
| | | | TAA | ddd | | | | |
| | | | GGT | ddd | | | | |
| | | | ATT | ddd | | | | |
| | | | ACA | dkk | | | | |
| | | | G | e | | | | |
| 612627 | 2431 | 2446 | AAA | ekk | 12 | 13900 | 13915 | 836 |
| | | | CTA | ddd | | | | |
| | | | AGG | ddd | | | | |
| | | | TAT | ddd | | | | |
| | | | TAC | dkk | | | | |
| | | | A | e | | | | |
| 612628 | 2432 | 2447 | AAA | ekk | 3 | 13901 | 13916 | 837 |
| | | | ACT | ddd | | | | |
| | | | AAG | ddd | | | | |
| | | | GTA | ddd | | | | |
| | | | TTA | dkk | | | | |
| | | | C | e | | | | |
| 612629 | 2438 | 2453 | GTG | ekk | 0 | 13907 | 13922 | 838 |
| | | | GAA | ddd | | | | |
| | | | AAA | ddd | | | | |
| | | | ACT | ddd | | | | |
| | | | AAG | dkk | | | | |
| | | | G | e | | | | |
| 612630 | 2447 | 2462 | CAA | ekk | 0 | 13916 | 13931 | 839 |
| | | | GCA | ddd | | | | |
| | | | TCT | ddd | | | | |
| | | | GTG | ddd | | | | |
| | | | GAA | dkk | | | | |
| | | | A | e | | | | |
| 612631 | 2449 | 2464 | CAC | ekk | 20 | 13918 | 13933 | 840 |
| | | | AAG | ddd | | | | |
| | | | CAT | ddd | | | | |
| | | | CTG | ddd | | | | |
| | | | TGG | dkk | | | | |
| | | | A | e | | | | |
| 612632 | 2450 | 2465 | TCA | ekk | 1 | 13919 | 13934 | 841 |
| | | | CAA | ddd | | | | |
| | | | GCA | ddd | | | | |
| | | | TCT | ddd | | | | |
| | | | GTG | dkk | | | | |
| | | | G | e | | | | |
| 612633 | 2451 | 2466 | ATC | ekk | 20 | 13920 | 13935 | 842 |
| | | | ACA | ddd | | | | |
| | | | AGC | ddd | | | | |
| | | | ATC | ddd | | | | |
| | | | TGT | dkk | | | | |
| | | | G | e | | | | |
| 612634 | 2452 | 2467 | AAT | ekk | 2 | 13921 | 13936 | 843 |
| | | | CAC | ddd | | | | |
| | | | AAG | ddd | | | | |
| | | | CAT | ddd | | | | |
| | | | CTG | dkk | | | | |
| | | | T | e | | | | |
| 612635 | 2464 | 2479 | GTA | ekk | 16 | 13933 | 13948 | 844 |
| | | | TTG | ddd | | | | |
| | | | TTC | ddd | | | | |
| | | | AAA | ddd | | | | |
| | | | AAT | dkk | | | | |
| | | | C | e | | | | |
| 612636 | 2465 | 2480 | CGT | ekk | 0 | 13934 | 13949 | 845 |
| | | | ATT | ddd | | | | |
| | | | GTT | ddd | | | | |
| | | | CAA | ddd | | | | |
| | | | AAA | dkk | | | | |
| | | | T | e | | | | |
| 612637 | 2482 | 2497 | GGT | ekk | 21 | 13951 | 13966 | 147 |
| | | | GCT | ddd | | | | |
| | | | TGC | ddd | | | | |
| | | | ATC | ddd | | | | |
| | | | TTT | dkk | | | | |
| | | | C | e | | | | |
| 612638 | 2483 | 2498 | AGG | ekk | 13 | 13952 | 13967 | 846 |
| | | | TGC | ddd | | | | |
| | | | TTG | ddd | | | | |
| | | | CAT | ddd | | | | |
| | | | CTT | dkk | | | | |
| | | | T | e | | | | |
| 612639 | 2484 | 2499 | CAG | ekk | 19 | 13953 | 13968 | 847 |
| | | | GTG | ddd | | | | |
| | | | CTT | ddd | | | | |
| | | | GCA | ddd | | | | |
| | | | TCT | dkk | | | | |
| | | | T | e | | | | |
| 612640 | 2485 | 2500 | TCA | ekk | 38 | 13954 | 13969 | 848 |
| | | | GGT | ddd | | | | |
| | | | GCT | ddd | | | | |
| | | | TGC | ddd | | | | |
| | | | ATC | dkk | | | | |
| | | | T | e | | | | |
| 612641 | 2486 | 2501 | TTC | ekk | 29 | 13955 | 13970 | 849 |
| | | | AGG | ddd | | | | |
| | | | TGC | ddd | | | | |
| | | | TTG | ddd | | | | |
| | | | CAT | dkk | | | | |
| | | | C | e | | | | |
| 612642 | 2487 | 2502 | ATT | ekk | 19 | 13956 | 13971 | 850 |
| | | | CAG | ddd | | | | |
| | | | GTG | ddd | | | | |
| | | | CTT | ddd | | | | |
| | | | GCA | dkk | | | | |
| | | | T | e | | | | |
| 612643 | 2488 | 2503 | AAT | ekk | 34 | 13957 | 13972 | 851 |
| | | | TCA | ddd | | | | |
| | | | GGT | ddd | | | | |
| | | | GCT | ddd | | | | |
| | | | TGC | dkk | | | | |
| | | | A | e | | | | |
| 612644 | 2489 | 2504 | AAA | ekk | 24 | 13958 | 13973 | 852 |
| | | | TTC | ddd | | | | |
| | | | AGG | ddd | | | | |
| | | | TGC | ddd | | | | |
| | | | TTG | dkk | | | | |
| | | | C | e | | | | |
| 612645 | 2490 | 2505 | GAA | ekk | 2 | 13959 | 13974 | 853 |
| | | | ATT | ddd | | | | |
| | | | CAG | ddd | | | | |
| | | | GTG | ddd | | | | |
| | | | CTT | dkk | | | | |
| | | | G | e | | | | |
| 612646 | 2491 | 2506 | AGA | ekk | 5 | 13960 | 13975 | 854 |
| | | | AAT | ddd | | | | |
| | | | TCA | ddd | | | | |
| | | | GGT | ddd | | | | |
| | | | GCT | dkk | | | | |
| | | | T | e | | | | |
| 612647 | 2493 | 2508 | ACA | ekk | 0 | 13962 | 13977 | 855 |
| | | | GAA | ddd | | | | |
| | | | ATT | ddd | | | | |
| | | | CAG | ddd | | | | |
| | | | GTG | dkk | | | | |
| | | | C | e | | | | |
| 612648 | 2502 | 2517 | CGC | ekk | 22 | 13971 | 13986 | 856 |
| | | | ATT | ddd | | | | |
| | | | CAA | ddd | | | | |
| | | | ACA | ddd | | | | |
| | | | GAA | dkk | | | | |
| | | | A | e | | | | |
| 612649 | 2503 | 2518 | CCG | ekk | 50 | 13972 | 13987 | 149 |
| | | | CAT | ddd | | | | |
| | | | TCA | ddd | | | | |
| | | | AAC | ddd | | | | |
| | | | AGA | dkk | | | | |
| | | | A | e | | | | |
| 612650 | 2504 | 2519 | TCC | ekk | 35 | 13973 | 13988 | 857 |
| | | | GCA | ddd | | | | |
| | | | TTC | ddd | | | | |
| | | | AAA | ddd | | | | |
| | | | CAG | dkk | | | | |
| | | | A | e | | | | |
| 612651 | 2505 | 2520 | TTC | ekk | 29 | 13974 | 13989 | 858 |
| | | | CGC | ddd | | | | |
| | | | ATT | ddd | | | | |
| | | | CAA | ddd | | | | |
| | | | ACA | dkk | | | | |
| | | | G | e | | | | |
| 612652 | 2506 | 2521 | GTT | ekk | 25 | 13975 | 13990 | 859 |
| | | | CCG | ddd | | | | |
| | | | CAT | ddd | | | | |
| | | | TCA | ddd | | | | |
| | | | AAC | dkk | | | | |
| | | | A | e | | | | |
| 612653 | 2507 | 2522 | GGT | ekk | 28 | 13976 | 13991 | 860 |
| | | | TCC | ddd | | | | |
| | | | GCA | ddd | | | | |
| | | | TTC | ddd | | | | |
| | | | AAA | dkk | | | | |
| | | | C | e | | | | |
| 612654 | 2508 | 2523 | TGG | ekk | 38 | 13977 | 13992 | 861 |
| | | | TTC | ddd | | | | |
| | | | CGC | ddd | | | | |
| | | | ATT | ddd | | | | |
| | | | CAA | dkk | | | | |
| | | | A | e | | | | |
| 612655 | 2509 | 2524 | ATG | ekk | 45 | 13978 | 13993 | 862 |
| | | | GTT | ddd | | | | |
| | | | CCG | ddd | | | | |
| | | | CAT | ddd | | | | |
| | | | TCA | dkk | | | | |
| | | | A | e | | | | |
| 612656 | 2510 | 2525 | TAT | ekk | 42 | 13979 | 13994 | 863 |
| | | | GGT | ddd | | | | |
| | | | TCC | ddd | | | | |
| | | | GCA | ddd | | | | |
| | | | TTC | dkk | | | | |
| | | | A | e | | | | |
| 612657 | 2511 | 2526 | CTA | ekk | 41 | 13980 | 13995 | 864 |
| | | | TGG | ddd | | | | |
| | | | TTC | ddd | | | | |
| | | | CGC | ddd | | | | |
| | | | ATT | dkk | | | | |
| | | | C | e | | | | |
| 612658 | 2512 | 2527 | GCT | ekk | 58 | 13981 | 13996 | 865 |
| | | | ATG | ddd | | | | |
| | | | GTT | ddd | | | | |
| | | | CCG | ddd | | | | |
| | | | CAT | dkk | | | | |
| | | | T | e | | | | |
| 612659 | 2513 | 2528 | AGC | ekk | 32 | 13982 | 13997 | 150 |
| | | | TAT | ddd | | | | |
| | | | GGT | ddd | | | | |
| | | | TCC | ddd | | | | |
| | | | GCA | dkk | | | | |
| | | | T | e | | | | |
| 612660 | 2514 | 2529 | CAG | ekk | 46 | 13983 | 13998 | 866 |
| | | | CTA | ddd | | | | |
| | | | TGG | ddd | | | | |
| | | | TTC | ddd | | | | |
| | | | CGC | dkk | | | | |
| | | | A | e | | | | |
| 612661 | 2515 | 2530 | CCA | ekk | 47 | 13984 | 13999 | 867 |
| | | | GCT | ddd | | | | |
| | | | ATG | ddd | | | | |
| | | | GTT | ddd | | | | |
| | | | CCG | dkk | | | | |
| | | | C | e | | | | |
| 612662 | 2516 | 2531 | ACC | ekk | 60 | 13985 | 14000 | 868 |
| | | | AGC | ddd | | | | |
| | | | TAT | ddd | | | | |
| | | | GGT | ddd | | | | |
| | | | TCC | dkk | | | | |
| | | | G | e | | | | |
| 612663 | 2517 | 2532 | AAC | ekk | 36 | 13986 | 14001 | 869 |
| | | | CAG | ddd | | | | |
| | | | CTA | ddd | | | | |
| | | | TGG | ddd | | | | |
| | | | TTC | dkk | | | | |
| | | | C | e | | | | |
| 612664 | 2518 | 2533 | TAA | ekk | 0 | 13987 | 14002 | 870 |
| | | | CCA | ddd | | | | |
| | | | GCT | ddd | | | | |
| | | | ATG | ddd | | | | |
| | | | GTT | dkk | | | | |
| | | | C | e | | | | |
| 612665 | 2519 | 2534 | ATA | ekk | 17 | 13988 | 14003 | 871 |
| | | | ACC | ddd | | | | |
| | | | AGC | ddd | | | | |
| | | | TAT | ddd | | | | |
| | | | GGT | dkk | | | | |
| | | | T | e | | | | |
| 612666 | 2521 | 2536 | AAA | ekk | 3 | 13990 | 14005 | 872 |
| | | | TAA | ddd | | | | |
| | | | CCA | ddd | | | | |
| | | | GCT | ddd | | | | |
| | | | ATG | dkk | | | | |
| | | | G | e | | | | |
| 612667 | 2522 | 2537 | GAA | ekk | 2 | 13991 | 14006 | 873 |
| | | | ATA | ddd | | | | |
| | | | ACC | ddd | | | | |
| | | | AGC | ddd | | | | |
| | | | TAT | dkk | | | | |
| | | | G | e | | | | |
| 612668 | 2523 | 2538 | AGA | ekk | 4 | 13992 | 14007 | 874 |
| | | | AAT | ddd | | | | |
| | | | AAC | ddd | | | | |
| | | | CAG | ddd | | | | |
| | | | CTA | dkk | | | | |
| | | | T | e | | | | |
| 612669 | 2535 | 2550 | CTA | ekk | 23 | 14004 | 14019 | 875 |
| | | | ACA | ddd | | | | |
| | | | CAA | ddd | | | | |
| | | | GGG | ddd | | | | |
| | | | AGA | dkk | | | | |
| | | | A | e | | | | |
| 612670 | 2536 | 2551 | ACT | ekk | 13 | 14005 | 14020 | 876 |
| | | | AAC | ddd | | | | |
| | | | ACA | ddd | | | | |
| | | | AGG | ddd | | | | |
| | | | GAG | dkk | | | | |
| | | | A | e | | | | |
| 612671 | 2537 | 2552 | TAC | ekk | 9 | 14006 | 14021 | 151 |
| | | | TAA | ddd | | | | |
| | | | CAC | ddd | | | | |
| | | | AAG | ddd | | | | |
| | | | GGA | dkk | | | | |
| | | | G | e | | | | |
| 612672 | 2538 | 2553 | TTA | ekk | 51 | 14007 | 14022 | 877 |
| | | | CTA | ddd | | | | |
| | | | ACA | ddd | | | | |
| | | | CAA | ddd | | | | |
| | | | GGG | dkk | | | | |
| | | | A | e | | | | |
| 612673 | 2539 | 2554 | ATT | ekk | 47 | 14008 | 14023 | 878 |
| | | | ACT | ddd | | | | |
| | | | AAC | ddd | | | | |
| | | | ACA | ddd | | | | |
| | | | AGG | dkk | | | | |
| | | | G | e | | | | |
| 612674 | 2540 | 2555 | TAT | ekk | 16 | 14009 | 14024 | 879 |
| | | | TAC | ddd | | | | |
| | | | TAA | ddd | | | | |
| | | | CAC | ddd | | | | |
| | | | AAG | dkk | | | | |
| | | | G | e | | | | |
| 612675 | 2541 | 2556 | TTA | ekk | 0 | 14010 | 14025 | 880 |
| | | | TTA | ddd | | | | |
| | | | CTA | ddd | | | | |
| | | | ACA | ddd | | | | |
| | | | CAA | dkk | | | | |
| | | | G | e | | | | |
| 612676 | 2543 | 2558 | GTT | ekk | 0 | 14012 | 14027 | 881 |
| | | | TAT | ddd | | | | |
| | | | TAC | ddd | | | | |
| | | | TAA | ddd | | | | |
| | | | CAC | dkk | | | | |
| | | | A | e | | | | |
| 612677 | 2544 | 2559 | CGT | ekk | 35 | 14013 | 14028 | 882 |
| | | | TTA | ddd | | | | |
| | | | TTA | ddd | | | | |
| | | | CTA | ddd | | | | |
| | | | ACA | dkk | | | | |
| | | | C | e | | | | |
| 612678 | 2558 | 2573 | TTA | ekk | 28 | 14027 | 14042 | 152 |
| | | | TTG | ddd | | | | |
| | | | TGG | ddd | | | | |
| | | | CAA | ddd | | | | |
| | | | GAC | dkk | | | | |
| | | | G | e | | | | |
| 612679 | 2559 | 2574 | CTT | ekk | 21 | 14028 | 14043 | 883 |
| | | | ATT | ddd | | | | |
| | | | GTG | ddd | | | | |
| | | | GCA | ddd | | | | |
| | | | AGA | dkk | | | | |
| | | | C | e | | | | |
| 612680 | 2560 | 2575 | GCT | ekk | 16 | 14029 | 14044 | 884 |
| | | | TAT | ddd | | | | |
| | | | TGT | ddd | | | | |
| | | | GGC | ddd | | | | |
| | | | AAG | dkk | | | | |
| | | | A | e | | | | |
| 612681 | 2561 | 2576 | GGC | ekk | 35 | 14030 | 14045 | 885 |
| | | | TTA | ddd | | | | |
| | | | TTG | ddd | | | | |
| | | | TGG | ddd | | | | |
| | | | CAA | dkk | | | | |
| | | | G | e | | | | |
| 612682 | 2562 | 2577 | AGG | ekk | 34 | 14031 | 14046 | 886 |
| | | | CTT | ddd | | | | |
| | | | ATT | ddd | | | | |
| | | | GTG | ddd | | | | |
| | | | GCA | dkk | | | | |
| | | | A | e | | | | |
| 612683 | 2563 | 2578 | GAG | ekk | 23 | 14032 | 14047 | 887 |
| | | | GCT | ddd | | | | |
| | | | TAT | ddd | | | | |
| | | | TGT | ddd | | | | |
| | | | GGC | dkk | | | | |
| | | | A | e | | | | |
| 612684 | 2564 | 2579 | GGA | ekk | 0 | 14033 | 14048 | 888 |
| | | | GGC | ddd | | | | |
| | | | TTA | ddd | | | | |
| | | | TTG | ddd | | | | |
| | | | TGG | dkk | | | | |
| | | | C | e | | | | |

Table 5 shows inhibition of AGT mRNA in HepG2 cells cultured at a density of 20,000 cells per well which were transfected using electroporation with 1000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 5
Inhibition of AGT mRNA by MOE and/or cEt
containing gapmers targeting
SEQ ID NO: 1 and/or 2

| | SEQ | SEQ | | | | SEQ | SEQ | |
|---|---|---|---|---|---|---|---|---|
| | ID: | ID: | | | % | ID: | ID | |
| | 1 | 1 | | | Inhi- | 2 | 2: | SEQ |
| ISIS | Start | Stop | Se- | Chem- | bi- | Start | Stop | ID |
| NO | Site | Site | quence | istry | tion | Site | Site | NO |
| 568637 | 2046 | 2061 | CGC | eek | 87 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 90 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 95 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 568637 | 2046 | 2061 | CGC | eek | 94 | 13515 | 13530 | 129 |
| | | | TGA | ddd | | | | |
| | | | TTT | ddd | | | | |
| | | | GTC | ddd | | | | |
| | | | CGG | dkk | | | | |
| | | | G | e | | | | |
| 594622 | 2027 | 2042 | GTT | kkk | 6 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 83 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 86 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594622 | 2027 | 2042 | GTT | kkk | 85 | 13496 | 13511 | 163 |
| | | | ATC | ddd | | | | |
| | | | TGC | ddd | | | | |
| | | | TGC | ddd | | | | |
| | | | TGG | dkk | | | | |
| | | | C | k | | | | |
| 594625 | 2047 | 2062 | TCG | kkk | 0 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 64 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 74 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 594625 | 2047 | 2062 | TCG | kkk | 70 | 13516 | 13531 | 165 |
| | | | CTG | ddd | | | | |
| | | | ATT | ddd | | | | |
| | | | TGT | ddd | | | | |
| | | | CCG | dkk | | | | |
| | | | G | k | | | | |
| 612129 | 965 | 980 | GCC | ekk | 29 | 6182 | 6197 | 889 |
| | | | TGT | ddd | | | | |
| | | | CAG | ddd | | | | |
| | | | CTG | ddd | | | | |
| | | | TGT | dkk | | | | |
| | | | G | e | | | | |
| 612130 | 968 | 983 | GTA | ekk | 44 | 6185 | 6200 | 890 |
| | | | GCC | ddd | | | | |
| | | | TGT | ddd | | | | |
| | | | CAG | ddd | | | | |
| | | | CTG | dkk | | | | |
| | | | T | e | | | | |
| 612131 | 971 | 986 | CCT | ekk | 21 | 6188 | 6203 | 891 |
| | | | GTA | ddd | | | | |
| | | | GCC | ddd | | | | |
| | | | TGT | ddd | | | | |
| | | | CAG | dkk | | | | |
| | | | C | e | | | | |
| 612132 | 974 | 989 | TTG | ekk | 38 | 6191 | 6206 | 892 |
| | | | CCT | ddd | | | | |
| | | | GTA | ddd | | | | |
| | | | GCC | ddd | | | | |
| | | | TGT | dkk | | | | |
| | | | C | e | | | | |
| 612133 | 977 | 992 | GGA | ekk | 14 | 6194 | 6209 | 893 |
| | | | TTG | ddd | | | | |
| | | | CCT | ddd | | | | |
| | | | GTA | ddd | | | | |
| | | | GCC | dkk | | | | |
| | | | T | e | | | | |
| 612134 | 980 | 995 | CCA | ekk | 46 | 6197 | 6212 | 894 |
| | | | GGA | ddd | | | | |
| | | | TTG | ddd | | | | |
| | | | CCT | ddd | | | | |
| | | | GTA | dkk | | | | |
| | | | G | e | | | | |
| 612135 | 983 | 998 | CAC | ekk | 23 | 6200 | 6215 | 68 |
| | | | CCA | ddd | | | | |
| | | | GGA | ddd | | | | |
| | | | TTG | ddd | | | | |
| | | | CCT | dkk | | | | |
| | | | G | e | | | | |
| 612136 | 986 | 1001 | GAA | ekk | 16 | 6203 | 6218 | 895 |
| | | | CAC | ddd | | | | |
| | | | CCA | ddd | | | | |
| | | | GGA | ddd | | | | |
| | | | TTG | dkk | | | | |
| | | | C | e | | | | |
| 612137 | 993 | 1008 | TTC | ekk | 26 | 6210 | 6225 | 69 |
| | | | CAA | ddd | | | | |
| | | | GGA | ddd | | | | |
| | | | ACA | ddd | | | | |
| | | | CCC | dkk | | | | |
| | | | A | e | | | | |
| 612138 | 997 | 1012 | GTC | ekk | 27 | 6214 | 6229 | 896 |
| | | | CTT | ddd | | | | |
| | | | CCA | ddd | | | | |
| | | | AGG | ddd | | | | |
| | | | AAC | dkk | | | | |
| | | | A | e | | | | |
| 612139 | 1000 | 1015 | CTT | ekk | 57 | 6217 | 6232 | 897 |
| | | | GTC | ddd | | | | |
| | | | CTT | ddd | | | | |
| | | | CCA | ddd | | | | |
| | | | AGG | dkk | | | | |
| | | | A | e | | | | |
| 612140 | 1003 | 1018 | GTT | ekk | 22 | 6220 | 6235 | 898 |
| | | | CTT | ddd | | | | |
| | | | GTC | ddd | | | | |
| | | | CTT | ddd | | | | |
| | | | CCA | dkk | | | | |
| | | | A | e | | | | |
| 612141 | 1006 | 1021 | GCA | ekk | 42 | 6223 | 6238 | 899 |
| | | | GTT | ddd | | | | |
| | | | CTT | ddd | | | | |
| | | | GTC | ddd | | | | |
| | | | CTT | dkk | | | | |
| | | | C | e | | | | |
| 612142 | 1009 | 1024 | GGT | ekk | 0 | 6226 | 6241 | 900 |
| | | | GCA | ddd | | | | |
| | | | GTT | ddd | | | | |
| | | | CTT | ddd | | | | |
| | | | GTC | dkk | | | | |
| | | | C | e | | | | |
| 612143 | 1012 | 1027 | GGA | ekk | 0 | 6229 | 6244 | 901 |
| | | | GGT | ddd | | | | |
| | | | GCA | ddd | | | | |
| | | | GTT | ddd | | | | |
| | | | CTT | dkk | | | | |
| | | | G | e | | | | |
| 612144 | 1015 | 1030 | CCG | ekk | 34 | 6232 | 6247 | 902 |
| | | | GGA | ddd | | | | |
| | | | GGT | ddd | | | | |
| | | | GCA | ddd | | | | |
| | | | GTT | dkk | | | | |
| | | | C | e | | | | |
| 612145 | 1018 | 1033 | CAG | ekk | 30 | 6235 | 6250 | 903 |
| | | | CCG | ddd | | | | |
| | | | GGA | ddd | | | | |
| | | | GGT | ddd | | | | |
| | | | GCA | dkk | | | | |
| | | | G | e | | | | |
| 612146 | 1021 | 1036 | ATC | ekk | 43 | 6238 | 6253 | 904 |
| | | | CAG | ddd | | | | |
| | | | CCG | ddd | | | | |
| | | | GGA | ddd | | | | |
| | | | GGT | dkk | | | | |
| | | | G | e | | | | |
| 612147 | 1024 | 1039 | CGC | ekk | 63 | 6241 | 6256 | 905 |
| | | | ATC | ddd | | | | |
| | | | CAG | ddd | | | | |
| | | | CCG | ddd | | | | |
| | | | GGA | dkk | | | | |
| | | | G | e | | | | |
| 612148 | 1027 | 1042 | GTG | ekk | 64 | 6244 | 6259 | 906 |
| | | | CGC | ddd | | | | |
| | | | ATC | ddd | | | | |
| | | | CAG | ddd | | | | |
| | | | CCG | dkk | | | | |
| | | | G | e | | | | |
| 612149 | 1030 | 1045 | CTT | ekk | 4 | 6247 | 6262 | 907 |
| | | | GTG | ddd | | | | |
| | | | CGC | ddd | | | | |
| | | | ATC | ddd | | | | |
| | | | CAG | dkk | | | | |
| | | | C | e | | | | |
| 612150 | 1033 | 1048 | GAC | ekk | 0 | 6250 | 6265 | 908 |
| | | | CTT | ddd | | | | |
| | | | GTG | ddd | | | | |
| | | | CGC | ddd | | | | |
| | | | ATC | dkk | | | | |
| | | | C | e | | | | |
| 612151 | 1036 | 1051 | CAG | ekk | 46 | 6253 | 6268 | 909 |
| | | | GAC | ddd | | | | |
| | | | CTT | ddd | | | | |
| | | | GTG | ddd | | | | |
| | | | CGC | dkk | | | | |
| | | | A | e | | | | |
| 612152 | 1039 | 1054 | AGA | ekk | 12 | 6256 | 6271 | 910 |
| | | | CAG | ddd | | | | |
| | | | GAC | ddd | | | | |
| | | | CTT | ddd | | | | |
| | | | GTG | dkk | | | | |
| | | | C | e | | | | |
| 612153 | 1042 | 1057 | GGC | ekk | 24 | 6259 | 6274 | 911 |
| | | | AGA | ddd | | | | |
| | | | CAG | ddd | | | | |
| | | | GAC | ddd | | | | |
| | | | CTT | dkk | | | | |
| | | | G | e | | | | |
| 612154 | 1060 | 1075 | GCC | ekk | 36 | 6277 | 6292 | 912 |
| | | | CTG | ddd | | | | |
| | | | TAC | ddd | | | | |
| | | | AGC | ddd | | | | |
| | | | CTG | dkk | | | | |
| | | | C | e | | | | |
| 612155 | 1064 | 1079 | GCA | ekk | 19 | 6281 | 6296 | 913 |
| | | | GGC | ddd | | | | |
| | | | CCT | ddd | | | | |
| | | | GTA | ddd | | | | |
| | | | CAG | dkk | | | | |
| | | | C | e | | | | |
| 612156 | 1067 | 1082 | CTA | ekk | 1 | 6284 | 6299 | 914 |
| | | | GCA | ddd | | | | |
| | | | GGC | ddd | | | | |
| | | | CCT | ddd | | | | |
| | | | GTA | dkk | | | | |
| | | | C | e | | | | |
| 612157 | 1071 | 1086 | GCC | ekk | 0 | 6288 | 6303 | 915 |
| | | | ACT | ddd | | | | |
| | | | AGC | ddd | | | | |
| | | | AGG | ddd | | | | |
| | | | CCC | dkk | | | | |
| | | | T | e | | | | |
| 612158 | 1074 | 1089 | TGG | ekk | 0 | 6291 | 6306 | 916 |
| | | | GCC | ddd | | | | |
| | | | ACT | ddd | | | | |
| | | | AGC | ddd | | | | |
| | | | AGG | dkk | | | | |
| | | | C | e | | | | |
| 612159 | 1077 | 1092 | CCC | ekk | 27 | 6294 | 6309 | 917 |
| | | | TGG | ddd | | | | |
| | | | GCC | ddd | | | | |
| | | | ACT | ddd | | | | |
| | | | AGC | dkk | | | | |
| | | | A | e | | | | |
| 612160 | 1080 | 1095 | CTG | ekk | 42 | 6297 | 6312 | 918 |
| | | | CCC | ddd | | | | |
| | | | TGG | ddd | | | | |
| | | | GCC | ddd | | | | |
| | | | ACT | dkk | | | | |
| | | | A | e | | | | |
| 612161 | 1088 | 1103 | TAT | ekk | 28 | 6305 | 6320 | 74 |
| | | | CAG | ddd | | | | |
| | | | CCC | ddd | | | | |
| | | | TGC | ddd | | | | |
| | | | CCT | dkk | | | | |
| | | | G | e | | | | |
| 612162 | 1091 | 1106 | GGC | ekk | 38 | 6308 | 6323 | 919 |
| | | | TAT | ddd | | | | |
| | | | CAG | ddd | | | | |
| | | | CCC | ddd | | | | |
| | | | TGC | dkk | | | | |
| | | | C | e | | | | |
| 612163 | 1094 | 1109 | CCT | ekk | 38 | 6311 | 6326 | 920 |
| | | | GGC | ddd | | | | |
| | | | TAT | ddd | | | | |
| | | | CAG | ddd | | | | |
| | | | CCC | dkk | | | | |
| | | | T | e | | | | |
| 612164 | 1097 | 1112 | GGG | ekk | 24 | 6314 | 6329 | 921 |
| | | | CCT | ddd | | | | |
| | | | GGC | ddd | | | | |
| | | | TAT | ddd | | | | |
| | | | CAG | dkk | | | | |
| | | | C | e | | | | |
| 612165 | 1100 | 1115 | GCT | ekk | 0 | 6317 | 6332 | 922 |
| | | | GGG | ddd | | | | |
| | | | CCT | ddd | | | | |
| | | | GGC | ddd | | | | |
| | | | TAT | dkk | | | | |
| | | | C | e | | | | |
| 612166 | 1115 | 1130 | CCG | ekk | 0 | 6332 | 6347 | 923 |
| | | | TGG | ddd | | | | |
| | | | ACA | ddd | | | | |
| | | | GCA | ddd | | | | |
| | | | GCA | dkk | | | | |
| | | | G | e | | | | |
| 612167 | 1118 | 1133 | CCA | ekk | 28 | 6335 | 6350 | 924 |
| | | | CCG | ddd | | | | |
| | | | TGG | ddd | | | | |
| | | | ACA | ddd | | | | |
| | | | GCA | dkk | | | | |
| | | | G | e | | | | |
| 612168 | 1121 | 1136 | CCA | ekk | 27 | 6338 | 6353 | 925 |
| | | | CCA | ddd | | | | |
| | | | CCG | ddd | | | | |
| | | | TGG | ddd | | | | |
| | | | ACA | dkk | | | | |
| | | | G | e | | | | |
| 612169 | 1124 | 1139 | CGC | ekk | 11 | 6341 | 6356 | 926 |
| | | | CCA | ddd | | | | |
| | | | CCA | ddd | | | | |
| | | | CCG | ddd | | | | |
| | | | TGG | dkk | | | | |
| | | | A | e | | | | |
| 612170 | 1127 | 1142 | ACA | ekk | 18 | 6344 | 6359 | 927 |
| | | | CGC | ddd | | | | |
| | | | CCA | ddd | | | | |
| | | | CCA | ddd | | | | |
| | | | CCG | dkk | | | | |
| | | | T | e | | | | |
| 612171 | 1130 | 1145 | TGA | ekk | 34 | 6347 | 6362 | 928 |
| | | | ACA | ddd | | | | |
| | | | CGC | ddd | | | | |
| | | | CCA | ddd | | | | |
| | | | CCA | dkk | | | | |
| | | | C | e | | | | |
| 612172 | 1133 | 1148 | CTG | ekk | 37 | 6350 | 6365 | 929 |
| | | | TGA | ddd | | | | |
| | | | ACA | ddd | | | | |
| | | | CGC | ddd | | | | |
| | | | CCA | dkk | | | | |
| | | | C | e | | | | |
| 612173 | 1136 | 1151 | GGG | ekk | 0 | 6353 | 6368 | 930 |
| | | | CTG | ddd | | | | |
| | | | TGA | ddd | | | | |
| | | | ACA | ddd | | | | |
| | | | CGC | dkk | | | | |
| | | | C | e | | | | |
| 612174 | 1151 | 1166 | TCA | ekk | 5 | 6368 | 6383 | 78 |
| | | | GGT | ddd | | | | |
| | | | GCA | ddd | | | | |
| | | | GGC | ddd | | | | |
| | | | CTG | dkk | | | | |
| | | | G | e | | | | |
| 612175 | 1154 | 1169 | GCT | ekk | 45 | 6371 | 6386 | 931 |
| | | | TCA | ddd | | | | |
| | | | GGT | ddd | | | | |
| | | | GCA | ddd | | | | |
| | | | GGC | dkk | | | | |
| | | | C | e | | | | |
| 612176 | 1157 | 1172 | GCT | ekk | 30 | 6374 | 6389 | 932 |
| | | | GCT | ddd | | | | |
| | | | TCA | ddd | | | | |
| | | | GGT | ddd | | | | |
| | | | GCA | dkk | | | | |
| | | | G | e | | | | |
| 612177 | 1160 | 1175 | ACG | ekk | 45 | 6377 | 6392 | 933 |
| | | | GCT | ddd | | | | |
| | | | GCT | ddd | | | | |
| | | | TCA | ddd | | | | |
| | | | GGT | dkk | | | | |
| | | | G | e | | | | |
| 612178 | 1163 | 1178 | CAA | ekk | 17 | 6380 | 6395 | 934 |
| | | | ACG | ddd | | | | |
| | | | GCT | ddd | | | | |
| | | | GCT | ddd | | | | |
| | | | TCA | dkk | | | | |
| | | | G | e | | | | |
| 612179 | 1166 | 1181 | GCA | ekk | 34 | 6383 | 6398 | 935 |
| | | | CAA | ddd | | | | |
| | | | ACG | ddd | | | | |
| | | | GCT | ddd | | | | |
| | | | GCT | dkk | | | | |
| | | | T | e | | | | |
| 612180 | 1169 | 1184 | CCT | ekk | 0 | 6386 | 6401 | 936 |
| | | | GCA | ddd | | | | |
| | | | CAA | ddd | | | | |
| | | | ACG | ddd | | | | |
| | | | GCT | dkk | | | | |
| | | | G | e | | | | |
| 612181 | 1172 | 1187 | GGC | ekk | 0 | 6389 | 6404 | 937 |
| | | | CCT | ddd | | | | |
| | | | GCA | ddd | | | | |
| | | | CAA | ddd | | | | |
| | | | ACG | dkk | | | | |
| | | | G | e | | | | |
| 612182 | 1182 | 1197 | TAG | ekk | 38 | 6399 | 6414 | 80 |
| | | | AGA | ddd | | | | |
| | | | GCC | ddd | | | | |
| | | | AGG | ddd | | | | |
| | | | CCC | dkk | | | | |
| | | | T | e | | | | |
| 612183 | 1185 | 1200 | GTA | ekk | 19 | 6402 | 6417 | 938 |
| | | | TAG | ddd | | | | |
| | | | AGA | ddd | | | | |
| | | | GCC | ddd | | | | |
| | | | AGG | dkk | | | | |
| | | | C | e | | | | |
| 612184 | 1203 | 1218 | CGT | ekk | 26 | 6420 | 6435 | 81 |
| | | | GGG | ddd | | | | |
| | | | AGG | ddd | | | | |
| | | | ACC | ddd | | | | |
| | | | ACA | dkk | | | | |
| | | | G | e | | | | |
| 612185 | 1217 | 1232 | TGA | ekk | 5 | 6434 | 6449 | 82 |
| | | | AGT | ddd | | | | |
| | | | CCA | ddd | | | | |
| | | | GAG | ddd | | | | |
| | | | AGC | dkk | | | | |
| | | | G | e | | | | |
| 612186 | 1220 | 1235 | CTG | ekk | 45 | 6437 | 6452 | 939 |
| | | | TGA | ddd | | | | |
| | | | AGT | ddd | | | | |
| | | | CCA | ddd | | | | |
| | | | GAG | dkk | | | | |
| | | | A | e | | | | |
| 612187 | 1223 | 1238 | GTT | ekk | 49 | 6440 | 6455 | 940 |
| | | | CTG | ddd | | | | |
| | | | TGA | ddd | | | | |
| | | | AGT | ddd | | | | |
| | | | CCA | dkk | | | | |
| | | | G | e | | | | |
| 612188 | 1226 | 1241 | CCA | ekk | 23 | 6443 | 6458 | 941 |
| | | | GTT | ddd | | | | |
| | | | CTG | ddd | | | | |
| | | | TGA | ddd | | | | |
| | | | AGT | dkk | | | | |
| | | | C | e | | | | |
| 612189 | 1229 | 1244 | CAT | ekk | 31 | 6446 | 6461 | 942 |
| | | | CCA | ddd | | | | |
| | | | GTT | ddd | | | | |
| | | | CTG | ddd | | | | |
| | | | TGA | dkk | | | | |
| | | | A | e | | | | |
| 612190 | 1232 | 1247 | CAA | ekk | 30 | 6449 | 6464 | 943 |
| | | | CAT | ddd | | | | |
| | | | CCA | ddd | | | | |
| | | | GTT | ddd | | | | |
| | | | CTG | dkk | | | | |
| | | | T | e | | | | |
| 612191 | 1235 | 1250 | CAG | ekk | 35 | 6452 | 6467 | 944 |
| | | | CAA | ddd | | | | |
| | | | CAT | ddd | | | | |
| | | | CCA | ddd | | | | |
| | | | GTT | dkk | | | | |
| | | | C | e | | | | |
| 612192 | 1244 | 1259 | TCT | ekk | 61 | 6461 | 6476 | 84 |
| | | | TCT | ddd | | | | |
| | | | CAG | ddd | | | | |
| | | | CAG | ddd | | | | |
| | | | CAA | dkk | | | | |
| | | | C | e | | | | |
| 612193 | 1247 | 1262 | CAA | ekk | 34 | 6464 | 6479 | 945 |
| | | | TCT | ddd | | | | |
| | | | TCT | ddd | | | | |
| | | | CAG | ddd | | | | |
| | | | CAG | dkk | | | | |
| | | | C | e | | | | |
| 612194 | 1250 | 1265 | TGT | ekk | 44 | 6467 | 6482 | 946 |
| | | | CAA | ddd | | | | |
| | | | TCT | ddd | | | | |
| | | | TCT | ddd | | | | |
| | | | CAG | dkk | | | | |
| | | | C | e | | | | |
| 612195 | 1253 | 1268 | ACC | ekk | 47 | 6470 | 6485 | 947 |
| | | | TGT | ddd | | | | |
| | | | CAA | ddd | | | | |
| | | | TCT | ddd | | | | |
| | | | TCT | dkk | | | | |
| | | | C | e | | | | |
| 612196 | 1256 | 1271 | TGA | ekk | 18 | 6473 | 6488 | 948 |
| | | | ACC | ddd | | | | |
| | | | TGT | ddd | | | | |
| | | | CAA | ddd | | | | |
| | | | TCT | dkk | | | | |
| | | | T | e | | | | |
| 612197 | 1259 | 1274 | GCA | ekk | 39 | 6476 | 6491 | 949 |
| | | | TGA | ddd | | | | |
| | | | ACC | ddd | | | | |
| | | | TGT | ddd | | | | |
| | | | CAA | dkk | | | | |
| | | | T | e | | | | |
| 612198 | 1262 | 1277 | CCT | ekk | 35 | 6479 | 6494 | 950 |
| | | | GCA | ddd | | | | |
| | | | TGA | ddd | | | | |
| | | | ACC | ddd | | | | |
| | | | TGT | dkk | | | | |
| | | | C | e | | | | |
| 612199 | 1265 | 1280 | CAG | ekk | 47 | 6482 | 6497 | 951 |
| | | | CCT | ddd | | | | |
| | | | GCA | ddd | | | | |
| | | | TGA | ddd | | | | |
| | | | ACC | dkk | | | | |
| | | | T | e | | | | |
| 612200 | 1267 | 1282 | CAC | ekk | 26 | 6484 | 6499 | 952 |
| | | | AGC | ddd | | | | |
| | | | CTG | ddd | | | | |
| | | | CAT | ddd | | | | |
| | | | GAA | dkk | | | | |
| | | | C | e | | | | |
| 612201 | 1268 | 1283 | TCA | ekk | 36 | 6485 | 6500 | 953 |
| | | | CAG | ddd | | | | |
| | | | CCT | ddd | | | | |
| | | | GCA | ddd | | | | |
| | | | TGA | dkk | | | | |
| | | | A | e | | | | |
| 612202 | 1274 | 1289 | ATC | ekk | 68 | 6491 | 6506 | 954 |
| | | | CTG | ddd | | | | |
| | | | TCA | ddd | | | | |
| | | | CAG | ddd | | | | |
| | | | CCT | dkk | | | | |
| | | | G | e | | | | |
| 612203 | 1276 | 1291 | CCA | ekk | 50 | 6493 | 6508 | 955 |
| | | | TCC | ddd | | | | |
| | | | TGT | ddd | | | | |
| | | | CAC | ddd | | | | |
| | | | AGC | dkk | | | | |
| | | | C | e | | | | |
| 612204 | 1277 | 1292 | TCC | ekk | 7 | 6494 | 6509 | 956 |
| | | | ATC | ddd | | | | |
| | | | CTG | ddd | | | | |
| | | | TCA | ddd | | | | |
| | | | CAG | dkk | | | | |
| | | | C | e | | | | |
| 612205 | 1279 | 1294 | CTT | ekk | 33 | 6496 | 6511 | 957 |
| | | | CCA | ddd | | | | |
| | | | TCC | ddd | | | | |
| | | | TGT | ddd | | | | |
| | | | CAC | dkk | | | | |
| | | | A | e | | | | |
| 612206 | 1282 | 1297 | AGT | ekk | 54 | 6499 | 6514 | 958 |
| | | | CTT | ddd | | | | |
| | | | CCA | ddd | | | | |
| | | | TCC | ddd | | | | |
| | | | TGT | dkk | | | | |
| | | | C | e | | | | |
| 612207 | 1286 | 1301 | AGC | ekk | 58 | 6503 | 6518 | 959 |
| | | | CAG | ddd | | | | |
| | | | TCT | ddd | | | | |
| | | | TCC | ddd | | | | |
| | | | ATC | dkk | | | | |
| | | | C | e | | | | |
| 612233 | 1399 | 1414 | CAC | ekk | 7 | 10410 | 10425 | 960 |
| | | | CCA | ddd | | | | |
| | | | GAA | ddd | | | | |
| | | | CTC | ddd | | | | |
| | | | CTG | dkk | | | | |
| | | | G | e | | | | |
| 612234 | 1402 | 1417 | GTC | ekk | 66 | 10413 | 10428 | 961 |
| | | | CAC | ddd | | | | |
| | | | CCA | ddd | | | | |
| | | | GAA | ddd | | | | |
| | | | CTC | dkk | | | | |
| | | | C | e | | | | |
| 612235 | 1405 | 1420 | GTT | ekk | 73 | 10416 | 10431 | 962 |
| | | | GTC | ddd | | | | |
| | | | CAC | ddd | | | | |
| | | | CCA | ddd | | | | |
| | | | GAA | dkk | | | | |
| | | | C | e | | | | |
| 612236 | 1408 | 1423 | GCT | ekk | 76 | 10419 | 10434 | 963 |
| | | | GTT | ddd | | | | |
| | | | GTC | ddd | | | | |
| | | | CAC | ddd | | | | |
| | | | CCA | dkk | | | | |
| | | | G | e | | | | |
| 612237 | 1411 | 1426 | GGT | ekk | 25 | 10422 | 10437 | 964 |
| | | | GCT | ddd | | | | |
| | | | GTT | ddd | | | | |
| | | | GTC | ddd | | | | |
| | | | CAC | dkk | | | | |
| | | | C | e | | | | |
| 612238 | 1414 | 1429 | TGA | ekk | 77 | 10425 | 10440 | 965 |
| | | | GGT | ddd | | | | |
| | | | GCT | ddd | | | | |
| | | | GTT | ddd | | | | |
| | | | GTC | dkk | | | | |
| | | | C | e | | | | |
| 612239 | 1417 | 1432 | CAC | ekk | 92 | 10428 | 10443 | 966 |
| | | | TGA | ddd | | | | |
| | | | GGT | ddd | | | | |
| | | | GCT | ddd | | | | |
| | | | GTT | dkk | | | | |
| | | | G | e | | | | |
| 612240 | 1421 | 1436 | CAG | ekk | 50 | 10432 | 10447 | 93 |
| | | | ACA | ddd | | | | |
| | | | CTG | ddd | | | | |
| | | | AGG | ddd | | | | |
| | | | TGC | dkk | | | | |
| | | | T | e | | | | |
| 612241 | 1429 | 1444 | CAT | ekk | 0 | 10440 | 10455 | 967 |
| | | | GGG | ddd | | | | |
| | | | AAC | ddd | | | | |
| | | | AGA | ddd | | | | |
| | | | CAC | dkk | | | | |
| | | | T | e | | | | |
| 612242 | 1432 | 1447 | GAG | ekk | 0 | 10443 | 10458 | 968 |
| | | | CAT | ddd | | | | |
| | | | GGG | ddd | | | | |
| | | | AAC | ddd | | | | |
| | | | AGA | dkk | | | | |
| | | | C | e | | | | |
| 612243 | 1435 | 1450 | AGA | ekk | 6 | 10446 | 10461 | 969 |
| | | | GAG | ddd | | | | |
| | | | CAT | ddd | | | | |
| | | | GGG | ddd | | | | |
| | | | AAC | dkk | | | | |
| | | | A | e | | | | |
| 612244 | 1438 | 1453 | GCC | ekk | 52 | 10449 | 10464 | 970 |
| | | | AGA | ddd | | | | |
| | | | GAG | ddd | | | | |
| | | | CAT | ddd | | | | |
| | | | GGG | dkk | | | | |
| | | | A | e | | | | |
| 612245 | 1441 | 1456 | CAT | ekk | 63 | 10452 | 10467 | 971 |
| | | | GCC | ddd | | | | |
| | | | AGA | ddd | | | | |
| | | | GAG | ddd | | | | |
| | | | CAT | dkk | | | | |
| | | | G | e | | | | |
| 612246 | 1444 | 1459 | GCC | ekk | 59 | 10455 | 10470 | 972 |
| | | | CAT | ddd | | | | |
| | | | GCC | ddd | | | | |
| | | | AGA | ddd | | | | |
| | | | GAG | dkk | | | | |
| | | | C | e | | | | |
| 612247 | 1447 | 1462 | GGT | ekk | 76 | 10458 | 10473 | 973 |
| | | | GCC | ddd | | | | |
| | | | CAT | ddd | | | | |
| | | | GCC | ddd | | | | |
| | | | AGA | dkk | | | | |
| | | | G | e | | | | |
| 612248 | 1450 | 1465 | GAA | ekk | 0 | 10461 | 10476 | 974 |
| | | | GGT | ddd | | | | |
| | | | GCC | ddd | | | | |
| | | | CAT | ddd | | | | |
| | | | GCC | dkk | | | | |
| | | | A | e | | | | |
| 612249 | 1453 | 1468 | CTG | ekk | 47 | 10464 | 10479 | 975 |
| | | | GAA | ddd | | | | |
| | | | GGT | ddd | | | | |
| | | | GCC | ddd | | | | |
| | | | CAT | dkk | | | | |
| | | | G | e | | | | |
| 612250 | 1457 | 1472 | AGT | ekk | 0 | 10468 | 10483 | 976 |
| | | | GCT | ddd | | | | |
| | | | GGA | ddd | | | | |
| | | | AGG | ddd | | | | |
| | | | TGC | dkk | | | | |
| | | | C | e | | | | |
| 612251 | 1460 | 1475 | TCC | ekk | 11 | 10471 | 10486 | 977 |
| | | | AGT | ddd | | | | |
| | | | GCT | ddd | | | | |
| | | | GGA | ddd | | | | |
| | | | AGG | dkk | | | | |
| | | | T | e | | | | |
| 612252 | 1462 | 1477 | ACT | ekk | 85 | 10473 | 10488 | 96 |
| | | | CCA | ddd | | | | |
| | | | GTG | ddd | | | | |
| | | | CTG | ddd | | | | |
| | | | GAA | dkk | | | | |
| | | | G | e | | | | |
| 612253 | 1463 | 1478 | CAC | ekk | 31 | 10474 | 10489 | 978 |
| | | | TCC | ddd | | | | |
| | | | AGT | ddd | | | | |
| | | | GCT | ddd | | | | |
| | | | GGA | dkk | | | | |
| | | | A | e | | | | |
| 612254 | 1465 | 1480 | GTC | ekk | 77 | 10476 | 10491 | 97 |
| | | | ACT | ddd | | | | |
| | | | CCA | ddd | | | | |
| | | | GTG | ddd | | | | |
| | | | CTG | dkk | | | | |
| | | | G | e | | | | |
| 612255 | 1466 | 1481 | TGT | ekk | 58 | 10477 | 10492 | 979 |
| | | | CAC | ddd | | | | |
| | | | TCC | ddd | | | | |
| | | | AGT | ddd | | | | |
| | | | GCT | dkk | | | | |
| | | | G | e | | | | |
| 612256 | 1467 | 1482 | ATG | ekk | 8 | 10478 | 10493 | 980 |
| | | | TCA | ddd | | | | |
| | | | CTC | ddd | | | | |
| | | | CAG | ddd | | | | |
| | | | TGC | dkk | | | | |
| | | | T | e | | | | |
| 612257 | 1468 | 1483 | GAT | ekk | 35 | 10479 | 10494 | 981 |
| | | | GTC | ddd | | | | |
| | | | ACT | ddd | | | | |
| | | | CCA | ddd | | | | |
| | | | GTG | dkk | | | | |
| | | | C | e | | | | |
| 612258 | 1469 | 1484 | GGA | ekk | 2 | 10480 | 10495 | 982 |
| | | | TGT | ddd | | | | |
| | | | CAC | ddd | | | | |
| | | | TCC | ddd | | | | |
| | | | AGT | dkk | | | | |
| | | | G | e | | | | |
| 612259 | 1470 | 1485 | TGG | ekk | 15 | 10481 | 10496 | 983 |
| | | | ATG | ddd | | | | |
| | | | TCA | ddd | | | | |
| | | | CTC | ddd | | | | |
| | | | CAG | dkk | | | | |
| | | | T | e | | | | |
| 612260 | 1472 | 1487 | CCT | ekk | 40 | 10483 | 10498 | 984 |
| | | | GGA | ddd | | | | |
| | | | TGT | ddd | | | | |
| | | | CAC | ddd | | | | |
| | | | TCC | dkk | | | | |
| | | | A | e | | | | |
| 612261 | 1475 | 1490 | TGT | ekk | 46 | 10486 | 10501 | 985 |
| | | | CCT | ddd | | | | |
| | | | GGA | ddd | | | | |
| | | | TGT | ddd | | | | |
| | | | CAC | dkk | | | | |
| | | | T | e | | | | |
| 612262 | 1478 | 1493 | AGT | ekk | 63 | 10489 | 10504 | 986 |
| | | | TGT | ddd | | | | |
| | | | CCT | ddd | | | | |
| | | | GGA | ddd | | | | |
| | | | TGT | dkk | | | | |
| | | | C | e | | | | |
| 612263 | 1481 | 1496 | AGA | ekk | 65 | 10492 | 10507 | 987 |
| | | | AGT | ddd | | | | |
| | | | TGT | ddd | | | | |
| | | | CCT | ddd | | | | |
| | | | GGA | dkk | | | | |
| | | | T | e | | | | |
| 612264 | 1484 | 1499 | CCG | ekk | 59 | 10495 | 10510 | 99 |
| | | | AGA | ddd | | | | |
| | | | AGT | ddd | | | | |
| | | | TGT | ddd | | | | |
| | | | CCT | dkk | | | | |
| | | | G | e | | | | |
| 612265 | 1487 | 1502 | TCA | ekk | 0 | 10498 | 10513 | 988 |
| | | | CCG | ddd | | | | |
| | | | AGA | ddd | | | | |
| | | | AGT | ddd | | | | |
| | | | TGT | dkk | | | | |
| | | | C | e | | | | |
| 612266 | 1490 | 1505 | GAG | ekk | 68 | 10501 | 10516 | 989 |
| | | | TCA | ddd | | | | |
| | | | CCG | ddd | | | | |
| | | | AGA | ddd | | | | |
| | | | AGT | dkk | | | | |
| | | | T | e | | | | |
| 612267 | 1493 | 1508 | CTT | ekk | 76 | 10504 | 10519 | 990 |
| | | | GAG | ddd | | | | |
| | | | TCA | ddd | | | | |
| | | | CCG | ddd | | | | |
| | | | AGA | dkk | | | | |
| | | | A | e | | | | |
| 612268 | 1496 | 1511 | GCA | ekk | 77 | 10507 | 10522 | 991 |
| | | | CTT | ddd | | | | |
| | | | GAG | ddd | | | | |
| | | | TCA | ddd | | | | |
| | | | CCG | dkk | | | | |
| | | | A | e | | | | |
| 612269 | 1499 | 1514 | AGG | ekk | 43 | 10510 | 10525 | 992 |
| | | | GCA | ddd | | | | |
| | | | CTT | ddd | | | | |
| | | | GAG | ddd | | | | |
| | | | TCA | dkk | | | | |
| | | | C | e | | | | |
| 612270 | 1502 | 1517 | TGA | ekk | 42 | 10513 | 10528 | 993 |
| | | | AGG | ddd | | | | |
| | | | GCA | ddd | | | | |
| | | | CTT | ddd | | | | |
| | | | GAG | dkk | | | | |
| | | | T | e | | | | |
| 612271 | 1505 | 1520 | CAG | ekk | 65 | 10516 | 10531 | 994 |
| | | | TGA | ddd | | | | |
| | | | AGG | ddd | | | | |
| | | | GCA | ddd | | | | |
| | | | CTT | dkk | | | | |
| | | | G | e | | | | |
| 612272 | 1508 | 1523 | TCT | ekk | 0 | 10519 | 10534 | 995 |
| | | | CAG | ddd | | | | |
| | | | TGA | ddd | | | | |
| | | | AGG | ddd | | | | |
| | | | GCA | dkk | | | | |
| | | | C | e | | | | |
| 612273 | 1511 | 1526 | CGC | ekk | 35 | 10522 | 10537 | 996 |
| | | | TCT | ddd | | | | |
| | | | CAG | ddd | | | | |
| | | | TGA | ddd | | | | |
| | | | AGG | dkk | | | | |
| | | | G | e | | | | |
| 612274 | 1524 | 1539 | AGC | ekk | 77 | 10535 | 10550 | 997 |
| | | | AGC | ddd | | | | |
| | | | AGG | ddd | | | | |
| | | | CAG | ddd | | | | |
| | | | GCG | dkk | | | | |
| | | | C | e | | | | |
| 612275 | 1528 | 1543 | GAT | ekk | 64 | 10539 | 10554 | 998 |
| | | | CAG | ddd | | | | |
| | | | CAG | ddd | | | | |
| | | | CAG | ddd | | | | |
| | | | GCA | dkk | | | | |
| | | | G | e | | | | |
| 612276 | 1532 | 1547 | GCT | ekk | 33 | 10543 | 10558 | 999 |
| | | | GGA | ddd | | | | |
| | | | TCA | ddd | | | | |
| | | | GCA | ddd | | | | |
| | | | GCA | dkk | | | | |
| | | | G | e | | | | |
| 612277 | 1535 | 1550 | GAG | ekk | 81 | 10546 | 10561 | 1000 |
| | | | GCT | ddd | | | | |
| | | | GGA | ddd | | | | |
| | | | TCA | ddd | | | | |
| | | | GCA | dkk | | | | |
| | | | G | e | | | | |
| 612278 | 1538 | 1553 | AGT | ekk | 79 | 10549 | 10564 | 1001 |
| | | | GAG | ddd | | | | |
| | | | GCT | ddd | | | | |
| | | | GGA | ddd | | | | |
| | | | TCA | dkk | | | | |
| | | | G | e | | | | |
| 612279 | 1541 | 1556 | CAT | ekk | 58 | 10552 | 10567 | 1002 |
| | | | AGT | ddd | | | | |
| | | | GAG | ddd | | | | |
| | | | GCT | ddd | | | | |
| | | | GGA | dkk | | | | |
| | | | T | e | | | | |
| 612280 | 1544 | 1559 | AGG | ekk | 20 | 10555 | 10570 | 1003 |
| | | | CAT | ddd | | | | |
| | | | AGT | ddd | | | | |
| | | | GAG | ddd | | | | |
| | | | GCT | dkk | | | | |
| | | | G | e | | | | |
| 612688 | N/A | N/A | CGG | ekk | 0 | 2483 | 2498 | 565 |
| | | | CTT | ddd | | | | |
| | | | ACC | ddd | | | | |
| | | | TTC | ddd | | | | |
| | | | TGC | dkk | | | | |
| | | | T | e | | | | |
| 612799 | N/A | N/A | AGA | ekk | 0 | 10783 | 10798 | 1004 |
| | | | CAC | ddd | | | | |
| | | | ACA | ddd | | | | |
| | | | GGC | ddd | | | | |
| | | | CGC | dkk | | | | |
| | | | C | e | | | | |
| 612800 | N/A | N/A | ACA | ekk | 29 | 10830 | 10845 | 1005 |
| | | | CTA | ddd | | | | |
| | | | ACT | ddd | | | | |
| | | | GGA | ddd | | | | |
| | | | GAG | dkk | | | | |
| | | | C | e | | | | |
| 612801 | N/A | N/A | AGA | ekk | 39 | 10939 | 10954 | 1006 |
| | | | GGG | ddd | | | | |
| | | | CGG | ddd | | | | |
| | | | ATT | ddd | | | | |
| | | | GCA | dkk | | | | |
| | | | A | e | | | | |
| 612802 | N/A | N/A | CAG | ekk | 37 | 10940 | 10955 | 1007 |
| | | | AGG | ddd | | | | |
| | | | GCG | ddd | | | | |
| | | | GAT | ddd | | | | |
| | | | TGC | dkk | | | | |
| | | | A | e | | | | |
| 612803 | N/A | N/A | TCT | ekk | 36 | 10943 | 10958 | 1008 |
| | | | CAG | ddd | | | | |
| | | | AGG | ddd | | | | |
| | | | GCG | ddd | | | | |
| | | | GAT | dkk | | | | |
| | | | T | e | | | | |
| 612804 | N/A | N/A | CTC | ekk | 55 | 10944 | 10959 | 1009 |
| | | | TCA | ddd | | | | |
| | | | GAG | ddd | | | | |
| | | | GGC | ddd | | | | |
| | | | GGA | dkk | | | | |
| | | | T | e | | | | |
| 612805 | N/A | N/A | TCT | ekk | 34 | 10945 | 10960 | 1010 |
| | | | CTC | ddd | | | | |
| | | | AGA | ddd | | | | |
| | | | GGG | ddd | | | | |
| | | | CGG | dkk | | | | |
| | | | A | e | | | | |
| 612806 | N/A | N/A | GCT | ekk | 71 | 10977 | 10992 | 1011 |
| | | | GTG | ddd | | | | |
| | | | TGT | ddd | | | | |
| | | | CAG | ddd | | | | |
| | | | GTG | dkk | | | | |
| | | | T | e | | | | |
| 612807 | N/A | N/A | AAG | ekk | 0 | 11003 | 11018 | 1012 |
| | | | AAG | ddd | | | | |
| | | | CTC | ddd | | | | |
| | | | TTG | ddd | | | | |
| | | | GAT | dkk | | | | |
| | | | G | e | | | | |
| 612808 | N/A | N/A | TCC | ekk | 52 | 11006 | 11021 | 1013 |
| | | | AAG | ddd | | | | |
| | | | AAG | ddd | | | | |
| | | | CTC | ddd | | | | |
| | | | TTG | dkk | | | | |
| | | | G | e | | | | |
| 612809 | N/A | N/A | CCA | ekk | 28 | 11109 | 11124 | 1014 |
| | | | GCC | ddd | | | | |
| | | | GCC | ddd | | | | |
| | | | AGC | ddd | | | | |
| | | | CGC | dkk | | | | |
| | | | C | e | | | | |
| 612810 | N/A | N/A | TTA | ekk | 69 | 11451 | 11466 | 1015 |
| | | | GTG | ddd | | | | |
| | | | TTT | ddd | | | | |
| | | | CAG | ddd | | | | |
| | | | CAG | dkk | | | | |
| | | | G | e | | | | |
| 612811 | N/A | N/A | AGT | ekk | 35 | 11453 | 11468 | 1016 |
| | | | TAG | ddd | | | | |
| | | | TGT | ddd | | | | |
| | | | TTC | ddd | | | | |
| | | | AGC | dkk | | | | |
| | | | A | e | | | | |
| 612812 | N/A | N/A | AAC | ekk | 37 | 11506 | 11521 | 1017 |
| | | | CTC | ddd | | | | |
| | | | GAG | ddd | | | | |
| | | | GAC | ddd | | | | |
| | | | ATC | dkk | | | | |
| | | | G | e | | | | |
| 612813 | N/A | N/A | ACT | ekk | 7 | 11696 | 11711 | 1018 |
| | | | TAT | ddd | | | | |
| | | | AAG | ddd | | | | |
| | | | AGC | ddd | | | | |
| | | | TGA | dkk | | | | |
| | | | C | e | | | | |
| 612814 | N/A | N/A | AGC | ekk | 21 | 11699 | 11714 | 1019 |
| | | | ACT | ddd | | | | |
| | | | TAT | ddd | | | | |
| | | | AAG | ddd | | | | |
| | | | AGC | dkk | | | | |
| | | | T | e | | | | |
| 612815 | N/A | N/A | GCA | ekk | 27 | 11866 | 11881 | 1020 |
| | | | GTG | ddd | | | | |
| | | | TTC | ddd | | | | |
| | | | TTG | ddd | | | | |
| | | | ATG | dkk | | | | |
| | | | A | e | | | | |
| 612816 | N/A | N/A | ACA | ekk | 67 | 11869 | 11884 | 1021 |
| | | | GCA | ddd | | | | |
| | | | GTG | ddd | | | | |
| | | | TTC | ddd | | | | |
| | | | TTG | dkk | | | | |
| | | | A | e | | | | |
| 612817 | N/A | N/A | ATA | ekk | 57 | 11895 | 11910 | 1022 |
| | | | ATG | ddd | | | | |
| | | | CAC | ddd | | | | |
| | | | TGT | ddd | | | | |
| | | | GTC | dkk | | | | |
| | | | T | e | | | | |
| 612818 | N/A | N/A | GAT | ekk | 48 | 11996 | 12011 | 1023 |
| | | | GAG | ddd | | | | |
| | | | GAC | ddd | | | | |
| | | | CTA | ddd | | | | |
| | | | GGA | dkk | | | | |
| | | | A | e | | | | |
| 612819 | N/A | N/A | CCG | ekk | 67 | 11998 | 12013 | 1024 |
| | | | ATG | ddd | | | | |
| | | | AGG | ddd | | | | |
| | | | ACC | ddd | | | | |
| | | | TAG | dkk | | | | |
| | | | G | e | | | | |
| 612820 | N/A | N/A | ACG | ekk | 21 | 12128 | 12143 | 1025 |
| | | | ACA | ddd | | | | |
| | | | GGG | ddd | | | | |
| | | | ATG | ddd | | | | |
| | | | TTT | dkk | | | | |
| | | | G | e | | | | |
| 612821 | N/A | N/A | GGT | ekk | 0 | 12398 | 12413 | 1026 |
| | | | CAG | ddd | | | | |
| | | | GCA | ddd | | | | |
| | | | CAG | ddd | | | | |
| | | | ACA | dkk | | | | |
| | | | C | e | | | | |
| 612822 | N/A | N/A | ATC | ekk | 45 | 12671 | 12686 | 1027 |
| | | | CCG | ddd | | | | |
| | | | GTT | ddd | | | | |
| | | | TCA | ddd | | | | |
| | | | ACT | dkk | | | | |
| | | | C | e | | | | |
| 612823 | N/A | N/A | TCC | ekk | 21 | 12866 | 12881 | 1028 |
| | | | CGC | ddd | | | | |
| | | | TGG | ddd | | | | |
| | | | CCC | ddd | | | | |
| | | | CCG | dkk | | | | |
| | | | T | e | | | | |
| 612824 | N/A | N/A | CTA | ekk | 13 | 12888 | 12903 | 1029 |
| | | | ACT | ddd | | | | |
| | | | TAG | ddd | | | | |
| | | | CAC | ddd | | | | |
| | | | AGA | dkk | | | | |
| | | | G | e | | | | |
| 612825 | N/A | N/A | CCA | ekk | 44 | 12915 | 12930 | 1030 |
| | | | TGG | ddd | | | | |
| | | | CCC | ddd | | | | |
| | | | ACC | ddd | | | | |
| | | | AGT | dkk | | | | |
| | | | G | e | | | | |
| 612826 | N/A | N/A | TTG | ekk | 30 | 12919 | 12934 | 1031 |
| | | | GCC | ddd | | | | |
| | | | ATG | ddd | | | | |
| | | | GCC | ddd | | | | |
| | | | CAC | dkk | | | | |
| | | | C | e | | | | |
| 612827 | N/A | N/A | GGC | ekk | 0 | 12938 | 12953 | 1032 |
| | | | AGA | ddd | | | | |
| | | | ATT | ddd | | | | |
| | | | CCT | ddd | | | | |
| | | | GGC | dkk | | | | |
| | | | T | e | | | | |
| 612828 | N/A | N/A | GCA | ekk | 13 | 13059 | 13074 | 1033 |
| | | | AGG | ddd | | | | |
| | | | GTG | ddd | | | | |
| | | | TGT | ddd | | | | |
| | | | CTG | dkk | | | | |
| | | | T | e | | | | |
| 612829 | N/A | N/A | GGC | ekk | 23 | 13060 | 13075 | 1034 |
| | | | AAG | ddd | | | | |
| | | | GGT | ddd | | | | |
| | | | GTG | ddd | | | | |
| | | | TCT | dkk | | | | |
| | | | G | e | | | | |
| 612830 | N/A | N/A | CTC | ekk | 60 | 13069 | 13084 | 1035 |
| | | | AGT | ddd | | | | |
| | | | GTA | ddd | | | | |
| | | | GGC | ddd | | | | |
| | | | AAG | dkk | | | | |
| | | | G | e | | | | |
| 612831 | N/A | N/A | GAG | ekk | 12 | 13094 | 13109 | 1036 |
| | | | GAT | ddd | | | | |
| | | | GCA | ddd | | | | |
| | | | CAG | ddd | | | | |
| | | | TGT | dkk | | | | |
| | | | A | e | | | | |
| 612832 | N/A | N/A | GCT | ekk | 22 | 13151 | 13166 | 1037 |
| | | | CAG | ddd | | | | |
| | | | GAC | ddd | | | | |
| | | | CTC | ddd | | | | |
| | | | TGT | dkk | | | | |
| | | | G | e | | | | |
| 612833 | N/A | N/A | GGC | ekk | 34 | 13152 | 13167 | 1038 |
| | | | TCA | ddd | | | | |
| | | | GGA | ddd | | | | |
| | | | CCT | ddd | | | | |
| | | | CTG | dkk | | | | |
| | | | T | e | | | | |
| 612834 | N/A | N/A | GGC | ekk | 38 | 13198 | 13213 | 1039 |
| | | | GCA | ddd | | | | |
| | | | CTG | ddd | | | | |
| | | | GGT | ddd | | | | |
| | | | GAC | dkk | | | | |
| | | | C | e | | | | |
| 612835 | N/A | N/A | TCT | ekk | 9 | 13204 | 13219 | 1040 |
| | | | GAG | ddd | | | | |
| | | | GGC | ddd | | | | |
| | | | GCA | ddd | | | | |
| | | | CTG | dkk | | | | |
| | | | G | e | | | | |
| 612836 | N/A | N/A | TCA | ekk | 1 | 13208 | 13223 | 1041 |
| | | | TTC | ddd | | | | |
| | | | TGA | ddd | | | | |
| | | | GGG | ddd | | | | |
| | | | CGC | dkk | | | | |
| | | | A | e | | | | |
| 612838 | N/A | N/A | GCT | ekk | 33 | 10636 | 10651 | 1042 |
| | | | CCT | ddd | | | | |
| | | | ACC | ddd | | | | |
| | | | GGG | ddd | | | | |
| | | | GAG | dkk | | | | |
| | | | A | e | | | | |
| 612839 | N/A | N/A | ACA | ekk | 0 | 12376 | 12391 | 1043 |
| | | | CAT | ddd | | | | |
| | | | ACC | ddd | | | | |
| | | | TCC | ddd | | | | |
| | | | CCC | dkk | | | | |
| | | | A | e | | | | |
| 612840 | N/A | N/A | CGC | ekk | 0 | 5715 | 5730 | 1044 |
| | | | ATA | ddd | | | | |
| | | | CCC | ddd | | | | |
| | | | TGA | ddd | | | | |
| | | | AAT | dkk | | | | |
| | | | A | e | | | | |
| 612842 | N/A | N/A | GGA | ekk | 13 | 12231 | 12246 | 1045 |
| | | | TGG | ddd | | | | |
| | | | TCC | ddd | | | | |
| | | | TGG | ddd | | | | |
| | | | GGA | dkk | | | | |
| | | | G | e | | | | |
| 612843 | N/A | N/A | TTC | ekk | 0 | 13239 | 13254 | 1046 |
| | | | AGC | ddd | | | | |
| | | | ACC | ddd | | | | |
| | | | TGC | ddd | | | | |
| | | | AAA | dkk | | | | |
| | | | G | e | | | | |
| 612844 | N/A | N/A | CCG | ekk | 9 | 2484 | 2499 | 1047 |
| | | | GCT | ddd | | | | |
| | | | TAC | ddd | | | | |
| | | | CTT | ddd | | | | |
| | | | CTG | dkk | | | | |
| | | | C | e | | | | |
| 612845 | N/A | N/A | CCC | ekk | 0 | 2487 | 2502 | 1048 |
| | | | CCG | ddd | | | | |
| | | | GCT | ddd | | | | |
| | | | TAC | ddd | | | | |
| | | | CTT | dkk | | | | |
| | | | C | e | | | | |
| 612846 | N/A | N/A | GGG | ekk | 0 | 2490 | 2505 | 1049 |
| | | | CCC | ddd | | | | |
| | | | CCG | ddd | | | | |
| | | | GCT | ddd | | | | |
| | | | TAC | dkk | | | | |
| | | | C | e | | | | |
| 612847 | N/A | N/A | GTG | ekk | 14 | 3361 | 3376 | 1050 |
| | | | AAT | ddd | | | | |
| | | | GTG | ddd | | | | |
| | | | AGC | ddd | | | | |
| | | | CCC | dkk | | | | |
| | | | G | e | | | | |
| 612848 | N/A | N/A | TCC | ekk | 0 | 3435 | 3450 | 1051 |
| | | | CTC | ddd | | | | |
| | | | CTT | ddd | | | | |
| | | | ATA | ddd | | | | |
| | | | ACC | dkk | | | | |
| | | | C | e | | | | |
| 612849 | N/A | N/A | CCG | ekk | 4 | 3471 | 3486 | 1052 |
| | | | GGC | ddd | | | | |
| | | | ACT | ddd | | | | |
| | | | CTC | ddd | | | | |
| | | | AAC | dkk | | | | |
| | | | T | e | | | | |
| 612850 | N/A | N/A | AGT | ekk | 4 | 3752 | 3767 | 1053 |
| | | | AAT | ddd | | | | |
| | | | GGT | ddd | | | | |
| | | | GCT | ddd | | | | |
| | | | CTG | dkk | | | | |
| | | | G | e | | | | |
| 612851 | N/A | N/A | TCC | ekk | 30 | 3759 | 3774 | 1054 |
| | | | TGG | ddd | | | | |
| | | | GAG | ddd | | | | |
| | | | TAA | ddd | | | | |
| | | | TGG | dkk | | | | |
| | | | T | e | | | | |
| 612852 | N/A | N/A | TCT | ekk | 31 | 3817 | 3832 | 1055 |
| | | | CAG | ddd | | | | |
| | | | TTG | ddd | | | | |
| | | | TGA | ddd | | | | |
| | | | TCT | dkk | | | | |
| | | | G | e | | | | |
| 612853 | N/A | N/A | TCC | ekk | 0 | 3868 | 3883 | 1056 |
| | | | AGA | ddd | | | | |
| | | | GAC | ddd | | | | |
| | | | GCA | ddd | | | | |
| | | | ATT | dkk | | | | |
| | | | C | e | | | | |
| 612854 | N/A | N/A | TCT | ekk | 11 | 3870 | 3885 | 1057 |
| | | | CCA | ddd | | | | |
| | | | GAG | ddd | | | | |
| | | | ACG | ddd | | | | |
| | | | CAA | dkk | | | | |
| | | | T | e | | | | |
| 612855 | N/A | N/A | ACC | ekk | 4 | 3983 | 3998 | 1058 |
| | | | TGT | ddd | | | | |
| | | | GGG | ddd | | | | |
| | | | AAC | ddd | | | | |
| | | | CGA | dkk | | | | |
| | | | C | e | | | | |
| 612856 | N/A | N/A | AAA | ekk | 0 | 3985 | 4000 | 1059 |
| | | | CCT | ddd | | | | |
| | | | GTG | ddd | | | | |
| | | | GGA | ddd | | | | |
| | | | ACC | dkk | | | | |
| | | | G | e | | | | |
| 612857 | N/A | N/A | CCT | ekk | 27 | 4340 | 4355 | 1060 |
| | | | AGA | ddd | | | | |
| | | | TTT | ddd | | | | |
| | | | TTC | ddd | | | | |
| | | | TGC | dkk | | | | |
| | | | T | e | | | | |
| 612858 | N/A | N/A | GCC | ekk | 57 | 4420 | 4435 | 1061 |
| | | | TTT | ddd | | | | |
| | | | TCT | ddd | | | | |
| | | | GTC | ddd | | | | |
| | | | CCC | dkk | | | | |
| | | | C | e | | | | |
| 612859 | N/A | N/A | CAT | ekk | 12 | 4464 | 4479 | 1062 |
| | | | TTC | ddd | | | | |
| | | | TTG | ddd | | | | |
| | | | TGG | ddd | | | | |
| | | | AGG | dkk | | | | |
| | | | G | e | | | | |
| 612860 | N/A | N/A | TGG | ekk | 2 | 4569 | 4584 | 1063 |
| | | | GCT | ddd | | | | |
| | | | GGC | ddd | | | | |
| | | | CCT | ddd | | | | |
| | | | GCT | dkk | | | | |
| | | | A | e | | | | |
| 612861 | N/A | N/A | GAG | ekk | 33 | 4822 | 4837 | 1064 |
| | | | CCC | ddd | | | | |
| | | | CAA | ddd | | | | |
| | | | AGG | ddd | | | | |
| | | | CAT | dkk | | | | |
| | | | G | e | | | | |
| 612862 | N/A | N/A | TCT | ekk | 43 | 5357 | 5372 | 1065 |
| | | | AAT | ddd | | | | |
| | | | ATG | ddd | | | | |
| | | | ACC | ddd | | | | |
| | | | TGT | dkk | | | | |
| | | | G | e | | | | |
| 612863 | N/A | N/A | TGA | ekk | 13 | 5360 | 5375 | 1066 |
| | | | TCT | ddd | | | | |
| | | | AAT | ddd | | | | |
| | | | ATG | ddd | | | | |
| | | | ACC | dkk | | | | |
| | | | T | e | | | | |
| 612864 | N/A | N/A | GTC | ekk | 0 | 5455 | 5470 | 1067 |
| | | | CTC | ddd | | | | |
| | | | AAC | ddd | | | | |
| | | | CCC | ddd | | | | |
| | | | AGG | dkk | | | | |
| | | | A | e | | | | |
| 612865 | N/A | N/A | GCT | ekk | 4 | 5553 | 5568 | 1068 |
| | | | CCA | ddd | | | | |
| | | | TGG | ddd | | | | |
| | | | AAA | ddd | | | | |
| | | | ATA | dkk | | | | |
| | | | T | e | | | | |
| 612866 | N/A | N/A | TCC | ekk | 19 | 5593 | 5608 | 1069 |
| | | | ATT | ddd | | | | |
| | | | CAT | ddd | | | | |
| | | | GTC | ddd | | | | |
| | | | TAC | dkk | | | | |
| | | | A | e | | | | |
| 612867 | N/A | N/A | TTA | ekk | 17 | 5660 | 5675 | 1070 |
| | | | AGT | ddd | | | | |
| | | | GCC | ddd | | | | |
| | | | ATC | ddd | | | | |
| | | | TAA | dkk | | | | |
| | | | C | e | | | | |
| 612868 | N/A | N/A | GCA | ekk | 0 | 5714 | 5729 | 1071 |
| | | | TAC | ddd | | | | |
| | | | CCT | ddd | | | | |
| | | | GAA | ddd | | | | |
| | | | ATA | dkk | | | | |
| | | | T | e | | | | |
| 612893 | N/A | N/A | TGT | ekk | 42 | 10707 | 10722 | 1072 |
| | | | CTA | ddd | | | | |
| | | | CTC | ddd | | | | |
| | | | CCC | ddd | | | | |
| | | | ACC | dkk | | | | |
| | | | C | e | | | | |
| 612894 | N/A | N/A | ACA | ekk | 28 | 10834 | 10849 | 1073 |
| | | | GAC | ddd | | | | |
| | | | ACT | ddd | | | | |
| | | | AAC | ddd | | | | |
| | | | TGG | dkk | | | | |
| | | | A | e | | | | |
| 612895 | N/A | N/A | GTG | ekk | 37 | 10974 | 10989 | 1074 |
| | | | TGT | ddd | | | | |
| | | | CAG | ddd | | | | |
| | | | GTG | ddd | | | | |
| | | | TGG | dkk | | | | |
| | | | G | e | | | | |
| 612896 | N/A | N/A | GCA | ekk | 35 | 11016 | 11031 | 1075 |
| | | | AGT | ddd | | | | |
| | | | CAG | ddd | | | | |
| | | | TTC | ddd | | | | |
| | | | CAA | dkk | | | | |
| | | | G | e | | | | |
| 612897 | N/A | N/A | CTC | ekk | 55 | 11336 | 11351 | 1076 |
| | | | GAA | ddd | | | | |
| | | | AAT | ddd | | | | |
| | | | GGT | ddd | | | | |
| | | | TAC | dkk | | | | |
| | | | G | e | | | | |
| 612898 | N/A | N/A | GGT | ekk | 53 | 11583 | 11598 | 1077 |
| | | | GGT | ddd | | | | |
| | | | AAC | ddd | | | | |
| | | | CAC | ddd | | | | |
| | | | ATG | dkk | | | | |
| | | | C | e | | | | |
| 612899 | N/A | N/A | ATG | ekk | 31 | 11892 | 11907 | 1078 |
| | | | CAC | ddd | | | | |
| | | | TGT | ddd | | | | |
| | | | GTC | ddd | | | | |
| | | | TTA | dkk | | | | |
| | | | C | e | | | | |
| 612900 | N/A | N/A | AAT | ekk | 39 | 11896 | 11911 | 1079 |
| | | | AAT | ddd | | | | |
| | | | GCA | ddd | | | | |
| | | | CTG | ddd | | | | |
| | | | TGT | dkk | | | | |
| | | | C | e | | | | |
| 612901 | N/A | N/A | GTT | ekk | 68 | 11930 | 11945 | 1080 |
| | | | ACT | ddd | | | | |
| | | | TGG | ddd | | | | |
| | | | GTA | ddd | | | | |
| | | | ATT | dkk | | | | |
| | | | T | e | | | | |
| 612902 | N/A | N/A | TCC | ekk | 19 | 11974 | 11989 | 1081 |
| | | | TTT | ddd | | | | |
| | | | GGT | ddd | | | | |
| | | | GCA | ddd | | | | |
| | | | TTC | dkk | | | | |
| | | | T | e | | | | |
| 612903 | N/A | N/A | CTA | ekk | 0 | 11987 | 12002 | 1082 |
| | | | GGA | ddd | | | | |
| | | | ATG | ddd | | | | |
| | | | GTT | ddd | | | | |
| | | | GTC | dkk | | | | |
| | | | C | e | | | | |
| 612904 | N/A | N/A | GAC | ekk | 20 | 12129 | 12144 | 1083 |
| | | | GAC | ddd | | | | |
| | | | AGG | ddd | | | | |
| | | | GAT | ddd | | | | |
| | | | GTT | dkk | | | | |
| | | | T | e | | | | |
| 612905 | N/A | N/A | CTG | ekk | 25 | 12131 | 12146 | 1084 |
| | | | ACG | ddd | | | | |
| | | | ACA | ddd | | | | |
| | | | GGG | ddd | | | | |
| | | | ATG | dkk | | | | |
| | | | T | e | | | | |
| 612906 | N/A | N/A | GCA | ekk | 60 | 12210 | 12225 | 1085 |
| | | | CAG | ddd | | | | |
| | | | TTA | ddd | | | | |
| | | | GGA | ddd | | | | |
| | | | AGG | dkk | | | | |
| | | | C | e | | | | |
| 612907 | N/A | N/A | TTA | ekk | 8 | 12892 | 12907 | 1086 |
| | | | GCT | ddd | | | | |
| | | | AAC | ddd | | | | |
| | | | TTA | ddd | | | | |
| | | | GCA | dkk | | | | |
| | | | C | e | | | | |
| 612908 | N/A | N/A | CAT | ekk | 41 | 12914 | 12929 | 1087 |
| | | | GGC | ddd | | | | |
| | | | CCA | ddd | | | | |
| | | | CCA | ddd | | | | |
| | | | GTG | dkk | | | | |
| | | | C | e | | | | |
| 612909 | N/A | N/A | CAC | ekk | 52 | 13087 | 13102 | 1088 |
| | | | AGT | ddd | | | | |
| | | | GTA | ddd | | | | |
| | | | TGC | ddd | | | | |
| | | | CTG | dkk | | | | |
| | | | C | e | | | | |
| 612910 | N/A | N/A | GCA | ekk | 0 | 13195 | 13210 | 1089 |
| | | | CTG | ddd | | | | |
| | | | GGT | ddd | | | | |
| | | | GAC | ddd | | | | |
| | | | CCA | dkk | | | | |
| | | | G | e | | | | |
| 612911 | N/A | N/A | TCA | ekk | 0 | 13238 | 13253 | 1090 |
| | | | GCA | ddd | | | | |
| | | | CCT | ddd | | | | |
| | | | GCA | ddd | | | | |
| | | | AAG | dkk | | | | |
| | | | C | e | | | | |

Table 6 shows inhibition of AGT mRNA in HepG2 cells cultured at a density of 20,000 cells per well which were transfected using electroporation with 1000 nM antisense oligonucleotide. After a treatment period of approximately 24 hours, RNA was isolated from the cells and AGT mRNA levels were measured by quantitative real-time PCR. Human primer probe set RTS4039 (forward sequence GGACAAGGTGGAGGGTCTCA, designated herein as SEQ ID NO: 11; reverse sequence AGATCCTTGCAGCACCAGTTG, designated herein as SEQ ID NO: 12; and probe sequence ATGAAGAAACTATCTCCCCGGACCATCCAX, where X is a fluorescent label, designated herein as SEQ ID NO: 13) was used to measure mRNA levels. AGT mRNA levels were adjusted according to total RNA content, as measured by RIBOGREEN®. Results are presented as percent inhibition of AGT, relative to untreated control cells.

TABLE 6
Inhibition of AGT mRNA by MOE and/or cEt containing gapmers targeting SEQ ID NO: 1 and/or 2

| | | SEQ | | | | | | |
|---|---|---|---|---|---|---|---|---|
| | SEQ | ID: | | | | SEQ | SEQ | |
| | ID: 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568637 | 2046 | 2061 | CGCTGATTTGTCCGGG | eekddddddddddkke | 74 | 13515 | 13530 | 129 |
| 594622 | 2027 | 2042 | GTTATCTGCTGCTGGC | kkkddddddddddkkk | 64 | 13496 | 13511 | 163 |
| 594625 | 2047 | 2062 | TCGCTGATTTGTCCGG | kkkddddddddddkkk | 57 | 13516 | 13531 | 165 |
| 612205 | 1279 | 1294 | CTTCCATCCTGTCACA | ekkddddddddddkke | 0 | 6496 | 6511 | 957 |
| 612206 | 1282 | 1297 | AGTCTTCCATCCTGTC | ekkddddddddddkke | 9 | 6499 | 6514 | 958 |
| 612207 | 1286 | 1301 | AGCCAGTCTTCCATCC | ekkddddddddddkke | 0 | 6503 | 6518 | 959 |
| 612208 | 1290 | 1305 | GAGCAGCCAGTCTTCC | ekkddddddddddkke | 0 | 6507 | 6522 | 1091 |
| 612209 | 1293 | 1308 | AGGGAGCAGCCAGTCT | ekkddddddddddkke | 0 | 6510 | 6525 | 1092 |
| 612210 | 1296 | 1311 | ATCAGGGAGCAGCCAG | ekkddddddddddkke | 0 | 6513 | 6528 | 1093 |
| 612211 | 1300 | 1315 | TCCCATCAGGGAGCAG | ekkddddddddddkke | 0 | 6517 | 6532 | 1094 |
| 612212 | 1303 | 1318 | GGCTCCCATCAGGGAG | ekkddddddddddkke | 0 | 6520 | 6535 | 1095 |
| 612213 | 1306 | 1321 | ACTGGCTCCCATCAGG | ekkddddddddddkke | 16 | 6523 | 6538 | 1096 |
| 612214 | 1310 | 1325 | CCACACTGGCTCCCAT | ekkddddddddddkke | 0 | 6527 | 6542 | 1097 |
| 612215 | 1315 | 1330 | GCTGTCCACACTGGCT | ekkddddddddddkke | 13 | 6532 | 6547 | 1098 |
| 612216 | 1318 | 1333 | GGTGCTGTCCACACTG | ekkddddddddddkke | 20 | 6535 | 6550 | 1099 |
| 612217 | 1321 | 1336 | CAGGGTGCTGTCCACA | ekkddddddddddkke | 0 | 6538 | 6553 | 1100 |
| 612218 | 1324 | 1339 | AGCCAGGGTGCTGTCC | ekkddddddddddkke | 14 | 6541 | 6556 | 1101 |
| 612219 | 1327 | 1342 | GAAAGCCAGGGTGCTG | ekkddddddddddkke | 0 | 6544 | 6559 | 1102 |
| 612220 | 1330 | 1345 | GTTGAAAGCCAGGGTG | ekkddddddddddkke | 6 | 6547 | 6562 | 1103 |
| 612221 | 1333 | 1348 | GGTGTTGAAAGCCAGG | ekkddddddddddkke | 34 | 6550 | 6565 | 1104 |
| 612222 | 1336 | 1351 | GTAGGTGTTGAAAGCC | ekkddddddddddkke | 9 | 6553 | 6568 | 1105 |
| 612223 | 1351 | 1366 | CCCTTGGAAGTGGACG | ekkddddddddddkke | 0 | N/A | N/A | 1106 |
| 612224 | 1354 | 1369 | CTTCCCTTGGAAGTGG | ekkddddddddddkke | 17 | N/A | N/A | 1107 |
| 612225 | 1357 | 1372 | CATCTTCCCTTGGAAG | ekkddddddddddkke | 11 | N/A | N/A | 1108 |
| 612226 | 1360 | 1375 | CTTCATCTTCCCTTGG | ekkddddddddddkke | 0 | N/A | N/A | 1109 |
| 612227 | 1364 | 1379 | AGCCCTTCATCTTCCC | ekkddddddddddkke | 5 | 10375 | 10390 | 1110 |
| 612228 | 1367 | 1382 | AGAAGCCCTTCATCTT | ekkddddddddddkke | 0 | 10378 | 10393 | 1111 |
| 612229 | 1370 | 1385 | GGGAGAAGCCCTTCAT | ekkddddddddddkke | 0 | 10381 | 10396 | 1112 |
| 612230 | 1373 | 1388 | GCAGGGAGAAGCCCTT | ekkddddddddddkke | 25 | 10384 | 10399 | 1113 |
| 612231 | 1380 | 1395 | TCGGCCAGCAGGGAGA | ekkddddddddddkke | 32 | 10391 | 10406 | 1114 |
| 612232 | 1383 | 1398 | GGCTCGGCCAGCAGGG | ekkddddddddddkke | 24 | 10394 | 10409 | 1115 |
| 612233 | 1399 | 1414 | CACCCAGAACTCCTGG | ekkddddddddddkke | 5 | 10410 | 10425 | 960 |
| 612234 | 1402 | 1417 | GTCCACCCAGAACTCC | ekkddddddddddkke | 0 | 10413 | 10428 | 961 |
| 612235 | 1405 | 1420 | GTTGTCCACCCAGAAC | ekkddddddddddkke | 0 | 10416 | 10431 | 962 |
| 612236 | 1408 | 1423 | GCTGTTGTCCACCCAG | ekkddddddddddkke | 14 | 10419 | 10434 | 963 |
| 612237 | 1411 | 1426 | GGTGCTGTTGTCCACC | ekkddddddddddkke | 20 | 10422 | 10437 | 964 |
| 612238 | 1414 | 1429 | TGAGGTGCTGTTGTCC | ekkddddddddddkke | 32 | 10425 | 10440 | 965 |
| 612239 | 1417 | 1432 | CACTGAGGTGCTGTTG | ekkddddddddddkke | 36 | 10428 | 10443 | 966 |
| 612240 | 1421 | 1436 | CAGACACTGAGGTGCT | ekkddddddddddkke | 1 | 10432 | 10447 | 93 |
| 612241 | 1429 | 1444 | CATGGGAACAGACACT | ekkddddddddddkke | 9 | 10440 | 10455 | 967 |
| 612242 | 1432 | 1447 | GAGCATGGGAACAGAC | ekkddddddddddkke | 0 | 10443 | 10458 | 968 |
| 612243 | 1435 | 1450 | AGAGAGCATGGGAACA | ekkddddddddddkke | 0 | 10446 | 10461 | 969 |
| 612244 | 1438 | 1453 | GCCAGAGAGCATGGGA | ekkddddddddddkke | 5 | 10449 | 10464 | 970 |
| 612245 | 1441 | 1456 | CATGCCAGAGAGCATG | ekkddddddddddkke | 27 | 10452 | 10467 | 971 |
| 612246 | 1444 | 1459 | GCCCATGCCAGAGAGC | ekkddddddddddkke | 0 | 10455 | 10470 | 972 |
| 612247 | 1447 | 1462 | GGTGCCCATGCCAGAG | ekkddddddddddkke | 36 | 10458 | 10473 | 973 |
| 612248 | 1450 | 1465 | GAAGGTGCCCATGCCA | ekkddddddddddkke | 0 | 10461 | 10476 | 974 |
| 612249 | 1453 | 1468 | CTGGAAGGTGCCCATG | ekkddddddddddkke | 24 | 10464 | 10479 | 975 |
| 612250 | 1457 | 1472 | AGTGCTGGAAGGTGCC | ekkddddddddddkke | 0 | 10468 | 10483 | 976 |
| 612251 | 1460 | 1475 | TCCAGTGCTGGAAGGT | ekkddddddddddkke | 3 | 10471 | 10486 | 977 |
| 612252 | 1462 | 1477 | ACTCCAGTGCTGGAAG | ekkddddddddddkke | 72 | 10473 | 10488 | 96 |
| 612253 | 1463 | 1478 | CACTCCAGTGCTGGAA | ekkddddddddddkke | 19 | 10474 | 10489 | 978 |
| 612254 | 1465 | 1480 | GTCACTCCAGTGCTGG | ekkddddddddddkke | 45 | 10476 | 10491 | 97 |
| 612255 | 1466 | 1481 | TGTCACTCCAGTGCTG | ekkddddddddddkke | 15 | 10477 | 10492 | 979 |
| 612256 | 1467 | 1482 | ATGTCACTCCAGTGCT | ekkddddddddddkke | 0 | 10478 | 10493 | 980 |
| 612257 | 1468 | 1483 | GATGTCACTCCAGTGC | ekkddddddddddkke | 16 | 10479 | 10494 | 981 |
| 612258 | 1469 | 1484 | GGATGTCACTCCAGTG | ekkddddddddddkke | 0 | 10480 | 10495 | 982 |
| 612259 | 1470 | 1485 | TGGATGTCACTCCAGT | ekkddddddddddkke | 3 | 10481 | 10496 | 983 |
| 612260 | 1472 | 1487 | CCTGGATGTCACTCCA | ekkddddddddddkke | 10 | 10483 | 10498 | 984 |
| 612261 | 1475 | 1490 | TGTCCTGGATGTCACT | ekkddddddddddkke | 8 | 10486 | 10501 | 985 |
| 612262 | 1478 | 1493 | AGTTGTCCTGGATGTC | ekkddddddddddkke | 0 | 10489 | 10504 | 986 |
| 612263 | 1481 | 1496 | AGAAGTTGTCCTGGAT | ekkddddddddddkke | 14 | 10492 | 10507 | 987 |
| 612264 | 1484 | 1499 | CCGAGAAGTTGTCCTG | ekkddddddddddkke | 10 | 10495 | 10510 | 99 |
| 612265 | 1487 | 1502 | TCACCGAGAAGTTGTC | ekkddddddddddkke | 0 | 10498 | 10513 | 988 |
| 612266 | 1490 | 1505 | GAGTCACCGAGAAGTT | ekkddddddddddkke | 33 | 10501 | 10516 | 989 |
| 612267 | 1493 | 1508 | CTTGAGTCACCGAGAA | ekkddddddddddkke | 35 | 10504 | 10519 | 990 |
| 612268 | 1496 | 1511 | GCACTTGAGTCACCGA | ekkddddddddddkke | 37 | 10507 | 10522 | 991 |
| 612269 | 1499 | 1514 | AGGGCACTTGAGTCAC | ekkddddddddddkke | 0 | 10510 | 10525 | 992 |
| 612270 | 1502 | 1517 | TGAAGGGCACTTGAGT | ekkddddddddddkke | 8 | 10513 | 10528 | 993 |
| 612271 | 1505 | 1520 | CAGTGAAGGGCACTTG | ekkddddddddddkke | 8 | 10516 | 10531 | 994 |
| 612272 | 1508 | 1523 | TCTCAGTGAAGGGCAC | ekkddddddddddkke | 0 | 10519 | 10534 | 995 |
| 612273 | 1511 | 1526 | CGCTCTCAGTGAAGGG | ekkddddddddddkke | 18 | 10522 | 10537 | 996 |
| 612274 | 1524 | 1539 | AGCAGCAGGCAGGCGC | ekkddddddddddkke | 27 | 10535 | 10550 | 997 |
| 612275 | 1528 | 1543 | GATCAGCAGCAGGCAG | ekkddddddddddkke | 39 | 10539 | 10554 | 998 |
| 612276 | 1532 | 1547 | GCTGGATCAGCAGCAG | ekkddddddddddkke | 21 | 10543 | 10558 | 999 |
| 612277 | 1535 | 1550 | GAGGCTGGATCAGCAG | ekkddddddddddkke | 34 | 10546 | 10561 | 1000 |
| 612278 | 1538 | 1553 | AGTGAGGCTGGATCAG | ekkddddddddddkke | 28 | 10549 | 10564 | 1001 |
| 612279 | 1541 | 1556 | CATAGTGAGGCTGGAT | ekkddddddddddkke | 13 | 10552 | 10567 | 1002 |
| 612280 | 1544 | 1559 | AGGCATAGTGAGGCTG | ekkddddddddddkke | 0 | 10555 | 10570 | 1003 |

Table 7 shows inhibition of AGT mRNA in HepG2 cells cultured at a density of 20,000 cells per well which were transfected using electroporation with 1000 nM antisense oligonucleotide. After a treatment period of approximately 24 hours, RNA was isolated from the cells and AGT mRNA levels were measured 5 by quantitative real-time PCR. Human primer probe set RTS4039 was used to measure mRNA levels. AGT mRNA levels were adjusted according to total RNA content, as measured by RIBOGREEN®. Results are presented as percent inhibition of AGT, relative to untreated control cells.

TABLE 7
Inhibition of AGT mRNA by MOE and/or cEt containing gapmers targeting SEQ ID NO: 1 and/or 2

| | | SEQ | | | | | | |
|---|---|---|---|---|---|---|---|---|
| | SEQ | ID: | | | | SEQ | SEQ | |
| | ID: 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568637 | 2046 | 2061 | CGCTGATTTGTCCGGG | eekddddddddddkke | 93 | 13515 | 13530 | 129 |
| 568637 | 2046 | 2061 | CGCTGATTTGTCCGGG | eekddddddddddkke | 90 | 13515 | 13530 | 129 |
| 568637 | 2046 | 2061 | CGCTGATTTGTCCGGG | eekddddddddddkke | 84 | 13515 | 13530 | 129 |
| 594622 | 2027 | 2042 | GTTATCTGCTGCTGGC | kkkddddddddddkkk | 86 | 13496 | 13511 | 163 |
| 594622 | 2027 | 2042 | GTTATCTGCTGCTGGC | kkkddddddddddkkk | 85 | 13496 | 13511 | 163 |
| 594622 | 2027 | 2042 | GTTATCTGCTGCTGGC | kkkddddddddddkkk | 5 | 13496 | 13511 | 163 |
| 594625 | 2047 | 2062 | TCGCTGATTTGTCCGG | kkkddddddddddkkk | 75 | 13516 | 13531 | 165 |
| 594625 | 2047 | 2062 | TCGCTGATTTGTCCGG | kkkddddddddddkkk | 70 | 13516 | 13531 | 165 |
| 594625 | 2047 | 2062 | TCGCTGATTTGTCCGG | kkkddddddddddkkk | 0 | 13516 | 13531 | 165 |
| 612129 | 965 | 980 | GCCTGTCAGCTGTGTG | ekkddddddddddkke | 35 | 6182 | 6197 | 889 |
| 612130 | 968 | 983 | GTAGCCTGTCAGCTGT | ekkddddddddddkke | 35 | 6185 | 6200 | 890 |
| 612131 | 971 | 986 | CCTGTAGCCTGTCAGC | ekkddddddddddkke | 30 | 6188 | 6203 | 891 |
| 612132 | 974 | 989 | TTGCCTGTAGCCTGTC | ekkddddddddddkke | 37 | 6191 | 6206 | 892 |
| 612133 | 977 | 992 | GGATTGCCTGTAGCCT | ekkddddddddddkke | 30 | 6194 | 6209 | 893 |
| 612134 | 980 | 995 | CCAGGATTGCCTGTAG | ekkddddddddddkke | 56 | 6197 | 6212 | 894 |
| 612135 | 983 | 998 | CACCCAGGATTGCCTG | ekkddddddddddkke | 0 | 6200 | 6215 | 68 |
| 612136 | 986 | 1001 | GAACACCCAGGATTGC | ekkddddddddddkke | 8 | 6203 | 6218 | 895 |
| 612137 | 993 | 1008 | TTCCAAGGAACACCCA | ekkddddddddddkke | 27 | 6210 | 6225 | 69 |
| 612138 | 997 | 1012 | GTCCTTCCAAGGAACA | ekkddddddddddkke | 26 | 6214 | 6229 | 896 |
| 612139 | 1000 | 1015 | CTTGTCCTTCCAAGGA | ekkddddddddddkke | 47 | 6217 | 6232 | 897 |
| 612140 | 1003 | 1018 | GTTCTTGTCCTTCCAA | ekkddddddddddkke | 36 | 6220 | 6235 | 898 |
| 612141 | 1006 | 1021 | GCAGTTCTTGTCCTTC | ekkddddddddddkke | 28 | 6223 | 6238 | 899 |
| 612142 | 1009 | 1024 | GGTGCAGTTCTTGTCC | ekkddddddddddkke | 13 | 6226 | 6241 | 900 |
| 612143 | 1012 | 1027 | GGAGGTGCAGTTCTTG | ekkddddddddddkke | 0 | 6229 | 6244 | 901 |
| 612144 | 1015 | 1030 | CCGGGAGGTGCAGTTC | ekkddddddddddkke | 27 | 6232 | 6247 | 902 |
| 612145 | 1018 | 1033 | CAGCCGGGAGGTGCAG | ekkddddddddddkke | 39 | 6235 | 6250 | 903 |
| 612146 | 1021 | 1036 | ATCCAGCCGGGAGGTG | ekkddddddddddkke | 24 | 6238 | 6253 | 904 |
| 612147 | 1024 | 1039 | CGCATCCAGCCGGGAG | ekkddddddddddkke | 55 | 6241 | 6256 | 905 |
| 612148 | 1027 | 1042 | GTGCGCATCCAGCCGG | ekkddddddddddkke | 37 | 6244 | 6259 | 906 |
| 612149 | 1030 | 1045 | CTTGTGCGCATCCAGC | ekkddddddddddkke | 11 | 6247 | 6262 | 907 |
| 612150 | 1033 | 1048 | GACCTTGTGCGCATCC | ekkddddddddddkke | 12 | 6250 | 6265 | 908 |
| 612151 | 1036 | 1051 | CAGGACCTTGTGCGCA | ekkddddddddddkke | 41 | 6253 | 6268 | 909 |
| 612152 | 1039 | 1054 | AGACAGGACCTTGTGC | ekkddddddddddkke | 9 | 6256 | 6271 | 910 |
| 612153 | 1042 | 1057 | GGCAGACAGGACCTTG | ekkddddddddddkke | 30 | 6259 | 6274 | 911 |
| 612154 | 1060 | 1075 | GCCCTGTACAGCCTGC | ekkddddddddddkke | 19 | 6277 | 6292 | 912 |
| 612155 | 1064 | 1079 | GCAGGCCCTGTACAGC | ekkddddddddddkke | 0 | 6281 | 6296 | 913 |
| 612156 | 1067 | 1082 | CTAGCAGGCCCTGTAC | ekkddddddddddkke | 21 | 6284 | 6299 | 914 |
| 612157 | 1071 | 1086 | GCCACTAGCAGGCCCT | ekkddddddddddkke | 0 | 6288 | 6303 | 915 |
| 612158 | 1074 | 1089 | TGGGCCACTAGCAGGC | ekkddddddddddkke | 13 | 6291 | 6306 | 916 |
| 612159 | 1077 | 1092 | CCCTGGGCCACTAGCA | ekkddddddddddkke | 23 | 6294 | 6309 | 917 |
| 612160 | 1080 | 1095 | CTGCCCTGGGCCACTA | ekkddddddddddkke | 28 | 6297 | 6312 | 918 |
| 612161 | 1088 | 1103 | TATCAGCCCTGCCCTG | ekkddddddddddkke | 0 | 6305 | 6320 | 74 |
| 612162 | 1091 | 1106 | GGCTATCAGCCCTGCC | ekkddddddddddkke | 27 | 6308 | 6323 | 919 |
| 612163 | 1094 | 1109 | CCTGGCTATCAGCCCT | ekkddddddddddkke | 13 | 6311 | 6326 | 920 |
| 612164 | 1097 | 1112 | GGGCCTGGCTATCAGC | ekkddddddddddkke | 3 | 6314 | 6329 | 921 |
| 612165 | 1100 | 1115 | GCTGGGCCTGGCTATC | ekkddddddddddkke | 10 | 6317 | 6332 | 922 |
| 612166 | 1115 | 1130 | CCGTGGACAGCAGCAG | ekkddddddddddkke | 12 | 6332 | 6347 | 923 |
| 612167 | 1118 | 1133 | CCACCGTGGACAGCAG | ekkddddddddddkke | 42 | 6335 | 6350 | 924 |
| 612168 | 1121 | 1136 | CCACCACCGTGGACAG | ekkddddddddddkke | 27 | 6338 | 6353 | 925 |
| 612169 | 1124 | 1139 | CGCCCACCACCGTGGA | ekkddddddddddkke | 29 | 6341 | 6356 | 926 |
| 612170 | 1127 | 1142 | ACACGCCCACCACCGT | ekkddddddddddkke | 9 | 6344 | 6359 | 927 |
| 612171 | 1130 | 1145 | TGAACACGCCCACCAC | ekkddddddddddkke | 25 | 6347 | 6362 | 928 |
| 612172 | 1133 | 1148 | CTGTGAACACGCCCAC | ekkddddddddddkke | 32 | 6350 | 6365 | 929 |
| 612173 | 1136 | 1151 | GGGCTGTGAACACGCC | ekkddddddddddkke | 0 | 6353 | 6368 | 930 |
| 612174 | 1151 | 1166 | TCAGGTGCAGGCCTGG | ekkddddddddddkke | 8 | 6368 | 6383 | 78 |
| 612175 | 1154 | 1169 | GCTTCAGGTGCAGGCC | ekkddddddddddkke | 30 | 6371 | 6386 | 931 |
| 612176 | 1157 | 1172 | GCTGCTTCAGGTGCAG | ekkddddddddddkke | 21 | 6374 | 6389 | 932 |
| 612177 | 1160 | 1175 | ACGGCTGCTTCAGGTG | ekkddddddddddkke | 46 | 6377 | 6392 | 933 |
| 612178 | 1163 | 1178 | CAAACGGCTGCTTCAG | ekkddddddddddkke | 7 | 6380 | 6395 | 934 |
| 612179 | 1166 | 1181 | GCACAAACGGCTGCTT | ekkddddddddddkke | 31 | 6383 | 6398 | 935 |
| 612180 | 1169 | 1184 | CCTGCACAAACGGCTG | ekkddddddddddkke | 10 | 6386 | 6401 | 936 |
| 612181 | 1172 | 1187 | GGCCCTGCACAAACGG | ekkddddddddddkke | 5 | 6389 | 6404 | 937 |
| 612182 | 1182 | 1197 | TAGAGAGCCAGGCCCT | ekkddddddddddkke | 29 | 6399 | 6414 | 80 |
| 612183 | 1185 | 1200 | GTATAGAGAGCCAGGC | ekkddddddddddkke | 0 | 6402 | 6417 | 938 |
| 612184 | 1203 | 1218 | CGTGGGAGGACCACAG | ekkddddddddddkke | 16 | 6420 | 6435 | 81 |
| 612185 | 1217 | 1232 | TGAAGTCCAGAGAGCG | ekkddddddddddkke | 27 | 6434 | 6449 | 82 |
| 612186 | 1220 | 1235 | CTGTGAAGTCCAGAGA | ekkddddddddddkke | 26 | 6437 | 6452 | 939 |
| 612187 | 1223 | 1238 | GTTCTGTGAAGTCCAG | ekkddddddddddkke | 44 | 6440 | 6455 | 940 |
| 612188 | 1226 | 1241 | CCAGTTCTGTGAAGTC | ekkddddddddddkke | 29 | 6443 | 6458 | 941 |
| 612189 | 1229 | 1244 | CATCCAGTTCTGTGAA | ekkddddddddddkke | 14 | 6446 | 6461 | 942 |
| 612190 | 1232 | 1247 | CAACATCCAGTTCTGT | ekkddddddddddkke | 0 | 6449 | 6464 | 943 |
| 612191 | 1235 | 1250 | CAGCAACATCCAGTTC | ekkddddddddddkke | 24 | 6452 | 6467 | 944 |
| 612192 | 1244 | 1259 | TCTTCTCAGCAGCAAC | ekkddddddddddkke | 62 | 6461 | 6476 | 84 |
| 612193 | 1247 | 1262 | CAATCTTCTCAGCAGC | ekkddddddddddkke | 27 | 6464 | 6479 | 945 |
| 612194 | 1250 | 1265 | TGTCAATCTTCTCAGC | ekkddddddddddkke | 18 | 6467 | 6482 | 946 |
| 612195 | 1253 | 1268 | ACCTGTCAATCTTCTC | ekkddddddddddkke | 33 | 6470 | 6485 | 947 |
| 612196 | 1256 | 1271 | TGAACCTGTCAATCTT | ekkddddddddddkke | 25 | 6473 | 6488 | 948 |
| 612197 | 1259 | 1274 | GCATGAACCTGTCAAT | ekkddddddddddkke | 27 | 6476 | 6491 | 949 |
| 612198 | 1262 | 1277 | CCTGCATGAACCTGTC | ekkddddddddddkke | 15 | 6479 | 6494 | 950 |
| 612199 | 1265 | 1280 | CAGCCTGCATGAACCT | ekkddddddddddkke | 42 | 6482 | 6497 | 951 |
| 612200 | 1267 | 1282 | CACAGCCTGCATGAAC | ekkddddddddddkke | 39 | 6484 | 6499 | 952 |
| 612201 | 1268 | 1283 | TCACAGCCTGCATGAA | ekkddddddddddkke | 27 | 6485 | 6500 | 953 |
| 612202 | 1274 | 1289 | ATCCTGTCACAGCCTG | ekkddddddddddkke | 44 | 6491 | 6506 | 954 |
| 612203 | 1276 | 1291 | CCATCCTGTCACAGCC | ekkddddddddddkke | 39 | 6493 | 6508 | 955 |
| 612204 | 1277 | 1292 | TCCATCCTGTCACAGC | ekkddddddddddkke | 27 | 6494 | 6509 | 956 |
| 612688 | N/A | N/A | CGGCTTACCTTCTGCT | ekkddddddddddkke | 7 | 2483 | 2498 | 565 |
| 612761 | N/A | N/A | CGAAGGGAGACCCATT | ekkddddddddddkke | 24 | 8270 | 8285 | 1116 |
| 612762 | N/A | N/A | TTCGAAGGGAGACCCA | ekkddddddddddkke | 9 | 8272 | 8287 | 1117 |
| 612763 | N/A | N/A | CTTTCGAAGGGAGACC | ekkddddddddddkke | 12 | 8274 | 8289 | 1118 |
| 612764 | N/A | N/A | CCGATCTCCTCACTGG | ekkddddddddddkke | 9 | 8497 | 8512 | 1119 |
| 612765 | N/A | N/A | CCCCGATCTCCTCACT | ekkddddddddddkke | 6 | 8499 | 8514 | 1120 |
| 612766 | N/A | N/A | ACAGCCCCCGATCTCC | ekkddddddddddkke | 35 | 8504 | 8519 | 1121 |
| 612767 | N/A | N/A | GAGACAGCCCCCGATC | ekkddddddddddkke | 3 | 8507 | 8522 | 1122 |
| 612768 | N/A | N/A | CCGAGACAGCCCCCGA | ekkddddddddddkke | 7 | 8509 | 8524 | 1123 |
| 612769 | N/A | N/A | CTAGCTGCCTGCTGAG | ekkddddddddddkke | 27 | 8569 | 8584 | 1124 |
| 612770 | N/A | N/A | TCTAGCTGCCTGCTGA | ekkddddddddddkke | 22 | 8570 | 8585 | 1125 |
| 612771 | N/A | N/A | GTGGGACACATCTAGC | ekkddddddddddkke | 16 | 8580 | 8595 | 1126 |
| 612772 | N/A | N/A | TCTAGTGGGACACATC | ekkddddddddddkke | 27 | 8584 | 8599 | 1127 |
| 612773 | N/A | N/A | TCTCTAGTGGGACACA | ekkddddddddddkke | 17 | 8586 | 8601 | 1128 |
| 612774 | N/A | N/A | CATGAGAGTGGCTGCC | ekkddddddddddkke | 29 | 8789 | 8804 | 1129 |
| 612775 | N/A | N/A | CTTTTAGTTTAGAGGG | ekkddddddddddkke | 25 | 8883 | 8898 | 1130 |
| 612776 | N/A | N/A | ATGTGAGCGGGAAACT | ekkddddddddddkke | 16 | 8961 | 8976 | 1131 |
| 612777 | N/A | N/A | CATGTGAGCGGGAAAC | ekkddddddddddkke | 38 | 8962 | 8977 | 1132 |
| 612778 | N/A | N/A | CGGAGCACTCAGTCTC | ekkddddddddddkke | 38 | 8985 | 9000 | 1133 |
| 612779 | N/A | N/A | GTCCTCAGTCCTCGGA | ekkddddddddddkke | 8 | 8997 | 9012 | 1134 |
| 612780 | N/A | N/A | CGTCCTCAGTCCTCGG | ekkddddddddddkke | 53 | 8998 | 9013 | 1135 |
| 612781 | N/A | N/A | GCAGTGGCAGACCTGG | ekkddddddddddkke | 23 | 9023 | 9038 | 1136 |
| 612782 | N/A | N/A | TAGAGATGGTTCAGAA | ekkddddddddddkke | 13 | 9166 | 9181 | 1137 |
| 612783 | N/A | N/A | TGAGTAGAGATGGTTC | ekkddddddddddkke | 25 | 9170 | 9185 | 1138 |
| 612784 | N/A | N/A | GGAGTCTGAGTAGAGA | ekkddddddddddkke | 21 | 9176 | 9191 | 1139 |
| 612785 | N/A | N/A | GCCCTCGGCTGTCCTC | ekkddddddddddkke | 24 | 9294 | 9309 | 1140 |
| 612786 | N/A | N/A | CTCGACCTTACACTAG | ekkddddddddddkke | 29 | 9319 | 9334 | 1141 |
| 612787 | N/A | N/A | CCTCTGCCTCGACCTT | ekkddddddddddkke | 49 | 9326 | 9341 | 1142 |
| 612788 | N/A | N/A | AACTCGGGAGAGCCCG | ekkddddddddddkke | 41 | 9410 | 9425 | 1143 |
| 612789 | N/A | N/A | AACGAGGGCTCCATTC | ekkddddddddddkke | 22 | 9557 | 9572 | 1144 |
| 612790 | N/A | N/A | GACACACTCACTTTTT | ekkddddddddddkke | 25 | 9999 | 10014 | 1145 |
| 612791 | N/A | N/A | CTGCCAGGTCAACTCA | ekkddddddddddkke | 39 | 10050 | 10065 | 1146 |
| 612792 | N/A | N/A | GTACCTGCCAGGTCAA | ekkddddddddddkke | 25 | 10054 | 10069 | 1147 |
| 612793 | N/A | N/A | CTGGTACCTGCCAGGT | ekkddddddddddkke | 32 | 10057 | 10072 | 1148 |
| 612794 | N/A | N/A | AGTTCACTGAGGCAGC | ekkddddddddddkke | 37 | 10156 | 10171 | 1149 |
| 612795 | N/A | N/A | CCATTTGAGTTCACTG | ekkddddddddddkke | 61 | 10163 | 10178 | 1150 |
| 612796 | N/A | N/A | GCAGCCATTTGAGTTC | ekkddddddddddkke | 42 | 10167 | 10182 | 1151 |
| 612797 | N/A | N/A | AAGGCCCAGATCCTGC | ekkddddddddddkke | 0 | 10286 | 10301 | 1152 |
| 612798 | N/A | N/A | GAAATCCAGACAGGAG | ekkddddddddddkke | 11 | 10358 | 10373 | 1153 |
| 612821 | N/A | N/A | GGTCAGGCACAGACAC | ekkddddddddddkke | 0 | 12398 | 12413 | 1026 |
| 612822 | N/A | N/A | ATCCCGGTTTCAACTC | ekkddddddddddkke | 14 | 12671 | 12686 | 1027 |
| 612823 | N/A | N/A | TCCCGCTGGCCCCCGT | ekkddddddddddkke | 36 | 12866 | 12881 | 1028 |
| 612824 | N/A | N/A | CTAACTTAGCACAGAG | ekkddddddddddkke | 22 | 12888 | 12903 | 1029 |
| 612825 | N/A | N/A | CCATGGCCCACCAGTG | ekkddddddddddkke | 35 | 12915 | 12930 | 1030 |
| 612826 | N/A | N/A | TTGGCCATGGCCCACC | ekkddddddddddkke | 23 | 12919 | 12934 | 1031 |
| 612827 | N/A | N/A | GGCAGAATTCCTGGCT | ekkddddddddddkke | 0 | 12938 | 12953 | 1032 |
| 612828 | N/A | N/A | GCAAGGGTGTGTCTGT | ekkddddddddddkke | 23 | 13059 | 13074 | 1033 |
| 612829 | N/A | N/A | GGCAAGGGTGTGTCTG | ekkddddddddddkke | 29 | 13060 | 13075 | 1034 |
| 612830 | N/A | N/A | CTCAGTGTAGGCAAGG | ekkddddddddddkke | 37 | 13069 | 13084 | 1035 |
| 612831 | N/A | N/A | GAGGATGCACAGTGTA | ekkddddddddddkke | 15 | 13094 | 13109 | 1036 |
| 612832 | N/A | N/A | GCTCAGGACCTCTGTG | ekkddddddddddkke | 20 | 13151 | 13166 | 1037 |
| 612833 | N/A | N/A | GGCTCAGGACCTCTGT | ekkddddddddddkke | 48 | 13152 | 13167 | 1038 |
| 612834 | N/A | N/A | GGCGCACTGGGTGACC | ekkddddddddddkke | 32 | 13198 | 13213 | 1039 |
| 612835 | N/A | N/A | TCTGAGGGCGCACTGG | ekkddddddddddkke | 24 | 13204 | 13219 | 1040 |
| 612836 | N/A | N/A | TCATTCTGAGGGCGCA | ekkddddddddddkke | 18 | 13208 | 13223 | 1041 |
| 612837 | N/A | N/A | TGCCTTACCTTGGAAG | ekkddddddddddkke | 1 | 6574 | 6589 | 1154 |
| 612839 | N/A | N/A | ACACATACCTCCCCCA | ekkddddddddddkke | 4 | 12376 | 12391 | 1043 |
| 612840 | N/A | N/A | CGCATACCCTGAAATA | ekkddddddddddkke | 1 | 5715 | 5730 | 1044 |
| 612841 | N/A | N/A | CATCTTCCCTGAAATC | ekkddddddddddkke | 0 | 10368 | 10383 | 1155 |
| 612843 | N/A | N/A | TTCAGCACCTGCAAAG | ekkddddddddddkke | 0 | 13239 | 13254 | 1046 |
| 612844 | N/A | N/A | CCGGCTTACCTTCTGC | ekkddddddddddkke | 21 | 2484 | 2499 | 1047 |
| 612845 | N/A | N/A | CCCCCGGCTTACCTTC | ekkddddddddddkke | 0 | 2487 | 2502 | 1048 |
| 612846 | N/A | N/A | GGGCCCCCGGCTTACC | ekkddddddddddkke | 9 | 2490 | 2505 | 1049 |
| 612847 | N/A | N/A | GTGAATGTGAGCCCCG | ekkddddddddddkke | 9 | 3361 | 3376 | 1050 |
| 612848 | N/A | N/A | TCCCTCCTTATAACCC | ekkddddddddddkke | 5 | 3435 | 3450 | 1051 |
| 612849 | N/A | N/A | CCGGGCACTCTCAACT | ekkddddddddddkke | 6 | 3471 | 3486 | 1052 |
| 612850 | N/A | N/A | AGTAATGGTGCTCTGG | ekkddddddddddkke | 13 | 3752 | 3767 | 1053 |
| 612851 | N/A | N/A | TCCTGGGAGTAATGGT | ekkddddddddddkke | 16 | 3759 | 3774 | 1054 |
| 612852 | N/A | N/A | TCTCAGTTGTGATCTG | ekkddddddddddkke | 19 | 3817 | 3832 | 1055 |
| 612853 | N/A | N/A | TCCAGAGACGCAATTC | ekkddddddddddkke | 0 | 3868 | 3883 | 1056 |
| 612854 | N/A | N/A | TCTCCAGAGACGCAAT | ekkddddddddddkke | 15 | 3870 | 3885 | 1057 |
| 612855 | N/A | N/A | ACCTGTGGGAACCGAC | ekkddddddddddkke | 17 | 3983 | 3998 | 1058 |
| 612856 | N/A | N/A | AAACCTGTGGGAACCG | ekkddddddddddkke | 7 | 3985 | 4000 | 1059 |
| 612857 | N/A | N/A | CCTAGATTTTTCTGCT | ekkddddddddddkke | 15 | 4340 | 4355 | 1060 |
| 612858 | N/A | N/A | GCCTTTTCTGTCCCCC | ekkddddddddddkke | 24 | 4420 | 4435 | 1061 |
| 612859 | N/A | N/A | CATTTCTTGTGGAGGG | ekkddddddddddkke | 3 | 4464 | 4479 | 1062 |
| 612860 | N/A | N/A | TGGGCTGGCCCTGCTA | ekkddddddddddkke | 0 | 4569 | 4584 | 1063 |
| 612861 | N/A | N/A | GAGCCCCAAAGGCATG | ekkddddddddddkke | 0 | 4822 | 4837 | 1064 |
| 612862 | N/A | N/A | TCTAATATGACCTGTG | ekkddddddddddkke | 25 | 5357 | 5372 | 1065 |
| 612863 | N/A | N/A | TGATCTAATATGACCT | ekkddddddddddkke | 6 | 5360 | 5375 | 1066 |
| 612864 | N/A | N/A | GTCCTCAACCCCAGGA | ekkddddddddddkke | 9 | 5455 | 5470 | 1067 |
| 612865 | N/A | N/A | GCTCCATGGAAAATAT | ekkddddddddddkke | 0 | 5553 | 5568 | 1068 |
| 612866 | N/A | N/A | TCCATTCATGTCTACA | ekkddddddddddkke | 11 | 5593 | 5608 | 1069 |
| 612867 | N/A | N/A | TTAAGTGCCATCTAAC | ekkddddddddddkke | 23 | 5660 | 5675 | 1070 |
| 612868 | N/A | N/A | GCATACCCTGAAATAT | ekkddddddddddkke | 0 | 5714 | 5729 | 1071 |
| 612869 | N/A | N/A | AGGTATGTCCGCAGGG | ekkddddddddddkke | 35 | 6679 | 6694 | 1156 |
| 612870 | N/A | N/A | TAGTAGGGCAGCAGGT | ekkddddddddddkke | 7 | 6765 | 6780 | 1157 |
| 612871 | N/A | N/A | TTGTTTCTCCGAGTCT | ekkddddddddddkke | 42 | 6879 | 6894 | 1158 |
| 612872 | N/A | N/A | AGGCACTTTGTTTCTC | ekkddddddddddkke | 5 | 6886 | 6901 | 1159 |
| 612873 | N/A | N/A | CAAGGCACTTTGTTTC | ekkddddddddddkke | 0 | 6888 | 6903 | 1160 |
| 612874 | N/A | N/A | TAGAACTGGGCTGTGG | ekkddddddddddkke | 0 | 6962 | 6977 | 1161 |
| 612875 | N/A | N/A | CCCTCCTAACATGAAA | ekkddddddddddkke | 0 | 7071 | 7086 | 1162 |
| 612876 | N/A | N/A | CTTACAAGTAGCAAAT | ekkddddddddddkke | 11 | 7332 | 7347 | 1163 |
| 612877 | N/A | N/A | GCCAGGCTTAAAGTCT | ekkddddddddddkke | 10 | 7346 | 7361 | 1164 |
| 612878 | N/A | N/A | ATTGACCTTTAAAAGC | ekkddddddddddkke | 5 | 7407 | 7422 | 1165 |
| 612879 | N/A | N/A | TCTGGTTCAACACTCA | ekkddddddddddkke | 39 | 7640 | 7655 | 1166 |
| 612880 | N/A | N/A | TTCCCGTGACTGTGTG | ekkddddddddddkke | 25 | 7813 | 7828 | 1167 |
| 612881 | N/A | N/A | CGAGCTGCTCCCTGAG | ekkddddddddddkke | 15 | 7835 | 7850 | 1168 |
| 612882 | N/A | N/A | CACCCCACCCATGGAT | ekkddddddddddkke | 0 | 7855 | 7870 | 1169 |
| 612883 | N/A | N/A | TCTCTGTCCCTCACGA | ekkddddddddddkke | 20 | 7925 | 7940 | 1170 |
| 612884 | N/A | N/A | TTTCGAAGGGAGACCC | ekkddddddddddkke | 9 | 8273 | 8288 | 1171 |
| 612885 | N/A | N/A | CATCTAGCTGCCTGCT | ekkddddddddddkke | 0 | 8572 | 8587 | 1172 |
| 612886 | N/A | N/A | TGGGACACATCTAGCT | ekkddddddddddkke | 9 | 8579 | 8594 | 1173 |
| 612887 | N/A | N/A | ATCCTCAGGTCCTCTC | ekkddddddddddkke | 14 | 8598 | 8613 | 1174 |
| 612888 | N/A | N/A | ATGGTTCAGAAACAGT | ekkddddddddddkke | 28 | 9161 | 9176 | 1175 |
| 612889 | N/A | N/A | GATTTGCACACTGGGC | ekkddddddddddkke | 0 | 9489 | 9504 | 1176 |
| 612890 | N/A | N/A | CCCCGTGATCAACATC | ekkddddddddddkke | 0 | 9874 | 9889 | 1177 |
| 612891 | N/A | N/A | ATCGAGCAGAAAGTAC | ekkddddddddddkke | 24 | 9932 | 9947 | 1178 |
| 612892 | N/A | N/A | ACTGGTACCTGCCAGG | ekkddddddddddkke | 0 | 10058 | 10073 | 1179 |
| 612907 | N/A | N/A | TTAGCTAACTTAGCAC | ekkddddddddddkke | 10 | 12892 | 12907 | 1086 |
| 612908 | N/A | N/A | CATGGCCCACCAGTGC | ekkddddddddddkke | 13 | 12914 | 12929 | 1087 |
| 612909 | N/A | N/A | CACAGTGTATGCCTGC | ekkddddddddddkke | 15 | 13087 | 13102 | 1088 |
| 612910 | N/A | N/A | GCACTGGGTGACCCAG | ekkddddddddddkke | 0 | 13195 | 13210 | 1089 |
| 612911 | N/A | N/A | TCAGCACCTGCAAAGC | ekkddddddddddkke | 0 | 13238 | 13253 | 1090 |

Table 8 shows inhibition of AGT mRNA in HepG2 cells cultured at a density of 20,000 cells per well which were transfected using electroporation with 4000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 8
Inhibition of AGT mRNA by MOE containing gapmers targeting SEQ ID NO: 1 and/or 2

| | SEQ | SEQ | | | | | | |
|---|---|---|---|---|---|---|---|---|
| | ID: | ID: | | | | SEQ | SEQ | |
| | 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 87 | 13518 | 13537 | 239 |
| 610015 | 2049 | 2068 | GACACATCGCTGATTTGTCC | eeeeeddddddddddeeeee | 81 | 13518 | 13537 | 239 |
| 610015 | 2049 | 2068 | GACACATCGCTGATTTGTCC | eeeeeddddddddddeeeee | 43 | 13518 | 13537 | 239 |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 88 | 13518 | 13537 | 239 |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 87 | 13518 | 13537 | 239 |
| 619461 | 1 | 20 | CTGCTGCCCGCTCATGGGAT | eeeeeddddddddddeeeee | 5 | 1986 | 2005 | 1180 |
| 619462 | 7 | 26 | CTGACCCTGCTGCCCGCTCA | eeeeeddddddddddeeeee | 30 | 1992 | 2011 | 1181 |
| 619463 | 13 | 32 | CCACTTCTGACCCTGCTGCC | eeeeeddddddddddeeeee | 31 | 1998 | 2017 | 1182 |
| 619464 | 35 | 54 | TCTTGCTTAGGCAACACGGG | eeeeeddddddddddeeeee | 31 | 2020 | 2039 | 1183 |
| 619465 | 41 | 60 | GGAGAGTCTTGCTTAGGCAA | eeeeeddddddddddeeeee | 16 | 2026 | 2045 | 1184 |
| 619466 | 66 | 85 | GGAGGTGCAGAGGGCAGAGG | eeeeeddddddddddeeeee | 5 | 2051 | 2070 | 1185 |
| 619467 | 72 | 91 | CAGGCCGGAGGTGCAGAGGG | eeeeeddddddddddeeeee | 11 | 2057 | 2076 | 1186 |
| 619468 | 78 | 97 | GACATGCAGGCCGGAGGTGC | eeeeeddddddddddeeeee | 15 | 2063 | 2082 | 1187 |
| 619469 | 84 | 103 | CACAGGGACATGCAGGCCGG | eeeeeddddddddddeeeee | 19 | 2069 | 2088 | 1188 |
| 619470 | 90 | 109 | AGAGGCCACAGGGACATGCA | eeeeeddddddddddeeeee | 26 | 2075 | 2094 | 1189 |
| 619471 | 96 | 115 | CCCCCAAGAGGCCACAGGGA | eeeeeddddddddddeeeee | 10 | 2081 | 2100 | 1190 |
| 619472 | 102 | 121 | GATGTACCCCCAAGAGGCCA | eeeeeddddddddddeeeee | 31 | 2087 | 2106 | 1191 |
| 619473 | 108 | 127 | CCGGGAGATGTACCCCCAAG | eeeeeddddddddddeeeee | 34 | 2093 | 2112 | 1192 |
| 619474 | 114 | 133 | CCAGCCCCGGGAGATGTACC | eeeeeddddddddddeeeee | 11 | 2099 | 2118 | 1193 |
| 619475 | 120 | 139 | TCTGACCCAGCCCCGGGAGA | eeeeeddddddddddeeeee | 35 | 2105 | 2124 | 1194 |
| 619476 | 126 | 145 | AGGCCTTCTGACCCAGCCCC | eeeeeddddddddddeeeee | 21 | 2111 | 2130 | 1195 |
| 619477 | 132 | 151 | CCACCCAGGCCTTCTGACCC | eeeeeddddddddddeeeee | 0 | 2117 | 2136 | 1196 |
| 619478 | 138 | 157 | GGCCAACCACCCAGGCCTTC | eeeeeddddddddddeeeee | 31 | 2123 | 2142 | 1197 |
| 619479 | 144 | 163 | GCCTGAGGCCAACCACCCAG | eeeeeddddddddddeeeee | 36 | 2129 | 2148 | 1198 |
| 619480 | 150 | 169 | GTGACAGCCTGAGGCCAACC | eeeeeddddddddddeeeee | 8 | 2135 | 2154 | 1199 |
| 619481 | 156 | 175 | AGGTGTGTGACAGCCTGAGG | eeeeeddddddddddeeeee | 45 | 2141 | 2160 | 1200 |
| 619482 | 162 | 181 | CTCCCTAGGTGTGTGACAGC | eeeeeddddddddddeeeee | 27 | 2147 | 2166 | 1201 |
| 619483 | 168 | 187 | GAGCATCTCCCTAGGTGTGT | eeeeeddddddddddeeeee | 21 | 2153 | 2172 | 1202 |
| 619484 | 174 | 193 | AAACGGGAGCATCTCCCTAG | eeeeeddddddddddeeeee | 27 | 2159 | 2178 | 1203 |
| 619485 | 180 | 199 | TCCCAGAAACGGGAGCATCT | eeeeeddddddddddeeeee | 29 | 2165 | 2184 | 1204 |
| 619486 | 186 | 205 | CAAGGTTCCCAGAAACGGGA | eeeeeddddddddddeeeee | 0 | 2171 | 2190 | 1205 |
| 619487 | 208 | 227 | CGAAGTTTGCAGGAGTCGGG | eeeeeddddddddddeeeee | 27 | 2193 | 2212 | 1206 |
| 619488 | 214 | 233 | ATTTACCGAAGTTTGCAGGA | eeeeeddddddddddeeeee | 40 | 2199 | 2218 | 1207 |
| 619489 | 220 | 239 | TTACACATTTACCGAAGTTT | eeeeeddddddddddeeeee | 10 | 2205 | 2224 | 1208 |
| 619490 | 226 | 245 | GTCGAGTTACACATTTACCG | eeeeeddddddddddeeeee | 29 | 2211 | 2230 | 1209 |
| 619491 | 232 | 251 | TGCAGGGTCGAGTTACACAT | eeeeeddddddddddeeeee | 24 | 2217 | 2236 | 1210 |
| 619492 | 238 | 257 | AGCCGGTGCAGGGTCGAGTT | eeeeeddddddddddeeeee | 20 | 2223 | 2242 | 1211 |
| 619493 | 244 | 263 | AGAGTGAGCCGGTGCAGGGT | eeeeeddddddddddeeeee | 20 | 2229 | 2248 | 1212 |
| 619494 | 250 | 269 | CTGAACAGAGTGAGCCGGTG | eeeeeddddddddddeeeee | 25 | 2235 | 2254 | 1213 |
| 619495 | 256 | 275 | TCACTGCTGAACAGAGTGAG | eeeeeddddddddddeeeee | 41 | 2241 | 2260 | 1214 |
| 619496 | 262 | 281 | AGAGTTTCACTGCTGAACAG | eeeeeddddddddddeeeee | 13 | 2247 | 2266 | 1215 |
| 619497 | 268 | 287 | CGATGCAGAGTTTCACTGCT | eeeeeddddddddddeeeee | 29 | 2253 | 2272 | 1216 |
| 619498 | 274 | 293 | AGTGATCGATGCAGAGTTTC | eeeeeddddddddddeeeee | 28 | 2259 | 2278 | 1217 |
| 619499 | 280 | 299 | AGTCTTAGTGATCGATGCAG | eeeeeddddddddddeeeee | 26 | 2265 | 2284 | 1218 |
| 619500 | 286 | 305 | CCAGGAAGTCTTAGTGATCG | eeeeeddddddddddeeeee | 26 | 2271 | 2290 | 1219 |
| 619501 | 292 | 311 | CCTCTTCCAGGAAGTCTTAG | eeeeeddddddddddeeeee | 28 | 2277 | 2296 | 1220 |
| 619502 | 298 | 317 | CTGGGACCTCTTCCAGGAAG | eeeeeddddddddddeeeee | 20 | 2283 | 2302 | 1221 |
| 619503 | 304 | 323 | CTCACGCTGGGACCTCTTCC | eeeeeddddddddddeeeee | 12 | 2289 | 2308 | 1222 |
| 619504 | 310 | 329 | GCGACACTCACGCTGGGACC | eeeeeddddddddddeeeee | 25 | 2295 | 2314 | 1223 |
| 619505 | 316 | 335 | CCAGAAGCGACACTCACGCT | eeeeeddddddddddeeeee | 13 | 2301 | 2320 | 1224 |
| 619506 | 322 | 341 | CAGATGCCAGAAGCGACACT | eeeeeddddddddddeeeee | 24 | 2307 | 2326 | 1225 |
| 619507 | 328 | 347 | GAAGGACAGATGCCAGAAGC | eeeeeddddddddddeeeee | 40 | 2313 | 2332 | 1226 |
| 619508 | 334 | 353 | TGGCCAGAAGGACAGATGCC | eeeeeddddddddddeeeee | 3 | 2319 | 2338 | 1227 |
| 619509 | 340 | 359 | ACAGGCTGGCCAGAAGGACA | eeeeeddddddddddeeeee | 31 | 2325 | 2344 | 1228 |
| 619510 | 346 | 365 | CAGACCACAGGCTGGCCAGA | eeeeeddddddddddeeeee | 17 | 2331 | 2350 | 1229 |
| 619511 | 352 | 371 | CTTGGCCAGACCACAGGCTG | eeeeeddddddddddeeeee | 20 | 2337 | 2356 | 1230 |
| 619512 | 358 | 377 | ACATCACTTGGCCAGACCAC | eeeeeddddddddddeeeee | 7 | 2343 | 2362 | 1231 |
| 619513 | 364 | 383 | AGGGTTACATCACTTGGCCA | eeeeeddddddddddeeeee | 19 | 2349 | 2368 | 1232 |
| 619514 | 370 | 389 | GAGAGGAGGGTTACATCACT | eeeeeddddddddddeeeee | 28 | 2355 | 2374 | 1233 |
| 619515 | 376 | 395 | AGGCTGGAGAGGAGGGTTAC | eeeeeddddddddddeeeee | 31 | 2361 | 2380 | 1234 |
| 619516 | 382 | 401 | GTGCACAGGCTGGAGAGGAG | eeeeeddddddddddeeeee | 5 | 2367 | 2386 | 1235 |
| 619517 | 388 | 407 | CTGCCTGTGCACAGGCTGGA | eeeeeddddddddddeeeee | 15 | 2373 | 2392 | 1236 |
| 619518 | 394 | 413 | CCCAGGCTGCCTGTGCACAG | eeeeeddddddddddeeeee | 23 | 2379 | 2398 | 1237 |
| 619519 | 400 | 419 | GCTGTTCCCAGGCTGCCTGT | eeeeeddddddddddeeeee | 40 | 2385 | 2404 | 1238 |
| 619520 | 406 | 425 | GATGGAGCTGTTCCCAGGCT | eeeeeddddddddddeeeee | 12 | 2391 | 2410 | 1239 |
| 619521 | 431 | 450 | GCCCTATTTATAGCTGAGGG | eeeeeddddddddddeeeee | 23 | 2416 | 2435 | 1240 |
| 619522 | 437 | 456 | CACGATGCCCTATTTATAGC | eeeeeddddddddddeeeee | 10 | 2422 | 2441 | 1241 |
| 619523 | 443 | 462 | CCGGGTCACGATGCCCTATT | eeeeeddddddddddeeeee | 24 | 2428 | 2447 | 1242 |
| 619524 | 449 | 468 | CCCCGGCCGGGTCACGATGC | eeeeeddddddddddeeeee | 37 | 2434 | 2453 | 1243 |
| 619525 | 452 | 471 | TTCCCCCGGCCGGGTCACGA | eeeeeddddddddddeeeee | 24 | 2437 | 2456 | 1244 |
| 619526 | 455 | 474 | TTCTTCCCCCGGCCGGGTCA | eeeeeddddddddddeeeee | 19 | 2440 | 2459 | 1245 |
| 619527 | 458 | 477 | AGCTTCTTCCCCCGGCCGGG | eeeeeddddddddddeeeee | 7 | 2443 | 2462 | 1246 |
| 619528 | 461 | 480 | GGCAGCTTCTTCCCCCGGCC | eeeeeddddddddddeeeee | 38 | 2446 | 2465 | 1247 |
| 619529 | 464 | 483 | AACGGCAGCTTCTTCCCCCG | eeeeeddddddddddeeeee | 31 | 2449 | 2468 | 1248 |
| 619530 | 467 | 486 | AACAACGGCAGCTTCTTCCC | eeeeeddddddddddeeeee | 40 | 2452 | 2471 | 1249 |
| 619531 | 470 | 489 | CAGAACAACGGCAGCTTCTT | eeeeeddddddddddeeeee | 53 | 2455 | 2474 | 1250 |
| 619532 | 473 | 492 | ACCCAGAACAACGGCAGCTT | eeeeeddddddddddeeeee | 56 | 2458 | 2477 | 1251 |
| 619533 | 476 | 495 | AGTACCCAGAACAACGGCAG | eeeeeddddddddddeeeee | 50 | 2461 | 2480 | 1252 |
| 619534 | 479 | 498 | TGTAGTACCCAGAACAACGG | eeeeeddddddddddeeeee | 31 | 2464 | 2483 | 1253 |
| 619535 | 482 | 501 | TGCTGTAGTACCCAGAACAA | eeeeeddddddddddeeeee | 39 | 2467 | 2486 | 1254 |
| 619536 | 485 | 504 | TTCTGCTGTAGTACCCAGAA | eeeeeddddddddddeeeee | 52 | 2470 | 2489 | 1255 |
| 619537 | 488 | 507 | CCCTTCTGCTGTAGTACCCA | eeeeeddddddddddeeeee | 55 | N/A | N/A | 1256 |
| 619538 | 491 | 510 | ATACCCTTCTGCTGTAGTAC | eeeeeddddddddddeeeee | 39 | N/A | N/A | 1257 |
| 619539 | 494 | 513 | CGCATACCCTTCTGCTGTAG | eeeeeddddddddddeeeee | 69 | N/A | N/A | 1258 |
| 619540 | 497 | 516 | TTCCGCATACCCTTCTGCTG | eeeeeddddddddddeeeee | 65 | N/A | N/A | 1259 |
| 619541 | 500 | 519 | CGCTTCCGCATACCCTTCTG | eeeeeddddddddddeeeee | 60 | N/A | N/A | 1260 |
| 619542 | 503 | 522 | GCTCGCTTCCGCATACCCTT | eeeeeddddddddddeeeee | 78 | N/A | N/A | 1261 |
| 619543 | 506 | 525 | GGTGCTCGCTTCCGCATACC | eeeeeddddddddddeeeee | 69 | 5723 | 5742 | 1262 |
| 619544 | 525 | 544 | AGGAGCCATCTCAGACTGGG | eeeeeddddddddddeeeee | 53 | 5742 | 5761 | 1263 |
| 619545 | 528 | 547 | GGCAGGAGCCATCTCAGACT | eeeeeddddddddddeeeee | 56 | 5745 | 5764 | 1264 |
| 619546 | 531 | 550 | ACCGGCAGGAGCCATCTCAG | eeeeeddddddddddeeeee | 47 | 5748 | 5767 | 1265 |
| 619547 | 534 | 553 | CACACCGGCAGGAGCCATCT | eeeeeddddddddddeeeee | 39 | 5751 | 5770 | 1266 |
| 619548 | 537 | 556 | GCTCACACCGGCAGGAGCCA | eeeeeddddddddddeeeee | 47 | 5754 | 5773 | 1267 |
| 619549 | 540 | 559 | CAGGCTCACACCGGCAGGAG | eeeeeddddddddddeeeee | 42 | 5757 | 5776 | 1268 |
| 619550 | 543 | 562 | CCTCAGGCTCACACCGGCAG | eeeeeddddddddddeeeee | 58 | 5760 | 5779 | 1269 |
| 619551 | 546 | 565 | GGCCCTCAGGCTCACACCGG | eeeeeddddddddddeeeee | 53 | 5763 | 5782 | 1270 |
| 619552 | 549 | 568 | GGTGGCCCTCAGGCTCACAC | eeeeeddddddddddeeeee | 31 | 5766 | 5785 | 1271 |
| 619553 | 552 | 571 | GATGGTGGCCCTCAGGCTCA | eeeeeddddddddddeeeee | 8 | 5769 | 5788 | 1272 |
| 619554 | 555 | 574 | GAGGATGGTGGCCCTCAGGC | eeeeeddddddddddeeeee | 35 | 5772 | 5791 | 1273 |
| 619555 | 558 | 577 | GCAGAGGATGGTGGCCCTCA | eeeeeddddddddddeeeee | 54 | 5775 | 5794 | 1274 |
| 619556 | 561 | 580 | GAGGCAGAGGATGGTGGCCC | eeeeeddddddddddeeeee | 37 | 5778 | 5797 | 1275 |
| 619557 | 564 | 583 | CAGGAGGCAGAGGATGGTGG | eeeeeddddddddddeeeee | 13 | 5781 | 5800 | 1276 |
| 619558 | 572 | 591 | GCCCAGGCCAGGAGGCAGAG | eeeeeddddddddddeeeee | 43 | 5789 | 5808 | 1277 |
| 619559 | 575 | 594 | CCAGCCCAGGCCAGGAGGCA | eeeeeddddddddddeeeee | 44 | 5792 | 5811 | 1278 |
| 619560 | 578 | 597 | AGGCCAGCCCAGGCCAGGAG | eeeeeddddddddddeeeee | 50 | 5795 | 5814 | 1279 |
| 619561 | 581 | 600 | GCCAGGCCAGCCCAGGCCAG | eeeeeddddddddddeeeee | 55 | 5798 | 5817 | 1280 |
| 619562 | 584 | 603 | GCAGCCAGGCCAGCCCAGGC | eeeeeddddddddddeeeee | 43 | 5801 | 5820 | 1281 |
| 619563 | 587 | 606 | CCTGCAGCCAGGCCAGCCCA | eeeeeddddddddddeeeee | 38 | 5804 | 5823 | 1282 |
| 619564 | 590 | 609 | TCACCTGCAGCCAGGCCAGC | eeeeeddddddddddeeeee | 33 | 5807 | 5826 | 1283 |
| 619565 | 593 | 612 | CGGTCACCTGCAGCCAGGCC | eeeeeddddddddddeeeee | 45 | 5810 | 5829 | 1284 |
| 619566 | 596 | 615 | ACCCGGTCACCTGCAGCCAG | eeeeeddddddddddeeeee | 42 | 5813 | 5832 | 1285 |
| 619567 | 599 | 618 | TACACCCGGTCACCTGCAGC | eeeeeddddddddddeeeee | 22 | 5816 | 5835 | 1286 |
| 619568 | 602 | 621 | ATGTACACCCGGTCACCTGC | eeeeeddddddddddeeeee | 37 | 5819 | 5838 | 1287 |
| 619569 | 605 | 624 | TGTATGTACACCCGGTCACC | eeeeeddddddddddeeeee | 18 | 5822 | 5841 | 1288 |
| 619570 | 608 | 627 | GGGTGTATGTACACCCGGTC | eeeeeddddddddddeeeee | 26 | 5825 | 5844 | 1289 |
| 619571 | 626 | 645 | TGGATGACGAGGTGGAAGGG | eeeeeddddddddddeeeee | 44 | 5843 | 5862 | 1290 |
| 619572 | 629 | 648 | TTGTGGATGACGAGGTGGAA | eeeeeddddddddddeeeee | 35 | 5846 | 5865 | 1291 |
| 619573 | 632 | 651 | TCATTGTGGATGACGAGGTG | eeeeeddddddddddeeeee | 39 | 5849 | 5868 | 1292 |
| 619574 | 635 | 654 | CTCTCATTGTGGATGACGAG | eeeeeddddddddddeeeee | 68 | 5852 | 5871 | 1293 |
| 619575 | 638 | 657 | GTACTCTCATTGTGGATGAC | eeeeeddddddddddeeeee | 65 | 5855 | 5874 | 1294 |
| 619576 | 641 | 660 | CAGGTACTCTCATTGTGGAT | eeeeeddddddddddeeeee | 54 | 5858 | 5877 | 1295 |
| 619577 | 644 | 663 | TCACAGGTACTCTCATTGTG | eeeeeddddddddddeeeee | 42 | 5861 | 5880 | 1296 |
| 619578 | 647 | 666 | TGCTCACAGGTACTCTCATT | eeeeeddddddddddeeeee | 59 | 5864 | 5883 | 1297 |
| 619579 | 650 | 669 | AGCTGCTCACAGGTACTCTC | eeeeeddddddddddeeeee | 57 | 5867 | 5886 | 1298 |
| 619580 | 653 | 672 | GCCAGCTGCTCACAGGTACT | eeeeeddddddddddeeeee | 70 | 5870 | 5889 | 1299 |
| 619581 | 656 | 675 | TTTGCCAGCTGCTCACAGGT | eeeeeddddddddddeeeee | 47 | 5873 | 5892 | 1300 |
| 619582 | 659 | 678 | GCCTTTGCCAGCTGCTCACA | eeeeeddddddddddeeeee | 49 | 5876 | 5895 | 1301 |
| 619583 | 662 | 681 | TTGGCCTTTGCCAGCTGCTC | eeeeeddddddddddeeeee | 58 | 5879 | 5898 | 1302 |
| 619584 | 665 | 684 | GCATTGGCCTTTGCCAGCTG | eeeeeddddddddddeeeee | 56 | 5882 | 5901 | 1303 |
| 619585 | 668 | 687 | CCGGCATTGGCCTTTGCCAG | eeeeeddddddddddeeeee | 45 | 5885 | 5904 | 1304 |
| 619586 | 671 | 690 | TTCCCGGCATTGGCCTTTGC | eeeeeddddddddddeeeee | 46 | 5888 | 5907 | 1305 |
| 619587 | 674 | 693 | GGCTTCCCGGCATTGGCCTT | eeeeeddddddddddeeeee | 39 | 5891 | 5910 | 1306 |
| 619588 | 677 | 696 | TTGGGCTTCCCGGCATTGGC | eeeeeddddddddddeeeee | 41 | 5894 | 5913 | 1307 |
| 619589 | 680 | 699 | TCTTTGGGCTTCCCGGCATT | eeeeeddddddddddeeeee | 28 | 5897 | 5916 | 1308 |
| 619590 | 701 | 720 | GGAGCAGGTATGAAGGTGGG | eeeeeddddddddddeeeee | 35 | 5918 | 5937 | 1309 |
| 619591 | 704 | 723 | ATTGGAGCAGGTATGAAGGT | eeeeeddddddddddeeeee | 49 | 5921 | 5940 | 1310 |
| 619592 | 707 | 726 | TGAATTGGAGCAGGTATGAA | eeeeeddddddddddeeeee | 32 | 5924 | 5943 | 1311 |
| 619593 | 710 | 729 | GCCTGAATTGGAGCAGGTAT | eeeeeddddddddddeeeee | 57 | 5927 | 5946 | 1312 |
| 619594 | 713 | 732 | TTGGCCTGAATTGGAGCAGG | eeeeeddddddddddeeeee | 51 | 5930 | 5949 | 1313 |
| 619595 | 716 | 735 | GTCTTGGCCTGAATTGGAGC | eeeeeddddddddddeeeee | 42 | 5933 | 5952 | 1314 |
| 619596 | 719 | 738 | GATGTCTTGGCCTGAATTGG | eeeeeddddddddddeeeee | 24 | 5936 | 5955 | 1315 |
| 619597 | 740 | 759 | AGGGCCTTTTCATCCACAGG | eeeeeddddddddddeeeee | 17 | 5957 | 5976 | 1316 |
| 619598 | 743 | 762 | TGTAGGGCCTTTTCATCCAC | eeeeeddddddddddeeeee | 33 | 5960 | 5979 | 1317 |
| 619599 | 746 | 765 | TCCTGTAGGGCCTTTTCATC | eeeeeddddddddddeeeee | 6 | 5963 | 5982 | 1318 |
| 619600 | 749 | 768 | TGGTCCTGTAGGGCCTTTTC | eeeeeddddddddddeeeee | 42 | 5966 | 5985 | 1319 |
| 619601 | 752 | 771 | AGCTGGTCCTGTAGGGCCTT | eeeeeddddddddddeeeee | 51 | 5969 | 5988 | 1320 |
| 619602 | 755 | 774 | ACCAGCTGGTCCTGTAGGGC | eeeeeddddddddddeeeee | 37 | 5972 | 5991 | 1321 |
| 619603 | 758 | 777 | AGCACCAGCTGGTCCTGTAG | eeeeeddddddddddeeeee | 44 | 5975 | 5994 | 1322 |
| 619604 | 761 | 780 | ACTAGCACCAGCTGGTCCTG | eeeeeddddddddddeeeee | 37 | 5978 | 5997 | 1323 |
| 619605 | 764 | 783 | GCGACTAGCACCAGCTGGTC | eeeeeddddddddddeeeee | 52 | 5981 | 6000 | 1324 |
| 619606 | 767 | 786 | GCAGCGACTAGCACCAGCTG | eeeeeddddddddddeeeee | 67 | 5984 | 6003 | 1325 |
| 619607 | 770 | 789 | TTTGCAGCGACTAGCACCAG | eeeeeddddddddddeeeee | 60 | 5987 | 6006 | 1326 |
| 619608 | 773 | 792 | AGTTTTGCAGCGACTAGCAC | eeeeeddddddddddeeeee | 43 | 5990 | 6009 | 1327 |
| 619609 | 776 | 795 | TCAAGTTTTGCAGCGACTAG | eeeeeddddddddddeeeee | 38 | 5993 | 6012 | 1328 |
| 619610 | 779 | 798 | GTGTCAAGTTTTGCAGCGAC | eeeeeddddddddddeeeee | 57 | 5996 | 6015 | 1329 |
| 619611 | 782 | 801 | TCGGTGTCAAGTTTTGCAGC | eeeeeddddddddddeeeee | 55 | 5999 | 6018 | 1330 |
| 619612 | 785 | 804 | TCTTCGGTGTCAAGTTTTGC | eeeeeddddddddddeeeee | 45 | 6002 | 6021 | 1331 |
| 619613 | 788 | 807 | TTGTCTTCGGTGTCAAGTTT | eeeeeddddddddddeeeee | 50 | 6005 | 6024 | 1332 |
| 619614 | 791 | 810 | AACTTGTCTTCGGTGTCAAG | eeeeeddddddddddeeeee | 48 | 6008 | 6027 | 1333 |
| 619615 | 794 | 813 | CTCAACTTGTCTTCGGTGTC | eeeeeddddddddddeeeee | 59 | 6011 | 6030 | 1334 |
| 619616 | 797 | 816 | GCCCTCAACTTGTCTTCGGT | eeeeeddddddddddeeeee | 41 | 6014 | 6033 | 1335 |
| 619617 | 800 | 819 | GCGGCCCTCAACTTGTCTTC | eeeeeddddddddddeeeee | 42 | 6017 | 6036 | 1336 |
| 619618 | 803 | 822 | ATTGCGGCCCTCAACTTGTC | eeeeeddddddddddeeeee | 32 | 6020 | 6039 | 1337 |
| 619619 | 806 | 825 | ACCATTGCGGCCCTCAACTT | eeeeeddddddddddeeeee | 34 | 6023 | 6042 | 1338 |
| 619620 | 809 | 828 | CCGACCATTGCGGCCCTCAA | eeeeeddddddddddeeeee | 55 | 6026 | 6045 | 1339 |
| 619621 | 812 | 831 | ATCCCGACCATTGCGGCCCT | eeeeeddddddddddeeeee | 37 | 6029 | 6048 | 1340 |
| 619622 | 815 | 834 | AGCATCCCGACCATTGCGGC | eeeeeddddddddddeeeee | 50 | 6032 | 6051 | 1341 |
| 619623 | 818 | 837 | GCCAGCATCCCGACCATTGC | eeeeeddddddddddeeeee | 58 | 6035 | 6054 | 1342 |
| 619624 | 821 | 840 | TTGGCCAGCATCCCGACCAT | eeeeeddddddddddeeeee | 38 | 6038 | 6057 | 1343 |
| 619625 | 824 | 843 | AAGTTGGCCAGCATCCCGAC | eeeeeddddddddddeeeee | 46 | 6041 | 6060 | 1344 |
| 619626 | 827 | 846 | AAGAAGTTGGCCAGCATCCC | eeeeeddddddddddeeeee | 24 | 6044 | 6063 | 1345 |
| 619627 | 830 | 849 | CCCAAGAAGTTGGCCAGCAT | eeeeeddddddddddeeeee | 55 | 6047 | 6066 | 1346 |
| 619628 | 833 | 852 | AAGCCCAAGAAGTTGGCCAG | eeeeeddddddddddeeeee | 48 | 6050 | 6069 | 1347 |
| 619629 | 836 | 855 | CGGAAGCCCAAGAAGTTGGC | eeeeeddddddddddeeeee | 36 | 6053 | 6072 | 1348 |
| 619630 | 839 | 858 | ATACGGAAGCCCAAGAAGTT | eeeeeddddddddddeeeee | 40 | 6056 | 6075 | 1349 |
| 619631 | 842 | 861 | TATATACGGAAGCCCAAGAA | eeeeeddddddddddeeeee | 29 | 6059 | 6078 | 1350 |
| 619632 | 845 | 864 | CCATATATACGGAAGCCCAA | eeeeeddddddddddeeeee | 48 | 6062 | 6081 | 1351 |
| 619633 | 848 | 867 | ATGCCATATATACGGAAGCC | eeeeeddddddddddeeeee | 58 | 6065 | 6084 | 1352 |
| 619634 | 851 | 870 | TGCATGCCATATATACGGAA | eeeeeddddddddddeeeee | 59 | 6068 | 6087 | 1353 |
| 619635 | 854 | 873 | CTGTGCATGCCATATATACG | eeeeeddddddddddeeeee | 66 | 6071 | 6090 | 1354 |
| 619636 | 857 | 876 | TCACTGTGCATGCCATATAT | eeeeeddddddddddeeeee | 72 | 6074 | 6093 | 1355 |
| 619637 | 860 | 879 | AGCTCACTGTGCATGCCATA | eeeeeddddddddddeeeee | 74 | 6077 | 6096 | 1356 |
| 619638 | 863 | 882 | CATAGCTCACTGTGCATGCC | eeeeeddddddddddeeeee | 69 | 6080 | 6099 | 1357 |
| 619639 | 866 | 885 | CCCCATAGCTCACTGTGCAT | eeeeeddddddddddeeeee | 43 | 6083 | 6102 | 1358 |
| 619640 | 869 | 888 | ACGCCCCATAGCTCACTGTG | eeeeeddddddddddeeeee | 48 | 6086 | 6105 | 1359 |
| 619641 | 872 | 891 | ACCACGCCCCATAGCTCACT | eeeeeddddddddddeeeee | 56 | 6089 | 6108 | 1360 |
| 619642 | 875 | 894 | TGGACCACGCCCCATAGCTC | eeeeeddddddddddeeeee | 40 | 6092 | 6111 | 1361 |
| 619643 | 878 | 897 | CCATGGACCACGCCCCATAG | eeeeeddddddddddeeeee | 24 | 6095 | 6114 | 1362 |
| 619644 | 881 | 900 | GCCCCATGGACCACGCCCCA | eeeeeddddddddddeeeee | 40 | 6098 | 6117 | 1363 |
| 619645 | 884 | 903 | GTGGCCCCATGGACCACGCC | eeeeeddddddddddeeeee | 26 | 6101 | 6120 | 1364 |
| 619646 | 887 | 906 | ACGGTGGCCCCATGGACCAC | eeeeeddddddddddeeeee | 35 | 6104 | 6123 | 1365 |
| 619647 | 890 | 909 | AGGACGGTGGCCCCATGGAC | eeeeeddddddddddeeeee | 35 | 6107 | 6126 | 1366 |
| 619648 | 893 | 912 | GAGAGGACGGTGGCCCCATG | eeeeeddddddddddeeeee | 44 | 6110 | 6129 | 1367 |
| 619649 | 913 | 932 | TGCCAAAGACAGCCGTTGGG | eeeeeddddddddddeeeee | 53 | 6130 | 6149 | 1368 |
| 619650 | 916 | 935 | GGGTGCCAAAGACAGCCGTT | eeeeeddddddddddeeeee | 40 | 6133 | 6152 | 1369 |
| 619651 | 919 | 938 | CCAGGGTGCCAAAGACAGCC | eeeeeddddddddddeeeee | 62 | 6136 | 6155 | 1370 |
| 619652 | 922 | 941 | AGGCCAGGGTGCCAAAGACA | eeeeeddddddddddeeeee | 44 | 6139 | 6158 | 1371 |
| 619653 | 925 | 944 | GAGAGGCCAGGGTGCCAAAG | eeeeeddddddddddeeeee | 58 | 6142 | 6161 | 1372 |
| 619654 | 928 | 947 | AGAGAGAGGCCAGGGTGCCA | eeeeeddddddddddeeeee | 34 | 6145 | 6164 | 1373 |
| 619655 | 931 | 950 | GATAGAGAGAGGCCAGGGTG | eeeeeddddddddddeeeee | 16 | 6148 | 6167 | 1374 |
| 619656 | 934 | 953 | CCAGATAGAGAGAGGCCAGG | eeeeeddddddddddeeeee | 41 | 6151 | 6170 | 1375 |
| 619657 | 937 | 956 | CTCCCAGATAGAGAGAGGCC | eeeeeddddddddddeeeee | 58 | 6154 | 6173 | 1376 |
| 619658 | 940 | 959 | AGGCTCCCAGATAGAGAGAG | eeeeeddddddddddeeeee | 21 | 6157 | 6176 | 1377 |
| 619659 | 943 | 962 | CCAAGGCTCCCAGATAGAGA | eeeeeddddddddddeeeee | 21 | 6160 | 6179 | 1378 |
| 619660 | 946 | 965 | GGTCCAAGGCTCCCAGATAG | eeeeeddddddddddeeeee | 43 | 6163 | 6182 | 1379 |
| 619661 | 949 | 968 | TGTGGTCCAAGGCTCCCAGA | eeeeeddddddddddeeeee | 45 | 6166 | 6185 | 1380 |
| 619662 | 952 | 971 | CTGTGTGGTCCAAGGCTCCC | eeeeeddddddddddeeeee | 33 | 6169 | 6188 | 1381 |
| 619663 | 955 | 974 | CAGCTGTGTGGTCCAAGGCT | eeeeeddddddddddeeeee | 52 | 6172 | 6191 | 1382 |
| 619664 | 958 | 977 | TGTCAGCTGTGTGGTCCAAG | eeeeeddddddddddeeeee | 44 | 6175 | 6194 | 1383 |
| 619665 | 961 | 980 | GCCTGTCAGCTGTGTGGTCC | eeeeeddddddddddeeeee | 66 | 6178 | 6197 | 1384 |
| 619666 | 964 | 983 | GTAGCCTGTCAGCTGTGTGG | eeeeeddddddddddeeeee | 47 | 6181 | 6200 | 1385 |
| 619667 | 967 | 986 | CCTGTAGCCTGTCAGCTGTG | eeeeeddddddddddeeeee | 59 | 6184 | 6203 | 1386 |
| 619668 | 970 | 989 | TTGCCTGTAGCCTGTCAGCT | eeeeeddddddddddeeeee | 57 | 6187 | 6206 | 1387 |
| 619669 | 973 | 992 | GGATTGCCTGTAGCCTGTCA | eeeeeddddddddddeeeee | 53 | 6190 | 6209 | 1388 |
| 619670 | 976 | 995 | CCAGGATTGCCTGTAGCCTG | eeeeeddddddddddeeeee | 57 | 6193 | 6212 | 1389 |
| 619671 | 979 | 998 | CACCCAGGATTGCCTGTAGC | eeeeeddddddddddeeeee | 52 | 6196 | 6215 | 1390 |
| 619672 | 982 | 1001 | GAACACCCAGGATTGCCTGT | eeeeeddddddddddeeeee | 63 | 6199 | 6218 | 1391 |
| 619673 | 985 | 1004 | AAGGAACACCCAGGATTGCC | eeeeeddddddddddeeeee | 47 | 6202 | 6221 | 1392 |
| 619674 | 988 | 1007 | TCCAAGGAACACCCAGGATT | eeeeeddddddddddeeeee | 63 | 6205 | 6224 | 1393 |
| 619675 | 991 | 1010 | CCTTCCAAGGAACACCCAGG | eeeeeddddddddddeeeee | 60 | 6208 | 6227 | 1394 |
| 619676 | 994 | 1013 | TGTCCTTCCAAGGAACACCC | eeeeeddddddddddeeeee | 62 | 6211 | 6230 | 1395 |
| 619677 | 997 | 1016 | TCTTGTCCTTCCAAGGAACA | eeeeeddddddddddeeeee | 48 | 6214 | 6233 | 1396 |
| 619678 | 1000 | 1019 | AGTTCTTGTCCTTCCAAGGA | eeeeeddddddddddeeeee | 35 | 6217 | 6236 | 1397 |
| 619679 | 1003 | 1022 | TGCAGTTCTTGTCCTTCCAA | eeeeeddddddddddeeeee | 56 | 6220 | 6239 | 1398 |
| 619680 | 1006 | 1025 | AGGTGCAGTTCTTGTCCTTC | eeeeeddddddddddeeeee | 41 | 6223 | 6242 | 1399 |
| 619681 | 1009 | 1028 | GGGAGGTGCAGTTCTTGTCC | eeeeeddddddddddeeeee | 26 | 6226 | 6245 | 1400 |
| 619682 | 1012 | 1031 | GCCGGGAGGTGCAGTTCTTG | eeeeeddddddddddeeeee | 44 | 6229 | 6248 | 1401 |
| 619683 | 1015 | 1034 | CCAGCCGGGAGGTGCAGTTC | eeeeeddddddddddeeeee | 36 | 6232 | 6251 | 1402 |
| 619684 | 1018 | 1037 | CATCCAGCCGGGAGGTGCAG | eeeeeddddddddddeeeee | 32 | 6235 | 6254 | 1403 |
| 619685 | 1021 | 1040 | GCGCATCCAGCCGGGAGGTG | eeeeeddddddddddeeeee | 21 | 6238 | 6257 | 1404 |
| 619686 | 1024 | 1043 | TGTGCGCATCCAGCCGGGAG | eeeeeddddddddddeeeee | 44 | 6241 | 6260 | 1405 |
| 619687 | 1027 | 1046 | CCTTGTGCGCATCCAGCCGG | eeeeeddddddddddeeeee | 60 | 6244 | 6263 | 1406 |
| 619688 | 1030 | 1049 | GGACCTTGTGCGCATCCAGC | eeeeeddddddddddeeeee | 61 | 6247 | 6266 | 1407 |
| 619689 | 1033 | 1052 | ACAGGACCTTGTGCGCATCC | eeeeeddddddddddeeeee | 65 | 6250 | 6269 | 1408 |
| 619690 | 1036 | 1055 | CAGACAGGACCTTGTGCGCA | eeeeeddddddddddeeeee | 59 | 6253 | 6272 | 1409 |
| 619691 | 1039 | 1058 | GGGCAGACAGGACCTTGTGC | eeeeeddddddddddeeeee | 45 | 6256 | 6275 | 1410 |
| 619692 | 1042 | 1061 | GCAGGGCAGACAGGACCTTG | eeeeeddddddddddeeeee | 46 | 6259 | 6278 | 1411 |
| 619693 | 1045 | 1064 | CCTGCAGGGCAGACAGGACC | eeeeeddddddddddeeeee | 38 | 6262 | 6281 | 1412 |
| 619694 | 1048 | 1067 | CAGCCTGCAGGGCAGACAGG | eeeeeddddddddddeeeee | 41 | 6265 | 6284 | 1413 |
| 619695 | 1051 | 1070 | GTACAGCCTGCAGGGCAGAC | eeeeeddddddddddeeeee | 43 | 6268 | 6287 | 1414 |
| 619696 | 1054 | 1073 | CCTGTACAGCCTGCAGGGCA | eeeeeddddddddddeeeee | 48 | 6271 | 6290 | 1415 |
| 619697 | 1057 | 1076 | GGCCCTGTACAGCCTGCAGG | eeeeeddddddddddeeeee | 35 | 6274 | 6293 | 1416 |
| 619698 | 1060 | 1079 | GCAGGCCCTGTACAGCCTGC | eeeeeddddddddddeeeee | 22 | 6277 | 6296 | 1417 |
| 619699 | 1063 | 1082 | CTAGCAGGCCCTGTACAGCC | eeeeeddddddddddeeeee | 1 | 6280 | 6299 | 1418 |
| 619700 | 1066 | 1085 | CCACTAGCAGGCCCTGTACA | eeeeeddddddddddeeeee | 29 | 6283 | 6302 | 1419 |
| 619701 | 1069 | 1088 | GGGCCACTAGCAGGCCCTGT | eeeeeddddddddddeeeee | 2 | 6286 | 6305 | 1420 |
| 619702 | 1072 | 1091 | CCTGGGCCACTAGCAGGCCC | eeeeeddddddddddeeeee | 25 | 6289 | 6308 | 1421 |
| 619703 | 1075 | 1094 | TGCCCTGGGCCACTAGCAGG | eeeeeddddddddddeeeee | 23 | 6292 | 6311 | 1422 |
| 619704 | 1078 | 1097 | CCCTGCCCTGGGCCACTAGC | eeeeeddddddddddeeeee | 46 | 6295 | 6314 | 1423 |
| 619705 | 1081 | 1100 | CAGCCCTGCCCTGGGCCACT | eeeeeddddddddddeeeee | 59 | 6298 | 6317 | 1424 |
| 619706 | 1084 | 1103 | TATCAGCCCTGCCCTGGGCC | eeeeeddddddddddeeeee | 36 | 6301 | 6320 | 1425 |
| 619707 | 1087 | 1106 | GGCTATCAGCCCTGCCCTGG | eeeeeddddddddddeeeee | 51 | 6304 | 6323 | 1426 |
| 619708 | 1090 | 1109 | CCTGGCTATCAGCCCTGCCC | eeeeeddddddddddeeeee | 34 | 6307 | 6326 | 1427 |
| 619709 | 1093 | 1112 | GGGCCTGGCTATCAGCCCTG | eeeeeddddddddddeeeee | 17 | 6310 | 6329 | 1428 |
| 619710 | 1096 | 1115 | GCTGGGCCTGGCTATCAGCC | eeeeeddddddddddeeeee | 31 | 6313 | 6332 | 1429 |
| 619711 | 1099 | 1118 | GCAGCTGGGCCTGGCTATCA | eeeeeddddddddddeeeee | 44 | 6316 | 6335 | 1430 |
| 619712 | 1102 | 1121 | GCAGCAGCTGGGCCTGGCTA | eeeeeddddddddddeeeee | 38 | 6319 | 6338 | 1431 |
| 619713 | 1105 | 1124 | ACAGCAGCAGCTGGGCCTGG | eeeeeddddddddddeeeee | 29 | 6322 | 6341 | 1432 |
| 619714 | 1108 | 1127 | TGGACAGCAGCAGCTGGGCC | eeeeeddddddddddeeeee | 50 | 6325 | 6344 | 1433 |
| 619715 | 1111 | 1130 | CCGTGGACAGCAGCAGCTGG | eeeeeddddddddddeeeee | 53 | 6328 | 6347 | 1434 |
| 619716 | 1114 | 1133 | CCACCGTGGACAGCAGCAGC | eeeeeddddddddddeeeee | 24 | 6331 | 6350 | 1435 |
| 619717 | 1117 | 1136 | CCACCACCGTGGACAGCAGC | eeeeeddddddddddeeeee | 34 | 6334 | 6353 | 1436 |
| 619718 | 1120 | 1139 | CGCCCACCACCGTGGACAGC | eeeeeddddddddddeeeee | 56 | 6337 | 6356 | 1437 |
| 619719 | 1123 | 1142 | ACACGCCCACCACCGTGGAC | eeeeeddddddddddeeeee | 27 | 6340 | 6359 | 1438 |
| 619720 | 1126 | 1145 | TGAACACGCCCACCACCGTG | eeeeeddddddddddeeeee | 16 | 6343 | 6362 | 1439 |
| 619721 | 1129 | 1148 | CTGTGAACACGCCCACCACC | eeeeeddddddddddeeeee | 40 | 6346 | 6365 | 1440 |
| 619722 | 1132 | 1151 | GGGCTGTGAACACGCCCACC | eeeeeddddddddddeeeee | 25 | 6349 | 6368 | 1441 |
| 619723 | 1150 | 1169 | GCTTCAGGTGCAGGCCTGGG | eeeeeddddddddddeeeee | 36 | 6367 | 6386 | 1442 |
| 619724 | 1153 | 1172 | GCTGCTTCAGGTGCAGGCCT | eeeeeddddddddddeeeee | 47 | 6370 | 6389 | 1443 |
| 619725 | 1156 | 1175 | ACGGCTGCTTCAGGTGCAGG | eeeeeddddddddddeeeee | 14 | 6373 | 6392 | 1444 |
| 619726 | 1159 | 1178 | CAAACGGCTGCTTCAGGTGC | eeeeeddddddddddeeeee | 37 | 6376 | 6395 | 1445 |
| 619727 | 1162 | 1181 | GCACAAACGGCTGCTTCAGG | eeeeeddddddddddeeeee | 19 | 6379 | 6398 | 1446 |
| 619728 | 1165 | 1184 | CCTGCACAAACGGCTGCTTC | eeeeeddddddddddeeeee | 33 | 6382 | 6401 | 1447 |
| 619729 | 1168 | 1187 | GGCCCTGCACAAACGGCTGC | eeeeeddddddddddeeeee | 48 | 6385 | 6404 | 1448 |
| 619730 | 1171 | 1190 | CCAGGCCCTGCACAAACGGC | eeeeeddddddddddeeeee | 27 | 6388 | 6407 | 1449 |
| 619731 | 1174 | 1193 | GAGCCAGGCCCTGCACAAAC | eeeeeddddddddddeeeee | 35 | 6391 | 6410 | 1450 |
| 619732 | 1177 | 1196 | AGAGAGCCAGGCCCTGCACA | eeeeeddddddddddeeeee | 51 | 6394 | 6413 | 1451 |
| 619733 | 1180 | 1199 | TATAGAGAGCCAGGCCCTGC | eeeeeddddddddddeeeee | 27 | 6397 | 6416 | 1452 |
| 619734 | 1183 | 1202 | GGGTATAGAGAGCCAGGCCC | eeeeeddddddddddeeeee | 41 | 6400 | 6419 | 1453 |
| 619735 | 1217 | 1236 | TCTGTGAAGTCCAGAGAGCG | eeeeeddddddddddeeeee | 22 | 6434 | 6453 | 1454 |
| 619736 | 1220 | 1239 | AGTTCTGTGAAGTCCAGAGA | eeeeeddddddddddeeeee | 48 | 6437 | 6456 | 1455 |
| 619737 | 1223 | 1242 | TCCAGTTCTGTGAAGTCCAG | eeeeeddddddddddeeeee | 26 | 6440 | 6459 | 1456 |
| 619738 | 1226 | 1245 | ACATCCAGTTCTGTGAAGTC | eeeeeddddddddddeeeee | 35 | 6443 | 6462 | 1457 |
| 619739 | 1229 | 1248 | GCAACATCCAGTTCTGTGAA | eeeeeddddddddddeeeee | 28 | 6446 | 6465 | 1458 |
| 619740 | 1232 | 1251 | GCAGCAACATCCAGTTCTGT | eeeeeddddddddddeeeee | 40 | 6449 | 6468 | 1459 |
| 619741 | 1235 | 1254 | TCAGCAGCAACATCCAGTTC | eeeeeddddddddddeeeee | 41 | 6452 | 6471 | 1460 |
| 619742 | 1238 | 1257 | TTCTCAGCAGCAACATCCAG | eeeeeddddddddddeeeee | 29 | 6455 | 6474 | 1461 |
| 619743 | 1241 | 1260 | ATCTTCTCAGCAGCAACATC | eeeeeddddddddddeeeee | 32 | 6458 | 6477 | 1462 |
| 619744 | 1244 | 1263 | TCAATCTTCTCAGCAGCAAC | eeeeeddddddddddeeeee | 38 | 6461 | 6480 | 1463 |
| 619745 | 1247 | 1266 | CTGTCAATCTTCTCAGCAGC | eeeeeddddddddddeeeee | 39 | 6464 | 6483 | 1464 |
| 619746 | 1250 | 1269 | AACCTGTCAATCTTCTCAGC | eeeeeddddddddddeeeee | 20 | 6467 | 6486 | 1465 |
| 619747 | 1253 | 1272 | ATGAACCTGTCAATCTTCTC | eeeeeddddddddddeeeee | 50 | 6470 | 6489 | 1466 |
| 619748 | 1256 | 1275 | TGCATGAACCTGTCAATCTT | eeeeeddddddddddeeeee | 61 | 6473 | 6492 | 1467 |
| 619749 | 1259 | 1278 | GCCTGCATGAACCTGTCAAT | eeeeeddddddddddeeeee | 62 | 6476 | 6495 | 1468 |
| 619750 | 1262 | 1281 | ACAGCCTGCATGAACCTGTC | eeeeeddddddddddeeeee | 56 | 6479 | 6498 | 1469 |
| 619751 | 1265 | 1284 | GTCACAGCCTGCATGAACCT | eeeeeddddddddddeeeee | 75 | 6482 | 6501 | 1470 |
| 619752 | 1268 | 1287 | CCTGTCACAGCCTGCATGAA | eeeeeddddddddddeeeee | 46 | 6485 | 6504 | 1471 |
| 619753 | 1271 | 1290 | CATCCTGTCACAGCCTGCAT | eeeeeddddddddddeeeee | 74 | 6488 | 6507 | 1472 |
| 619754 | 1274 | 1293 | TTCCATCCTGTCACAGCCTG | eeeeeddddddddddeeeee | 71 | 6491 | 6510 | 1473 |
| 619755 | 1277 | 1296 | GTCTTCCATCCTGTCACAGC | eeeeeddddddddddeeeee | 65 | 6494 | 6513 | 1474 |
| 619756 | 1280 | 1299 | CCAGTCTTCCATCCTGTCAC | eeeeeddddddddddeeeee | 56 | 6497 | 6516 | 1475 |
| 619757 | 1283 | 1302 | CAGCCAGTCTTCCATCCTGT | eeeeeddddddddddeeeee | 63 | 6500 | 6519 | 1476 |

Table 9 shows inhibition of AGT mRNA in HepG2 cells cultured at a density of 20,000 cells per well which were transfected using electroporation with 4000 nM antisense oligonucleotide. After a treatment period of approximately 24 hours, RNA was isolated from the cells and AGT mRNA levels were measured by quantitative real-time PCR. Human primer probe set RTS4039 was used to measure mRNA levels. AGT mRNA levels were adjusted according to total RNA content, as measured by RIBOGREEN®. Results are presented as percent inhibition of AGT, relative to untreated control cells.

TABLE 9
Inhibition of AGT mRNA by MOE containing gapmers targeting SEQ ID NO: 1 and/or 2

| | | SEQ | | | | | | |
|---|---|---|---|---|---|---|---|---|
| | SEQ | ID: | | | | SEQ | SEQ | |
| | ID: 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 86 | 13518 | 13537 | 239 |
| 610015 | 2049 | 2068 | GACACATCGCTGATTTGTCC | eeeeeddddddddddeeeee | 88 | 13518 | 13537 | 239 |
| 610015 | 2049 | 2068 | GACACATCGCTGATTTGTCC | eeeeeddddddddddeeeee | 85 | 13518 | 13537 | 239 |
| 619692 | 1042 | 1061 | GCAGGGCAGACAGGACCTTG | eeeeeddddddddddeeeee | 17 | 6259 | 6278 | 1411 |
| 619693 | 1045 | 1064 | CCTGCAGGGCAGACAGGACC | eeeeeddddddddddeeeee | 25 | 6262 | 6281 | 1412 |
| 619694 | 1048 | 1067 | CAGCCTGCAGGGCAGACAGG | eeeeeddddddddddeeeee | 32 | 6265 | 6284 | 1413 |
| 619695 | 1051 | 1070 | GTACAGCCTGCAGGGCAGAC | eeeeeddddddddddeeeee | 25 | 6268 | 6287 | 1414 |
| 619696 | 1054 | 1073 | CCTGTACAGCCTGCAGGGCA | eeeeeddddddddddeeeee | 48 | 6271 | 6290 | 1415 |
| 619697 | 1057 | 1076 | GGCCCTGTACAGCCTGCAGG | eeeeeddddddddddeeeee | 32 | 6274 | 6293 | 1416 |
| 619698 | 1060 | 1079 | GCAGGCCCTGTACAGCCTGC | eeeeeddddddddddeeeee | 17 | 6277 | 6296 | 1417 |
| 619699 | 1063 | 1082 | CTAGCAGGCCCTGTACAGCC | eeeeeddddddddddeeeee | 13 | 6280 | 6299 | 1418 |
| 619700 | 1066 | 1085 | CCACTAGCAGGCCCTGTACA | eeeeeddddddddddeeeee | 36 | 6283 | 6302 | 1419 |
| 619701 | 1069 | 1088 | GGGCCACTAGCAGGCCCTGT | eeeeeddddddddddeeeee | 6 | 6286 | 6305 | 1420 |
| 619702 | 1072 | 1091 | CCTGGGCCACTAGCAGGCCC | eeeeeddddddddddeeeee | 16 | 6289 | 6308 | 1421 |
| 619703 | 1075 | 1094 | TGCCCTGGGCCACTAGCAGG | eeeeeddddddddddeeeee | 26 | 6292 | 6311 | 1422 |
| 619704 | 1078 | 1097 | CCCTGCCCTGGGCCACTAGC | eeeeeddddddddddeeeee | 41 | 6295 | 6314 | 1423 |
| 619705 | 1081 | 1100 | CAGCCCTGCCCTGGGCCACT | eeeeeddddddddddeeeee | 36 | 6298 | 6317 | 1424 |
| 619706 | 1084 | 1103 | TATCAGCCCTGCCCTGGGCC | eeeeeddddddddddeeeee | 21 | 6301 | 6320 | 1425 |
| 619707 | 1087 | 1106 | GGCTATCAGCCCTGCCCTGG | eeeeeddddddddddeeeee | 27 | 6304 | 6323 | 1426 |
| 619708 | 1090 | 1109 | CCTGGCTATCAGCCCTGCCC | eeeeeddddddddddeeeee | 30 | 6307 | 6326 | 1427 |
| 619709 | 1093 | 1112 | GGGCCTGGCTATCAGCCCTG | eeeeeddddddddddeeeee | 9 | 6310 | 6329 | 1428 |
| 619710 | 1096 | 1115 | GCTGGGCCTGGCTATCAGCC | eeeeeddddddddddeeeee | 15 | 6313 | 6332 | 1429 |
| 619711 | 1099 | 1118 | GCAGCTGGGCCTGGCTATCA | eeeeeddddddddddeeeee | 26 | 6316 | 6335 | 1430 |
| 619712 | 1102 | 1121 | GCAGCAGCTGGGCCTGGCTA | eeeeeddddddddddeeeee | 61 | 6319 | 6338 | 1431 |
| 619713 | 1105 | 1124 | ACAGCAGCAGCTGGGCCTGG | eeeeeddddddddddeeeee | 44 | 6322 | 6341 | 1432 |
| 619714 | 1108 | 1127 | TGGACAGCAGCAGCTGGGCC | eeeeeddddddddddeeeee | 47 | 6325 | 6344 | 1433 |
| 619715 | 1111 | 1130 | CCGTGGACAGCAGCAGCTGG | eeeeeddddddddddeeeee | 41 | 6328 | 6347 | 1434 |
| 619716 | 1114 | 1133 | CCACCGTGGACAGCAGCAGC | eeeeeddddddddddeeeee | 35 | 6331 | 6350 | 1435 |
| 619717 | 1117 | 1136 | CCACCACCGTGGACAGCAGC | eeeeeddddddddddeeeee | 34 | 6334 | 6353 | 1436 |
| 619718 | 1120 | 1139 | CGCCCACCACCGTGGACAGC | eeeeeddddddddddeeeee | 37 | 6337 | 6356 | 1437 |
| 619719 | 1123 | 1142 | ACACGCCCACCACCGTGGAC | eeeeeddddddddddeeeee | 17 | 6340 | 6359 | 1438 |
| 619720 | 1126 | 1145 | TGAACACGCCCACCACCGTG | eeeeeddddddddddeeeee | 20 | 6343 | 6362 | 1439 |
| 619721 | 1129 | 1148 | CTGTGAACACGCCCACCACC | eeeeeddddddddddeeeee | 36 | 6346 | 6365 | 1440 |
| 619722 | 1132 | 1151 | GGGCTGTGAACACGCCCACC | eeeeeddddddddddeeeee | 14 | 6349 | 6368 | 1441 |
| 619723 | 1150 | 1169 | GCTTCAGGTGCAGGCCTGGG | eeeeeddddddddddeeeee | 32 | 6367 | 6386 | 1442 |
| 619724 | 1153 | 1172 | GCTGCTTCAGGTGCAGGCCT | eeeeeddddddddddeeeee | 47 | 6370 | 6389 | 1443 |
| 619725 | 1156 | 1175 | ACGGCTGCTTCAGGTGCAGG | eeeeeddddddddddeeeee | 27 | 6373 | 6392 | 1444 |
| 619726 | 1159 | 1178 | CAAACGGCTGCTTCAGGTGC | eeeeeddddddddddeeeee | 20 | 6376 | 6395 | 1445 |
| 619727 | 1162 | 1181 | GCACAAACGGCTGCTTCAGG | eeeeeddddddddddeeeee | 13 | 6379 | 6398 | 1446 |
| 619728 | 1165 | 1184 | CCTGCACAAACGGCTGCTTC | eeeeeddddddddddeeeee | 25 | 6382 | 6401 | 1447 |
| 619729 | 1168 | 1187 | GGCCCTGCACAAACGGCTGC | eeeeeddddddddddeeeee | 29 | 6385 | 6404 | 1448 |
| 619730 | 1171 | 1190 | CCAGGCCCTGCACAAACGGC | eeeeeddddddddddeeeee | 27 | 6388 | 6407 | 1449 |
| 619731 | 1174 | 1193 | GAGCCAGGCCCTGCACAAAC | eeeeeddddddddddeeeee | 18 | 6391 | 6410 | 1450 |
| 619732 | 1177 | 1196 | AGAGAGCCAGGCCCTGCACA | eeeeeddddddddddeeeee | 33 | 6394 | 6413 | 1451 |
| 619733 | 1180 | 1199 | TATAGAGAGCCAGGCCCTGC | eeeeeddddddddddeeeee | 0 | 6397 | 6416 | 1452 |
| 619734 | 1183 | 1202 | GGGTATAGAGAGCCAGGCCC | eeeeeddddddddddeeeee | 14 | 6400 | 6419 | 1453 |
| 619735 | 1217 | 1236 | TCTGTGAAGTCCAGAGAGCG | eeeeeddddddddddeeeee | 17 | 6434 | 6453 | 1454 |
| 619736 | 1220 | 1239 | AGTTCTGTGAAGTCCAGAGA | eeeeeddddddddddeeeee | 41 | 6437 | 6456 | 1455 |
| 619737 | 1223 | 1242 | TCCAGTTCTGTGAAGTCCAG | eeeeeddddddddddeeeee | 31 | 6440 | 6459 | 1456 |
| 619738 | 1226 | 1245 | ACATCCAGTTCTGTGAAGTC | eeeeeddddddddddeeeee | 35 | 6443 | 6462 | 1457 |
| 619739 | 1229 | 1248 | GCAACATCCAGTTCTGTGAA | eeeeeddddddddddeeeee | 29 | 6446 | 6465 | 1458 |
| 619740 | 1232 | 1251 | GCAGCAACATCCAGTTCTGT | eeeeeddddddddddeeeee | 35 | 6449 | 6468 | 1459 |
| 619741 | 1235 | 1254 | TCAGCAGCAACATCCAGTTC | eeeeeddddddddddeeeee | 35 | 6452 | 6471 | 1460 |
| 619742 | 1238 | 1257 | TTCTCAGCAGCAACATCCAG | eeeeeddddddddddeeeee | 5 | 6455 | 6474 | 1461 |
| 619743 | 1241 | 1260 | ATCTTCTCAGCAGCAACATC | eeeeeddddddddddeeeee | 22 | 6458 | 6477 | 1462 |
| 619744 | 1244 | 1263 | TCAATCTTCTCAGCAGCAAC | eeeeeddddddddddeeeee | 45 | 6461 | 6480 | 1463 |
| 619745 | 1247 | 1266 | CTGTCAATCTTCTCAGCAGC | eeeeeddddddddddeeeee | 21 | 6464 | 6483 | 1464 |
| 619746 | 1250 | 1269 | AACCTGTCAATCTTCTCAGC | eeeeeddddddddddeeeee | 8 | 6467 | 6486 | 1465 |
| 619747 | 1253 | 1272 | ATGAACCTGTCAATCTTCTC | eeeeeddddddddddeeeee | 43 | 6470 | 6489 | 1466 |
| 619748 | 1256 | 1275 | TGCATGAACCTGTCAATCTT | eeeeeddddddddddeeeee | 31 | 6473 | 6492 | 1467 |
| 619749 | 1259 | 1278 | GCCTGCATGAACCTGTCAAT | eeeeeddddddddddeeeee | 44 | 6476 | 6495 | 1468 |
| 619750 | 1262 | 1281 | ACAGCCTGCATGAACCTGTC | eeeeeddddddddddeeeee | 41 | 6479 | 6498 | 1469 |
| 619751 | 1265 | 1284 | GTCACAGCCTGCATGAACCT | eeeeeddddddddddeeeee | 69 | 6482 | 6501 | 1470 |
| 619752 | 1268 | 1287 | CCTGTCACAGCCTGCATGAA | eeeeeddddddddddeeeee | 43 | 6485 | 6504 | 1471 |
| 619753 | 1271 | 1290 | CATCCTGTCACAGCCTGCAT | eeeeeddddddddddeeeee | 59 | 6488 | 6507 | 1472 |
| 619754 | 1274 | 1293 | TTCCATCCTGTCACAGCCTG | eeeeeddddddddddeeeee | 49 | 6491 | 6510 | 1473 |
| 619755 | 1277 | 1296 | GTCTTCCATCCTGTCACAGC | eeeeeddddddddddeeeee | 42 | 6494 | 6513 | 1474 |
| 619756 | 1280 | 1299 | CCAGTCTTCCATCCTGTCAC | eeeeeddddddddddeeeee | 20 | 6497 | 6516 | 1475 |
| 619757 | 1283 | 1302 | CAGCCAGTCTTCCATCCTGT | eeeeeddddddddddeeeee | 41 | 6500 | 6519 | 1476 |
| 619758 | 1286 | 1305 | GAGCAGCCAGTCTTCCATCC | eeeeeddddddddddeeeee | 41 | 6503 | 6522 | 1477 |
| 619759 | 1289 | 1308 | AGGGAGCAGCCAGTCTTCCA | eeeeeddddddddddeeeee | 29 | 6506 | 6525 | 1478 |
| 619760 | 1292 | 1311 | ATCAGGGAGCAGCCAGTCTT | eeeeeddddddddddeeeee | 29 | 6509 | 6528 | 1479 |
| 619761 | 1295 | 1314 | CCCATCAGGGAGCAGCCAGT | eeeeeddddddddddeeeee | 7 | 6512 | 6531 | 1480 |
| 619762 | 1298 | 1317 | GCTCCCATCAGGGAGCAGCC | eeeeeddddddddddeeeee | 4 | 6515 | 6534 | 1481 |
| 619763 | 1301 | 1320 | CTGGCTCCCATCAGGGAGCA | eeeeeddddddddddeeeee | 8 | 6518 | 6537 | 1482 |
| 619764 | 1304 | 1323 | ACACTGGCTCCCATCAGGGA | eeeeeddddddddddeeeee | 0 | 6521 | 6540 | 1483 |
| 619765 | 1307 | 1326 | TCCACACTGGCTCCCATCAG | eeeeeddddddddddeeeee | 27 | 6524 | 6543 | 1484 |
| 619766 | 1310 | 1329 | CTGTCCACACTGGCTCCCAT | eeeeeddddddddddeeeee | 27 | 6527 | 6546 | 1485 |
| 619767 | 1313 | 1332 | GTGCTGTCCACACTGGCTCC | eeeeeddddddddddeeeee | 42 | 6530 | 6549 | 1486 |
| 619768 | 1316 | 1335 | AGGGTGCTGTCCACACTGGC | eeeeeddddddddddeeeee | 39 | 6533 | 6552 | 1487 |
| 619769 | 1319 | 1338 | GCCAGGGTGCTGTCCACACT | eeeeeddddddddddeeeee | 65 | 6536 | 6555 | 1488 |
| 619770 | 1322 | 1341 | AAAGCCAGGGTGCTGTCCAC | eeeeeddddddddddeeeee | 65 | 6539 | 6558 | 1489 |
| 619771 | 1325 | 1344 | TTGAAAGCCAGGGTGCTGTC | eeeeeddddddddddeeeee | 48 | 6542 | 6561 | 1490 |
| 619772 | 1328 | 1347 | GTGTTGAAAGCCAGGGTGCT | eeeeeddddddddddeeeee | 44 | 6545 | 6564 | 1491 |
| 619773 | 1331 | 1350 | TAGGTGTTGAAAGCCAGGGT | eeeeeddddddddddeeeee | 16 | 6548 | 6567 | 1492 |
| 619774 | 1351 | 1370 | TCTTCCCTTGGAAGTGGACG | eeeeeddddddddddeeeee | 40 | N/A | N/A | 1493 |
| 619775 | 1354 | 1373 | TCATCTTCCCTTGGAAGTGG | eeeeeddddddddddeeeee | 41 | N/A | N/A | 1494 |
| 619776 | 1357 | 1376 | CCTTCATCTTCCCTTGGAAG | eeeeeddddddddddeeeee | 30 | N/A | N/A | 1495 |
| 619777 | 1360 | 1379 | AGCCCTTCATCTTCCCTTGG | eeeeeddddddddddeeeee | 53 | N/A | N/A | 1496 |
| 619778 | 1363 | 1382 | AGAAGCCCTTCATCTTCCCT | eeeeeddddddddddeeeee | 33 | 10374 | 10393 | 1497 |
| 619779 | 1366 | 1385 | GGGAGAAGCCCTTCATCTTC | eeeeeddddddddddeeeee | 56 | 10377 | 10396 | 1498 |
| 619780 | 1369 | 1388 | GCAGGGAGAAGCCCTTCATC | eeeeeddddddddddeeeee | 42 | 10380 | 10399 | 1499 |
| 619781 | 1372 | 1391 | CCAGCAGGGAGAAGCCCTTC | eeeeeddddddddddeeeee | 63 | 10383 | 10402 | 1500 |
| 619782 | 1375 | 1394 | CGGCCAGCAGGGAGAAGCCC | eeeeeddddddddddeeeee | 52 | 10386 | 10405 | 1501 |
| 619783 | 1378 | 1397 | GCTCGGCCAGCAGGGAGAAG | eeeeeddddddddddeeeee | 37 | 10389 | 10408 | 1502 |
| 619784 | 1398 | 1417 | GTCCACCCAGAACTCCTGGG | eeeeeddddddddddeeeee | 67 | 10409 | 10428 | 1503 |
| 619785 | 1401 | 1420 | GTTGTCCACCCAGAACTCCT | eeeeeddddddddddeeeee | 65 | 10412 | 10431 | 1504 |
| 619786 | 1404 | 1423 | GCTGTTGTCCACCCAGAACT | eeeeeddddddddddeeeee | 43 | 10415 | 10434 | 1505 |
| 619787 | 1407 | 1426 | GGTGCTGTTGTCCACCCAGA | eeeeeddddddddddeeeee | 49 | 10418 | 10437 | 1506 |
| 619788 | 1410 | 1429 | TGAGGTGCTGTTGTCCACCC | eeeeeddddddddddeeeee | 50 | 10421 | 10440 | 1507 |
| 619789 | 1413 | 1432 | CACTGAGGTGCTGTTGTCCA | eeeeeddddddddddeeeee | 47 | 10424 | 10443 | 1508 |
| 619790 | 1416 | 1435 | AGACACTGAGGTGCTGTTGT | eeeeeddddddddddeeeee | 50 | 10427 | 10446 | 1509 |
| 619791 | 1419 | 1438 | AACAGACACTGAGGTGCTGT | eeeeeddddddddddeeeee | 58 | 10430 | 10449 | 1510 |
| 619792 | 1422 | 1441 | GGGAACAGACACTGAGGTGC | eeeeeddddddddddeeeee | 56 | 10433 | 10452 | 1511 |
| 619793 | 1425 | 1444 | CATGGGAACAGACACTGAGG | eeeeeddddddddddeeeee | 45 | 10436 | 10455 | 1512 |
| 619794 | 1428 | 1447 | GAGCATGGGAACAGACACTG | eeeeeddddddddddeeeee | 49 | 10439 | 10458 | 1513 |
| 619795 | 1431 | 1450 | AGAGAGCATGGGAACAGACA | eeeeeddddddddddeeeee | 32 | 10442 | 10461 | 1514 |
| 619796 | 1434 | 1453 | GCCAGAGAGCATGGGAACAG | eeeeeddddddddddeeeee | 32 | 10445 | 10464 | 1515 |
| 619797 | 1437 | 1456 | CATGCCAGAGAGCATGGGAA | eeeeeddddddddddeeeee | 35 | 10448 | 10467 | 1516 |
| 619798 | 1440 | 1459 | GCCCATGCCAGAGAGCATGG | eeeeeddddddddddeeeee | 23 | 10451 | 10470 | 1517 |
| 619799 | 1443 | 1462 | GGTGCCCATGCCAGAGAGCA | eeeeeddddddddddeeeee | 48 | 10454 | 10473 | 1518 |
| 619800 | 1446 | 1465 | GAAGGTGCCCATGCCAGAGA | eeeeeddddddddddeeeee | 46 | 10457 | 10476 | 1519 |
| 619801 | 1449 | 1468 | CTGGAAGGTGCCCATGCCAG | eeeeeddddddddddeeeee | 55 | 10460 | 10479 | 1520 |
| 619802 | 1452 | 1471 | GTGCTGGAAGGTGCCCATGC | eeeeeddddddddddeeeee | 43 | 10463 | 10482 | 1521 |
| 619803 | 1455 | 1474 | CCAGTGCTGGAAGGTGCCCA | eeeeeddddddddddeeeee | 58 | 10466 | 10485 | 1522 |
| 619804 | 1458 | 1477 | ACTCCAGTGCTGGAAGGTGC | eeeeeddddddddddeeeee | 50 | 10469 | 10488 | 1523 |
| 619805 | 1461 | 1480 | GTCACTCCAGTGCTGGAAGG | eeeeeddddddddddeeeee | 53 | 10472 | 10491 | 1524 |
| 619806 | 1464 | 1483 | GATGTCACTCCAGTGCTGGA | eeeeeddddddddddeeeee | 46 | 10475 | 10494 | 1525 |
| 619807 | 1467 | 1486 | CTGGATGTCACTCCAGTGCT | eeeeeddddddddddeeeee | 70 | 10478 | 10497 | 1526 |
| 619808 | 1470 | 1489 | GTCCTGGATGTCACTCCAGT | eeeeeddddddddddeeeee | 49 | 10481 | 10500 | 1527 |
| 619809 | 1473 | 1492 | GTTGTCCTGGATGTCACTCC | eeeeeddddddddddeeeee | 51 | 10484 | 10503 | 1528 |
| 619810 | 1476 | 1495 | GAAGTTGTCCTGGATGTCAC | eeeeeddddddddddeeeee | 51 | 10487 | 10506 | 1529 |
| 619811 | 1479 | 1498 | CGAGAAGTTGTCCTGGATGT | eeeeeddddddddddeeeee | 33 | 10490 | 10509 | 1530 |
| 619812 | 1482 | 1501 | CACCGAGAAGTTGTCCTGGA | eeeeeddddddddddeeeee | 49 | 10493 | 10512 | 1531 |
| 619813 | 1485 | 1504 | AGTCACCGAGAAGTTGTCCT | eeeeeddddddddddeeeee | 53 | 10496 | 10515 | 1532 |
| 619814 | 1488 | 1507 | TTGAGTCACCGAGAAGTTGT | eeeeeddddddddddeeeee | 41 | 10499 | 10518 | 1533 |
| 619815 | 1491 | 1510 | CACTTGAGTCACCGAGAAGT | eeeeeddddddddddeeeee | 32 | 10502 | 10521 | 1534 |
| 619816 | 1494 | 1513 | GGGCACTTGAGTCACCGAGA | eeeeeddddddddddeeeee | 69 | 10505 | 10524 | 1535 |
| 619817 | 1497 | 1516 | GAAGGGCACTTGAGTCACCG | eeeeeddddddddddeeeee | 63 | 10508 | 10527 | 1536 |
| 619818 | 1500 | 1519 | AGTGAAGGGCACTTGAGTCA | eeeeeddddddddddeeeee | 37 | 10511 | 10530 | 1537 |
| 619819 | 1503 | 1522 | CTCAGTGAAGGGCACTTGAG | eeeeeddddddddddeeeee | 35 | 10514 | 10533 | 1538 |
| 619820 | 1506 | 1525 | GCTCTCAGTGAAGGGCACTT | eeeeeddddddddddeeeee | 65 | 10517 | 10536 | 1539 |
| 619821 | 1524 | 1543 | GATCAGCAGCAGGCAGGCGC | eeeeeddddddddddeeeee | 58 | 10535 | 10554 | 1540 |
| 619822 | 1527 | 1546 | CTGGATCAGCAGCAGGCAGG | eeeeeddddddddddeeeee | 55 | 10538 | 10557 | 1541 |
| 619823 | 1530 | 1549 | AGGCTGGATCAGCAGCAGGC | eeeeeddddddddddeeeee | 72 | 10541 | 10560 | 1542 |
| 619824 | 1533 | 1552 | GTGAGGCTGGATCAGCAGCA | eeeeeddddddddddeeeee | 70 | 10544 | 10563 | 1543 |
| 619825 | 1536 | 1555 | ATAGTGAGGCTGGATCAGCA | eeeeeddddddddddeeeee | 17 | 10547 | 10566 | 1544 |
| 619826 | 1539 | 1558 | GGCATAGTGAGGCTGGATCA | eeeeeddddddddddeeeee | 67 | 10550 | 10569 | 1545 |
| 619827 | 1542 | 1561 | AGAGGCATAGTGAGGCTGGA | eeeeeddddddddddeeeee | 51 | 10553 | 10572 | 1546 |
| 619828 | 1545 | 1564 | GTCAGAGGCATAGTGAGGCT | eeeeeddddddddddeeeee | 46 | 10556 | 10575 | 1547 |

Table 10 shows inhibition of AGT mRNA in HepG2 cells cultured at a density of 20,000 cells per well which were transfected using electroporation with 4000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 10
Inhibition of AGT mRNA by MOE containing gapmers targeting SEQ ID NO: 1 and/or 2

| | | SEQ | | | | | | |
|---|---|---|---|---|---|---|---|---|
| | SEQ | ID: | | | | SEQ | SEQ | |
| | ID: 1 | 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 84 | 13518 | 13537 | 239 |
| 610015 | 2049 | 2068 | GACACATCGCTGATTTGTCC | eeeeeddddddddddeeeee | 91 | 13518 | 13537 | 239 |
| 610015 | 2049 | 2068 | GACACATCGCTGATTTGTCC | eeeeeddddddddddeeeee | 78 | 13518 | 13537 | 239 |
| 610015 | 2049 | 2068 | GACACATCGCTGATTTGTCC | eeeeeddddddddddeeeee | 89 | 13518 | 13537 | 239 |
| 610015 | 2049 | 2068 | GACACATCGCTGATTTGTCC | eeeeeddddddddddeeeee | 81 | 13518 | 13537 | 239 |
| 610015 | 2049 | 2068 | GACACATCGCTGATTTGTCC | eeeeeddddddddddeeeee | 92 | 13518 | 13537 | 239 |
| 610015 | 2049 | 2068 | GACACATCGCTGATTTGTCC | eeeeeddddddddddeeeee | 90 | 13518 | 13537 | 239 |
| 619784 | 1398 | 1417 | GTCCACCCAGAACTCCTGGG | eeeeeddddddddddeeeee | 73 | 10409 | 10428 | 1503 |
| 619785 | 1401 | 1420 | GTTGTCCACCCAGAACTCCT | eeeeeddddddddddeeeee | 76 | 10412 | 10431 | 1504 |
| 619786 | 1404 | 1423 | GCTGTTGTCCACCCAGAACT | eeeeeddddddddddeeeee | 54 | 10415 | 10434 | 1505 |
| 619787 | 1407 | 1426 | GGTGCTGTTGTCCACCCAGA | eeeeeddddddddddeeeee | 55 | 10418 | 10437 | 1506 |
| 619788 | 1410 | 1429 | TGAGGTGCTGTTGTCCACCC | eeeeeddddddddddeeeee | 51 | 10421 | 10440 | 1507 |
| 619789 | 1413 | 1432 | CACTGAGGTGCTGTTGTCCA | eeeeeddddddddddeeeee | 46 | 10424 | 10443 | 1508 |
| 619790 | 1416 | 1435 | AGACACTGAGGTGCTGTTGT | eeeeeddddddddddeeeee | 51 | 10427 | 10446 | 1509 |
| 619791 | 1419 | 1438 | AACAGACACTGAGGTGCTGT | eeeeeddddddddddeeeee | 36 | 10430 | 10449 | 1510 |
| 619792 | 1422 | 1441 | GGGAACAGACACTGAGGTGC | eeeeeddddddddddeeeee | 57 | 10433 | 10452 | 1511 |
| 619793 | 1425 | 1444 | CATGGGAACAGACACTGAGG | eeeeeddddddddddeeeee | 42 | 10436 | 10455 | 1512 |
| 619794 | 1428 | 1447 | GAGCATGGGAACAGACACTG | eeeeeddddddddddeeeee | 45 | 10439 | 10458 | 1513 |
| 619795 | 1431 | 1450 | AGAGAGCATGGGAACAGACA | eeeeeddddddddddeeeee | 25 | 10442 | 10461 | 1514 |
| 619796 | 1434 | 1453 | GCCAGAGAGCATGGGAACAG | eeeeeddddddddddeeeee | 45 | 10445 | 10464 | 1515 |
| 619797 | 1437 | 1456 | CATGCCAGAGAGCATGGGAA | eeeeeddddddddddeeeee | 38 | 10448 | 10467 | 1516 |
| 619798 | 1440 | 1459 | GCCCATGCCAGAGAGCATGG | eeeeeddddddddddeeeee | 27 | 10451 | 10470 | 1517 |
| 619799 | 1443 | 1462 | GGTGCCCATGCCAGAGAGCA | eeeeeddddddddddeeeee | 50 | 10454 | 10473 | 1518 |
| 619800 | 1446 | 1465 | GAAGGTGCCCATGCCAGAGA | eeeeeddddddddddeeeee | 39 | 10457 | 10476 | 1519 |
| 619801 | 1449 | 1468 | CTGGAAGGTGCCCATGCCAG | eeeeeddddddddddeeeee | 54 | 10460 | 10479 | 1520 |
| 619802 | 1452 | 1471 | GTGCTGGAAGGTGCCCATGC | eeeeeddddddddddeeeee | 42 | 10463 | 10482 | 1521 |
| 619803 | 1455 | 1474 | CCAGTGCTGGAAGGTGCCCA | eeeeeddddddddddeeeee | 83 | 10466 | 10485 | 1522 |
| 619804 | 1458 | 1477 | ACTCCAGTGCTGGAAGGTGC | eeeeeddddddddddeeeee | 42 | 10469 | 10488 | 1523 |
| 619805 | 1461 | 1480 | GTCACTCCAGTGCTGGAAGG | eeeeeddddddddddeeeee | 66 | 10472 | 10491 | 1524 |
| 619806 | 1464 | 1483 | GATGTCACTCCAGTGCTGGA | eeeeeddddddddddeeeee | 55 | 10475 | 10494 | 1525 |
| 619807 | 1467 | 1486 | CTGGATGTCACTCCAGTGCT | eeeeeddddddddddeeeee | 68 | 10478 | 10497 | 1526 |
| 619808 | 1470 | 1489 | GTCCTGGATGTCACTCCAGT | eeeeeddddddddddeeeee | 49 | 10481 | 10500 | 1527 |
| 619809 | 1473 | 1492 | GTTGTCCTGGATGTCACTCC | eeeeeddddddddddeeeee | 61 | 10484 | 10503 | 1528 |
| 619810 | 1476 | 1495 | GAAGTTGTCCTGGATGTCAC | eeeeeddddddddddeeeee | 47 | 10487 | 10506 | 1529 |
| 619811 | 1479 | 1498 | CGAGAAGTTGTCCTGGATGT | eeeeeddddddddddeeeee | 44 | 10490 | 10509 | 1530 |
| 619812 | 1482 | 1501 | CACCGAGAAGTTGTCCTGGA | eeeeeddddddddddeeeee | 56 | 10493 | 10512 | 1531 |
| 619813 | 1485 | 1504 | AGTCACCGAGAAGTTGTCCT | eeeeeddddddddddeeeee | 48 | 10496 | 10515 | 1532 |
| 619814 | 1488 | 1507 | TTGAGTCACCGAGAAGTTGT | eeeeeddddddddddeeeee | 45 | 10499 | 10518 | 1533 |
| 619815 | 1491 | 1510 | CACTTGAGTCACCGAGAAGT | eeeeeddddddddddeeeee | 33 | 10502 | 10521 | 1534 |
| 619816 | 1494 | 1513 | GGGCACTTGAGTCACCGAGA | eeeeeddddddddddeeeee | 70 | 10505 | 10524 | 1535 |
| 619817 | 1497 | 1516 | GAAGGGCACTTGAGTCACCG | eeeeeddddddddddeeeee | 72 | 10508 | 10527 | 1536 |
| 619818 | 1500 | 1519 | AGTGAAGGGCACTTGAGTCA | eeeeeddddddddddeeeee | 41 | 10511 | 10530 | 1537 |
| 619819 | 1503 | 1522 | CTCAGTGAAGGGCACTTGAG | eeeeeddddddddddeeeee | 39 | 10514 | 10533 | 1538 |
| 619820 | 1506 | 1525 | GCTCTCAGTGAAGGGCACTT | eeeeeddddddddddeeeee | 57 | 10517 | 10536 | 1539 |
| 619821 | 1524 | 1543 | GATCAGCAGCAGGCAGGCGC | eeeeeddddddddddeeeee | 58 | 10535 | 10554 | 1540 |
| 619822 | 1527 | 1546 | CTGGATCAGCAGCAGGCAGG | eeeeeddddddddddeeeee | 59 | 10538 | 10557 | 1541 |
| 619823 | 1530 | 1549 | AGGCTGGATCAGCAGCAGGC | eeeeeddddddddddeeeee | 82 | 10541 | 10560 | 1542 |
| 619824 | 1533 | 1552 | GTGAGGCTGGATCAGCAGCA | eeeeeddddddddddeeeee | 65 | 10544 | 10563 | 1543 |
| 619825 | 1536 | 1555 | ATAGTGAGGCTGGATCAGCA | eeeeeddddddddddeeeee | 7 | 10547 | 10566 | 1544 |
| 619826 | 1539 | 1558 | GGCATAGTGAGGCTGGATCA | eeeeeddddddddddeeeee | 71 | 10550 | 10569 | 1545 |
| 619827 | 1542 | 1561 | AGAGGCATAGTGAGGCTGGA | eeeeeddddddddddeeeee | 58 | 10553 | 10572 | 1546 |
| 619828 | 1545 | 1564 | GTCAGAGGCATAGTGAGGCT | eeeeeddddddddddeeeee | 57 | 10556 | 10575 | 1547 |
| 619829 | 1548 | 1567 | CAGGTCAGAGGCATAGTGAG | eeeeeddddddddddeeeee | 46 | 10559 | 10578 | 1548 |
| 619830 | 1551 | 1570 | GTCCAGGTCAGAGGCATAGT | eeeeeddddddddddeeeee | 6 | 10562 | 10581 | 1549 |
| 619831 | 1554 | 1573 | CTTGTCCAGGTCAGAGGCAT | eeeeeddddddddddeeeee | 54 | 10565 | 10584 | 1550 |
| 619832 | 1557 | 1576 | CACCTTGTCCAGGTCAGAGG | eeeeeddddddddddeeeee | 47 | 10568 | 10587 | 1551 |
| 619833 | 1560 | 1579 | CTCCACCTTGTCCAGGTCAG | eeeeeddddddddddeeeee | 33 | 10571 | 10590 | 1552 |
| 619834 | 1563 | 1582 | ACCCTCCACCTTGTCCAGGT | eeeeeddddddddddeeeee | 59 | 10574 | 10593 | 1553 |
| 619835 | 1566 | 1585 | GAGACCCTCCACCTTGTCCA | eeeeeddddddddddeeeee | 47 | 10577 | 10596 | 1554 |
| 619836 | 1569 | 1588 | AGTGAGACCCTCCACCTTGT | eeeeeddddddddddeeeee | 52 | 10580 | 10599 | 1555 |
| 619837 | 1572 | 1591 | GAAAGTGAGACCCTCCACCT | eeeeeddddddddddeeeee | 40 | 10583 | 10602 | 1556 |
| 619838 | 1575 | 1594 | CTGGAAAGTGAGACCCTCCA | eeeeeddddddddddeeeee | 55 | 10586 | 10605 | 1557 |
| 619839 | 1578 | 1597 | TTGCTGGAAAGTGAGACCCT | eeeeeddddddddddeeeee | 44 | 10589 | 10608 | 1558 |
| 619840 | 1581 | 1600 | GTTTTGCTGGAAAGTGAGAC | eeeeeddddddddddeeeee | 50 | 10592 | 10611 | 1559 |
| 619841 | 1584 | 1603 | GGAGTTTTGCTGGAAAGTGA | eeeeeddddddddddeeeee | 54 | 10595 | 10614 | 1560 |
| 619842 | 1587 | 1606 | GAGGGAGTTTTGCTGGAAAG | eeeeeddddddddddeeeee | 35 | 10598 | 10617 | 1561 |
| 619843 | 1590 | 1609 | GTTGAGGGAGTTTTGCTGGA | eeeeeddddddddddeeeee | 40 | 10601 | 10620 | 1562 |
| 619844 | 1593 | 1612 | CCAGTTGAGGGAGTTTTGCT | eeeeeddddddddddeeeee | 32 | 10604 | 10623 | 1563 |
| 619845 | 1596 | 1615 | CATCCAGTTGAGGGAGTTTT | eeeeeddddddddddeeeee | 52 | 10607 | 10626 | 1564 |
| 619846 | 1599 | 1618 | CTTCATCCAGTTGAGGGAGT | eeeeeddddddddddeeeee | 56 | 10610 | 10629 | 1565 |
| 619847 | 1602 | 1621 | TTTCTTCATCCAGTTGAGGG | eeeeeddddddddddeeeee | 38 | 10613 | 10632 | 1566 |
| 619848 | 1605 | 1624 | TAGTTTCTTCATCCAGTTGA | eeeeeddddddddddeeeee | 29 | 10616 | 10635 | 1567 |
| 619849 | 1608 | 1627 | AGATAGTTTCTTCATCCAGT | eeeeeddddddddddeeeee | 12 | 10619 | 10638 | 1568 |
| 619850 | 1611 | 1630 | GGGAGATAGTTTCTTCATCC | eeeeeddddddddddeeeee | 32 | 10622 | 10641 | 1569 |
| 619851 | 1629 | 1648 | GGTCAGGTGGATGGTCCGGG | eeeeeddddddddddeeeee | 43 | N/A | N/A | 1570 |
| 619852 | 1632 | 1651 | CATGGTCAGGTGGATGGTCC | eeeeeddddddddddeeeee | 41 | 12238 | 12257 | 1571 |
| 619853 | 1635 | 1654 | GGGCATGGTCAGGTGGATGG | eeeeeddddddddddeeeee | 57 | 12241 | 12260 | 1572 |
| 619854 | 1653 | 1672 | TCCTTGCAGCACCAGTTGGG | eeeeeddddddddddeeeee | 46 | 12259 | 12278 | 1573 |
| 619855 | 1656 | 1675 | AGATCCTTGCAGCACCAGTT | eeeeeddddddddddeeeee | 36 | 12262 | 12281 | 1574 |
| 619856 | 1659 | 1678 | ATAAGATCCTTGCAGCACCA | eeeeeddddddddddeeeee | 37 | 12265 | 12284 | 1575 |
| 619857 | 1662 | 1681 | GTCATAAGATCCTTGCAGCA | eeeeeddddddddddeeeee | 35 | 12268 | 12287 | 1576 |
| 619858 | 1665 | 1684 | CAGGTCATAAGATCCTTGCA | eeeeeddddddddddeeeee | 41 | 12271 | 12290 | 1577 |
| 619859 | 1668 | 1687 | CTGCAGGTCATAAGATCCTT | eeeeeddddddddddeeeee | 32 | 12274 | 12293 | 1578 |
| 619860 | 1671 | 1690 | GTCCTGCAGGTCATAAGATC | eeeeeddddddddddeeeee | 47 | 12277 | 12296 | 1579 |
| 619861 | 1674 | 1693 | CAGGTCCTGCAGGTCATAAG | eeeeeddddddddddeeeee | 33 | 12280 | 12299 | 1580 |
| 619862 | 1677 | 1696 | GAGCAGGTCCTGCAGGTCAT | eeeeeddddddddddeeeee | 53 | 12283 | 12302 | 1581 |
| 619863 | 1680 | 1699 | GGCGAGCAGGTCCTGCAGGT | eeeeeddddddddddeeeee | 51 | 12286 | 12305 | 1582 |
| 619864 | 1683 | 1702 | CTGGGCGAGCAGGTCCTGCA | eeeeeddddddddddeeeee | 50 | 12289 | 12308 | 1583 |
| 619865 | 1686 | 1705 | AGCCTGGGCGAGCAGGTCCT | eeeeeddddddddddeeeee | 49 | 12292 | 12311 | 1584 |
| 619866 | 1689 | 1708 | CTCAGCCTGGGCGAGCAGGT | eeeeeddddddddddeeeee | 63 | 12295 | 12314 | 1585 |
| 619867 | 1692 | 1711 | CAGCTCAGCCTGGGCGAGCA | eeeeeddddddddddeeeee | 45 | 12298 | 12317 | 1586 |
| 619868 | 1699 | 1718 | TGGCGGGCAGCTCAGCCTGG | eeeeeddddddddddeeeee | 46 | 12305 | 12324 | 1587 |
| 619869 | 1702 | 1721 | GAATGGCGGGCAGCTCAGCC | eeeeeddddddddddeeeee | 46 | 12308 | 12327 | 1588 |
| 619870 | 1705 | 1724 | GCAGAATGGCGGGCAGCTCA | eeeeeddddddddddeeeee | 41 | 12311 | 12330 | 1589 |
| 619871 | 1708 | 1727 | TGTGCAGAATGGCGGGCAGC | eeeeeddddddddddeeeee | 44 | 12314 | 12333 | 1590 |
| 619872 | 1711 | 1730 | CGGTGTGCAGAATGGCGGGC | eeeeeddddddddddeeeee | 36 | 12317 | 12336 | 1591 |
| 619873 | 1714 | 1733 | GCTCGGTGTGCAGAATGGCG | eeeeeddddddddddeeeee | 63 | 12320 | 12339 | 1592 |
| 619874 | 1717 | 1736 | TCAGCTCGGTGTGCAGAATG | eeeeeddddddddddeeeee | 42 | 12323 | 12342 | 1593 |
| 619875 | 1720 | 1739 | GGTTCAGCTCGGTGTGCAGA | eeeeeddddddddddeeeee | 62 | 12326 | 12345 | 1594 |
| 619876 | 1723 | 1742 | GCAGGTTCAGCTCGGTGTGC | eeeeeddddddddddeeeee | 73 | 12329 | 12348 | 1595 |
| 619877 | 1726 | 1745 | TTTGCAGGTTCAGCTCGGTG | eeeeeddddddddddeeeee | 52 | 12332 | 12351 | 1596 |
| 619878 | 1729 | 1748 | ATTTTTGCAGGTTCAGCTCG | eeeeeddddddddddeeeee | 43 | 12335 | 12354 | 1597 |
| 619879 | 1732 | 1751 | TCAATTTTTGCAGGTTCAGC | eeeeeddddddddddeeeee | 29 | 12338 | 12357 | 1598 |
| 619880 | 1735 | 1754 | TGCTCAATTTTTGCAGGTTC | eeeeeddddddddddeeeee | 72 | 12341 | 12360 | 1599 |
| 619881 | 1738 | 1757 | CATTGCTCAATTTTTGCAGG | eeeeeddddddddddeeeee | 36 | 12344 | 12363 | 1600 |
| 619882 | 1741 | 1760 | GGTCATTGCTCAATTTTTGC | eeeeeddddddddddeeeee | 56 | 12347 | 12366 | 1601 |
| 619883 | 1744 | 1763 | TGCGGTCATTGCTCAATTTT | eeeeeddddddddddeeeee | 45 | 12350 | 12369 | 1602 |
| 619884 | 1747 | 1766 | TGATGCGGTCATTGCTCAAT | eeeeeddddddddddeeeee | 51 | 12353 | 12372 | 1603 |
| 619885 | 1750 | 1769 | CCCTGATGCGGTCATTGCTC | eeeeeddddddddddeeeee | 77 | 12356 | 12375 | 1604 |
| 619886 | 1753 | 1772 | CCACCCTGATGCGGTCATTG | eeeeeddddddddddeeeee | 56 | 12359 | 12378 | 1605 |
| 619887 | 1756 | 1775 | CCCCCACCCTGATGCGGTCA | eeeeeddddddddddeeeee | 52 | 12362 | 12381 | 1606 |
| 619888 | 1759 | 1778 | CCTCCCCCACCCTGATGCGG | eeeeeddddddddddeeeee | 36 | 12365 | 12384 | 1607 |
| 619889 | 1762 | 1781 | GCACCTCCCCCACCCTGATG | eeeeeddddddddddeeeee | 36 | N/A | N/A | 1608 |
| 619890 | 1765 | 1784 | TCAGCACCTCCCCCACCCTG | eeeeeddddddddddeeeee | 57 | N/A | N/A | 1609 |
| 619891 | 1768 | 1787 | TGTTCAGCACCTCCCCCACC | eeeeeddddddddddeeeee | 60 | N/A | N/A | 1610 |
| 619892 | 1771 | 1790 | TGCTGTTCAGCACCTCCCCC | eeeeeddddddddddeeeee | 65 | N/A | N/A | 1611 |
| 619893 | 1774 | 1793 | AAATGCTGTTCAGCACCTCC | eeeeeddddddddddeeeee | 68 | N/A | N/A | 1612 |
| 619894 | 1777 | 1796 | AAAAAATGCTGTTCAGCACC | eeeeeddddddddddeeeee | 41 | 13246 | 13265 | 1613 |
| 619895 | 1780 | 1799 | CAAAAAAAATGCTGTTCAGC | eeeeeddddddddddeeeee | 40 | 13249 | 13268 | 1614 |
| 619896 | 1783 | 1802 | GCTCAAAAAAAATGCTGTTC | eeeeeddddddddddeeeee | 64 | 13252 | 13271 | 1615 |
| 619897 | 1786 | 1805 | CAAGCTCAAAAAAAATGCTG | eeeeeddddddddddeeeee | 44 | 13255 | 13274 | 1616 |
| 619898 | 1789 | 1808 | CTTCAAGCTCAAAAAAAATG | eeeeeddddddddddeeeee | 15 | 13258 | 13277 | 1617 |
| 619899 | 1792 | 1811 | CCGCTTCAAGCTCAAAAAAA | eeeeeddddddddddeeeee | 62 | 13261 | 13280 | 1618 |
| 619900 | 1795 | 1814 | CATCCGCTTCAAGCTCAAAA | eeeeeddddddddddeeeee | 62 | 13264 | 13283 | 1619 |
| 619901 | 1798 | 1817 | TCTCATCCGCTTCAAGCTCA | eeeeeddddddddddeeeee | 72 | 13267 | 13286 | 1620 |
| 619902 | 1801 | 1820 | CTCTCTCATCCGCTTCAAGC | eeeeeddddddddddeeeee | 66 | 13270 | 13289 | 1621 |
| 619903 | 1804 | 1823 | GCTCTCTCTCATCCGCTTCA | eeeeeddddddddddeeeee | 68 | 13273 | 13292 | 1622 |
| 619904 | 1807 | 1826 | TGGGCTCTCTCTCATCCGCT | eeeeeddddddddddeeeee | 83 | 13276 | 13295 | 1623 |
| 619905 | 1810 | 1829 | CTGTGGGCTCTCTCTCATCC | eeeeeddddddddddeeeee | 80 | 13279 | 13298 | 1624 |
| 619906 | 1813 | 1832 | ACTCTGTGGGCTCTCTCTCA | eeeeeddddddddddeeeee | 54 | 13282 | 13301 | 1625 |
| 619907 | 1816 | 1835 | TAGACTCTGTGGGCTCTCTC | eeeeeddddddddddeeeee | 75 | 13285 | 13304 | 1626 |
| 619908 | 1824 | 1843 | CTGTTGGGTAGACTCTGTGG | eeeeeddddddddddeeeee | 46 | 13293 | 13312 | 1627 |
| 619909 | 1827 | 1846 | AAGCTGTTGGGTAGACTCTG | eeeeeddddddddddeeeee | 63 | 13296 | 13315 | 1628 |
| 619910 | 1830 | 1849 | GTTAAGCTGTTGGGTAGACT | eeeeeddddddddddeeeee | 61 | 13299 | 13318 | 1629 |
| 619911 | 1833 | 1852 | CTTGTTAAGCTGTTGGGTAG | eeeeeddddddddddeeeee | 47 | 13302 | 13321 | 1630 |
| 619912 | 1836 | 1855 | AGGCTTGTTAAGCTGTTGGG | eeeeeddddddddddeeeee | 69 | 13305 | 13324 | 1631 |
| 619913 | 1839 | 1858 | CTCAGGCTTGTTAAGCTGTT | eeeeeddddddddddeeeee | 62 | 13308 | 13327 | 1632 |
| 619914 | 1842 | 1861 | GACCTCAGGCTTGTTAAGCT | eeeeeddddddddddeeeee | 55 | 13311 | 13330 | 1633 |
| 619915 | 1845 | 1864 | CAAGACCTCAGGCTTGTTAA | eeeeeddddddddddeeeee | 50 | 13314 | 13333 | 1634 |
| 619916 | 1848 | 1867 | CTCCAAGACCTCAGGCTTGT | eeeeeddddddddddeeeee | 60 | 13317 | 13336 | 1635 |
| 619917 | 1851 | 1870 | CACCTCCAAGACCTCAGGCT | eeeeeddddddddddeeeee | 61 | 13320 | 13339 | 1636 |
| 619918 | 1854 | 1873 | GGTCACCTCCAAGACCTCAG | eeeeeddddddddddeeeee | 67 | 13323 | 13342 | 1637 |
| 619919 | 1857 | 1876 | CAGGGTCACCTCCAAGACCT | eeeeeddddddddddeeeee | 54 | 13326 | 13345 | 1638 |
| 619920 | 1860 | 1879 | GTTCAGGGTCACCTCCAAGA | eeeeeddddddddddeeeee | 54 | 13329 | 13348 | 1639 |
| 619921 | 1863 | 1882 | GCGGTTCAGGGTCACCTCCA | eeeeeddddddddddeeeee | 70 | 13332 | 13351 | 1640 |
| 619922 | 1873 | 1892 | ACAGGAATGGGCGGTTCAGG | eeeeeddddddddddeeeee | 34 | 13342 | 13361 | 1641 |
| 619926 | 1876 | 1895 | CAAACAGGAATGGGCGGTTC | eeeeeddddddddddeeeee | 40 | 13345 | 13364 | 1642 |
| 619927 | 1879 | 1898 | CAGCAAACAGGAATGGGCGG | eeeeeddddddddddeeeee | 49 | 13348 | 13367 | 1643 |
| 619928 | 1882 | 1901 | ACACAGCAAACAGGAATGGG | eeeeeddddddddddeeeee | 28 | 13351 | 13370 | 1644 |
| 619929 | 1885 | 1904 | CATACACAGCAAACAGGAAT | eeeeeddddddddddeeeee | 29 | 13354 | 13373 | 1645 |
| 619930 | 1888 | 1907 | GATCATACACAGCAAACAGG | eeeeeddddddddddeeeee | 49 | 13357 | 13376 | 1646 |
| 619931 | 1891 | 1910 | TTTGATCATACACAGCAAAC | eeeeeddddddddddeeeee | 22 | 13360 | 13379 | 1647 |
| 619932 | 1894 | 1913 | CGCTTTGATCATACACAGCA | eeeeeddddddddddeeeee | 56 | 13363 | 13382 | 1648 |
| 619933 | 1911 | 1930 | GAAGTGCAGGGCAGTGGCGC | eeeeeddddddddddeeeee | 44 | 13380 | 13399 | 1649 |
| 619934 | 1914 | 1933 | CAGGAAGTGCAGGGCAGTGG | eeeeeddddddddddeeeee | 39 | 13383 | 13402 | 1650 |
| 619935 | 1917 | 1936 | GCCCAGGAAGTGCAGGGCAG | eeeeeddddddddddeeeee | 20 | 13386 | 13405 | 1651 |
| 619936 | 1920 | 1939 | GCGGCCCAGGAAGTGCAGGG | eeeeeddddddddddeeeee | 19 | 13389 | 13408 | 1652 |
| 619937 | 1923 | 1942 | CACGCGGCCCAGGAAGTGCA | eeeeeddddddddddeeeee | 34 | 13392 | 13411 | 1653 |
| 619938 | 1926 | 1945 | GGCCACGCGGCCCAGGAAGT | eeeeeddddddddddeeeee | 21 | 13395 | 13414 | 1654 |
| 619939 | 1929 | 1948 | GTTGGCCACGCGGCCCAGGA | eeeeeddddddddddeeeee | 34 | 13398 | 13417 | 1655 |
| 619940 | 1932 | 1951 | CGGGTTGGCCACGCGGCCCA | eeeeeddddddddddeeeee | 38 | 13401 | 13420 | 1656 |
| 619941 | 1935 | 1954 | CAGCGGGTTGGCCACGCGGC | eeeeeddddddddddeeeee | 42 | 13404 | 13423 | 1657 |
| 619942 | 1938 | 1957 | GCTCAGCGGGTTGGCCACGC | eeeeeddddddddddeeeee | 64 | 13407 | 13426 | 1658 |
| 619943 | 1941 | 1960 | TGTGCTCAGCGGGTTGGCCA | eeeeeddddddddddeeeee | 43 | 13410 | 13429 | 1659 |
| 619944 | 1944 | 1963 | TGCTGTGCTCAGCGGGTTGG | eeeeeddddddddddeeeee | 29 | 13413 | 13432 | 1660 |
| 619945 | 1947 | 1966 | TCATGCTGTGCTCAGCGGGT | eeeeeddddddddddeeeee | 49 | 13416 | 13435 | 1661 |
| 619946 | 1950 | 1969 | GCCTCATGCTGTGCTCAGCG | eeeeeddddddddddeeeee | 74 | 13419 | 13438 | 1662 |
| 619947 | 1953 | 1972 | CTGGCCTCATGCTGTGCTCA | eeeeeddddddddddeeeee | 56 | 13422 | 13441 | 1663 |
| 619948 | 1956 | 1975 | GCCCTGGCCTCATGCTGTGC | eeeeeddddddddddeeeee | 44 | 13425 | 13444 | 1664 |
| 619949 | 1976 | 1995 | GCCAGGCACTGTGTTCTGGG | eeeeeddddddddddeeeee | 65 | 13445 | 13464 | 1665 |
| 619950 | 1979 | 1998 | CTTGCCAGGCACTGTGTTCT | eeeeeddddddddddeeeee | 71 | 13448 | 13467 | 1666 |
| 619951 | 1982 | 2001 | GGCCTTGCCAGGCACTGTGT | eeeeeddddddddddeeeee | 80 | 13451 | 13470 | 1667 |
| 619952 | 2114 | 2133 | AGGAGAAACGGCTGCTTTCC | eeeeeddddddddddeeeee | 61 | 13583 | 13602 | 1668 |
| 619953 | 2117 | 2136 | CCAAGGAGAAACGGCTGCTT | eeeeeddddddddddeeeee | 75 | 13586 | 13605 | 1669 |
| 619954 | 2120 | 2139 | AGACCAAGGAGAAACGGCTG | eeeeeddddddddddeeeee | 76 | 13589 | 13608 | 1670 |
| 619955 | 2123 | 2142 | CTTAGACCAAGGAGAAACGG | eeeeeddddddddddeeeee | 67 | 13592 | 13611 | 1671 |
| 619956 | 2126 | 2145 | ACACTTAGACCAAGGAGAAA | eeeeeddddddddddeeeee | 45 | 13595 | 13614 | 1672 |
| 619957 | 2129 | 2148 | AGCACACTTAGACCAAGGAG | eeeeeddddddddddeeeee | 74 | 13598 | 13617 | 1673 |
| 619958 | 2132 | 2151 | TGCAGCACACTTAGACCAAG | eeeeeddddddddddeeeee | 55 | 13601 | 13620 | 1674 |
| 619959 | 2135 | 2154 | CCATGCAGCACACTTAGACC | eeeeeddddddddddeeeee | 56 | 13604 | 13623 | 1675 |
| 619960 | 2138 | 2157 | ACTCCATGCAGCACACTTAG | eeeeeddddddddddeeeee | 66 | 13607 | 13626 | 1676 |
| 619961 | 2141 | 2160 | CTCACTCCATGCAGCACACT | eeeeeddddddddddeeeee | 63 | 13610 | 13629 | 1677 |
| 619962 | 2159 | 2178 | CGCTGCAGGCTTCTACTGCT | eeeeeddddddddddeeeee | 64 | 13628 | 13647 | 1678 |
| 619963 | 2162 | 2181 | TGCCGCTGCAGGCTTCTACT | eeeeeddddddddddeeeee | 60 | 13631 | 13650 | 1679 |
| 619964 | 2165 | 2184 | TTGTGCCGCTGCAGGCTTCT | eeeeeddddddddddeeeee | 45 | 13634 | 13653 | 1680 |
| 619965 | 2168 | 2187 | CATTTGTGCCGCTGCAGGCT | eeeeeddddddddddeeeee | 62 | 13637 | 13656 | 1681 |
| 619966 | 2171 | 2190 | GTGCATTTGTGCCGCTGCAG | eeeeeddddddddddeeeee | 85 | 13640 | 13659 | 1682 |
| 619967 | 2174 | 2193 | GAGGTGCATTTGTGCCGCTG | eeeeeddddddddddeeeee | 80 | 13643 | 13662 | 1683 |
| 619968 | 2177 | 2196 | TGGGAGGTGCATTTGTGCCG | eeeeeddddddddddeeeee | 53 | 13646 | 13665 | 1684 |
| 619969 | 2180 | 2199 | AACTGGGAGGTGCATTTGTG | eeeeeddddddddddeeeee | 34 | 13649 | 13668 | 1685 |
| 619970 | 2183 | 2202 | GCAAACTGGGAGGTGCATTT | eeeeeddddddddddeeeee | 62 | 13652 | 13671 | 1686 |
| 619971 | 2186 | 2205 | CCAGCAAACTGGGAGGTGCA | eeeeeddddddddddeeeee | 76 | 13655 | 13674 | 1687 |
| 619972 | 2189 | 2208 | AACCCAGCAAACTGGGAGGT | eeeeeddddddddddeeeee | 56 | 13658 | 13677 | 1688 |
| 619973 | 2192 | 2211 | ATAAACCCAGCAAACTGGGA | eeeeeddddddddddeeeee | 56 | 13661 | 13680 | 1689 |
| 619974 | 2195 | 2214 | AAAATAAACCCAGCAAACTG | eeeeeddddddddddeeeee | 33 | 13664 | 13683 | 1690 |
| 619975 | 2198 | 2217 | TCTAAAATAAACCCAGCAAA | eeeeeddddddddddeeeee | 29 | 13667 | 13686 | 1691 |
| 619976 | 2201 | 2220 | TTCTCTAAAATAAACCCAGC | eeeeeddddddddddeeeee | 58 | 13670 | 13689 | 1692 |
| 619977 | 2204 | 2223 | CCATTCTCTAAAATAAACCC | eeeeeddddddddddeeeee | 55 | 13673 | 13692 | 1693 |
| 619978 | 2207 | 2226 | CCCCCATTCTCTAAAATAAA | eeeeeddddddddddeeeee | 49 | 13676 | 13695 | 1694 |
| 619979 | 2210 | 2229 | CCACCCCCATTCTCTAAAAT | eeeeeddddddddddeeeee | 19 | 13679 | 13698 | 1695 |
| 619980 | 2213 | 2232 | TCCCCACCCCCATTCTCTAA | eeeeeddddddddddeeeee | 41 | 13682 | 13701 | 1696 |
| 619981 | 2216 | 2235 | GCCTCCCCACCCCCATTCTC | eeeeeddddddddddeeeee | 53 | 13685 | 13704 | 1697 |
| 619982 | 2219 | 2238 | CTTGCCTCCCCACCCCCATT | eeeeeddddddddddeeeee | 56 | 13688 | 13707 | 1698 |
| 619983 | 2222 | 2241 | GTTCTTGCCTCCCCACCCCC | eeeeeddddddddddeeeee | 72 | 13691 | 13710 | 1699 |
| 619984 | 2225 | 2244 | CTGGTTCTTGCCTCCCCACC | eeeeeddddddddddeeeee | 82 | 13694 | 13713 | 1700 |
| 619985 | 2228 | 2247 | ACACTGGTTCTTGCCTCCCC | eeeeeddddddddddeeeee | 74 | 13697 | 13716 | 1701 |
| 619986 | 2231 | 2250 | TAAACACTGGTTCTTGCCTC | eeeeeddddddddddeeeee | 72 | 13700 | 13719 | 1702 |
| 619987 | 2234 | 2253 | CGCTAAACACTGGTTCTTGC | eeeeeddddddddddeeeee | 93 | 13703 | 13722 | 1703 |
| 619988 | 2237 | 2256 | CCGCGCTAAACACTGGTTCT | eeeeeddddddddddeeeee | 82 | 13706 | 13725 | 1704 |
| 619989 | 2240 | 2259 | GTCCCGCGCTAAACACTGGT | eeeeeddddddddddeeeee | 75 | 13709 | 13728 | 1705 |
| 619990 | 2243 | 2262 | GTAGTCCCGCGCTAAACACT | eeeeeddddddddddeeeee | 73 | 13712 | 13731 | 1706 |
| 619991 | 2246 | 2265 | ACAGTAGTCCCGCGCTAAAC | eeeeeddddddddddeeeee | 64 | 13715 | 13734 | 1707 |
| 619992 | 2249 | 2268 | GGAACAGTAGTCCCGCGCTA | eeeeeddddddddddeeeee | 85 | 13718 | 13737 | 1708 |
| 619993 | 2252 | 2271 | TTTGGAACAGTAGTCCCGCG | eeeeeddddddddddeeeee | 65 | 13721 | 13740 | 1709 |
| 619994 | 2255 | 2274 | CTTTTTGGAACAGTAGTCCC | eeeeeddddddddddeeeee | 69 | 13724 | 13743 | 1710 |
| 619995 | 2258 | 2277 | ATTCTTTTTGGAACAGTAGT | eeeeeddddddddddeeeee | 53 | 13727 | 13746 | 1711 |
| 619996 | 2261 | 2280 | GGAATTCTTTTTGGAACAGT | eeeeeddddddddddeeeee | 70 | 13730 | 13749 | 1712 |
| 619997 | 2264 | 2283 | GTTGGAATTCTTTTTGGAAC | eeeeeddddddddddeeeee | 57 | 13733 | 13752 | 1713 |
| 619998 | 2267 | 2286 | TCGGTTGGAATTCTTTTTGG | eeeeeddddddddddeeeee | 83 | 13736 | 13755 | 1714 |
| 619999 | 2270 | 2289 | TGGTCGGTTGGAATTCTTTT | eeeeeddddddddddeeeee | 74 | 13739 | 13758 | 1715 |
| 620000 | 2273 | 2292 | AGCTGGTCGGTTGGAATTCT | eeeeeddddddddddeeeee | 78 | 13742 | 13761 | 1716 |
| 620001 | 2276 | 2295 | ACAAGCTGGTCGGTTGGAAT | eeeeeddddddddddeeeee | 61 | 13745 | 13764 | 1717 |
| 620002 | 2279 | 2298 | CAAACAAGCTGGTCGGTTGG | eeeeeddddddddddeeeee | 61 | 13748 | 13767 | 1718 |
| 620003 | 2282 | 2301 | TCACAAACAAGCTGGTCGGT | eeeeeddddddddddeeeee | 88 | 13751 | 13770 | 1719 |
| 620004 | 2285 | 2304 | GTTTCACAAACAAGCTGGTC | eeeeeddddddddddeeeee | 91 | 13754 | 13773 | 1720 |
| 620005 | 2288 | 2307 | TTTGTTTCACAAACAAGCTG | eeeeeddddddddddeeeee | 73 | 13757 | 13776 | 1721 |
| 620006 | 2304 | 2323 | GAAAAGGGAACACTTTTTTG | eeeeeddddddddddeeeee | 59 | 13773 | 13792 | 1722 |
| 620007 | 2307 | 2326 | CTTGAAAAGGGAACACTTTT | eeeeeddddddddddeeeee | 57 | 13776 | 13795 | 1723 |
| 620008 | 2310 | 2329 | CAACTTGAAAAGGGAACACT | eeeeeddddddddddeeeee | 88 | 13779 | 13798 | 1724 |
| 620009 | 2313 | 2332 | TCTCAACTTGAAAAGGGAAC | eeeeeddddddddddeeeee | 88 | 13782 | 13801 | 1725 |
| 620010 | 2316 | 2335 | TGTTCTCAACTTGAAAAGGG | eeeeeddddddddddeeeee | 93 | 13785 | 13804 | 1726 |
| 620011 | 2319 | 2338 | TTTTGTTCTCAACTTGAAAA | eeeeeddddddddddeeeee | 49 | 13788 | 13807 | 1727 |
| 620012 | 2322 | 2341 | AATTTTTGTTCTCAACTTGA | eeeeeddddddddddeeeee | 63 | 13791 | 13810 | 1728 |
| 620013 | 2325 | 2344 | CCCAATTTTTGTTCTCAACT | eeeeeddddddddddeeeee | 89 | 13794 | 13813 | 1729 |
| 620014 | 2328 | 2347 | AAACCCAATTTTTGTTCTCA | eeeeeddddddddddeeeee | 78 | 13797 | 13816 | 1730 |
| 620015 | 2331 | 2350 | TTAAAACCCAATTTTTGTTC | eeeeeddddddddddeeeee | 68 | 13800 | 13819 | 1731 |
| 620016 | 2334 | 2353 | ATTTTAAAACCCAATTTTTG | eeeeeddddddddddeeeee | 15 | 13803 | 13822 | 1732 |
| 620017 | 2337 | 2356 | TTAATTTTAAAACCCAATTT | eeeeeddddddddddeeeee | 15 | 13806 | 13825 | 1733 |
| 620018 | 2353 | 2372 | TGCAAAAATGTATACTTTAA | eeeeeddddddddddeeeee | 32 | 13822 | 13841 | 1734 |
| 620019 | 2356 | 2375 | CAATGCAAAAATGTATACTT | eeeeeddddddddddeeeee | 64 | 13825 | 13844 | 1735 |
| 620020 | 2359 | 2378 | AGGCAATGCAAAAATGTATA | eeeeeddddddddddeeeee | 76 | 13828 | 13847 | 1736 |
| 620021 | 2362 | 2381 | CGAAGGCAATGCAAAAATGT | eeeeeddddddddddeeeee | 50 | 13831 | 13850 | 1737 |
| 620022 | 2365 | 2384 | AACCGAAGGCAATGCAAAAA | eeeeeddddddddddeeeee | 55 | 13834 | 13853 | 1738 |
| 620023 | 2368 | 2387 | ACAAACCGAAGGCAATGCAA | eeeeeddddddddddeeeee | 68 | 13837 | 13856 | 1739 |
| 620024 | 2371 | 2390 | AATACAAACCGAAGGCAATG | eeeeeddddddddddeeeee | 68 | 13840 | 13859 | 1740 |
| 620025 | 2374 | 2393 | CTAAATACAAACCGAAGGCA | eeeeeddddddddddeeeee | 64 | 13843 | 13862 | 1741 |
| 620026 | 2377 | 2396 | ACACTAAATACAAACCGAAG | eeeeeddddddddddeeeee | 49 | 13846 | 13865 | 1742 |
| 620027 | 2380 | 2399 | AAGACACTAAATACAAACCG | eeeeeddddddddddeeeee | 53 | 13849 | 13868 | 1743 |
| 620028 | 2383 | 2402 | TTCAAGACACTAAATACAAA | eeeeeddddddddddeeeee | 31 | 13852 | 13871 | 1744 |
| 620029 | 2386 | 2405 | ACATTCAAGACACTAAATAC | eeeeeddddddddddeeeee | 35 | 13855 | 13874 | 1745 |
| 620030 | 2389 | 2408 | CTTACATTCAAGACACTAAA | eeeeeddddddddddeeeee | 57 | 13858 | 13877 | 1746 |
| 620031 | 2392 | 2411 | GTTCTTACATTCAAGACACT | eeeeeddddddddddeeeee | 54 | 13861 | 13880 | 1747 |
| 620032 | 2395 | 2414 | CATGTTCTTACATTCAAGAC | eeeeeddddddddddeeeee | 39 | 13864 | 13883 | 1748 |
| 620033 | 2398 | 2417 | GGTCATGTTCTTACATTCAA | eeeeeddddddddddeeeee | 58 | 13867 | 13886 | 1749 |
| 620034 | 2401 | 2420 | GGAGGTCATGTTCTTACATT | eeeeeddddddddddeeeee | 51 | 13870 | 13889 | 1750 |
| 620035 | 2404 | 2423 | CACGGAGGTCATGTTCTTAC | eeeeeddddddddddeeeee | 61 | 13873 | 13892 | 1751 |
| 620036 | 2407 | 2426 | CTACACGGAGGTCATGTTCT | eeeeeddddddddddeeeee | 53 | 13876 | 13895 | 1752 |
| 620037 | 2410 | 2429 | ACACTACACGGAGGTCATGT | eeeeeddddddddddeeeee | 44 | 13879 | 13898 | 1753 |
| 620038 | 2413 | 2432 | CAGACACTACACGGAGGTCA | eeeeeddddddddddeeeee | 50 | 13882 | 13901 | 1754 |
| 620039 | 2416 | 2435 | TTACAGACACTACACGGAGG | eeeeeddddddddddeeeee | 66 | 13885 | 13904 | 1755 |
| 620040 | 2419 | 2438 | GTATTACAGACACTACACGG | eeeeeddddddddddeeeee | 53 | 13888 | 13907 | 1756 |
| 620041 | 2422 | 2441 | AAGGTATTACAGACACTACA | eeeeeddddddddddeeeee | 57 | 13891 | 13910 | 1757 |
| 620042 | 2425 | 2444 | ACTAAGGTATTACAGACACT | eeeeeddddddddddeeeee | 50 | 13894 | 13913 | 1758 |
| 620043 | 2428 | 2447 | AAAACTAAGGTATTACAGAC | eeeeeddddddddddeeeee | 28 | 13897 | 13916 | 1759 |
| 620044 | 2431 | 2450 | GAAAAAACTAAGGTATTACA | eeeeeddddddddddeeeee | 19 | 13900 | 13919 | 1760 |
| 620045 | 2434 | 2453 | GTGGAAAAAACTAAGGTATT | eeeeeddddddddddeeeee | 36 | 13903 | 13922 | 1761 |
| 620046 | 2437 | 2456 | TCTGTGGAAAAAACTAAGGT | eeeeeddddddddddeeeee | 38 | 13906 | 13925 | 1762 |
| 620047 | 2440 | 2459 | GCATCTGTGGAAAAAACTAA | eeeeeddddddddddeeeee | 29 | 13909 | 13928 | 1763 |
| 620048 | 2443 | 2462 | CAAGCATCTGTGGAAAAAAC | eeeeeddddddddddeeeee | 21 | 13912 | 13931 | 1764 |
| 620049 | 2446 | 2465 | TCACAAGCATCTGTGGAAAA | eeeeeddddddddddeeeee | 30 | 13915 | 13934 | 1765 |
| 620050 | 2449 | 2468 | AAATCACAAGCATCTGTGGA | eeeeeddddddddddeeeee | 36 | 13918 | 13937 | 1766 |
| 620051 | 2452 | 2471 | CAAAAATCACAAGCATCTGT | eeeeeddddddddddeeeee | 19 | 13921 | 13940 | 1767 |
| 620052 | 2455 | 2474 | GTTCAAAAATCACAAGCATC | eeeeeddddddddddeeeee | 32 | 13924 | 13943 | 1768 |
| 620053 | 2458 | 2477 | ATTGTTCAAAAATCACAAGC | eeeeeddddddddddeeeee | 16 | 13927 | 13946 | 1769 |
| 620054 | 2461 | 2480 | CGTATTGTTCAAAAATCACA | eeeeeddddddddddeeeee | 30 | 13930 | 13949 | 1770 |
| 620055 | 2479 | 2498 | AGGTGCTTGCATCTTTCACG | eeeeeddddddddddeeeee | 61 | 13948 | 13967 | 1771 |
| 620056 | 2482 | 2501 | TTCAGGTGCTTGCATCTTTC | eeeeeddddddddddeeeee | 58 | 13951 | 13970 | 1772 |
| 620057 | 2485 | 2504 | AAATTCAGGTGCTTGCATCT | eeeeeddddddddddeeeee | 35 | 13954 | 13973 | 1773 |
| 620058 | 2488 | 2507 | CAGAAATTCAGGTGCTTGCA | eeeeeddddddddddeeeee | 58 | 13957 | 13976 | 1774 |
| 620059 | 2491 | 2510 | AAACAGAAATTCAGGTGCTT | eeeeeddddddddddeeeee | 51 | 13960 | 13979 | 1775 |
| 620060 | 2494 | 2513 | TTCAAACAGAAATTCAGGTG | eeeeeddddddddddeeeee | 46 | 13963 | 13982 | 1776 |
| 620061 | 2497 | 2516 | GCATTCAAACAGAAATTCAG | eeeeeddddddddddeeeee | 40 | 13966 | 13985 | 1777 |
| 620062 | 2500 | 2519 | TCCGCATTCAAACAGAAATT | eeeeeddddddddddeeeee | 73 | 13969 | 13988 | 1778 |
| 620063 | 2503 | 2522 | GGTTCCGCATTCAAACAGAA | eeeeeddddddddddeeeee | 54 | 13972 | 13991 | 1779 |
| 620064 | 2506 | 2525 | TATGGTTCCGCATTCAAACA | eeeeeddddddddddeeeee | 44 | 13975 | 13994 | 1780 |
| 620065 | 2509 | 2528 | AGCTATGGTTCCGCATTCAA | eeeeeddddddddddeeeee | 67 | 13978 | 13997 | 1781 |
| 620066 | 2512 | 2531 | ACCAGCTATGGTTCCGCATT | eeeeeddddddddddeeeee | 60 | 13981 | 14000 | 1782 |
| 620067 | 2515 | 2534 | ATAACCAGCTATGGTTCCGC | eeeeeddddddddddeeeee | 70 | 13984 | 14003 | 1783 |
| 620068 | 2518 | 2537 | GAAATAACCAGCTATGGTTC | eeeeeddddddddddeeeee | 50 | 13987 | 14006 | 1784 |
| 620069 | 2521 | 2540 | GGAGAAATAACCAGCTATGG | eeeeeddddddddddeeeee | 50 | 13990 | 14009 | 1785 |
| 620070 | 2524 | 2543 | AAGGGAGAAATAACCAGCTA | eeeeeddddddddddeeeee | 56 | 13993 | 14012 | 1786 |
| 620071 | 2527 | 2546 | CACAAGGGAGAAATAACCAG | eeeeeddddddddddeeeee | 53 | 13996 | 14015 | 1787 |
| 620072 | 2530 | 2549 | TAACACAAGGGAGAAATAAC | eeeeeddddddddddeeeee | 27 | 13999 | 14018 | 1788 |
| 620073 | 2533 | 2552 | TACTAACACAAGGGAGAAAT | eeeeeddddddddddeeeee | 39 | 14002 | 14021 | 1789 |
| 620074 | 2536 | 2555 | TATTACTAACACAAGGGAGA | eeeeeddddddddddeeeee | 52 | 14005 | 14024 | 1790 |
| 620075 | 2539 | 2558 | GTTTATTACTAACACAAGGG | eeeeeddddddddddeeeee | 56 | 14008 | 14027 | 1791 |
| 620076 | 2558 | 2577 | AGGCTTATTGTGGCAAGACG | eeeeeddddddddddeeeee | 50 | 14027 | 14046 | 1792 |
| 620077 | 2561 | 2580 | TGGAGGCTTATTGTGGCAAG | eeeeeddddddddddeeeee | 38 | 14030 | 14049 | 1793 |
| 620078 | 2564 | 2583 | TTTTGGAGGCTTATTGTGGC | eeeeeddddddddddeeeee | 22 | 14033 | 14052 | 1794 |
| 620079 | 2567 | 2586 | TTTTTTTGGAGGCTTATTGT | eeeeeddddddddddeeeee | 48 | N/A | N/A | 1795 |

Table 11 shows inhibition of AGT mRNA in HepG2 cells cultured at a density of 20,000 cells per well which were transfected using electroporation with 500 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 11
Inhibition of AGT mRNA by MOE and/or cEt containing
gapmers targeting SEQ ID NO: 1 and/or 2

| | SEQ | SEQ | | | | SEQ | SEQ | |
|---|---|---|---|---|---|---|---|---|
| | ID: 1 | ID: 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568637 | 2046 | 2061 | CGCTGATTTGTCCGGG | eekddddddddddkke | 62 | 13515 | 13530 | 129 |
| 594621 | 2022 | 2037 | CTGCTGCTGGCCTTTG | kkkddddddddddkkk | 16 | 13491 | 13506 | 162 |
| 594622 | 2027 | 2042 | GTTATCTGCTGCTGGC | kkkddddddddddkkk | 44 | 13496 | 13511 | 163 |
| 594623 | 2032 | 2047 | GGGTTGTTATCTGCTG | kkkddddddddddkkk | 32 | 13501 | 13516 | 164 |
| 594624 | 2046 | 2061 | CGCTGATTTGTCCGGG | kkkddddddddddkkk | 62 | 13515 | 13530 | 129 |
| 594625 | 2047 | 2062 | TCGCTGATTTGTCCGG | kkkddddddddddkkk | 49 | 13516 | 13531 | 165 |
| 594626 | 2049 | 2064 | CATCGCTGATTTGTCC | kkkddddddddddkkk | 36 | 13518 | 13533 | 166 |
| 594627 | 2053 | 2068 | GACACATCGCTGATTT | kkkddddddddddkkk | 0 | 13522 | 13537 | 167 |
| 594628 | 2073 | 2088 | AAAGGTGGGAGACTGG | kkkddddddddddkkk | 51 | 13542 | 13557 | 168 |
| 609078 | 2020 | 2035 | GCTGCTGGCCTTTGCC | kkkddddddddddkkk | 26 | 13489 | 13504 | 173 |
| 609079 | 2021 | 2036 | TGCTGCTGGCCTTTGC | kkkddddddddddkkk | 31 | 13490 | 13505 | 174 |
| 609080 | 2023 | 2038 | TCTGCTGCTGGCCTTT | kkkddddddddddkkk | 41 | 13492 | 13507 | 175 |
| 609081 | 2024 | 2039 | ATCTGCTGCTGGCCTT | kkkddddddddddkkk | 29 | 13493 | 13508 | 176 |
| 609082 | 2025 | 2040 | TATCTGCTGCTGGCCT | kkkddddddddddkkk | 43 | 13494 | 13509 | 177 |
| 609083 | 2026 | 2041 | TTATCTGCTGCTGGCC | kkkddddddddddkkk | 19 | 13495 | 13510 | 178 |
| 609084 | 2028 | 2043 | TGTTATCTGCTGCTGG | kkkddddddddddkkk | 0 | 13497 | 13512 | 179 |
| 609085 | 2029 | 2044 | TTGTTATCTGCTGCTG | kkkddddddddddkkk | 40 | 13498 | 13513 | 180 |
| 609086 | 2030 | 2045 | GTTGTTATCTGCTGCT | kkkddddddddddkkk | 67 | 13499 | 13514 | 181 |
| 609087 | 2031 | 2046 | GGTTGTTATCTGCTGC | kkkddddddddddkkk | 73 | 13500 | 13515 | 182 |
| 609088 | 2048 | 2063 | ATCGCTGATTTGTCCG | kkkddddddddddkkk | 59 | 13517 | 13532 | 183 |
| 609089 | 2050 | 2065 | ACATCGCTGATTTGTC | kkkddddddddddkkk | 47 | 13519 | 13534 | 184 |
| 609090 | 2051 | 2066 | CACATCGCTGATTTGT | kkkddddddddddkkk | 34 | 13520 | 13535 | 185 |
| 609091 | 2052 | 2067 | ACACATCGCTGATTTG | kkkddddddddddkkk | 59 | 13521 | 13536 | 186 |
| 609092 | 2054 | 2069 | TGACACATCGCTGATT | kkkddddddddddkkk | 27 | 13523 | 13538 | 187 |
| 609093 | 2055 | 2070 | GTGACACATCGCTGAT | kkkddddddddddkkk | 38 | 13524 | 13539 | 188 |
| 609094 | 2056 | 2071 | GGTGACACATCGCTGA | kkkddddddddddkkk | 51 | 13525 | 13540 | 130 |
| 609095 | 2057 | 2072 | GGGTGACACATCGCTG | kkkddddddddddkkk | 59 | 13526 | 13541 | 189 |
| 609096 | 2074 | 2089 | AAAAGGTGGGAGACTG | kkkddddddddddkkk | 20 | 13543 | 13558 | 190 |
| 609097 | 2075 | 2090 | GAAAAGGTGGGAGACT | kkkddddddddddkkk | 19 | 13544 | 13559 | 131 |
| 609098 | 2076 | 2091 | AGAAAAGGTGGGAGAC | kkkddddddddddkkk | 12 | 13545 | 13560 | 191 |
| 622201 | 2020 | 2035 | GCTGCTGGCCTTTGCC | ekkddddddddddkke | 29 | 13489 | 13504 | 173 |
| 622202 | 2021 | 2036 | TGCTGCTGGCCTTTGC | ekkddddddddddkke | 17 | 13490 | 13505 | 174 |
| 622203 | 2022 | 2037 | CTGCTGCTGGCCTTTG | ekkddddddddddkke | 28 | 13491 | 13506 | 162 |
| 622204 | 2023 | 2038 | TCTGCTGCTGGCCTTT | ekkddddddddddkke | 23 | 13492 | 13507 | 175 |
| 622205 | 2024 | 2039 | ATCTGCTGCTGGCCTT | ekkddddddddddkke | 0 | 13493 | 13508 | 176 |
| 622206 | 2025 | 2040 | TATCTGCTGCTGGCCT | ekkddddddddddkke | 22 | 13494 | 13509 | 177 |
| 622207 | 2026 | 2041 | TTATCTGCTGCTGGCC | ekkddddddddddkke | 16 | 13495 | 13510 | 178 |
| 622208 | 2027 | 2042 | GTTATCTGCTGCTGGC | ekkddddddddddkke | 29 | 13496 | 13511 | 163 |
| 622209 | 2028 | 2043 | TGTTATCTGCTGCTGG | ekkddddddddddkke | 37 | 13497 | 13512 | 179 |
| 622210 | 2029 | 2044 | TTGTTATCTGCTGCTG | ekkddddddddddkke | 44 | 13498 | 13513 | 180 |
| 622211 | 2030 | 2045 | GTTGTTATCTGCTGCT | ekkddddddddddkke | 61 | 13499 | 13514 | 181 |
| 622212 | 2031 | 2046 | GGTTGTTATCTGCTGC | ekkddddddddddkke | 51 | 13500 | 13515 | 182 |
| 622213 | 2032 | 2047 | GGGTTGTTATCTGCTG | ekkddddddddddkke | 44 | 13501 | 13516 | 164 |
| 622214 | 2046 | 2061 | CGCTGATTTGTCCGGG | ekkddddddddddkke | 62 | 13515 | 13530 | 129 |
| 622215 | 2047 | 2062 | TCGCTGATTTGTCCGG | ekkddddddddddkke | 47 | 13516 | 13531 | 165 |
| 622216 | 2048 | 2063 | ATCGCTGATTTGTCCG | ekkddddddddddkke | 55 | 13517 | 13532 | 183 |
| 622217 | 2049 | 2064 | CATCGCTGATTTGTCC | ekkddddddddddkke | 11 | 13518 | 13533 | 166 |
| 622218 | 2050 | 2065 | ACATCGCTGATTTGTC | ekkddddddddddkke | 33 | 13519 | 13534 | 184 |
| 622219 | 2051 | 2066 | CACATCGCTGATTTGT | ekkddddddddddkke | 41 | 13520 | 13535 | 185 |
| 622220 | 2052 | 2067 | ACACATCGCTGATTTG | ekkddddddddddkke | 49 | 13521 | 13536 | 186 |
| 622221 | 2053 | 2068 | GACACATCGCTGATTT | ekkddddddddddkke | 52 | 13522 | 13537 | 167 |
| 622222 | 2054 | 2069 | TGACACATCGCTGATT | ekkddddddddddkke | 34 | 13523 | 13538 | 187 |
| 622223 | 2055 | 2070 | GTGACACATCGCTGAT | ekkddddddddddkke | 32 | 13524 | 13539 | 188 |
| 622224 | 2056 | 2071 | GGTGACACATCGCTGA | ekkddddddddddkke | 45 | 13525 | 13540 | 130 |
| 622225 | 2057 | 2072 | GGGTGACACATCGCTG | ekkddddddddddkke | 58 | 13526 | 13541 | 189 |
| 622226 | 2073 | 2088 | AAAGGTGGGAGACTGG | ekkddddddddddkke | 18 | 13542 | 13557 | 168 |
| 622227 | 2074 | 2089 | AAAAGGTGGGAGACTG | ekkddddddddddkke | 0 | 13543 | 13558 | 190 |
| 622228 | 2075 | 2090 | GAAAAGGTGGGAGACT | ekkddddddddddkke | 0 | 13544 | 13559 | 131 |
| 622229 | 2076 | 2091 | AGAAAAGGTGGGAGAC | ekkddddddddddkke | 0 | 13545 | 13560 | 191 |
| 622230 | 2080 | 2095 | TAGAAGAAAAGGTGGG | ekkddddddddddkke | 12 | 13549 | 13564 | 192 |
| 622231 | 2081 | 2096 | TTAGAAGAAAAGGTGG | ekkddddddddddkke | 22 | 13550 | 13565 | 193 |
| 622232 | 2082 | 2097 | ATTAGAAGAAAAGGTG | ekkddddddddddkke | 7 | 13551 | 13566 | 169 |
| 622233 | 2083 | 2098 | CATTAGAAGAAAAGGT | ekkddddddddddkke | 0 | 13552 | 13567 | 194 |
| 622234 | 2084 | 2099 | TCATTAGAAGAAAAGG | ekkddddddddddkke | 20 | 13553 | 13568 | 195 |
| 622235 | 2085 | 2100 | CTCATTAGAAGAAAAG | ekkddddddddddkke | 4 | 13554 | 13569 | 196 |
| 622236 | 2086 | 2101 | ACTCATTAGAAGAAAA | ekkddddddddddkke | 0 | 13555 | 13570 | 197 |
| 622237 | 2087 | 2102 | GACTCATTAGAAGAAA | ekkddddddddddkke | 22 | 13556 | 13571 | 198 |
| 622238 | 2088 | 2103 | CGACTCATTAGAAGAA | ekkddddddddddkke | 46 | 13557 | 13572 | 132 |
| 622239 | 2089 | 2104 | TCGACTCATTAGAAGA | ekkddddddddddkke | 33 | 13558 | 13573 | 199 |
| 622240 | 2090 | 2105 | GTCGACTCATTAGAAG | ekkddddddddddkke | 6 | 13559 | 13574 | 170 |
| 622241 | 2091 | 2106 | AGTCGACTCATTAGAA | ekkddddddddddkke | 33 | 13560 | 13575 | 200 |
| 622242 | 2092 | 2107 | AAGTCGACTCATTAGA | ekkddddddddddkke | 31 | 13561 | 13576 | 201 |
| 622243 | 2093 | 2108 | AAAGTCGACTCATTAG | ekkddddddddddkke | 16 | 13562 | 13577 | 202 |
| 622244 | 2094 | 2109 | CAAAGTCGACTCATTA | ekkddddddddddkke | 28 | 13563 | 13578 | 203 |
| 622245 | 2095 | 2110 | TCAAAGTCGACTCATT | ekkddddddddddkke | 16 | 13564 | 13579 | 171 |
| 622246 | 2096 | 2111 | CTCAAAGTCGACTCAT | ekkddddddddddkke | 25 | 13565 | 13580 | 204 |
| 622247 | 2097 | 2112 | GCTCAAAGTCGACTCA | ekkddddddddddkke | 43 | 13566 | 13581 | 205 |
| 622248 | 2098 | 2113 | AGCTCAAAGTCGACTC | ekkddddddddddkke | 39 | 13567 | 13582 | 206 |
| 622249 | 2099 | 2114 | CAGCTCAAAGTCGACT | ekkddddddddddkke | 17 | 13568 | 13583 | 172 |

Table 12 shows the percent inhibition of AGT mRNA by antisense oligonucleotides. Cultured HepG2 cells at a density of about 20,000 cells per well were transfected using electroporation with 3,000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 12
Inhibition of AGT mRNA by MOE and/or cEt containing
gapmers targeting SEQ ID NO: 1 and/or 2

| | SEQ | SEQ | | | | SEQ | SEQ | |
|---|---|---|---|---|---|---|---|---|
| | ID: 1 | ID: 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568637 | 2046 | 2061 | CGCTGATTTGTCCGGG | eekddddddddddkke | 88 | 13515 | 13530 | 129 |
| 610006 | 2023 | 2042 | GTTATCTGCTGCTGGCCTTT | eeeeeddddddddddeeeee | 60 | 13492 | 13511 | 230 |
| 610009 | 2026 | 2045 | GTTGTTATCTGCTGCTGGCC | eeeeeddddddddddeeeee | 38 | 13495 | 13514 | 233 |
| 610010 | 2027 | 2046 | GGTTGTTATCTGCTGCTGGC | eeeeeddddddddddeeeee | 66 | 13496 | 13515 | 234 |
| 610015 | 2049 | 2068 | GACACATCGCTGATTTGTCC | eeeeeddddddddddeeeee | 72 | 13518 | 13537 | 239 |
| 610015 | 2049 | 2068 | GACACATCGCTGATTTGTCC | eeeeeddddddddddeeeee | 25 | 13518 | 13537 | 239 |
| 654354 | 636 | 655 | ACTCTCATTGTGGATGACGA | eeeeeddddddddddeeeee | 41 | 5853 | 5872 | 1796 |
| 654355 | 640 | 659 | AGGTACTCTCATTGTGGATG | eeeeeddddddddddeeeee | 26 | 5857 | 5876 | 1797 |
| 654356 | 642 | 661 | ACAGGTACTCTCATTGTGGA | eeeeeddddddddddeeeee | 16 | 5859 | 5878 | 1798 |
| 654357 | 646 | 665 | GCTCACAGGTACTCTCATTG | eeeeeddddddddddeeeee | 18 | 5863 | 5882 | 1799 |
| 654358 | 757 | 776 | GCACCAGCTGGTCCTGTAGG | eeeeeddddddddddeeeee | 10 | 5974 | 5993 | 1800 |
| 654359 | 759 | 778 | TAGCACCAGCTGGTCCTGTA | eeeeeddddddddddeeeee | 24 | 5976 | 5995 | 1801 |
| 654360 | 760 | 779 | CTAGCACCAGCTGGTCCTGT | eeeeeddddddddddeeeee | 13 | 5977 | 5996 | 1802 |
| 654361 | 763 | 782 | CGACTAGCACCAGCTGGTCC | eeeeeddddddddddeeeee | 1 | 5980 | 5999 | 1803 |
| 654362 | 765 | 784 | AGCGACTAGCACCAGCTGGT | eeeeeddddddddddeeeee | 28 | 5982 | 6001 | 1804 |
| 654363 | 769 | 788 | TTGCAGCGACTAGCACCAGC | eeeeeddddddddddeeeee | 18 | 5986 | 6005 | 1805 |
| 654364 | 771 | 790 | TTTTGCAGCGACTAGCACCA | eeeeeddddddddddeeeee | 9 | 5988 | 6007 | 1806 |
| 654365 | 775 | 794 | CAAGTTTTGCAGCGACTAGC | eeeeeddddddddddeeeee | 0 | 5992 | 6011 | 1807 |
| 654366 | 1267 | 1286 | CTGTCACAGCCTGCATGAAC | eeeeeddddddddddeeeee | 15 | 6484 | 6503 | 1808 |
| 654367 | 1269 | 1288 | TCCTGTCACAGCCTGCATGA | eeeeeddddddddddeeeee | 34 | 6486 | 6505 | 1809 |
| 654368 | 1270 | 1289 | ATCCTGTCACAGCCTGCATG | eeeeeddddddddddeeeee | 34 | 6487 | 6506 | 1810 |
| 654369 | 1273 | 1292 | TCCATCCTGTCACAGCCTGC | eeeeeddddddddddeeeee | 32 | 6490 | 6509 | 1811 |
| 654370 | 1275 | 1294 | CTTCCATCCTGTCACAGCCT | eeeeeddddddddddeeeee | 50 | 6492 | 6511 | 1812 |
| 654371 | 1460 | 1479 | TCACTCCAGTGCTGGAAGGT | eeeeeddddddddddeeeee | 0 | 10471 | 10490 | 1813 |
| 654372 | 1462 | 1481 | TGTCACTCCAGTGCTGGAAG | eeeeeddddddddddeeeee | 18 | 10473 | 10492 | 1814 |
| 654373 | 1463 | 1482 | ATGTCACTCCAGTGCTGGAA | eeeeeddddddddddeeeee | 6 | 10474 | 10493 | 1815 |
| 654374 | 1466 | 1485 | TGGATGTCACTCCAGTGCTG | eeeeeddddddddddeeeee | 26 | 10477 | 10496 | 1816 |
| 654375 | 1468 | 1487 | CCTGGATGTCACTCCAGTGC | eeeeeddddddddddeeeee | 20 | 10479 | 10498 | 1817 |
| 654376 | 2115 | 2134 | AAGGAGAAACGGCTGCTTTC | eeeeeddddddddddeeeee | 19 | 13584 | 13603 | 1818 |
| 654377 | 2116 | 2135 | CAAGGAGAAACGGCTGCTTT | eeeeeddddddddddeeeee | 40 | 13585 | 13604 | 1819 |
| 654378 | 2118 | 2137 | ACCAAGGAGAAACGGCTGCT | eeeeeddddddddddeeeee | 48 | 13587 | 13606 | 1820 |
| 654379 | 2119 | 2138 | GACCAAGGAGAAACGGCTGC | eeeeeddddddddddeeeee | 57 | 13588 | 13607 | 1821 |
| 654380 | 2121 | 2140 | TAGACCAAGGAGAAACGGCT | eeeeeddddddddddeeeee | 46 | 13590 | 13609 | 1822 |
| 654381 | 2122 | 2141 | TTAGACCAAGGAGAAACGGC | eeeeeddddddddddeeeee | 32 | 13591 | 13610 | 1823 |
| 654382 | 2124 | 2143 | ACTTAGACCAAGGAGAAACG | eeeeeddddddddddeeeee | 42 | 13593 | 13612 | 1824 |
| 654383 | 2125 | 2144 | CACTTAGACCAAGGAGAAAC | eeeeeddddddddddeeeee | 29 | 13594 | 13613 | 1825 |
| 654384 | 2127 | 2146 | CACACTTAGACCAAGGAGAA | eeeeeddddddddddeeeee | 21 | 13596 | 13615 | 1826 |
| 654385 | 2128 | 2147 | GCACACTTAGACCAAGGAGA | eeeeeddddddddddeeeee | 65 | 13597 | 13616 | 1827 |
| 654386 | 2130 | 2149 | CAGCACACTTAGACCAAGGA | eeeeeddddddddddeeeee | 39 | 13599 | 13618 | 1828 |
| 654387 | 2131 | 2150 | GCAGCACACTTAGACCAAGG | eeeeeddddddddddeeeee | 39 | 13600 | 13619 | 1829 |
| 654388 | 2133 | 2152 | ATGCAGCACACTTAGACCAA | eeeeeddddddddddeeeee | 27 | 13602 | 13621 | 1830 |
| 654389 | 2134 | 2153 | CATGCAGCACACTTAGACCA | eeeeeddddddddddeeeee | 26 | 13603 | 13622 | 1831 |
| 654390 | 2136 | 2155 | TCCATGCAGCACACTTAGAC | eeeeeddddddddddeeeee | 2 | 13605 | 13624 | 1832 |
| 654391 | 2137 | 2156 | CTCCATGCAGCACACTTAGA | eeeeeddddddddddeeeee | 48 | 13606 | 13625 | 1833 |
| 654392 | 2139 | 2158 | CACTCCATGCAGCACACTTA | eeeeeddddddddddeeeee | 60 | 13608 | 13627 | 1834 |
| 654393 | 2140 | 2159 | TCACTCCATGCAGCACACTT | eeeeeddddddddddeeeee | 45 | 13609 | 13628 | 1835 |
| 654394 | 2142 | 2161 | GCTCACTCCATGCAGCACAC | eeeeeddddddddddeeeee | 72 | 13611 | 13630 | 1836 |
| 654395 | 2160 | 2179 | CCGCTGCAGGCTTCTACTGC | eeeeeddddddddddeeeee | 34 | 13629 | 13648 | 1837 |
| 654396 | 2161 | 2180 | GCCGCTGCAGGCTTCTACTG | eeeeeddddddddddeeeee | 32 | 13630 | 13649 | 1838 |
| 654397 | 2163 | 2182 | GTGCCGCTGCAGGCTTCTAC | eeeeeddddddddddeeeee | 38 | 13632 | 13651 | 1839 |
| 654398 | 2164 | 2183 | TGTGCCGCTGCAGGCTTCTA | eeeeeddddddddddeeeee | 17 | 13633 | 13652 | 1840 |
| 654399 | 2166 | 2185 | TTTGTGCCGCTGCAGGCTTC | eeeeeddddddddddeeeee | 16 | 13635 | 13654 | 1841 |
| 654400 | 2167 | 2186 | ATTTGTGCCGCTGCAGGCTT | eeeeeddddddddddeeeee | 27 | 13636 | 13655 | 1842 |
| 654401 | 2169 | 2188 | GCATTTGTGCCGCTGCAGGC | eeeeeddddddddddeeeee | 75 | 13638 | 13657 | 1843 |
| 654402 | 2170 | 2189 | TGCATTTGTGCCGCTGCAGG | eeeeeddddddddddeeeee | 64 | 13639 | 13658 | 1844 |
| 654403 | 2172 | 2191 | GGTGCATTTGTGCCGCTGCA | eeeeeddddddddddeeeee | 64 | 13641 | 13660 | 1845 |
| 654404 | 2173 | 2192 | AGGTGCATTTGTGCCGCTGC | eeeeeddddddddddeeeee | 68 | 13642 | 13661 | 1846 |
| 654405 | 2175 | 2194 | GGAGGTGCATTTGTGCCGCT | eeeeeddddddddddeeeee | 42 | 13644 | 13663 | 1847 |
| 654406 | 2176 | 2195 | GGGAGGTGCATTTGTGCCGC | eeeeeddddddddddeeeee | 36 | 13645 | 13664 | 1848 |
| 654407 | 2178 | 2197 | CTGGGAGGTGCATTTGTGCC | eeeeeddddddddddeeeee | 26 | 13647 | 13666 | 1849 |
| 654408 | 2179 | 2198 | ACTGGGAGGTGCATTTGTGC | eeeeeddddddddddeeeee | 10 | 13648 | 13667 | 1850 |
| 654409 | 2181 | 2200 | AAACTGGGAGGTGCATTTGT | eeeeeddddddddddeeeee | 15 | 13650 | 13669 | 1851 |
| 654410 | 2182 | 2201 | CAAACTGGGAGGTGCATTTG | eeeeeddddddddddeeeee | 7 | 13651 | 13670 | 1852 |
| 654411 | 2184 | 2203 | AGCAAACTGGGAGGTGCATT | eeeeeddddddddddeeeee | 34 | 13653 | 13672 | 1853 |
| 654412 | 2185 | 2204 | CAGCAAACTGGGAGGTGCAT | eeeeeddddddddddeeeee | 33 | 13654 | 13673 | 1854 |
| 654413 | 2187 | 2206 | CCCAGCAAACTGGGAGGTGC | eeeeeddddddddddeeeee | 57 | 13656 | 13675 | 1855 |
| 654414 | 2188 | 2207 | ACCCAGCAAACTGGGAGGTG | eeeeeddddddddddeeeee | 53 | 13657 | 13676 | 1856 |
| 654415 | 2193 | 2212 | AATAAACCCAGCAAACTGGG | eeeeeddddddddddeeeee | 17 | 13662 | 13681 | 1857 |
| 654416 | 2194 | 2213 | AAATAAACCCAGCAAACTGG | eeeeeddddddddddeeeee | 20 | 13663 | 13682 | 1858 |
| 654417 | 2196 | 2215 | TAAAATAAACCCAGCAAACT | eeeeeddddddddddeeeee | 13 | 13665 | 13684 | 1859 |
| 654418 | 2197 | 2216 | CTAAAATAAACCCAGCAAAC | eeeeeddddddddddeeeee | 2 | 13666 | 13685 | 1860 |
| 654419 | 2199 | 2218 | CTCTAAAATAAACCCAGCAA | eeeeeddddddddddeeeee | 12 | 13668 | 13687 | 1861 |
| 654420 | 2200 | 2219 | TCTCTAAAATAAACCCAGCA | eeeeeddddddddddeeeee | 47 | 13669 | 13688 | 1862 |
| 654421 | 2202 | 2221 | ATTCTCTAAAATAAACCCAG | eeeeeddddddddddeeeee | 23 | 13671 | 13690 | 1863 |
| 654422 | 2203 | 2222 | CATTCTCTAAAATAAACCCA | eeeeeddddddddddeeeee | 22 | 13672 | 13691 | 1864 |
| 654423 | 2205 | 2224 | CCCATTCTCTAAAATAAACC | eeeeeddddddddddeeeee | 12 | 13674 | 13693 | 1865 |
| 654424 | 2206 | 2225 | CCCCATTCTCTAAAATAAAC | eeeeeddddddddddeeeee | 20 | 13675 | 13694 | 1866 |
| 654425 | 2208 | 2227 | ACCCCCATTCTCTAAAATAA | eeeeeddddddddddeeeee | 21 | 13677 | 13696 | 1867 |
| 654426 | 2209 | 2228 | CACCCCCATTCTCTAAAATA | eeeeeddddddddddeeeee | 32 | 13678 | 13697 | 1868 |
| 654427 | 2211 | 2230 | CCCACCCCCATTCTCTAAAA | eeeeeddddddddddeeeee | 18 | 13680 | 13699 | 1869 |

Table 13 shows inhibition of AGT mRNA in HepG2 cells cultured at a density of 20,000 cells per well which were transfected using electroporation with 4000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 13
Inhibition of AGT mRNA by MOE and/or cEt containing
gapmers targeting SEQ ID NO: 1 and/or 2

| | SEQ | SEQ | | | | SEQ | SEQ | |
|---|---|---|---|---|---|---|---|---|
| | ID: 1 | ID: 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |
| 568637 | 2046 | 2061 | CGCTGATTTGTCCGGG | eekddddddddddkke | 95 | 13515 | 13530 | 129 |
| 568637 | 2046 | 2061 | CGCTGATTTGTCCGGG | eekddddddddddkke | 98 | 13515 | 13530 | 129 |
| 610012 | 2046 | 2065 | ACATCGCTGATTTGTCCGGG | eeeeeddddddddddeeeee | 74 | 13515 | 13534 | 236 |
| 610013 | 2047 | 2066 | CACATCGCTGATTTGTCCGG | eeeeeddddddddddeeeee | 76 | 13516 | 13535 | 237 |
| 610014 | 2048 | 2067 | ACACATCGCTGATTTGTCCG | eeeeeddddddddddeeeee | 85 | 13517 | 13536 | 238 |
| 610015 | 2049 | 2068 | GACACATCGCTGATTTGTCC | eeeeeddddddddddeeeee | 85 | 13518 | 13537 | 239 |
| 610015 | 2049 | 2068 | GACACATCGCTGATTTGTCC | eeeeeddddddddddeeeee | 3 | 13518 | 13537 | 239 |
| 610015 | 2049 | 2068 | GACACATCGCTGATTTGTCC | eeeeeddddddddddeeeee | 17 | 13518 | 13537 | 239 |
| 610043 | 2096 | 2115 | CCAGCTCAAAGTCGACTCAT | eeeeeddddddddddeeeee | 87 | 13565 | 13584 | 267 |
| 619998 | 2267 | 2286 | TCGGTTGGAATTCTTTTTGG | eeeeeddddddddddeeeee | 80 | 13736 | 13755 | 1714 |
| 620000 | 2273 | 2292 | AGCTGGTCGGTTGGAATTCT | eeeeeddddddddddeeeee | 69 | 13742 | 13761 | 1716 |
| 654428 | 2212 | 2231 | CCCCACCCCCATTCTCTAAA | eeeeeddddddddddeeeee | 49 | 13681 | 13700 | 1870 |
| 654429 | 2214 | 2233 | CTCCCCACCCCCATTCTCTA | eeeeeddddddddddeeeee | 58 | 13683 | 13702 | 1871 |
| 654430 | 2217 | 2236 | TGCCTCCCCACCCCCATTCT | eeeeeddddddddddeeeee | 61 | 13686 | 13705 | 1872 |
| 654431 | 2218 | 2237 | TTGCCTCCCCACCCCCATTC | eeeeeddddddddddeeeee | 51 | 13687 | 13706 | 1873 |
| 654432 | 2220 | 2239 | TCTTGCCTCCCCACCCCCAT | eeeeeddddddddddeeeee | 67 | 13689 | 13708 | 1874 |
| 654433 | 2223 | 2242 | GGTTCTTGCCTCCCCACCCC | eeeeeddddddddddeeeee | 84 | 13692 | 13711 | 1875 |
| 654434 | 2224 | 2243 | TGGTTCTTGCCTCCCCACCC | eeeeeddddddddddeeeee | 83 | 13693 | 13712 | 1876 |
| 654435 | 2226 | 2245 | ACTGGTTCTTGCCTCCCCAC | eeeeeddddddddddeeeee | 75 | 13695 | 13714 | 1877 |
| 654436 | 2227 | 2246 | CACTGGTTCTTGCCTCCCCA | eeeeeddddddddddeeeee | 84 | 13696 | 13715 | 1878 |
| 654437 | 2229 | 2248 | AACACTGGTTCTTGCCTCCC | eeeeeddddddddddeeeee | 76 | 13698 | 13717 | 1879 |
| 654438 | 2230 | 2249 | AAACACTGGTTCTTGCCTCC | eeeeeddddddddddeeeee | 75 | 13699 | 13718 | 1880 |
| 654439 | 2232 | 2251 | CTAAACACTGGTTCTTGCCT | eeeeeddddddddddeeeee | 70 | 13701 | 13720 | 1881 |
| 654440 | 2233 | 2252 | GCTAAACACTGGTTCTTGCC | eeeeeddddddddddeeeee | 79 | 13702 | 13721 | 1882 |
| 654441 | 2235 | 2254 | GCGCTAAACACTGGTTCTTG | eeeeeddddddddddeeeee | 79 | 13704 | 13723 | 1883 |
| 654442 | 2236 | 2255 | CGCGCTAAACACTGGTTCTT | eeeeeddddddddddeeeee | 81 | 13705 | 13724 | 1884 |
| 654443 | 2238 | 2257 | CCCGCGCTAAACACTGGTTC | eeeeeddddddddddeeeee | 80 | 13707 | 13726 | 1885 |
| 654444 | 2239 | 2258 | TCCCGCGCTAAACACTGGTT | eeeeeddddddddddeeeee | 89 | 13708 | 13727 | 1886 |
| 654445 | 2241 | 2260 | AGTCCCGCGCTAAACACTGG | eeeeeddddddddddeeeee | 75 | 13710 | 13729 | 1887 |
| 654446 | 2242 | 2261 | TAGTCCCGCGCTAAACACTG | eeeeeddddddddddeeeee | 73 | 13711 | 13730 | 1888 |
| 654447 | 2244 | 2263 | AGTAGTCCCGCGCTAAACAC | eeeeeddddddddddeeeee | 59 | 13713 | 13732 | 1889 |
| 654448 | 2245 | 2264 | CAGTAGTCCCGCGCTAAACA | eeeeeddddddddddeeeee | 67 | 13714 | 13733 | 1890 |
| 654449 | 2247 | 2266 | AACAGTAGTCCCGCGCTAAA | eeeeeddddddddddeeeee | 60 | 13716 | 13735 | 1891 |
| 654450 | 2248 | 2267 | GAACAGTAGTCCCGCGCTAA | eeeeeddddddddddeeeee | 69 | 13717 | 13736 | 1892 |
| 654451 | 2250 | 2269 | TGGAACAGTAGTCCCGCGCT | eeeeeddddddddddeeeee | 87 | 13719 | 13738 | 1893 |
| 654452 | 2251 | 2270 | TTGGAACAGTAGTCCCGCGC | eeeeeddddddddddeeeee | 87 | 13720 | 13739 | 1894 |
| 654453 | 2253 | 2272 | TTTTGGAACAGTAGTCCCGC | eeeeeddddddddddeeeee | 73 | 13722 | 13741 | 1895 |
| 654454 | 2254 | 2273 | TTTTTGGAACAGTAGTCCCG | eeeeeddddddddddeeeee | 51 | 13723 | 13742 | 1896 |
| 654455 | 2256 | 2275 | TCTTTTTGGAACAGTAGTCC | eeeeeddddddddddeeeee | 74 | 13725 | 13744 | 1897 |
| 654456 | 2257 | 2276 | TTCTTTTTGGAACAGTAGTC | eeeeeddddddddddeeeee | 66 | 13726 | 13745 | 1898 |
| 654457 | 2259 | 2278 | AATTCTTTTTGGAACAGTAG | eeeeeddddddddddeeeee | 46 | 13728 | 13747 | 1899 |
| 654458 | 2260 | 2279 | GAATTCTTTTTGGAACAGTA | eeeeeddddddddddeeeee | 74 | 13729 | 13748 | 1900 |
| 654459 | 2262 | 2281 | TGGAATTCTTTTTGGAACAG | eeeeeddddddddddeeeee | 41 | 13731 | 13750 | 1901 |
| 654460 | 2263 | 2282 | TTGGAATTCTTTTTGGAACA | eeeeeddddddddddeeeee | 34 | 13732 | 13751 | 1902 |
| 654461 | 2265 | 2284 | GGTTGGAATTCTTTTTGGAA | eeeeeddddddddddeeeee | 58 | 13734 | 13753 | 1903 |
| 654462 | 2266 | 2285 | CGGTTGGAATTCTTTTTGGA | eeeeeddddddddddeeeee | 77 | 13735 | 13754 | 1904 |
| 654463 | 2268 | 2287 | GTCGGTTGGAATTCTTTTTG | eeeeeddddddddddeeeee | 74 | 13737 | 13756 | 1905 |
| 654464 | 2269 | 2288 | GGTCGGTTGGAATTCTTTTT | eeeeeddddddddddeeeee | 81 | 13738 | 13757 | 1906 |
| 654465 | 2271 | 2290 | CTGGTCGGTTGGAATTCTTT | eeeeeddddddddddeeeee | 78 | 13740 | 13759 | 1907 |
| 654466 | 2272 | 2291 | GCTGGTCGGTTGGAATTCTT | eeeeeddddddddddeeeee | 81 | 13741 | 13760 | 1908 |
| 654467 | 2274 | 2293 | AAGCTGGTCGGTTGGAATTC | eeeeeddddddddddeeeee | 61 | 13743 | 13762 | 1909 |
| 654468 | 2275 | 2294 | CAAGCTGGTCGGTTGGAATT | eeeeeddddddddddeeeee | 62 | 13744 | 13763 | 1910 |
| 654469 | 2277 | 2296 | AACAAGCTGGTCGGTTGGAA | eeeeeddddddddddeeeee | 70 | 13746 | 13765 | 1911 |
| 654470 | 2278 | 2297 | AAACAAGCTGGTCGGTTGGA | eeeeeddddddddddeeeee | 62 | 13747 | 13766 | 1912 |
| 654471 | 2280 | 2299 | ACAAACAAGCTGGTCGGTTG | eeeeeddddddddddeeeee | 62 | 13749 | 13768 | 1913 |
| 654472 | 2281 | 2300 | CACAAACAAGCTGGTCGGTT | eeeeeddddddddddeeeee | 88 | 13750 | 13769 | 1914 |
| 654473 | 2283 | 2302 | TTCACAAACAAGCTGGTCGG | eeeeeddddddddddeeeee | 76 | 13752 | 13771 | 1915 |
| 654474 | 2284 | 2303 | TTTCACAAACAAGCTGGTCG | eeeeeddddddddddeeeee | 77 | 13753 | 13772 | 1916 |
| 654475 | 2286 | 2305 | TGTTTCACAAACAAGCTGGT | eeeeeddddddddddeeeee | 80 | 13755 | 13774 | 1917 |
| 654476 | 2287 | 2306 | TTGTTTCACAAACAAGCTGG | eeeeeddddddddddeeeee | 83 | 13756 | 13775 | 1918 |
| 654477 | 2289 | 2308 | TTTTGTTTCACAAACAAGCT | eeeeeddddddddddeeeee | 66 | 13758 | 13777 | 1919 |
| 654478 | 2290 | 2309 | TTTTTGTTTCACAAACAAGC | eeeeeddddddddddeeeee | 70 | 13759 | 13778 | 1920 |
| 654479 | 2309 | 2328 | AACTTGAAAAGGGAACACTT | eeeeeddddddddddeeeee | 69 | 13778 | 13797 | 1921 |
| 654480 | 2311 | 2330 | TCAACTTGAAAAGGGAACAC | eeeeeddddddddddeeeee | 84 | 13780 | 13799 | 1922 |
| 654481 | 2312 | 2331 | CTCAACTTGAAAAGGGAACA | eeeeeddddddddddeeeee | 90 | 13781 | 13800 | 1923 |
| 654482 | 2314 | 2333 | TTCTCAACTTGAAAAGGGAA | eeeeeddddddddddeeeee | 67 | 13783 | 13802 | 1924 |
| 654483 | 2315 | 2334 | GTTCTCAACTTGAAAAGGGA | eeeeeddddddddddeeeee | 92 | 13784 | 13803 | 1925 |
| 654484 | 2317 | 2336 | TTGTTCTCAACTTGAAAAGG | eeeeeddddddddddeeeee | 82 | 13786 | 13805 | 1926 |
| 654485 | 2318 | 2337 | TTTGTTCTCAACTTGAAAAG | eeeeeddddddddddeeeee | 61 | 13787 | 13806 | 1927 |
| 654486 | 2320 | 2339 | TTTTTGTTCTCAACTTGAAA | eeeeeddddddddddeeeee | 35 | 13789 | 13808 | 1928 |
| 654487 | 2321 | 2340 | ATTTTTGTTCTCAACTTGAA | eeeeeddddddddddeeeee | 44 | 13790 | 13809 | 1929 |
| 654488 | 2323 | 2342 | CAATTTTTGTTCTCAACTTG | eeeeeddddddddddeeeee | 54 | 13792 | 13811 | 1930 |
| 654489 | 2324 | 2343 | CCAATTTTTGTTCTCAACTT | eeeeeddddddddddeeeee | 79 | 13793 | 13812 | 1931 |
| 654490 | 2326 | 2345 | ACCCAATTTTTGTTCTCAAC | eeeeeddddddddddeeeee | 85 | 13795 | 13814 | 1932 |
| 654491 | 2327 | 2346 | AACCCAATTTTTGTTCTCAA | eeeeeddddddddddeeeee | 82 | 13796 | 13815 | 1933 |
| 654492 | 2330 | 2349 | TAAAACCCAATTTTTGTTCT | eeeeeddddddddddeeeee | 52 | 13799 | 13818 | 1934 |
| 654493 | 2332 | 2351 | TTTAAAACCCAATTTTTGTT | eeeeeddddddddddeeeee | 13 | 13801 | 13820 | 1935 |
| 654494 | 2355 | 2374 | AATGCAAAAATGTATACTTT | eeeeeddddddddddeeeee | 53 | 13824 | 13843 | 1936 |
| 654495 | 2357 | 2376 | GCAATGCAAAAATGTATACT | eeeeeddddddddddeeeee | 73 | 13826 | 13845 | 1937 |
| 654496 | 2360 | 2379 | AAGGCAATGCAAAAATGTAT | eeeeeddddddddddeeeee | 56 | 13829 | 13848 | 1938 |
| 654497 | 2361 | 2380 | GAAGGCAATGCAAAAATGTA | eeeeeddddddddddeeeee | 70 | 13830 | 13849 | 1939 |
| 654498 | 2363 | 2382 | CCGAAGGCAATGCAAAAATG | eeeeeddddddddddeeeee | 60 | 13832 | 13851 | 1940 |
| 654521 | 495 | 511 | CATACCCTTCTGCTGTA | eeeddddddddddeeee | 46 | N/A | N/A | 1941 |
| 654522 | 498 | 514 | CCGCATACCCTTCTGCT | eeeddddddddddeeee | 44 | N/A | N/A | 1942 |
| 654523 | 504 | 520 | TCGCTTCCGCATACCCT | eeeddddddddddeeee | 69 | 5721 | 5737 | 1943 |
| 654524 | 507 | 523 | TGCTCGCTTCCGCATAC | eeeddddddddddeeee | 58 | 5724 | 5740 | 1944 |
| 654525 | 636 | 652 | CTCATTGTGGATGACGA | eeeddddddddddeeee | 53 | 5853 | 5869 | 1945 |
| 654526 | 639 | 655 | ACTCTCATTGTGGATGA | eeeddddddddddeeee | 48 | 5856 | 5872 | 1946 |
| 654527 | 654 | 670 | CAGCTGCTCACAGGTAC | eeeddddddddddeeee | 47 | 5871 | 5887 | 1947 |
| 654528 | 768 | 784 | AGCGACTAGCACCAGCT | eeeddddddddddeeee | 56 | 5985 | 6001 | 1948 |
| 654529 | 1266 | 1282 | CACAGCCTGCATGAACC | eeeddddddddddeeee | 48 | 6483 | 6499 | 1949 |
| 654530 | 1272 | 1288 | TCCTGTCACAGCCTGCA | eeeddddddddddeeee | 68 | 6489 | 6505 | 1950 |
| 654531 | 1275 | 1291 | CCATCCTGTCACAGCCT | eeeddddddddddeeee | 65 | 6492 | 6508 | 1951 |
| 654532 | 1456 | 1472 | AGTGCTGGAAGGTGCCC | eeeddddddddddeeee | 41 | 10467 | 10483 | 1952 |
| 654533 | 1531 | 1547 | GCTGGATCAGCAGCAGG | eeeddddddddddeeee | 61 | 10542 | 10558 | 1953 |
| 654534 | 1751 | 1767 | CTGATGCGGTCATTGCT | eeeddddddddddeeee | 52 | 12357 | 12373 | 1954 |
| 654535 | 1808 | 1824 | GGCTCTCTCTCATCCGC | eeeddddddddddeeee | 74 | 13277 | 13293 | 1955 |
| 654536 | 1811 | 1827 | GTGGGCTCTCTCTCATC | eeeddddddddddeeee | 60 | 13280 | 13296 | 1956 |
| 654537 | 1983 | 1999 | CCTTGCCAGGCACTGTG | eeeddddddddddeeee | 69 | 13452 | 13468 | 1957 |
| 654538 | 1984 | 2000 | GCCTTGCCAGGCACTGT | eeeddddddddddeeee | 77 | 13453 | 13469 | 1958 |
| 654539 | 1986 | 2002 | AGGCCTTGCCAGGCACT | eeeddddddddddeeee | 78 | 13455 | 13471 | 1959 |
| 654540 | 1987 | 2003 | GAGGCCTTGCCAGGCAC | eeeddddddddddeeee | 44 | 13456 | 13472 | 1960 |
| 654541 | 2019 | 2035 | GCTGCTGGCCTTTGCCT | eeeddddddddddeeee | 54 | 13488 | 13504 | 1961 |
| 654542 | 2024 | 2040 | TATCTGCTGCTGGCCTT | eeeddddddddddeeee | 59 | 13493 | 13509 | 1962 |
| 654543 | 2025 | 2041 | TTATCTGCTGCTGGCCT | eeeddddddddddeeee | 4 | 13494 | 13510 | 1963 |
| 654544 | 2027 | 2043 | TGTTATCTGCTGCTGGC | eeeddddddddddeeee | 67 | 13496 | 13512 | 1964 |
| 654545 | 2028 | 2044 | TTGTTATCTGCTGCTGG | eeeddddddddddeeee | 56 | 13497 | 13513 | 1965 |
| 654546 | 2029 | 2045 | GTTGTTATCTGCTGCTG | eeeddddddddddeeee | 77 | 13498 | 13514 | 1966 |
| 654547 | 2047 | 2063 | ATCGCTGATTTGTCCGG | eeeddddddddddeeee | 80 | 13516 | 13532 | 1967 |
| 654548 | 2048 | 2064 | CATCGCTGATTTGTCCG | eeeddddddddddeeee | 59 | 13517 | 13533 | 1968 |
| 654549 | 2049 | 2065 | ACATCGCTGATTTGTCC | eeeddddddddddeeee | 65 | 13518 | 13534 | 1969 |
| 654550 | 2050 | 2066 | CACATCGCTGATTTGTC | eeeddddddddddeeee | 81 | 13519 | 13535 | 1970 |
| 654551 | 2051 | 2067 | ACACATCGCTGATTTGT | eeeddddddddddeeee | 74 | 13520 | 13536 | 1971 |
| 654552 | 2053 | 2069 | TGACACATCGCTGATTT | eeeddddddddddeeee | 53 | 13522 | 13538 | 1972 |
| 654553 | 2054 | 2070 | GTGACACATCGCTGATT | eeeddddddddddeeee | 74 | 13523 | 13539 | 1973 |
| 654554 | 2082 | 2098 | CATTAGAAGAAAAGGTG | eeeddddddddddeeee | 18 | 13551 | 13567 | 1974 |
| 654555 | 2083 | 2099 | TCATTAGAAGAAAAGGT | eeeddddddddddeeee | 23 | 13552 | 13568 | 1975 |
| 654556 | 2087 | 2103 | CGACTCATTAGAAGAAA | eeeddddddddddeeee | 51 | 13556 | 13572 | 1976 |
| 654557 | 2096 | 2112 | GCTCAAAGTCGACTCAT | eeeddddddddddeeee | 70 | 13565 | 13581 | 1977 |
| 654558 | 2097 | 2113 | AGCTCAAAGTCGACTCA | eeeddddddddddeeee | 82 | 13566 | 13582 | 1978 |
| 654559 | 2098 | 2114 | CAGCTCAAAGTCGACTC | eeeddddddddddeeee | 88 | 13567 | 13583 | 1979 |
| 654560 | 2099 | 2115 | CCAGCTCAAAGTCGACT | eeeddddddddddeeee | 84 | 13568 | 13584 | 1980 |
| 654561 | 2100 | 2116 | TCCAGCTCAAAGTCGAC | eeeddddddddddeeee | 81 | 13569 | 13585 | 1981 |
| 654562 | 2103 | 2119 | CTTTCCAGCTCAAAGTC | eeeddddddddddeeee | 53 | 13572 | 13588 | 1982 |
| 654563 | 2114 | 2130 | AGAAACGGCTGCTTTCC | eeeddddddddddeeee | 54 | 13583 | 13599 | 1983 |
| 654564 | 2121 | 2137 | ACCAAGGAGAAACGGCT | eeeddddddddddeeee | 66 | 13590 | 13606 | 1984 |
| 654565 | 2172 | 2188 | GCATTTGTGCCGCTGCA | eeeddddddddddeeee | 82 | 13641 | 13657 | 1985 |
| 654566 | 2175 | 2191 | GGTGCATTTGTGCCGCT | eeeddddddddddeeee | 85 | 13644 | 13660 | 1986 |
| 654567 | 2187 | 2203 | AGCAAACTGGGAGGTGC | eeeddddddddddeeee | 70 | 13656 | 13672 | 1987 |
| 654568 | 2226 | 2242 | GGTTCTTGCCTCCCCAC | eeeddddddddddeeee | 88 | 13695 | 13711 | 1988 |
| 654569 | 2235 | 2251 | CTAAACACTGGTTCTTG | eeeddddddddddeeee | 64 | 13704 | 13720 | 1989 |
| 654570 | 2238 | 2254 | GCGCTAAACACTGGTTC | eeeddddddddddeeee | 85 | 13707 | 13723 | 1990 |
| 654571 | 2250 | 2266 | AACAGTAGTCCCGCGCT | eeeddddddddddeeee | 83 | 13719 | 13735 | 1991 |
| 654572 | 2268 | 2284 | GGTTGGAATTCTTTTTG | eeeddddddddddeeee | 38 | 13737 | 13753 | 1992 |
| 654573 | 2274 | 2290 | CTGGTCGGTTGGAATTC | eeeddddddddddeeee | 67 | 13743 | 13759 | 1993 |
| 654574 | 2283 | 2299 | ACAAACAAGCTGGTCGG | eeeddddddddddeeee | 70 | 13752 | 13768 | 1994 |
| 654575 | 2286 | 2302 | TTCACAAACAAGCTGGT | eeeddddddddddeeee | 67 | 13755 | 13771 | 1995 |
| 654576 | 2311 | 2327 | ACTTGAAAAGGGAACAC | eeeddddddddddeeee | 72 | 13780 | 13796 | 1996 |
| 654577 | 2314 | 2330 | TCAACTTGAAAAGGGAA | eeeddddddddddeeee | 29 | 13783 | 13799 | 1997 |
| 654578 | 2317 | 2333 | TTCTCAACTTGAAAAGG | eeeddddddddddeeee | 46 | 13786 | 13802 | 1998 |
| 654579 | 2326 | 2342 | CAATTTTTGTTCTCAAC | eeeddddddddddeeee | 11 | 13795 | 13811 | 1999 |
| 654580 | 2329 | 2345 | ACCCAATTTTTGTTCTC | eeeddddddddddeeee | 70 | 13798 | 13814 | 2000 |
| 654582 | 2024 | 2040 | TATCTGCTGCTGGCCTT | eeeddddddddeeeeee | 58 | 13493 | 13509 | 1962 |
| 654585 | 2027 | 2043 | TGTTATCTGCTGCTGGC | eeeddddddddeeeeee | 66 | 13496 | 13512 | 1964 |
| 654586 | 2028 | 2044 | TTGTTATCTGCTGCTGG | eeeddddddddeeeeee | 66 | 13497 | 13513 | 1965 |
| 654609 | 2024 | 2040 | TATCTGCTGCTGGCCTT | eeeeddddddddeeeee | 62 | 13493 | 13509 | 1962 |
| 654612 | 2027 | 2043 | TGTTATCTGCTGCTGGC | eeeeddddddddeeeee | 61 | 13496 | 13512 | 1964 |
| 654636 | 2024 | 2040 | TATCTGCTGCTGGCCTT | eeeeeddddddddeeee | 70 | 13493 | 13509 | 1962 |
| 654639 | 2027 | 2043 | TGTTATCTGCTGCTGGC | eeeeeddddddddeeee | 69 | 13496 | 13512 | 1964 |
| 654689 | 2023 | 2039 | ATCTGCTGCTGGCCTTT | eeeddddddddddeeee | 60 | 13492 | 13508 | 2001 |
| 654690 | 2026 | 2042 | GTTATCTGCTGCTGGCC | eeeddddddddddeeee | 77 | 13495 | 13511 | 2002 |
| 654691 | 2046 | 2062 | TCGCTGATTTGTCCGGG | eeeddddddddddeeee | 90 | 13515 | 13531 | 2003 |
| 654692 | 2249 | 2265 | ACAGTAGTCCCGCGCTA | eeeddddddddddeeee | 76 | 13718 | 13734 | 2004 |
| 654693 | 2251 | 2267 | GAACAGTAGTCCCGCGC | eeeddddddddddeeee | 74 | 13720 | 13736 | 2005 |
| 654694 | 2267 | 2283 | GTTGGAATTCTTTTTGG | eeeddddddddddeeee | 41 | 13736 | 13752 | 2006 |
| 654695 | 2269 | 2285 | CGGTTGGAATTCTTTTT | eeeddddddddddeeee | 65 | 13738 | 13754 | 2007 |
| 654696 | 2273 | 2289 | TGGTCGGTTGGAATTCT | eeeddddddddddeeee | 61 | 13742 | 13758 | 2008 |
| 654697 | 2275 | 2291 | GCTGGTCGGTTGGAATT | eeeddddddddddeeee | 61 | 13744 | 13760 | 2009 |
| 654698 | 2282 | 2298 | CAAACAAGCTGGTCGGT | eeeddddddddddeeee | 84 | 13751 | 13767 | 2010 |
| 654699 | 2284 | 2300 | CACAAACAAGCTGGTCG | eeeddddddddddeeee | 78 | 13753 | 13769 | 2011 |

Table 14 shows inhibition of AGT mRNA in HepG2 cells cultured at a density of 20,000 cells per well which were transfected using electroporation with 4000 nM antisense oligonucleotide. After a treatment period of approximately 24 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells.

TABLE 14
Inhibition of AGT mRNA by MOE and/or cEt containing
gapmers targeting SEQ ID NO: 1 and/or 2

| | | | | | | | | |
|---|---|---|---|---|---|---|---|---|
| | SEQ | SEQ | | | | SEQ | SEQ | |
| | ID: 1 | ID: 1 | | | | ID: 2 | ID 2: | SEQ |
| ISIS | Start | Stop | | | % | Start | Stop | ID |
| NO | Site | Site | Sequence | Chemistry | Inhibition | Site | Site | NO |

| | | | | | | | | |
|---|---|---|---|---|---|---|---|---|
| 568637 | 2046 | 2061 | CGCTGATTTGTCCGGG | eekddddddddddkke | 96 | 13515 | 13530 | 129 |
| 568637 | 2046 | 2061 | CGCTGATTTGTCCGGG | eekddddddddddkke | 98 | 13515 | 13530 | 129 |
| 568637 | 2046 | 2061 | CGCTGATTTGTCCGGG | eekddddddddddkke | 97 | 13515 | 13530 | 129 |
| 610006 | 2023 | 2042 | GTTATCTGCTGCTGGCCTTT | eeeeeddddddddddeeeee | 77 | 13492 | 13511 | 230 |
| 610009 | 2026 | 2045 | GTTGTTATCTGCTGCTGGCC | eeeeeddddddddddeeeee | 73 | 13495 | 13514 | 233 |
| 610010 | 2027 | 2046 | GGTTGTTATCTGCTGCTGGC | eeeeeddddddddddeeeee | 82 | 13496 | 13515 | 234 |
| 610012 | 2046 | 2065 | ACATCGCTGATTTGTCCGGG | eeeeeddddddddddeeeee | 84 | 13515 | 13534 | 236 |
| 610013 | 2047 | 2066 | CACATCGCTGATTTGTCCGG | eeeeeddddddddddeeeee | 76 | 13516 | 13535 | 237 |
| 610014 | 2048 | 2067 | ACACATCGCTGATTTGTCCG | eeeeeddddddddddeeeee | 89 | 13517 | 13536 | 238 |
| 610015 | 2049 | 2068 | GACACATCGCTGATTTGTCC | eeeeeddddddddddeeeee | 35 | 13518 | 13537 | 239 |
| 610015 | 2049 | 2068 | GACACATCGCTGATTTGTCC | eeeeeddddddddddeeeee | 28 | 13518 | 13537 | 239 |
| 610015 | 2049 | 2068 | GACACATCGCTGATTTGTCC | eeeeeddddddddddeeeee | 89 | 13518 | 13537 | 239 |
| 610015 | 2049 | 2068 | GACACATCGCTGATTTGTCC | eeeeeddddddddddeeeee | 22 | 13518 | 13537 | 239 |
| 610043 | 2096 | 2115 | CCAGCTCAAAGTCGACTCAT | eeeeeddddddddddeeeee | 82 | 13565 | 13584 | 267 |
| 619992 | 2249 | 2268 | GGAACAGTAGTCCCGCGCTA | eeeeeddddddddddeeeee | 88 | 13718 | 13737 | 1708 |
| 619998 | 2267 | 2286 | TCGGTTGGAATTCTTTTTGG | eeeeeddddddddddeeeee | 69 | 13736 | 13755 | 1714 |
| 620000 | 2273 | 2292 | AGCTGGTCGGTTGGAATTCT | eeeeeddddddddddeeeee | 76 | 13742 | 13761 | 1716 |
| 620003 | 2282 | 2301 | TCACAAACAAGCTGGTCGGT | eeeeeddddddddddeeeee | 85 | 13751 | 13770 | 1719 |
| 654701 | 2024 | 2041 | TTATCTGCTGCTGGCCTT | eeeeddddddddddeeee | 50 | 13493 | 13510 | 2012 |
| 654704 | 2027 | 2044 | TTGTTATCTGCTGCTGGC | eeeeddddddddddeeee | 46 | 13496 | 13513 | 2013 |
| 654705 | 2028 | 2045 | GTTGTTATCTGCTGCTGG | eeeeddddddddddeeee | 60 | 13497 | 13514 | 2014 |
| 654707 | 2046 | 2063 | ATCGCTGATTTGTCCGGG | eeeeddddddddddeeee | 91 | 13515 | 13532 | 2015 |
| 654708 | 2047 | 2064 | CATCGCTGATTTGTCCGG | eeeeddddddddddeeee | 78 | 13516 | 13533 | 2016 |
| 654709 | 2048 | 2065 | ACATCGCTGATTTGTCCG | eeeeddddddddddeeee | 66 | 13517 | 13534 | 2017 |
| 654710 | 2049 | 2066 | CACATCGCTGATTTGTCC | eeeeddddddddddeeee | 80 | 13518 | 13535 | 2018 |
| 654711 | 2050 | 2067 | ACACATCGCTGATTTGTC | eeeeddddddddddeeee | 77 | 13519 | 13536 | 2019 |
| 654713 | 2097 | 2114 | CAGCTCAAAGTCGACTCA | eeeeddddddddddeeee | 77 | 13566 | 13583 | 2020 |
| 654716 | 2250 | 2267 | GAACAGTAGTCCCGCGCT | eeeeddddddddddeeee | 80 | 13719 | 13736 | 2021 |
| 654719 | 2268 | 2285 | CGGTTGGAATTCTTTTTG | eeeeddddddddddeeee | 65 | 13737 | 13754 | 2022 |
| 654722 | 2274 | 2291 | GCTGGTCGGTTGGAATTC | eeeeddddddddddeeee | 74 | 13743 | 13760 | 2023 |
| 654724 | 2282 | 2299 | ACAAACAAGCTGGTCGGT | eeeeddddddddddeeee | 81 | 13751 | 13768 | 2024 |
| 654725 | 2283 | 2300 | CACAAACAAGCTGGTCGG | eeeeddddddddddeeee | 80 | 13752 | 13769 | 2025 |
| 654728 | 2024 | 2041 | TTATCTGCTGCTGGCCTT | eeeeddddddddeeeeee | 53 | 13493 | 13510 | 2012 |
| 654731 | 2027 | 2044 | TTGTTATCTGCTGCTGGC | eeeeddddddddeeeeee | 56 | 13496 | 13513 | 2013 |
| 654732 | 2028 | 2045 | GTTGTTATCTGCTGCTGG | eeeeddddddddeeeeee | 71 | 13497 | 13514 | 2014 |
| 654735 | 2047 | 2064 | CATCGCTGATTTGTCCGG | eeeeddddddddeeeeee | 71 | 13516 | 13533 | 2016 |
| 654736 | 2048 | 2065 | ACATCGCTGATTTGTCCG | eeeeddddddddeeeeee | 72 | 13517 | 13534 | 2017 |
| 654737 | 2049 | 2066 | CACATCGCTGATTTGTCC | eeeeddddddddeeeeee | 82 | 13518 | 13535 | 2018 |
| 654740 | 2097 | 2114 | CAGCTCAAAGTCGACTCA | eeeeddddddddeeeeee | 88 | 13566 | 13583 | 2020 |
| 654743 | 2250 | 2267 | GAACAGTAGTCCCGCGCT | eeeeddddddddeeeeee | 75 | 13719 | 13736 | 2021 |
| 654745 | 2267 | 2284 | GGTTGGAATTCTTTTTGG | eeeeddddddddeeeeee | 49 | 13736 | 13753 | 2026 |
| 654746 | 2268 | 2285 | CGGTTGGAATTCTTTTTG | eeeeddddddddeeeeee | 62 | 13737 | 13754 | 2022 |
| 654749 | 2274 | 2291 | GCTGGTCGGTTGGAATTC | eeeeddddddddeeeeee | 55 | 13743 | 13760 | 2023 |
| 654752 | 2283 | 2300 | CACAAACAAGCTGGTCGG | eeeeddddddddeeeeee | 74 | 13752 | 13769 | 2025 |
| 654755 | 2024 | 2041 | TTATCTGCTGCTGGCCTT | eeeeeddddddddeeeee | 47 | 13493 | 13510 | 2012 |
| 654758 | 2027 | 2044 | TTGTTATCTGCTGCTGGC | eeeeeddddddddeeeee | 51 | 13496 | 13513 | 2013 |
| 654759 | 2028 | 2045 | GTTGTTATCTGCTGCTGG | eeeeeddddddddeeeee | 56 | 13497 | 13514 | 2014 |
| 654761 | 2046 | 2063 | ATCGCTGATTTGTCCGGG | eeeeeddddddddeeeee | 74 | 13515 | 13532 | 2015 |
| 654762 | 2047 | 2064 | CATCGCTGATTTGTCCGG | eeeeeddddddddeeeee | 62 | 13516 | 13533 | 2016 |
| 654763 | 2048 | 2065 | ACATCGCTGATTTGTCCG | eeeeeddddddddeeeee | 61 | 13517 | 13534 | 2017 |
| 654764 | 2049 | 2066 | CACATCGCTGATTTGTCC | eeeeeddddddddeeeee | 68 | 13518 | 13535 | 2018 |
| 654765 | 2050 | 2067 | ACACATCGCTGATTTGTC | eeeeeddddddddeeeee | 72 | 13519 | 13536 | 2019 |
| 654767 | 2097 | 2114 | CAGCTCAAAGTCGACTCA | eeeeeddddddddeeeee | 63 | 13566 | 13583 | 2020 |
| 654768 | 2098 | 2115 | CCAGCTCAAAGTCGACTC | eeeeeddddddddeeeee | 86 | 13567 | 13584 | 2027 |
| 654770 | 2250 | 2267 | GAACAGTAGTCCCGCGCT | eeeeeddddddddeeeee | 55 | 13719 | 13736 | 2021 |
| 654771 | 2251 | 2268 | GGAACAGTAGTCCCGCGC | eeeeeddddddddeeeee | 82 | 13720 | 13737 | 2028 |
| 654773 | 2268 | 2285 | CGGTTGGAATTCTTTTTG | eeeeeddddddddeeeee | 58 | 13737 | 13754 | 2022 |
| 654776 | 2274 | 2291 | GCTGGTCGGTTGGAATTC | eeeeeddddddddeeeee | 37 | 13743 | 13760 | 2023 |
| 654778 | 2282 | 2299 | ACAAACAAGCTGGTCGGT | eeeeeddddddddeeeee | 71 | 13751 | 13768 | 2024 |
| 654779 | 2283 | 2300 | CACAAACAAGCTGGTCGG | eeeeeddddddddeeeee | 63 | 13752 | 13769 | 2025 |
| 654781 | 2023 | 2040 | TATCTGCTGCTGGCCTTT | eeeeeeddddddddeeee | 56 | 13492 | 13509 | 2029 |
| 654782 | 2024 | 2041 | TTATCTGCTGCTGGCCTT | eeeeeeddddddddeeee | 63 | 13493 | 13510 | 2012 |
| 654784 | 2026 | 2043 | TGTTATCTGCTGCTGGCC | eeeeeeddddddddeeee | 65 | 13495 | 13512 | 2030 |
| 654785 | 2027 | 2044 | TTGTTATCTGCTGCTGGC | eeeeeeddddddddeeee | 55 | 13496 | 13513 | 2013 |
| 654786 | 2028 | 2045 | GTTGTTATCTGCTGCTGG | eeeeeeddddddddeeee | 48 | 13497 | 13514 | 2014 |
| 654789 | 2047 | 2064 | CATCGCTGATTTGTCCGG | eeeeeeddddddddeeee | 73 | 13516 | 13533 | 2016 |
| 654790 | 2048 | 2065 | ACATCGCTGATTTGTCCG | eeeeeeddddddddeeee | 69 | 13517 | 13534 | 2017 |
| 654791 | 2049 | 2066 | CACATCGCTGATTTGTCC | eeeeeeddddddddeeee | 61 | 13518 | 13535 | 2018 |
| 654794 | 2097 | 2114 | CAGCTCAAAGTCGACTCA | eeeeeeddddddddeeee | 79 | 13566 | 13583 | 2020 |
| 654797 | 2250 | 2267 | GAACAGTAGTCCCGCGCT | eeeeeeddddddddeeee | 37 | 13719 | 13736 | 2021 |
| 654800 | 2268 | 2285 | CGGTTGGAATTCTTTTTG | eeeeeeddddddddeeee | 63 | 13737 | 13754 | 2022 |
| 654801 | 2269 | 2286 | TCGGTTGGAATTCTTTTT | eeeeeeddddddddeeee | 59 | 13738 | 13755 | 2031 |
| 654803 | 2274 | 2291 | GCTGGTCGGTTGGAATTC | eeeeeeddddddddeeee | 61 | 13743 | 13760 | 2023 |
| 654806 | 2283 | 2300 | CACAAACAAGCTGGTCGG | eeeeeeddddddddeeee | 54 | 13752 | 13769 | 2025 |
| 654809 | 2023 | 2041 | TTATCTGCTGCTGGCCTTT | eeeeddddddddddeeeee | 45 | 13492 | 13510 | 2032 |
| 654812 | 2026 | 2044 | TTGTTATCTGCTGCTGGCC | eeeeddddddddddeeeee | 57 | 13495 | 13513 | 2033 |
| 654813 | 2027 | 2045 | GTTGTTATCTGCTGCTGGC | eeeeddddddddddeeeee | 64 | 13496 | 13514 | 2034 |
| 654815 | 2046 | 2064 | CATCGCTGATTTGTCCGGG | eeeeddddddddddeeeee | 83 | 13515 | 13533 | 2035 |
| 654816 | 2047 | 2065 | ACATCGCTGATTTGTCCGG | eeeeddddddddddeeeee | 68 | 13516 | 13534 | 2036 |
| 654817 | 2048 | 2066 | CACATCGCTGATTTGTCCG | eeeeddddddddddeeeee | 82 | 13517 | 13535 | 2037 |
| 654818 | 2049 | 2067 | ACACATCGCTGATTTGTCC | eeeeddddddddddeeeee | 44 | 13518 | 13536 | 2038 |
| 654820 | 2096 | 2114 | CAGCTCAAAGTCGACTCAT | eeeeddddddddddeeeee | 80 | 13565 | 13583 | 2039 |
| 654822 | 2248 | 2266 | AACAGTAGTCCCGCGCTAA | eeeeddddddddddeeeee | 63 | 13717 | 13735 | 2040 |
| 654823 | 2249 | 2267 | GAACAGTAGTCCCGCGCTA | eeeeddddddddddeeeee | 77 | 13718 | 13736 | 2041 |
| 654826 | 2267 | 2285 | CGGTTGGAATTCTTTTTGG | eeeeddddddddddeeeee | 76 | 13736 | 13754 | 2042 |
| 654829 | 2273 | 2291 | GCTGGTCGGTTGGAATTCT | eeeeddddddddddeeeee | 78 | 13742 | 13760 | 2043 |
| 654832 | 2282 | 2300 | CACAAACAAGCTGGTCGGT | eeeeddddddddddeeeee | 82 | 13751 | 13769 | 2044 |
| 654833 | 2283 | 2301 | TCACAAACAAGCTGGTCGG | eeeeddddddddddeeeee | 28 | 13752 | 13770 | 2045 |
| 654834 | 2022 | 2040 | TATCTGCTGCTGGCCTTTG | eeeeddddddddeeeeeee | 3 | 13491 | 13509 | 2046 |
| 654835 | 2023 | 2041 | TTATCTGCTGCTGGCCTTT | eeeeddddddddeeeeeee | 48 | 13492 | 13510 | 2032 |
| 654837 | 2025 | 2043 | TGTTATCTGCTGCTGGCCT | eeeeddddddddeeeeeee | 64 | 13494 | 13512 | 2047 |
| 654838 | 2026 | 2044 | TTGTTATCTGCTGCTGGCC | eeeeddddddddeeeeeee | 38 | 13495 | 13513 | 2033 |
| 654839 | 2027 | 2045 | GTTGTTATCTGCTGCTGGC | eeeeddddddddeeeeeee | 60 | 13496 | 13514 | 2034 |
| 654841 | 2046 | 2064 | CATCGCTGATTTGTCCGGG | eeeeddddddddeeeeeee | 72 | 13515 | 13533 | 2035 |
| 654842 | 2047 | 2065 | ACATCGCTGATTTGTCCGG | eeeeddddddddeeeeeee | 70 | 13516 | 13534 | 2036 |
| 654843 | 2048 | 2066 | CACATCGCTGATTTGTCCG | eeeeddddddddeeeeeee | 85 | 13517 | 13535 | 2037 |
| 654845 | 2095 | 2113 | AGCTCAAAGTCGACTCATT | eeeeddddddddeeeeeee | 44 | 13564 | 13582 | 2048 |
| 654846 | 2096 | 2114 | CAGCTCAAAGTCGACTCAT | eeeeddddddddeeeeeee | 84 | 13565 | 13583 | 2039 |
| 654849 | 2249 | 2267 | GAACAGTAGTCCCGCGCTA | eeeeddddddddeeeeeee | 43 | 13718 | 13736 | 2041 |
| 654852 | 2267 | 2285 | CGGTTGGAATTCTTTTTGG | eeeeddddddddeeeeeee | 73 | 13736 | 13754 | 2042 |
| 654855 | 2273 | 2291 | GCTGGTCGGTTGGAATTCT | eeeeddddddddeeeeeee | 59 | 13742 | 13760 | 2043 |
| 654858 | 2282 | 2300 | CACAAACAAGCTGGTCGGT | eeeeddddddddeeeeeee | 72 | 13751 | 13769 | 2044 |
| 654861 | 2023 | 2041 | TTATCTGCTGCTGGCCTTT | eeeeeddddddddeeeeee | 40 | 13492 | 13510 | 2032 |
| 654864 | 2026 | 2044 | TTGTTATCTGCTGCTGGCC | eeeeeddddddddeeeeee | 57 | 13495 | 13513 | 2033 |
| 654865 | 2027 | 2045 | GTTGTTATCTGCTGCTGGC | eeeeeddddddddeeeeee | 52 | 13496 | 13514 | 2034 |
| 654867 | 2046 | 2064 | CATCGCTGATTTGTCCGGG | eeeeeddddddddeeeeee | 71 | 13515 | 13533 | 2035 |
| 654868 | 2047 | 2065 | ACATCGCTGATTTGTCCGG | eeeeeddddddddeeeeee | 69 | 13516 | 13534 | 2036 |
| 654869 | 2048 | 2066 | CACATCGCTGATTTGTCCG | eeeeeddddddddeeeeee | 69 | 13517 | 13535 | 2037 |
| 654872 | 2096 | 2114 | CAGCTCAAAGTCGACTCAT | eeeeeddddddddeeeeee | 63 | 13565 | 13583 | 2039 |
| 654875 | 2249 | 2267 | GAACAGTAGTCCCGCGCTA | eeeeeddddddddeeeeee | 55 | 13718 | 13736 | 2041 |
| 654877 | 2266 | 2284 | GGTTGGAATTCTTTTTGGA | eeeeeddddddddeeeeee | 43 | 13735 | 13753 | 2049 |
| 654878 | 2267 | 2285 | CGGTTGGAATTCTTTTTGG | eeeeeddddddddeeeeee | 61 | 13736 | 13754 | 2042 |
| 654881 | 2273 | 2291 | GCTGGTCGGTTGGAATTCT | eeeeeddddddddeeeeee | 49 | 13742 | 13760 | 2043 |
| 654883 | 2281 | 2299 | ACAAACAAGCTGGTCGGTT | eeeeeddddddddeeeeee | 40 | 13750 | 13768 | 2050 |
| 654884 | 2282 | 2300 | CACAAACAAGCTGGTCGGT | eeeeeddddddddeeeeee | 73 | 13751 | 13769 | 2044 |
| 654887 | 2023 | 2041 | TTATCTGCTGCTGGCCTTT | eeeeeeddddddddeeeee | 60 | 13492 | 13510 | 2032 |
| 654890 | 2026 | 2044 | TTGTTATCTGCTGCTGGCC | eeeeeeddddddddeeeee | 44 | 13495 | 13513 | 2033 |
| 654891 | 2027 | 2045 | GTTGTTATCTGCTGCTGGC | eeeeeeddddddddeeeee | 60 | 13496 | 13514 | 2034 |
| 654893 | 2046 | 2064 | CATCGCTGATTTGTCCGGG | eeeeeeddddddddeeeee | 74 | 13515 | 13533 | 2035 |
| 654894 | 2047 | 2065 | ACATCGCTGATTTGTCCGG | eeeeeeddddddddeeeee | 64 | 13516 | 13534 | 2036 |
| 654895 | 2048 | 2066 | CACATCGCTGATTTGTCCG | eeeeeeddddddddeeeee | 62 | 13517 | 13535 | 2037 |
| 654898 | 2096 | 2114 | CAGCTCAAAGTCGACTCAT | eeeeeeddddddddeeeee | 67 | 13565 | 13583 | 2039 |
| 654899 | 2097 | 2115 | CCAGCTCAAAGTCGACTCA | eeeeeeddddddddeeeee | 63 | 13566 | 13584 | 2051 |
| 654901 | 2249 | 2267 | GAACAGTAGTCCCGCGCTA | eeeeeeddddddddeeeee | 55 | 13718 | 13736 | 2041 |
| 654904 | 2267 | 2285 | CGGTTGGAATTCTTTTTGG | eeeeeeddddddddeeeee | 45 | 13736 | 13754 | 2042 |
| 654907 | 2273 | 2291 | GCTGGTCGGTTGGAATTCT | eeeeeeddddddddeeeee | 51 | 13742 | 13760 | 2043 |
| 654910 | 2282 | 2300 | CACAAACAAGCTGGTCGGT | eeeeeeddddddddeeeee | 47 | 13751 | 13769 | 2044 |
| 654911 | 2283 | 2301 | TCACAAACAAGCTGGTCGG | eeeeeeddddddddeeeee | 72 | 13752 | 13770 | 2045 |
| 654917 | 2027 | 2045 | GTTGTTATCTGCTGCTGGC | eeeeeeeddddddddeeee | 45 | 13496 | 13514 | 2034 |
| 654920 | 2047 | 2065 | ACATCGCTGATTTGTCCGG | eeeeeeeddddddddeeee | 77 | 13516 | 13534 | 2036 |
| 654939 | 2023 | 2042 | GTTATCTGCTGCTGGCCTTT | eeeeeeeddddddddeeeee | 65 | 13492 | 13511 | 230 |
| 654941 | 2025 | 2044 | TTGTTATCTGCTGCTGGCCT | eeeeeeeddddddddeeeee | 55 | 13494 | 13513 | 232 |
| 654942 | 2026 | 2045 | GTTGTTATCTGCTGCTGGCC | eeeeeeeddddddddeeeee | 48 | 13495 | 13514 | 233 |
| 654943 | 2027 | 2046 | GGTTGTTATCTGCTGCTGGC | eeeeeeeddddddddeeeee | 67 | 13496 | 13515 | 234 |
| 654944 | 2028 | 2047 | GGGTTGTTATCTGCTGCTGG | eeeeeeeddddddddeeeee | 56 | 13497 | 13516 | 235 |
| 654945 | 2046 | 2065 | ACATCGCTGATTTGTCCGGG | eeeeeeeddddddddeeeee | 77 | 13515 | 13534 | 236 |
| 654946 | 2047 | 2066 | CACATCGCTGATTTGTCCGG | eeeeeeeddddddddeeeee | 67 | 13516 | 13535 | 237 |
| 654947 | 2048 | 2067 | ACACATCGCTGATTTGTCCG | eeeeeeeddddddddeeeee | 56 | 13517 | 13536 | 238 |
| 654950 | 2096 | 2115 | CCAGCTCAAAGTCGACTCAT | eeeeeeeddddddddeeeee | 75 | 13565 | 13584 | 267 |
| 654951 | 2097 | 2116 | TCCAGCTCAAAGTCGACTCA | eeeeeeeddddddddeeeee | 50 | 13566 | 13585 | 268 |
| 654952 | 2248 | 2267 | GAACAGTAGTCCCGCGCTAA | eeeeeeeddddddddeeeee | 53 | 13717 | 13736 | 1892 |
| 654953 | 2249 | 2268 | GGAACAGTAGTCCCGCGCTA | eeeeeeeddddddddeeeee | 44 | 13718 | 13737 | 1708 |
| 654955 | 2266 | 2285 | CGGTTGGAATTCTTTTTGGA | eeeeeeeddddddddeeeee | 58 | 13735 | 13754 | 1904 |
| 654956 | 2267 | 2286 | TCGGTTGGAATTCTTTTTGG | eeeeeeeddddddddeeeee | 66 | 13736 | 13755 | 1714 |
| 654959 | 2273 | 2292 | AGCTGGTCGGTTGGAATTCT | eeeeeeeddddddddeeeee | 56 | 13742 | 13761 | 1716 |
| 654962 | 2282 | 2301 | TCACAAACAAGCTGGTCGGT | eeeeeeeddddddddeeeee | 55 | 13751 | 13770 | 1719 |
| 654963 | 2283 | 2302 | TTCACAAACAAGCTGGTCGG | eeeeeeeddddddddeeeee | 63 | 13752 | 13771 | 1915 |
| 654964 | 2022 | 2041 | TTATCTGCTGCTGGCCTTTG | eeeeeeddddddddeeeeee | 43 | 13491 | 13510 | 229 |
| 654965 | 2023 | 2042 | GTTATCTGCTGCTGGCCTTT | eeeeeeddddddddeeeeee | 65 | 13492 | 13511 | 230 |
| 654968 | 2026 | 2045 | GTTGTTATCTGCTGCTGGCC | eeeeeeddddddddeeeeee | 44 | 13495 | 13514 | 233 |
| 654969 | 2027 | 2046 | GGTTGTTATCTGCTGCTGGC | eeeeeeddddddddeeeeee | 64 | 13496 | 13515 | 234 |
| 654970 | 2028 | 2047 | GGGTTGTTATCTGCTGCTGG | eeeeeeddddddddeeeeee | 76 | 13497 | 13516 | 235 |
| 654971 | 2046 | 2065 | ACATCGCTGATTTGTCCGGG | eeeeeeddddddddeeeeee | 60 | 13515 | 13534 | 236 |
| 654972 | 2047 | 2066 | CACATCGCTGATTTGTCCGG | eeeeeeddddddddeeeeee | 74 | 13516 | 13535 | 237 |
| 654973 | 2048 | 2067 | ACACATCGCTGATTTGTCCG | eeeeeeddddddddeeeeee | 54 | 13517 | 13536 | 238 |
| 654974 | 2049 | 2068 | GACACATCGCTGATTTGTCC | eeeeeeddddddddeeeeee | 78 | 13518 | 13537 | 239 |
| 654976 | 2096 | 2115 | CCAGCTCAAAGTCGACTCAT | eeeeeeddddddddeeeeee | 62 | 13565 | 13584 | 267 |
| 654979 | 2249 | 2268 | GGAACAGTAGTCCCGCGCTA | eeeeeeddddddddeeeeee | 59 | 13718 | 13737 | 1708 |
| 654982 | 2267 | 2286 | TCGGTTGGAATTCTTTTTGG | eeeeeeddddddddeeeeee | 63 | 13736 | 13755 | 1714 |
| 654985 | 2273 | 2292 | AGCTGGTCGGTTGGAATTCT | eeeeeeddddddddeeeeee | 57 | 13742 | 13761 | 1716 |
| 654988 | 2282 | 2301 | TCACAAACAAGCTGGTCGGT | eeeeeeddddddddeeeeee | 70 | 13751 | 13770 | 1719 |
| 654989 | 2283 | 2302 | TTCACAAACAAGCTGGTCGG | eeeeeeddddddddeeeeee | 77 | 13752 | 13771 | 1915 |
| 654990 | 2022 | 2041 | TTATCTGCTGCTGGCCTTTG | eeeeeddddddddeeeeeee | 41 | 13491 | 13510 | 229 |
| 654991 | 2023 | 2042 | GTTATCTGCTGCTGGCCTTT | eeeeeddddddddeeeeeee | 70 | 13492 | 13511 | 230 |
| 654994 | 2026 | 2045 | GTTGTTATCTGCTGCTGGCC | eeeeeddddddddeeeeeee | 33 | 13495 | 13514 | 233 |
| 654995 | 2027 | 2046 | GGTTGTTATCTGCTGCTGGC | eeeeeddddddddeeeeeee | 79 | 13496 | 13515 | 234 |
| 654997 | 2046 | 2065 | ACATCGCTGATTTGTCCGGG | eeeeeddddddddeeeeeee | 64 | 13515 | 13534 | 236 |
| 654998 | 2047 | 2066 | CACATCGCTGATTTGTCCGG | eeeeeddddddddeeeeeee | 70 | 13516 | 13535 | 237 |
| 654999 | 2048 | 2067 | ACACATCGCTGATTTGTCCG | eeeeeddddddddeeeeeee | 85 | 13517 | 13536 | 238 |
| 655002 | 2096 | 2115 | CCAGCTCAAAGTCGACTCAT | eeeeeddddddddeeeeeee | 85 | 13565 | 13584 | 267 |
| 655005 | 2249 | 2268 | GGAACAGTAGTCCCGCGCTA | eeeeeddddddddeeeeeee | 73 | 13718 | 13737 | 1708 |
| 655008 | 2267 | 2286 | TCGGTTGGAATTCTTTTTGG | eeeeeddddddddeeeeeee | 67 | 13736 | 13755 | 1714 |
| 655011 | 2273 | 2292 | AGCTGGTCGGTTGGAATTCT | eeeeeddddddddeeeeeee | 31 | 13742 | 13761 | 1716 |
| 655014 | 2282 | 2301 | TCACAAACAAGCTGGTCGGT | eeeeeddddddddeeeeeee | 76 | 13751 | 13770 | 1719 |
| 655044 | 2024 | 2041 | TTATCTGCTGCTGGCCTT | eeeedddddddddeeeee | 55 | 13493 | 13510 | 2012 |
| 655045 | 2027 | 2044 | TTGTTATCTGCTGCTGGC | eeeedddddddddeeeee | 46 | 13496 | 13513 | 2013 |
| 655046 | 2028 | 2045 | GTTGTTATCTGCTGCTGG | eeeedddddddddeeeee | 54 | 13497 | 13514 | 2014 |
| 655047 | 2047 | 2064 | CATCGCTGATTTGTCCGG | eeeedddddddddeeeee | 61 | 13516 | 13533 | 2016 |
| 655048 | 2048 | 2065 | ACATCGCTGATTTGTCCG | eeeedddddddddeeeee | 59 | 13517 | 13534 | 2017 |
| 655049 | 2049 | 2066 | CACATCGCTGATTTGTCC | eeeedddddddddeeeee | 84 | 13518 | 13535 | 2018 |
| 655050 | 2097 | 2114 | CAGCTCAAAGTCGACTCA | eeeedddddddddeeeee | 75 | 13566 | 13583 | 2020 |
| 655051 | 2250 | 2267 | GAACAGTAGTCCCGCGCT | eeeedddddddddeeeee | 74 | 13719 | 13736 | 2021 |
| 655052 | 2268 | 2285 | CGGTTGGAATTCTTTTTG | eeeedddddddddeeeee | 58 | 13737 | 13754 | 2022 |
| 655053 | 2274 | 2291 | GCTGGTCGGTTGGAATTC | eeeedddddddddeeeee | 58 | 13743 | 13760 | 2023 |
| 655054 | 2283 | 2300 | CACAAACAAGCTGGTCGG | eeeedddddddddeeeee | 76 | 13752 | 13769 | 2025 |
| 655055 | 2024 | 2041 | TTATCTGCTGCTGGCCTT | eeeeedddddddddeeee | 57 | 13493 | 13510 | 2012 |
| 655056 | 2027 | 2044 | TTGTTATCTGCTGCTGGC | eeeeedddddddddeeee | 50 | 13496 | 13513 | 2013 |
| 655057 | 2028 | 2045 | GTTGTTATCTGCTGCTGG | eeeeedddddddddeeee | 63 | 13497 | 13514 | 2014 |
| 655058 | 2047 | 2064 | CATCGCTGATTTGTCCGG | eeeeedddddddddeeee | 80 | 13516 | 13533 | 2016 |
| 655059 | 2048 | 2065 | ACATCGCTGATTTGTCCG | eeeeedddddddddeeee | 60 | 13517 | 13534 | 2017 |
| 655060 | 2049 | 2066 | CACATCGCTGATTTGTCC | eeeeedddddddddeeee | 68 | 13518 | 13535 | 2018 |
| 655061 | 2097 | 2114 | CAGCTCAAAGTCGACTCA | eeeeedddddddddeeee | 79 | 13566 | 13583 | 2020 |
| 655062 | 2250 | 2267 | GAACAGTAGTCCCGCGCT | eeeeedddddddddeeee | 51 | 13719 | 13736 | 2021 |
| 655063 | 2268 | 2285 | CGGTTGGAATTCTTTTTG | eeeeedddddddddeeee | 74 | 13737 | 13754 | 2022 |
| 655064 | 2274 | 2291 | GCTGGTCGGTTGGAATTC | eeeeedddddddddeeee | 65 | 13743 | 13760 | 2023 |
| 655065 | 2283 | 2300 | CACAAACAAGCTGGTCGG | eeeeedddddddddeeee | 69 | 13752 | 13769 | 2025 |
| 655066 | 2023 | 2041 | TTATCTGCTGCTGGCCTTT | eeeedddddddddeeeeee | 50 | 13492 | 13510 | 2032 |
| 655067 | 2026 | 2044 | TTGTTATCTGCTGCTGGCC | eeeedddddddddeeeeee | 60 | 13495 | 13513 | 2033 |
| 655068 | 2027 | 2045 | GTTGTTATCTGCTGCTGGC | eeeedddddddddeeeeee | 65 | 13496 | 13514 | 2034 |
| 655069 | 2046 | 2064 | CATCGCTGATTTGTCCGGG | eeeedddddddddeeeeee | 71 | 13515 | 13533 | 2035 |
| 655070 | 2047 | 2065 | ACATCGCTGATTTGTCCGG | eeeedddddddddeeeeee | 65 | 13516 | 13534 | 2036 |
| 655071 | 2048 | 2066 | CACATCGCTGATTTGTCCG | eeeedddddddddeeeeee | 87 | 13517 | 13535 | 2037 |
| 655072 | 2096 | 2114 | CAGCTCAAAGTCGACTCAT | eeeedddddddddeeeeee | 75 | 13565 | 13583 | 2039 |
| 655073 | 2249 | 2267 | GAACAGTAGTCCCGCGCTA | eeeedddddddddeeeeee | 73 | 13718 | 13736 | 2041 |
| 655074 | 2267 | 2285 | CGGTTGGAATTCTTTTTGG | eeeedddddddddeeeeee | 70 | 13736 | 13754 | 2042 |
| 655075 | 2273 | 2291 | GCTGGTCGGTTGGAATTCT | eeeedddddddddeeeeee | 65 | 13742 | 13760 | 2043 |
| 655076 | 2282 | 2300 | CACAAACAAGCTGGTCGGT | eeeedddddddddeeeeee | 65 | 13751 | 13769 | 2044 |
| 655077 | 2023 | 2041 | TTATCTGCTGCTGGCCTTT | eeeeedddddddddeeeee | 40 | 13492 | 13510 | 2032 |
| 655078 | 2026 | 2044 | TTGTTATCTGCTGCTGGCC | eeeeedddddddddeeeee | 57 | 13495 | 13513 | 2033 |
| 655079 | 2027 | 2045 | GTTGTTATCTGCTGCTGGC | eeeeedddddddddeeeee | 66 | 13496 | 13514 | 2034 |
| 655080 | 2046 | 2064 | CATCGCTGATTTGTCCGGG | eeeeedddddddddeeeee | 70 | 13515 | 13533 | 2035 |
| 655081 | 2047 | 2065 | ACATCGCTGATTTGTCCGG | eeeeedddddddddeeeee | 66 | 13516 | 13534 | 2036 |
| 655082 | 2048 | 2066 | CACATCGCTGATTTGTCCG | eeeeedddddddddeeeee | 73 | 13517 | 13535 | 2037 |
| 655083 | 2096 | 2114 | CAGCTCAAAGTCGACTCAT | eeeeedddddddddeeeee | 81 | 13565 | 13583 | 2039 |
| 655084 | 2249 | 2267 | GAACAGTAGTCCCGCGCTA | eeeeedddddddddeeeee | 65 | 13718 | 13736 | 2041 |
| 655085 | 2267 | 2285 | CGGTTGGAATTCTTTTTGG | eeeeedddddddddeeeee | 70 | 13736 | 13754 | 2042 |
| 655086 | 2273 | 2291 | GCTGGTCGGTTGGAATTCT | eeeeedddddddddeeeee | 69 | 13742 | 13760 | 2043 |
| 655087 | 2282 | 2300 | CACAAACAAGCTGGTCGGT | eeeeedddddddddeeeee | 79 | 13751 | 13769 | 2044 |
| 655088 | 2023 | 2042 | GTTATCTGCTGCTGGCCTTT | eeeeedddddddddeeeeee | 70 | 13492 | 13511 | 230 |
| 655089 | 2026 | 2045 | GTTGTTATCTGCTGCTGGCC | eeeeedddddddddeeeeee | 42 | 13495 | 13514 | 233 |
| 655090 | 2027 | 2046 | GGTTGTTATCTGCTGCTGGC | eeeeedddddddddeeeeee | 82 | 13496 | 13515 | 234 |
| 655091 | 2046 | 2065 | ACATCGCTGATTTGTCCGGG | eeeeedddddddddeeeeee | 66 | 13515 | 13534 | 236 |
| 655092 | 2047 | 2066 | CACATCGCTGATTTGTCCGG | eeeeedddddddddeeeeee | 78 | 13516 | 13535 | 237 |
| 655093 | 2048 | 2067 | ACACATCGCTGATTTGTCCG | eeeeedddddddddeeeeee | 90 | 13517 | 13536 | 238 |
| 655094 | 2096 | 2115 | CCAGCTCAAAGTCGACTCAT | eeeeedddddddddeeeeee | 80 | 13565 | 13584 | 267 |
| 655095 | 2249 | 2268 | GGAACAGTAGTCCCGCGCTA | eeeeedddddddddeeeeee | 84 | 13718 | 13737 | 1708 |
| 655096 | 2267 | 2286 | TCGGTTGGAATTCTTTTTGG | eeeeedddddddddeeeeee | 76 | 13736 | 13755 | 1714 |
| 655097 | 2273 | 2292 | AGCTGGTCGGTTGGAATTCT | eeeeedddddddddeeeeee | 63 | 13742 | 13761 | 1716 |
| 655098 | 2282 | 2301 | TCACAAACAAGCTGGTCGGT | eeeeedddddddddeeeeee | 79 | 13751 | 13770 | 1719 |
| 655099 | 2023 | 2042 | GTTATCTGCTGCTGGCCTTT | eeeeeedddddddddeeeee | 75 | 13492 | 13511 | 230 |
| 655100 | 2026 | 2045 | GTTGTTATCTGCTGCTGGCC | eeeeeedddddddddeeeee | 67 | 13495 | 13514 | 233 |
| 655101 | 2027 | 2046 | GGTTGTTATCTGCTGCTGGC | eeeeeedddddddddeeeee | 78 | 13496 | 13515 | 234 |
| 655102 | 2046 | 2065 | ACATCGCTGATTTGTCCGGG | eeeeeedddddddddeeeee | 82 | 13515 | 13534 | 236 |
| 655103 | 2047 | 2066 | CACATCGCTGATTTGTCCGG | eeeeeedddddddddeeeee | 74 | 13516 | 13535 | 237 |
| 655104 | 2048 | 2067 | ACACATCGCTGATTTGTCCG | eeeeeedddddddddeeeee | 71 | 13517 | 13536 | 238 |
| 655105 | 2096 | 2115 | CCAGCTCAAAGTCGACTCAT | eeeeeedddddddddeeeee | 82 | 13565 | 13584 | 267 |
| 655106 | 2249 | 2268 | GGAACAGTAGTCCCGCGCTA | eeeeeedddddddddeeeee | 68 | 13718 | 13737 | 1708 |
| 655107 | 2267 | 2286 | TCGGTTGGAATTCTTTTTGG | eeeeeedddddddddeeeee | 79 | 13736 | 13755 | 1714 |
| 655108 | 2273 | 2292 | AGCTGGTCGGTTGGAATTCT | eeeeeedddddddddeeeee | 65 | 13742 | 13761 | 1716 |
| 655109 | 2282 | 2301 | TCACAAACAAGCTGGTCGGT | eeeeeedddddddddeeeee | 82 | 13751 | 13770 | 1719 |

### Example 2: Dose-Dependent Antisense Inhibition of Human Angiotensinogen (AGT) in HepG2 Cells

Of over 2000 antisense oligonucleotides designed and tested in single dose in vitro assays described in Example 1, several of those exhibiting significant inhibition of AGT mRNA were selected and further tested at various doses in HepG2 cells. The results for exemplary antisense oligonucleotides tested in several series of experiment are presented in tables shown below.

Cells were plated at a density of 20,000 cells per well and transfected using electroporation with 0.406 μM, 0.813 μM, 1.63 μM, 3.25 μM, 6.5 μM and 13.0 μM concentrations of antisense oligonucleotide, as specified in Table 15 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells. The half maximal inhibitory concentration (IC₅₀) of each oligonucleotide is also presented. AGT mRNA levels were significantly reduced in a dose-dependent manner in antisense oligonucleotide treated cells.

TABLE 15

| | 0.406 | 0.813 | 1.63 | 3.25 | 6.5 | 13.0 | IC₅₀ | SEQ |
|---|---|---|---|---|---|---|---|---|
| ISIS NO | μM | μM | μM | μM | μM | μM | (μM) | ID NO |
| 568550 | 34 | 36 | 55 | 68 | 78 | 83 | 1.3 | 46 |
| 568557 | 32 | 42 | 61 | 71 | 69 | 72 | 1.2 | 53 |
| 568558 | 30 | 31 | 54 | 67 | 72 | 80 | 1.6 | 54 |
| 568565 | 19 | 32 | 45 | 60 | 72 | 75 | 2.2 | 61 |
| 568572 | 29 | 17 | 56 | 53 | 65 | 63 | 2.9 | 68 |
| 568580 | 13 | 12 | 51 | 56 | 67 | 69 | 3 | 76 |
| 568589 | 32 | 46 | 61 | 69 | 78 | 88 | 1.1 | 85 |
| 568601 | 23 | 16 | 40 | 56 | 71 | 73 | 2.8 | 93 |
| 568605 | 37 | 45 | 61 | 68 | 76 | 77 | 1 | 97 |
| 568617 | 12 | 28 | 52 | 57 | 76 | 76 | 2.3 | 109 |
| 568635 | 21 | 27 | 40 | 61 | 82 | 90 | 2 | 127 |
| 568637 | 69 | 82 | 95 | 94 | 98 | 97 | <0.4 | 129 |
| 568637 | 15 | 9 | 35 | 43 | 59 | 67 | 4.6 | 129 |
| 568638 | 31 | 60 | 74 | 86 | 93 | 90 | 0.6 | 130 |
| 568640 | 41 | 47 | 61 | 84 | 90 | 97 | 0.8 | 132 |
| 568642 | 30 | 41 | 71 | 83 | 94 | 97 | 0.9 | 134 |
| 568643 | 33 | 51 | 74 | 83 | 92 | 93 | 0.7 | 135 |
| 568645 | 26 | 38 | 55 | 74 | 88 | 92 | 1.3 | 137 |
| 568646 | 15 | 37 | 57 | 72 | 88 | 94 | 1.4 | 138 |
| 568647 | 32 | 50 | 71 | 85 | 94 | 96 | 0.8 | 139 |
| 568650 | 44 | 51 | 70 | 79 | 87 | 90 | 0.6 | 142 |

Cells were plated at a density of 20,000 cells per well and transfected using electroporation with 39.1 nM, 156.3 nM, 625.0 nM, 2500 nM and 10,000 nM concentrations of antisense oligonucleotide, as specified in Table 16 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells, and are an average of two trials. The half maximal inhibitory concentration (IC₅₀) of each oligonucleotide is also presented. AGT mRNA levels were significantly reduced in a dose-dependent manner in antisense oligonucleotide treated cells.

TABLE 16

| | 39.1 | 156.3 | 625.0 | 2500 | 10,000 | IC₅₀ | SEQ |
|---|---|---|---|---|---|---|---|
| ISIS NO | nM | nM | nM | nM | nM | (μM) | ID NO |
| 568637 | −2 | 33 | 77 | 92 | 98 | 0.4 | 129 |
| 594622 | 15 | 52 | 84 | 96 | 97 | 0.3 | 163 |
| 594623 | 16 | 30 | 65 | 87 | 96 | 0.4 | 164 |
| 594624 | 13 | 37 | 74 | 92 | 96 | 0.4 | 129 |
| 594625 | 14 | 31 | 74 | 90 | 95 | 0.4 | 165 |
| 594626 | 11 | 20 | 58 | 84 | 94 | 0.6 | 166 |
| 594627 | 11 | 36 | 72 | 93 | 95 | 0.3 | 167 |
| 594628 | −30 | 4 | 51 | 78 | 87 | 1.1 | 168 |
| 594629 | −20 | −1 | 39 | 67 | 94 | 1.4 | 169 |
| 594630 | −10 | 13 | 35 | 52 | 78 | 2.4 | 170 |
| 594631 | 13 | 13 | 49 | 81 | 94 | 0.6 | 171 |
| 594632 | 2 | 27 | 60 | 85 | 97 | 0.6 | 172 |

Cells were plated at a density of 20,000 cells per well and transfected using electroporation with 312.5 nM, 625 nM, 1250 nM, 2500 nM and 5000 nM concentrations of antisense oligonucleotide, as specified in Tables 17 and 18 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells, and are an average of two trials. The half maximal inhibitory concentration (IC₅₀) of each oligonucleotide is also presented. AGT mRNA levels were significantly reduced in a dose-dependent manner in antisense oligonucleotide treated cells.

TABLE 17

| | 312.5 | 625 | 1250 | 2500 | 5000 | IC₅₀ | SEQ |
|---|---|---|---|---|---|---|---|
| ISIS NO | nM | nM | nM | nM | nM | (μM) | ID NO |
| 568637 | 51 | 71 | 84 | 89 | 93 | 0.2 | 129 |
| 594625 | 52 | 73 | 84 | 92 | 95 | 0.2 | 165 |
| 611933 | −7 | 7 | 5 | 1 | −2 | >5 | 313 |
| 612024 | 22 | 39 | 48 | 73 | 80 | 1.1 | 46 |
| 612025 | 21 | 15 | 36 | 59 | 64 | 2.2 | 654 |
| 612058 | 49 | 52 | 53 | 72 | 74 | 0.5 | 53 |
| 612063 | 22 | 38 | 56 | 63 | 65 | 1.3 | 689 |
| 612077 | 35 | 37 | 45 | 67 | 74 | 1.1 | 702 |
| 612101 | 32 | 59 | 68 | 83 | 93 | 0.6 | 725 |
| 612102 | 53 | 67 | 80 | 85 | 91 | 0.2 | 726 |
| 612104 | 41 | 51 | 50 | 72 | 83 | 0.7 | 728 |
| 612117 | 25 | 47 | 56 | 68 | 73 | 1.0 | 738 |
| 612134 | 40 | 43 | 49 | 67 | 79 | 0.9 | 894 |
| 612147 | 30 | 48 | 74 | 76 | 82 | 0.7 | 905 |
| 612151 | 33 | 38 | 51 | 71 | 81 | 1.0 | 909 |
| 612202 | 33 | 49 | 62 | 83 | 87 | 0.7 | 954 |
| 612315 | 7 | 33 | 55 | 72 | 76 | 1.3 | 779 |
| 612322 | 29 | 48 | 61 | 78 | 87 | 0.8 | 786 |
| 612323 | 42 | 60 | 82 | 87 | 91 | 0.4 | 787 |
| 612336 | 31 | 59 | 72 | 83 | 89 | 0.5 | 800 |
| 612344 | 31 | 39 | 69 | 76 | 85 | 0.8 | 808 |
| 612346 | 13 | 42 | 55 | 74 | 86 | 1.1 | 810 |
| 612347 | 29 | 46 | 71 | 83 | 90 | 0.7 | 811 |
| 612448 | 15 | 26 | 59 | 76 | 86 | 1.1 | 411 |
| 612491 | 16 | 14 | 33 | 29 | 49 | 8.0 | 452 |
| 612502 | 28 | 37 | 58 | 75 | 89 | 0.9 | 463 |
| 612503 | 44 | 55 | 75 | 83 | 91 | 0.4 | 464 |
| 612504 | 17 | 44 | 63 | 68 | 88 | 1.0 | 465 |
| 612505 | 43 | 50 | 66 | 76 | 90 | 0.5 | 466 |
| 612506 | 32 | 44 | 70 | 81 | 91 | 0.7 | 467 |
| 612507 | 24 | 45 | 49 | 70 | 81 | 1.0 | 468 |
| 612509 | 25 | 43 | 60 | 77 | 88 | 0.9 | 470 |
| 612514 | 44 | 41 | 59 | 79 | 92 | 0.6 | 475 |
| 612515 | 21 | 38 | 48 | 61 | 78 | 1.3 | 476 |
| 612516 | 38 | 47 | 74 | 79 | 93 | 0.6 | 477 |
| 612517 | 33 | 37 | 60 | 75 | 86 | 0.8 | 478 |
| 612519 | 14 | 16 | 38 | 54 | 64 | 2.4 | 480 |
| 612540 | 38 | 53 | 76 | 80 | 91 | 0.5 | 500 |
| 612541 | 38 | 51 | 58 | 83 | 90 | 0.6 | 501 |
| 612542 | 43 | 61 | 73 | 83 | 94 | 0.4 | 502 |
| 612543 | 34 | 53 | 64 | 81 | 91 | 0.6 | 503 |
| 612553 | 44 | 64 | 78 | 87 | 91 | 0.3 | 512 |
| 612559 | 36 | 59 | 74 | 89 | 95 | 0.5 | 517 |
| 612560 | 49 | 57 | 68 | 80 | 95 | 0.4 | 518 |
| 612567 | 38 | 50 | 57 | 83 | 85 | 0.6 | 524 |
| 612568 | 32 | 67 | 73 | 86 | 92 | 0.5 | 525 |
| 612569 | 27 | 54 | 71 | 78 | 93 | 0.7 | 526 |
| 612615 | 44 | 64 | 65 | 70 | 75 | 0.3 | 825 |
| 612658 | 19 | 23 | 43 | 57 | 58 | 2.3 | 865 |
| 612662 | 39 | 47 | 62 | 77 | 75 | 0.6 | 868 |

TABLE 18

| | 312.5 | 625 | 1250 | 2500 | 5000 | IC₅₀ | SEQ |
|---|---|---|---|---|---|---|---|
| ISIS NO | nM | nM | nM | nM | nM | (μM) | ID NO |
| 568637 | 57 | 79 | 89 | 95 | 97 | <0.3 | 129 |
| 594625 | 72 | 80 | 91 | 97 | 97 | <0.3 | 165 |
| 610015 | 41 | 70 | 72 | 84 | 92 | 0.3 | 239 |
| 612129 | 28 | 40 | 67 | 71 | 84 | 0.9 | 889 |
| 612135 | 41 | 40 | 47 | 62 | 73 | 1.0 | 68 |
| 612145 | 22 | 48 | 54 | 61 | 65 | 1.3 | 903 |
| 612185 | 16 | 29 | 36 | 45 | 62 | 2.7 | 83 |
| 612239 | 42 | 57 | 65 | 66 | 72 | 0.5 | 966 |
| 612252 | 23 | 22 | 30 | 61 | 60 | 2.4 | 96 |
| 612806 | 52 | 73 | 67 | 76 | 73 | <0.3 | 1011 |
| 612810 | 24 | 36 | 57 | 73 | 79 | 1.1 | 1015 |
| 612816 | 14 | 30 | 24 | 51 | 61 | 2.9 | 1021 |
| 612819 | 31 | 40 | 53 | 64 | 67 | 1.2 | 1024 |
| 612901 | 40 | 44 | 54 | 72 | 80 | 0.8 | 1080 |
| 612906 | 4 | 9 | 21 | 37 | 39 | 8.8 | 1085 |

Cells were plated at a density of 20,000 cells per well and transfected using electroporation with 37 nM, 111 nM, 333 nM, 1,000 nM and 3,000 nM concentrations of antisense oligonucleotide, as specified in Table 19 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells, and are an average of two trials. The half maximal inhibitory concentration (IC₅₀) of each oligonucleotide is also presented. AGT mRNA levels were significantly reduced in a dose-dependent manner in antisense oligonucleotide treated cells.

TABLE 19

| | 37 | 111 | 333 | 1000 | 3000 | IC₅₀ | SEQ |
|---|---|---|---|---|---|---|---|
| ISIS NO | nM | nM | nM | nM | nM | (μM) | ID NO |
| 568637 | 10 | 59 | 74 | 88 | 98 | 0.1 | 129 |
| 594622 | 46 | 58 | 65 | 89 | 96 | 0.1 | 163 |
| 594625 | 24 | 46 | 68 | 85 | 94 | 0.1 | 165 |
| 594628 | 13 | 48 | 53 | 74 | 91 | 0.2 | 168 |
| 609089 | 44 | 27 | 61 | 72 | 92 | 0.2 | 184 |
| 609094 | −3 | 41 | 67 | 87 | 96 | 0.2 | 130 |
| 622210 | 18 | 36 | 51 | 74 | 95 | 0.3 | 180 |
| 622212 | 38 | 51 | 85 | 88 | 97 | 0.1 | 182 |
| 622213 | 41 | 51 | 69 | 89 | 97 | 0.1 | 164 |
| 622215 | 36 | 40 | 61 | 84 | 89 | 0.1 | 165 |
| 622216 | 18 | 51 | 60 | 85 | 96 | 0.2 | 183 |
| 622220 | 48 | 51 | 63 | 81 | 90 | 0.1 | 186 |
| 622221 | 28 | 46 | 62 | 76 | 88 | 0.2 | 167 |
| 622224 | 8 | 32 | 55 | 77 | 91 | 0.3 | 130 |
| 622238 | 45 | 33 | 60 | 67 | 91 | 0.2 | 132 |

Cells were plated at a density of 20,000 cells per well and transfected using electroporation with 12.3 nM, 37 nM, 111 nM, 333 nM, 1,000 nM and 3,000 nM concentrations of antisense oligonucleotide, as specified in Table 20 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells, and are an average of two trials. The half maximal inhibitory concentration (IC₅₀) of each oligonucleotide is also presented. AGT mRNA levels were significantly reduced in a dose-dependent manner in antisense oligonucleotide treated cells.

TABLE 20

| | 12.3 | 37 | 111 | 333 | 1000 | 3000 | IC₅₀ | SEQ |
|---|---|---|---|---|---|---|---|---|
| ISIS NO | nM | nM | nM | nM | nM | nM | (μM) | ID NO |
| 568637 | −5 | 6 | 24 | 69 | 86 | 95 | 0.2 | 129 |
| 594622 | 1 | −1 | 32 | 63 | 88 | 97 | 0.2 | 163 |
| 594624 | 9 | 0 | 54 | 57 | 87 | 92 | 0.2 | 129 |
| 594625 | 14 | 11 | 6 | 47 | 81 | 93 | 0.3 | 165 |
| 609086 | 26 | 3 | 35 | 72 | 92 | 97 | 0.1 | 181 |
| 609087 | −9 | 16 | 38 | 63 | 81 | 90 | 0.2 | 182 |
| 609088 | 11 | 9 | 44 | 61 | 86 | 97 | 0.2 | 183 |
| 609091 | 3 | 7 | 27 | 58 | 75 | 92 | 0.3 | 186 |
| 609095 | −4 | −15 | 20 | 67 | 88 | 98 | 0.3 | 189 |
| 622211 | 21 | 7 | 3 | 50 | 85 | 94 | 0.3 | 181 |
| 622214 | 8 | 19 | 39 | 69 | 89 | 96 | 0.1 | 129 |
| 622225 | 5 | 19 | 30 | 59 | 82 | 97 | 0.2 | 189 |

Cells were plated at a density of 20,000 cells per well and transfected using electroporation with 0.33 μM, 1.0 μM, 3.0 μM and 9.0 μM concentrations of antisense oligonucleotide, as specified in Table 21 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells. The half maximal inhibitory concentration (IC₅₀) of each oligonucleotide is also presented. AGT mRNA levels were significantly reduced in a dose-dependent manner in antisense oligonucleotide treated cells.

TABLE 21

| | 0.33 | 1.0 | 3.0 | 9.0 | IC₅₀ | SEQ |
|---|---|---|---|---|---|---|
| ISIS NO | μM | μM | μM | μM | (μM) | ID NO |
| 568637 | 74 | 74 | 95 | 97 | 0.03 | 129 |
| 568637 | 52 | 80 | 89 | 95 | 0.2 | 129 |
| 610015 | 47 | 64 | 90 | 92 | 0.4 | 239 |
| 610015 | 25 | 51 | 79 | 92 | 1 | 239 |
| 654385 | 2 | 38 | 71 | 82 | 1.9 | 1827 |
| 654394 | 24 | 47 | 80 | 90 | 1.1 | 1836 |
| 654401 | 27 | 57 | 85 | 89 | 0.8 | 1843 |
| 654402 | 11 | 38 | 72 | 90 | 1.5 | 1844 |
| 654404 | 16 | 47 | 79 | 82 | 1.3 | 1846 |
| 654444 | 18 | 48 | 78 | 91 | 1.2 | 1886 |
| 654451 | 34 | 59 | 83 | 93 | 0.7 | 1893 |
| 654452 | 35 | 50 | 82 | 92 | 0.8 | 1894 |
| 654472 | 23 | 49 | 79 | 93 | 1 | 1914 |
| 654481 | 22 | 53 | 79 | 93 | 1 | 1923 |
| 654483 | 28 | 63 | 80 | 95 | 0.8 | 1925 |
| 654490 | 31 | 55 | 68 | 95 | 0.9 | 1932 |
| 654559 | 16 | 44 | 75 | 92 | 1.3 | 1979 |
| 654566 | 20 | 40 | 78 | 84 | 1.3 | 1986 |
| 654568 | 37 | 58 | 81 | 92 | 0.6 | 1988 |
| 654570 | 19 | 39 | 71 | 89 | 1.4 | 1990 |
| 654691 | 31 | 57 | 86 | 92 | 0.7 | 2003 |
| 654707 | 32 | 72 | 90 | 95 | 0.5 | 2015 |
| 654737 | 31 | 69 | 83 | 96 | 0.6 | 2018 |
| 654740 | 36 | 67 | 82 | 94 | 0.5 | 2020 |
| 654768 | 29 | 64 | 82 | 95 | 0.7 | 2027 |
| 654771 | 43 | 72 | 84 | 89 | 0.3 | 2028 |
| 654815 | 25 | 51 | 78 | 91 | 1 | 2035 |
| 654817 | 23 | 55 | 89 | 95 | 0.9 | 2037 |
| 654832 | 12 | 46 | 75 | 94 | 1.3 | 2044 |
| 654843 | 20 | 57 | 85 | 87 | 1 | 2037 |
| 654846 | 26 | 57 | 84 | 92 | 0.8 | 2039 |
| 654999 | 48 | 63 | 82 | 93 | 0.4 | 238 |
| 655002 | 29 | 64 | 86 | 94 | 0.7 | 267 |
| 655049 | 38 | 67 | 88 | 95 | 0.5 | 2018 |
| 655071 | 47 | 64 | 84 | 96 | 0.4 | 2037 |
| 655093 | 35 | 71 | 86 | 93 | 0.5 | 238 |
| 655095 | 28 | 54 | 80 | 86 | 0.9 | 1708 |
| 655102 | 42 | 54 | 77 | 90 | 0.6 | 236 |

Cells were plated at a density of 20,000 cells per well and transfected using electroporation with 0.44 μM, 1.33 μM, 4.0 UM and 12.0 μM concentrations of antisense oligonucleotide, as specified in Table 22 below. After a treatment period of approximately 16 hours, RNA was isolated from the cells and AGT mRNA levels were measured by quantitative real-time PCR. Human primer probe set RTS3721 was used to measure mRNA levels. AGT mRNA levels were adjusted according to total RNA content, as measured by RIBOGREEN®. Results are presented as percent inhibition of AGT, relative to untreated control cells. The half maximal inhibitory concentration (IC₅₀) of each oligonucleotide is also presented. AGT mRNA levels were significantly reduced in a dose-dependent manner in antisense oligonucleotide treated cells.

TABLE 22

| | 0.44 | 1.33 | 4.0 | 12.0 | IC₅₀ | SEQ |
|---|---|---|---|---|---|---|
| ISIS NO | μM | μM | μM | μM | (μM) | ID NO |
| 610010 | 15 | 67 | 84 | 96 | 1.2 | 234 |
| 610010 | 20 | 64 | 85 | 97 | 1.1 | 234 |
| 610015 | 39 | 76 | 90 | 94 | 0.5 | 239 |
| 610015 | 43 | 73 | 91 | 98 | 0.5 | 239 |
| 619539 | 21 | 33 | 45 | 74 | 3.5 | 1258 |
| 619540 | 7 | 22 | 40 | 70 | 5.3 | 1259 |
| 619542 | 22 | 34 | 69 | 84 | 2.1 | 1261 |
| 619543 | 29 | 33 | 59 | 70 | 2.8 | 1262 |
| 619574 | 34 | 43 | 56 | 80 | 1.8 | 1293 |
| 619575 | 20 | 35 | 59 | 74 | 2.8 | 1294 |
| 619580 | 19 | 37 | 53 | 79 | 2.8 | 1299 |
| 619606 | 24 | 39 | 48 | 57 | 5.1 | 1325 |
| 619751 | 2 | 20 | 49 | 77 | 4.0 | 1470 |
| 619753 | 6 | 23 | 57 | 83 | 3.2 | 1472 |
| 619754 | 7 | 22 | 52 | 72 | 4.1 | 1473 |
| 619803 | 74 | 82 | 87 | 92 | <0.4 | 1522 |
| 619823 | 47 | 64 | 72 | 86 | 0.5 | 1542 |
| 619885 | 20 | 34 | 61 | 80 | 2.4 | 1604 |
| 619904 | 30 | 45 | 70 | 87 | 1.5 | 1623 |
| 619905 | 11 | 34 | 65 | 78 | 2.7 | 1624 |
| 619951 | 49 | 68 | 94 | 99 | 0.4 | 1667 |
| 619954 | 7 | 68 | 82 | 95 | 1.4 | 1670 |
| 619966 | 33 | 73 | 90 | 96 | 0.7 | 1682 |
| 619967 | 42 | 67 | 89 | 92 | 0.6 | 1683 |
| 619971 | 1 | 44 | 76 | 90 | 2.1 | 1687 |
| 619984 | 35 | 63 | 91 | 95 | 0.8 | 1700 |
| 619987 | 73 | 84 | 96 | 98 | <0.4 | 1703 |
| 619988 | 40 | 71 | 92 | 95 | 0.6 | 1704 |
| 619992 | 42 | 71 | 90 | 97 | 0.5 | 1708 |
| 619998 | 31 | 64 | 90 | 98 | 0.8 | 1714 |
| 620000 | 29 | 61 | 82 | 94 | 1.0 | 1716 |
| 620003 | 45 | 77 | 93 | 98 | 0.4 | 1719 |
| 620004 | 52 | 78 | 93 | 98 | 0.3 | 1720 |
| 620008 | 46 | 72 | 88 | 96 | 0.4 | 1724 |
| 620009 | 61 | 82 | 96 | 98 | <0.4 | 1725 |
| 620010 | 58 | 83 | 97 | 96 | <0.4 | 1726 |
| 620013 | 46 | 77 | 90 | 98 | 0.4 | 1729 |
| 620014 | 26 | 31 | 76 | 92 | 1.7 | 1730 |

### Example 3: Tolerability and Efficacy of Single Dose Treatment of Antisense Oligonucleotides Targeting Human AGT in Transgenic Mouse Model

A transgenic (Tg) mouse model “huAGT” was generated and the efficacy of antisense oligonucleotides was evaluated in this huAGT Tg model. Selected AGT antisense oligonucleotides from the in vitro studies were assessed in huAGT mice.

The huAGT transgenic mice were maintained on a 12-hour light/dark cycle and were fed ad libitum normal mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

### Treatment #1

Transgenic huAGT female mice, 10 weeks old, were divided into groups of 4 mice each. Eight groups received subcutaneous injections of antisense oligonucleotide at a dose of 20 mg/kg once per week over a course of 2.5 weeks (for three treatments). One group of mice received subcutaneous injections of PBS once per week for 2.5 weeks. The saline-injected group served as the control group to which oligonucleotide-treated groups were compared.

### RNA Analysis, Treatment #1

On day 17, total RNA was extracted from liver and kidney of the transgenic mice for real-time PCR analysis and measurement of human AGT mRNA expression. Results are presented as percent inhibition, relative to PBS control, normalized with RIBOGREEN®. As shown in Table 23, treatment with most antisense oligonucleotides resulted in significant reduction of human AGT mRNA in comparison to the PBS control.

TABLE 23
Percent inhibition of huAGT mRNA in transgenic
mouse liver and kidney relative to PBS control

| ISIS NO | liver | kidney | SEQ ID NO |
|---|---|---|---|
| 568605 | 42 | 20 | 97 |
| 568637 | 77 | 39 | 129 |
| 568638 | 56 | 11 | 130 |
| 568640 | 38 | 49 | 132 |
| 568642 | 0 | 7 | 134 |
| 568643 | 41 | 8 | 135 |
| 568647 | 49 | 32 | 139 |
| 568650 | 34 | 13 | 142 |

### Plasma Chemistry Markers, Treatment #1

To evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, NY). The results are presented in Table 24. Antisense oligonucleotides causing changes in the levels of any of the liver or kidney function markers outside the expected range for antisense oligonucleotides were excluded from further studies.

TABLE 24
Plasma chemistry markers in female transgenic huAGT mice

| | ALT | AST | T. Bilirubin | BUN |
|---|---|---|---|---|
| ISIS NO | (U/L) | (U/L) | (mg/dL) | (mg/dL) |
| PBS | 22 | 54 | 0.18 | 28 |
| 568605 | 40 | 82 | 0.19 | 28 |
| 568637 | 30 | 57 | 0.19 | 30 |
| 568638 | 39 | 67 | 0.21 | 27 |
| 568640 | 78 | 141 | 0.28 | 31 |
| 568642 | 127 | 227 | 0.39 | 25 |
| 568643 | 35 | 66 | 0.16 | 31 |
| 568647 | 26 | 46 | 0.18 | 27 |
| 568650 | 71 | 105 | 0.18 | 27 |

### Body and Organ Weights, Treatment #1

Body weights of transgenic mice were measured at day 15 and the average body weight for each group is presented in the table below. Liver, spleen and kidney weights were measured at the end of the study, and are presented in Table 25. Antisense oligonucleotides that caused any changes in organ weights outside the expected range for antisense oligonucleotides were excluded from further studies.

TABLE 25
Body and organ weights (in grams)

| ISIS NO | body (g) | kidney (g) | liver (g) | spleen (g) |
|---|---|---|---|---|
| PBS | 18.8 | 0.3 | 0.9 | 0.08 |
| 568605 | 19.0 | 0.2 | 1.0 | 0.09 |
| 568637 | 19.3 | 0.3 | 1.0 | 0.08 |
| 568638 | 20.5 | 0.3 | 0.9 | 0.11 |
| 568640 | 19.7 | 0.3 | 1.0 | 0.09 |
| 568642 | 19.3 | 0.3 | 1.0 | 0.08 |
| 568643 | 19.9 | 0.3 | 1.0 | 0.09 |
| 568647 | 20.6 | 0.3 | 1.0 | 0.09 |
| 568650 | 20.0 | 0.3 | 1.0 | 0.09 |

### Treatment #2

Groups of two huAGT mice each received subcutaneous injections of antisense oligonucleotide at doses of 25 mg/kg/wk over the course of two weeks. One group of huAGT mice received subcutaneous injections of PBS as the control group to which oligonucleotide-treated groups were compared.

### RNA Analysis, Treatment #2

On day 10, total RNA was extracted from livers of the transgenic mice for real-time PCR analysis and measurement of human AGT mRNA expression. The results were averaged for each group of two mice, and are presented as percent inhibition, relative to PBS control, normalized with RIBOGREEN®. As shown in Table 26, treatment with most antisense antisense oligonucleotides resulted in significant reduction of human AGT mRNA in comparison to the PBS control.

TABLE 26
Percent inhibition of human AGT mRNA in the transgenic
mouse liver relative to the PBS control

| ISIS NO | % inhibit | SEQ ID NO |
|---|---|---|
| 568637 | 96 | 129 |
| 610010 | 66 | 234 |
| 610015 | 29 | 239 |
| 619967 | 59 | 1683 |
| 619984 | 56 | 1700 |
| 619987 | 25 | 1703 |
| 619988 | 38 | 1704 |
| 619992 | 70 | 1708 |
| 619998 | 75 | 1714 |
| 620000 | 75 | 1716 |
| 620003 | 56 | 1719 |
| 620004 | 27 | 1720 |
| 620008 | 4 | 1724 |
| 620009 | 41 | 1725 |
| 620010 | 72 | 1726 |
| 620013 | 65 | 1729 |

### Plasma Chemistry Markers, Treatment #2

To evaluate the effect of antisense oligonucleotides on liver function, plasma levels of transaminases were measured using an automated clinical chemistry analyzer (Hitachi Olympus AU400e, Melville, NY). The results were averaged for each group of two mice, and are presented in Table 27. Antisense oligonucleotides causing changes in the levels of any of the liver function markers outside the expected range for antisense oligonucleotides were excluded from further studies.

TABLE 27
Plasma chemistry markers in female transgenic huAGT mice

| ISIS NO | ALT (U/L) | AST (U/L) |
|---|---|---|
| PBS | 29 | 58 |
| 568637 | 29 | 82 |
| 610010 | 28 | 72 |
| 610015 | 71 | 103 |
| 619967 | 58 | 179 |
| 619984 | 23 | 41 |
| 619987 | 24 | 39 |
| 619988 | 29 | 107 |
| 619992 | 26 | 43 |
| 619998 | 25 | 71 |
| 620000 | 31 | 106 |
| 620003 | 24 | 46 |
| 620004 | 24 | 105 |
| 620008 | 24 | 51 |
| 620009 | 28 | 53 |
| 620010 | 24 | 38 |
| 620013 | 41 | 130 |

### Body and Organ Weights, Treatment #2

Body weights of all treatment groups of huAGT mice were measured at day 1 and day 8, and animals were sacrificed and their livers harvested and weighed at day 10. The results were averaged for each group of two mice, and are presented in Table 28. Antisense oligonucleotides that caused any changes in organ weights outside the expected range for antisense oligonucleotides were excluded from further studies.

TABLE 28
Body and liver weights (in grams)

| ISIS NO | Day 1 body (g) | Day 8 body (g) | liver (g) |
|---|---|---|---|
| PBS | 18.3 | 18.8 | 1.0 |
| 568637 | 20.0 | 20.5 | 1.1 |
| 610010 | 19.0 | 19.4 | 1.1 |
| 610015 | 19.9 | 20.7 | 1.2 |
| 619967 | 19.8 | 19.9 | 1.0 |
| 619984 | 18.9 | 19.3 | 1.0 |
| 619987 | 20.2 | 20.5 | 1.2 |
| 619988 | 17.3 | 18.2 | 0.9 |
| 619992 | 18.3 | 19.4 | 1.0 |
| 619998 | 18.8 | 19.0 | 1.0 |
| 620000 | 19.7 | 20.4 | 1.1 |
| 620003 | 19.8 | 20.2 | 1.0 |
| 620004 | 21.0 | 21.6 | 1.1 |
| 620008 | 20.0 | 19.8 | 1.0 |
| 620009 | 18.9 | 19.0 | 1.0 |
| 620010 | 18.9 | 19.6 | 1.0 |
| 620013 | 19.7 | 20.3 | 1.1 |

### Treatment #3

Groups of two huAGT mice each received subcutaneous injections of antisense oligonucleotide at doses of 25 mg/kg/wk over the course of two weeks. One group of four huAGT mice received subcutaneous injections of PBS as the control group to which oligonucleotide-treated groups were compared.

### RNA Analysis, Treatment #3

On day 10, total RNA was extracted from livers of the transgenic mice for real-time PCR analysis and measurement of human AGT mRNA expression. The results were averaged for each group of two mice, and are presented as percent inhibition, relative to PBS control, normalized with RIBOGREEN®. As shown in Table 29, treatment with most antisense antisense oligonucleotides resulted in significant reduction of human AGT mRNA in comparison to the PBS control.

TABLE 29
Percent inhibition of human AGT mRNA in the transgenic
mouse liver relative to the PBS control

| ISIS NO | % inhibit | SEQ ID NO |
|---|---|---|
| 568637 | 93 | 129 |
| 654401 | 63 | 1843 |
| 654451 | 43 | 1893 |
| 654452 | 48 | 1894 |
| 654472 | 69 | 1914 |
| 654481 | 0 | 1923 |
| 654483 | 58 | 1925 |
| 654490 | 80 | 1932 |
| 654568 | 70 | 1988 |
| 654691 | 81 | 2003 |
| 654707 | 32 | 2015 |
| 654740 | 0 | 2020 |
| 654771 | 0 | 2028 |
| 654999 | 76 | 238 |
| 655049 | 75 | 2018 |
| 655071 | 81 | 2037 |
| 655093 | 59 | 238 |

### Plasma Chemistry Markers, Treatment #3

To evaluate the effect of antisense oligonucleotides on liver function, plasma levels of transaminases were measured using an automated clinical chemistry analyzer (Hitachi Olympus AU400e, Melville, NY). The results were averaged for each group of two mice, and are presented in Table 30. Antisense oligonucleotides causing changes in the levels of any of the liver function markers outside the expected range for antisense oligonucleotides were excluded from further studies.

TABLE 30
Plasma chemistry markers in female transgenic huAGT mice

| ISIS NO | ALT (U/L) | AST (U/L) |
|---|---|---|
| PBS | 32 | 44 |
| 568637 | 36 | 41 |
| 654401 | 34 | 44 |
| 654451 | 52 | 82 |
| 654452 | 29 | 54 |
| 654472 | 50 | 78 |
| 654481 | 35 | 43 |
| 654483 | 28 | 62 |
| 654490 | 28 | 75 |
| 654568 | 35 | 60 |
| 654691 | 32 | 54 |
| 654707 | 48 | 65 |
| 654740 | 43 | 55 |
| 654771 | 59 | 166 |
| 654999 | 31 | 60 |
| 655049 | 27 | 61 |
| 655071 | 42 | 67 |
| 655093 | 26 | 50 |

### Body and Organ Weights, Treatment #3

Body weights of all treatment groups of huAGT mice were measured at day 1 and day 8, and animals were sacrificed and their livers harvested and weighed at day 10. The results were averaged for each group of two mice, and are presented in Table 31. Antisense oligonucleotides that caused any changes in weights outside the expected range for antisense oligonucleotides were excluded from further studies.

TABLE 31
Body and liver weights

| ISIS NO | Day 1 body (g) | Day 8 body (g) | liver (g) |
|---|---|---|---|
| PBS | 26.7 | 27.4 | 1.5 |
| 568637 | 28.6 | 29.9 | 1.7 |
| 654401 | 29.1 | 30.9 | 1.9 |
| 654451 | 27.0 | 27.4 | 1.4 |
| 654452 | 26.6 | 27.2 | 1.4 |
| 654472 | 29.7 | 30.8 | 1.8 |
| 654481 | 28.3 | 29.4 | 1.6 |
| 654483 | 25.8 | 26.4 | 1.3 |
| 654490 | 28.6 | 28.7 | 1.5 |
| 654568 | 28.6 | 29.6 | 1.7 |
| 654691 | 29.6 | 31.1 | 1.7 |
| 654707 | 29.3 | 30.4 | 1.9 |
| 654740 | 29.1 | 29.8 | 1.7 |
| 654771 | 29.1 | 30.3 | 1.7 |
| 654999 | 28.2 | 29.0 | 1.6 |
| 655049 | 29.8 | 32.2 | 1.8 |
| 655071 | 28.5 | 30.4 | 1.8 |
| 655093 | 28.0 | 29.7 | 1.6 |

### Treatment #4

Transgenic huAGT male mice, six weeks old, were divided into groups of 3-4 mice each. Eight groups received subcutaneous injections of antisense oligonucleotide at a dose of 5 mg/kg once per week over a course of 2 weeks. One group of mice received subcutaneous injections of PBS once per week for 2 weeks. The saline-injected group served as the control group to which oligonucleotide-treated groups were compared.

### RNA Analysis, Treatment #4

On day 17, total RNA was extracted from liver and kidney of the transgenic mice for real-time PCR analysis and measurement of human AGT mRNA expression. Results are presented as percent inhibition, relative to PBS control, normalized with RIBOGREEN®. As shown in Table 32, treatment with most antisense oligonucleotides resulted in significant reduction of human AGT mRNA in comparison to the PBS control.

TABLE 32
Percent inhibition of huAGT mRNA in transgenic
mouse liver and kidney relative to PBS control

| ISIS NO | liver | kidney | SEQ ID NO |
|---|---|---|---|
| 594622 | 81 | 90 | 163 |
| 594623 | 32 | 55 | 164 |
| 594624 | 79 | 67 | 129 |
| 594625 | 91 | 70 | 165 |
| 594626 | 76 | 81 | 166 |
| 594627 | 82 | 88 | 167 |
| 594628 | 28 | 22 | 168 |
| 594629 | 17 | 20 | 169 |
| 594630 | 37 | 35 | 170 |
| 594631 | 45 | 75 | 171 |
| 594632 | 50 | 51 | 172 |
| 568637 | 67 | 54 | 129 |

### Plasma Chemistry Markers, Treatment #4

On day 15, to evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, NY). The results are presented in Table 33. Antisense oligonucleotides causing changes in the levels of any of the liver function markers outside the expected range for antisense oligonucleotides were excluded from further studies.

TABLE 33
Plasma chemistry markers in female transgenic huAGT mice

| | ALT | AST | T. Bilirubin | BUN |
|---|---|---|---|---|
| ISIS NO | (U/L) | (U/L) | (mg/dL) | (mg/dL) |
| PBS | 77 | 118 | 0.18 | 40 |
| 594622 | 71 | 152 | 0.24 | 34 |
| 594623 | 57 | 92 | 0.18 | 36 |
| 594624 | 53 | 72 | 0.14 | 40 |
| 594625 | 92 | 116 | 0.17 | 36 |
| 594626 | 43 | 68 | 0.15 | 37 |
| 594627 | 50 | 67 | 0.17 | 35 |
| 594628 | 86 | 210 | 0.24 | 34 |
| 594629 | 55 | 68 | 0.16 | 31 |
| 594630 | 55 | 59 | 0.16 | 32 |
| 594631 | 32 | 44 | 0.15 | 36 |
| 594632 | 58 | 59 | 0.15 | 35 |
| 568637 | 110 | 371 | 0.22 | 31 |

### Body and Organ Weights, Treatment #4

Body weights of transgenic mice were measured at days 1, 8 and 13 and the averages for each group are presented in the table below. On day 15, liver, spleen and kidney weights were also measured, and are presented in Table 34. Antisense oligonucleotides that caused any changes in weights outside the expected range for antisense oligonucleotides were excluded from further studies.

TABLE 34
Body and organ weights (in grams)

| | body (g) | | | | | |
|---|---|---|---|---|---|---|
| ISIS NO | Day 1 | Day 8 | Day 13 | kidney (g) | liver (g) | spleen (g) |
| PBS | 20.4 | 21.6 | 21.5 | 0.3 | 1.2 | 0.08 |
| 594622 | 18.5 | 21.6 | 21.6 | 0.3 | 1.5 | 0.11 |
| 594623 | 18.1 | 20.4 | 20.3 | 0.3 | 1.2 | 0.06 |
| 594624 | 19.8 | 22.8 | 22.6 | 0.3 | 1.3 | 0.08 |
| 594625 | 20.3 | 22.2 | 22.1 | 0.3 | 1.3 | 0.06 |
| 594626 | 21.6 | 22.9 | 22.7 | 0.3 | 1.2 | 0.07 |
| 594627 | 21.9 | 22.8 | 22.7 | 0.3 | 1.2 | 0.07 |
| 594628 | 20.6 | 22.2 | 21.9 | 0.3 | 1.2 | 0.07 |
| 594629 | 20.8 | 22.1 | 22.0 | 0.3 | 1.2 | 0.07 |
| 594630 | 22.2 | 24.0 | 23.7 | 0.3 | 1.2 | 0.08 |
| 594631 | 20.2 | 21.9 | 21.6 | 0.3 | 1.1 | 0.07 |
| 594632 | 21.3 | 22.5 | 22.4 | 0.3 | 1.3 | 0.07 |
| 568637 | 20.1 | 21.4 | 21.5 | 0.3 | 1.2 | 0.05 |

### Example 4: Tolerability and Efficacy of Multiple Dose Treatment of Antisense Oligonucleotides Targeting Human AGT in Transgenic Mouse Model

Selected AGT antisense oligonucleotides from the single dose studies in huAGT transgenic mice were further assessed in dose response studies in huAGT transgenic mice.

The huAGT transgenic mice were maintained on a 12-hour light/dark cycle and were fed ad libitum normal mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

### Treatment #1

For a four point dose-response study, male huAGT mice were divided into 37 groups of four mice each. 36 groups received subcutaneous injections of antisense oligonucleotide at doses of 5, 10, 25 and 50 mg/kg/week for 2.5 weeks (three doses in total). One group of huAGT mice received subcutaneous injections of saline as a control group, to which oligonucleotide-treated groups were compared.

### RNA Analysis, Treatment #1

On day 17, the huAGT mice were sacrificed, and total RNA was extracted from liver and kidney for real-time PCR analysis and measurement of human AGT mRNA expression. RT-PCR results are presented as average percent inhibition relative to the saline-treated control group, and normalized with RIBOGREEN®. As shown in Table 35, treatment with the selected antisense oligonucleotides resulted in significant reduction of human AGT mRNA in comparison to the saline control.

TABLE 35
Percent inhibition of human AGT mRNA in organs
of huAGT mice treated with nine lead ASOs

| ISIS NO | mg/kg/wk | ED50 | Liver | Kidney | SEQ ID NO |
|---|---|---|---|---|---|
| 619998 | 50 | 7 | 96 | 71 | 1714 |
| | 25 | | 89 | 59 | |
| | 10 | | 80 | 69 | |
| | 5 | | 45 | 47 | |
| 620003 | 50 | 10 | 80 | 69 | 1719 |
| | 25 | | 91 | 68 | |
| | 10 | | 51 | 52 | |
| | 5 | | 35 | 58 | |
| 654451 | 50 | 9 | 94 | 56 | 1893 |
| | 25 | | 81 | 48 | |
| | 10 | | 36 | 43 | |
| | 5 | | 11 | 48 | |
| 654452 | 50 | 8 | 82 | 53 | 1894 |
| | 25 | | 77 | 59 | |
| | 10 | | 69 | 62 | |
| | 5 | | 0 | 54 | |
| 654472 | 50 | 5 | 81 | 41 | 1914 |
| | 25 | | 82 | 62 | |
| | 10 | | 51 | 50 | |
| | 5 | | 46 | 51 | |
| 654481 | 50 | ~47 | 84 | 70 | 1923 |
| | 25 | | 31 | 54 | |
| | 10 | | 47 | 59 | |
| | 5 | | 52 | 67 | |
| 654483 | 50 | 18 | 78 | 33 | 1925 |
| | 25 | | 77 | 45 | |
| | 10 | | 84 | 73 | |
| | 5 | | 11 | 41 | |
| 654691 | 50 | 6 | 93 | 70 | 2003 |
| | 25 | | 87 | 78 | |
| | 10 | | 43 | 70 | |
| | 5 | | 54 | 70 | |
| 654999 | 50 | 1 | 99 | 87 | 238 |
| | 25 | | 95 | 76 | |
| | 10 | | 74 | 78 | |
| | 5 | | 69 | 81 | |

### Body and Organ Weights, Treatment #1

Body weights of all treatment groups of huAGT mice were measured at days 1, 8 and 15 of the experiment. The results were averaged for each group of mice, and are presented in Table 36.

TABLE 36
Body Weight (BW) of huAGT mice treated with nine lead ASOs

BW (grams)

| ISIS NO | mg/kg/wk | day 1 | day 8 | day 15 |
|---|---|---|---|---|
| saline | n/a | 28 | 29 | 29 |
| 619998 | 50 | 30 | 30 | 31 |
| | 25 | 29 | 29 | 30 |
| | 10 | 32 | 32 | 32 |
| | 5 | 32 | 32 | 31 |
| 620003 | 50 | 31 | 32 | 32 |
| | 25 | 32 | 32 | 33 |
| | 10 | 30 | 30 | 30 |
| | 5 | 32 | 32 | 32 |
| 654451 | 50 | 27 | 28 | 28 |
| | 25 | 28 | 28 | 28 |
| | 10 | 26 | 27 | 27 |
| | 5 | 27 | 28 | 29 |
| 654452 | 50 | 28 | 29 | 28 |
| | 25 | 27 | 28 | 28 |
| | 10 | 27 | 28 | 27 |
| | 5 | 28 | 28 | 29 |
| 654472 | 50 | 26 | 27 | 28 |
| | 25 | 27 | 27 | 28 |
| | 10 | 25 | 26 | 27 |
| | 5 | 27 | 27 | 28 |
| 654481 | 50 | 28 | 29 | 29 |
| | 25 | 33 | 34 | 34 |
| | 10 | 32 | 33 | 33 |
| | 5 | 30 | 32 | 32 |
| 654483 | 50 | 34 | 36 | 36 |
| | 25 | 31 | 31 | 32 |
| | 10 | 29 | 30 | 30 |
| | 5 | 31 | 32 | 32 |
| 654691 | 50 | 29 | 30 | 30 |
| | 25 | 30 | 31 | 31 |
| | 10 | 30 | 31 | 32 |
| | 5 | 30 | 30 | 30 |
| 654999 | 50 | 33 | 33 | 34 |
| | 25 | 33 | 32 | 32 |
| | 10 | 31 | 31 | 31 |
| | 5 | 31 | 31 | 31 |

### Treatment #2

Five potent antisense oligonucleotides targeting human AGT from previous studies (ISIS NOs. 620003, 654451, 654472, 654691 and 654999) were selected for another four point dose-response study and compared to ISIS 568637 which had been potent in vitro and potent and tolerable in single dose huAGT transgenic mice studies. In this study, huAGT mice were divided into 25 groups of three mice each. Groups received subcutaneous injections of antisense oligonucleotide at doses of 1, 4, 10 and 40 mg/kg for two injections over ten days. One group of three huAGT mice received subcutaneous injections of saline as a control group, to which oligonucleotide-treated groups were compared.

### RNA Analysis, Treatment #2

On day 10, the antisense oligonucleotide treated huAGT mice were sacrificed, and total RNA was extracted from liver and kidney for real-time PCR analysis and measurement of human AGT mRNA expression. Results are presented as average percent inhibition of mRNA, relative to the PBS control group, and normalized with RIBOGREEN®. As shown in Table 37, treatment with the antisense oligonucleotides resulted in significant reduction of human AGT mRNA in comparison to the saline control.

TABLE 37
Percent inhibition of human AGT mRNA in organs
of huAGT mice treated with five lead ASOs

| ISIS NO | mg/kg | ED50 AGT mRNA | Liver | Kidney | SEQ ID NO |
|---|---|---|---|---|---|
| 568637 | 1 | 4.1 | 16 | 54 | 129 |
| | 4 | | 42 | 69 | |
| | 10 | | 82 | 82 | |
| | 40 | | 96 | 90 | |
| 620003 | 1 | 9.5 | 22 | 25 | 1719 |
| | 4 | | 29 | 32 | |
| | 10 | | 54 | 50 | |
| | 40 | | 81 | 49 | |
| 654451 | 1 | 8.0 | 18 | 31 | 1893 |
| | 4 | | 24 | 32 | |
| | 10 | | 59 | 49 | |
| | 40 | | 87 | 58 | |
| 654472 | 1 | 5.6 | 15 | 13 | 1914 |
| | 4 | | 26 | 38 | |
| | 10 | | 64 | 59 | |
| | 40 | | 82 | 66 | |
| 654691 | 1 | 7.2 | 10 | 18 | 2003 |
| | 4 | | 28 | 53 | |
| | 10 | | 63 | 61 | |
| | 40 | | 95 | 65 | |
| 654999 | 1 | 3.4 | 0 | 13 | 238 |
| | 4 | | 37 | 62 | |
| | 10 | | 70 | 65 | |
| | 40 | | 93 | 67 | |

### Plasma Chemistry Markers, Treatment #2

On day 10, plasma levels of transaminases, bilirubin and BUN were measured using an automated clinical chemistry analyzer (Hitachi Olympus AU400e, Melville, NY) to evaluate the effect of antisense oligonucleotides on liver and kidney function. The results are presented in Table 38.

TABLE 38
Plasma chemistry markers in transgenic huAGT mice

| | | ALT | AST | BUN | T. Bil |
|---|---|---|---|---|---|
| ISIS NO | mg/kg | (U/L) | (U/L) | (mg/dL) | (mg/dL) |
| PBS | n/a | 34 | 62 | 29 | 0.12 |
| 568637 | 1 | 29 | 50 | 25 | 0.08 |
| | 4 | 37 | 54 | 31 | 0.11 |
| | 10 | 44 | 54 | 29 | 0.12 |
| | 40 | 39 | 52 | 27 | 0.12 |
| 620003 | 1 | 32 | 59 | 27 | 0.16 |
| | 4 | 44 | 53 | 34 | 0.10 |
| | 10 | 40 | 60 | 29 | 0.14 |
| | 40 | 33 | 34 | 26 | 0.11 |
| 654451 | 1 | 38 | 49 | 30 | 0.12 |
| | 4 | 33 | 49 | 30 | 0.13 |
| | 10 | 35 | 45 | 29 | 0.11 |
| | 40 | 33 | 38 | 29 | 0.12 |
| 654472 | 1 | 39 | 69 | 28 | 0.17 |
| | 4 | 31 | 54 | 30 | 0.11 |
| | 10 | 30 | 70 | 30 | 0.15 |
| | 40 | 33 | 41 | 30 | 0.10 |
| 654691 | 1 | 39 | 79 | 32 | 0.11 |
| | 4 | 35 | 54 | 29 | 0.12 |
| | 10 | 34 | 44 | 32 | 0.12 |
| | 40 | 37 | 43 | 30 | 0.14 |
| 654999 | 1 | 34 | 56 | 31 | 0.11 |
| | 4 | 38 | 51 | 32 | 0.13 |
| | 10 | 29 | 53 | 33 | 0.09 |
| | 40 | 30 | 42 | 28 | 0.09 |

### Treatment #3

Five potent antisense oligonucleotides targeting human AGT from a previous dose response study (ISIS NOs. 568637, 594622, 594624, 594625 and 594627) were selected for a three-point dose-response study. In this study, huAGT mice were divided into 16 groups of three mice each. Groups received subcutaneous injections of antisense oligonucleotide at doses of 1, 5 and 15 mg/kg for two injections over the course of a week. One group of three huAGT mice received subcutaneous injections of saline as a control group, to which oligonucleotide-treated groups were compared.

### RNA Analysis, Treatment #3

On day 8, total RNA was extracted from liver and kidneys of the transgenic mice for real-time PCR analysis and measurement of human AGT mRNA expression. Results are presented as percent inhibition, relative to PBS control, normalized with RIBOGREEN®. As shown in Table 39, treatment with most antisense oligonucleotides resulted in significant reduction of human AGT mRNA in comparison to the PBS control.

TABLE 39
Percent inhibition of huAGT mRNA in transgenic
mouse liver and kidney relative to PBS control

| | | Liver ED50 | | | | SEQ |
|---|---|---|---|---|---|---|
| | ISIS NO | AGT mRNA | mg/kg | liver | kidney | ID NO |
| males | 594622 | 2.4 | 1 | 35 | 76 | 163 |
| | | | 5 | 84 | 89 | |
| | | | 15 | 98 | 92 | |
| | 594624 | 3.9 | 1 | 23 | 10 | 129 |
| | | | 5 | 84 | 70 | |
| | | | 15 | 96 | 83 | |
| | 594625 | 1.8 | 1 | 34 | 15 | 165 |
| | | | 5 | 82 | 59 | |
| | | | 15 | 96 | 76 | |
| | 594627 | 1.4 | 1 | 17 | 71 | 167 |
| | | | 5 | 78 | 87 | |
| | | | 15 | 91 | 91 | |
| | 568637 | 3.8 | 1 | 21 | 10 | 129 |
| | | | 5 | 75 | 49 | |
| | | | 15 | 91 | 74 | |
| females | 594625 | 1.7 | 1 | 45 | 77 | 165 |
| | | | 5 | 86 | 88 | |
| | | | 15 | 98 | 96 | |

### Plasma Chemistry Markers, Treatment #3

On day 8, to evaluate the effect of antisense oligonucleotides on liver and kidney function, plasma levels of transaminases, total bilirubin and blood urea nitrogen (BUN) were measured using an automated clinical chemistry analyzer (Hitachi Olympus AU400e, Melville, NY). The results are presented in Table 40.

TABLE 40
Plasma chemistry markers in male
and female transgenic huAGT mice

| | | | ALT | AST | BUN | T. Bilirubin |
|---|---|---|---|---|---|---|
| | ISIS NO | mg/kg | (U/L) | (U/L) | (mg/dL) | (mg/dL) |
| males | | PBS | 67 | 101 | 34 | 0.26 |
| | 594622 | 1 | 34 | 43 | 36 | 0.14 |
| | | 5 | 92 | 151 | 32 | 0.29 |
| | | 15 | 132 | 167 | 30 | 0.30 |
| | 594624 | 1 | 40 | 57 | 31 | 0.15 |
| | | 5 | 46 | 83 | 35 | 0.12 |
| | | 15 | 37 | 74 | 32 | 0.15 |
| | 594625 | 1 | 74 | 166 | 33 | 0.23 |
| | | 5 | 55 | 67 | 34 | 0.18 |
| | | 15 | 63 | 89 | 34 | 0.15 |
| | 594627 | 1 | 36 | 96 | 34 | 0.12 |
| | | 5 | 40 | 67 | 33 | 0.13 |
| | | 15 | 57 | 62 | 30 | 0.13 |
| | 568637 | 1 | 38 | 69 | 33 | 0.14 |
| | | 5 | 33 | 48 | 32 | 0.15 |
| | | 15 | 74 | 81 | 28 | 0.14 |
| females | | PBS | 39 | 61 | 33 | 0.17 |
| | 594625 | 1 | 53 | 91 | 29 | 0.22 |
| | | 5 | 276 | 304 | 28 | 0.25 |
| | | 15 | 60 | 77 | 29 | 0.21 |

### Body and Organ Weights, Treatment #3

Body weights of transgenic mice were measured at days 1, 8 and 13 and the averages for each group are presented in the table below. On day 15, liver, spleen and kidney weights were also measured, and are presented in Table 41.

TABLE 41
Body and organ weights (in grams)

| | | mg/ | body (g) | | kidney | liver | spleen |
|---|---|---|---|---|---|---|---|
| | ISIS NO | kg | Day 1 | Day 6 | (g) | (g) | (g) |
| males | | PBS | 23.6 | 23.9 | 0.33 | 1.3 | 0.08 |
| | 594622 | 1 | 23.3 | 23.7 | 0.30 | 1.3 | 0.07 |
| | | 5 | 22.7 | 23.5 | 0.34 | 1.4 | 0.09 |
| | | 15 | 23.9 | 24.4 | 0.33 | 1.8 | 0.08 |
| | 594624 | 1 | 24.9 | 26.0 | 0.35 | 1.4 | 0.08 |
| | | 5 | 23.8 | 24.6 | 0.33 | 1.4 | 0.09 |
| | | 15 | 23.7 | 24.2 | 0.33 | 1.4 | 0.07 |
| | 594625 | 1 | 23.3 | 23.7 | 0.31 | 1.3 | 0.07 |
| | | 5 | 22.1 | 23.0 | 0.30 | 1.4 | 0.08 |
| | | 15 | 23.8 | 24.6 | 0.32 | 1.6 | 0.09 |
| | 594627 | 1 | 22.8 | 23.8 | 0.31 | 1.3 | 0.07 |
| | | 5 | 23.8 | 23.9 | 0.32 | 1.4 | 0.08 |
| | | 15 | 21.2 | 21.7 | 0.29 | 1.4 | 0.07 |
| | 568637 | 1 | 22.6 | 23.3 | 0.30 | 1.3 | 0.08 |
| | | 5 | 22.7 | 22.9 | 0.31 | 1.2 | 0.07 |
| | | 15 | 23.0 | 23.6 | 0.31 | 1.4 | 0.08 |
| females | | PBS | 17.6 | 18.0 | 0.25 | 1.0 | 0.07 |
| | 594625 | 1 | 18.2 | 18.4 | 0.24 | 1.0 | 0.08 |
| | | 5 | 18.0 | 18.8 | 0.25 | 1.1 | 0.08 |
| | | 15 | 19.2 | 19.7 | 0.28 | 1.2 | 0.09 |

### Example 5: Viscosity Assessment of Nine Lead Antisense Oligonucleotides Targeting AGT

The viscosity of the 9 antisense oligonucleotides was measured with the aim of screening out antisense oligonucleotides which have a viscosity more than 40 cP. Oligonucleotides having a viscosity greater than 40 cP are considered too viscous to be administered to any subject.

Antisense oligonucleotides (32-35 mg) were weighed into a glass vial, 120 μL of water was added and the antisense oligonucleotide was dissolved into solution by heating the vial at 50° C. Part of (75 μL) the pre-heated sample was pipetted to a micro-viscometer (Cambridge). The temperature of the micro-viscometer was set to 25° C. and the viscosity of the sample was measured. Another part (20 μL) of the pre-heated sample was pipetted into 10 mL of water for UV reading at 260 nM at 85° C. (Cary UV instrument). The results are presented in Table 42 and indicate that the antisense oligonucleotides tested do not exceed a viscosity of 40 cP.

TABLE 42
Viscosity Data for ASOs targeting AGT

| ISIS NO | Chemistry | cP |
|---|---|---|
| 619998 | 5-10-5 MOE | 29 |
| 620003 | 5-10-5 MOE | 12 |
| 654451 | 5-10-5 MOE | 25 |
| 654452 | 5-10-5 MOE | 13 |
| 654472 | 5-10-5 MOE | 11 |
| 654481 | 5-10-5 MOE | 12 |
| 654483 | 5-10-5 MOE | 28 |
| 654691 | 3-10-4 MOE | 23 |
| 654999 | 5-8-7 MOE | 34 |

### Example 6: Tolerability of Nine Lead Antisense Oligonucleotides (ASOs) Targeting Human AGT in CD1 Mice

CD1® mice (Charles River, MA) are a multipurpose mice model, frequently utilized for safety and efficacy testing. The mice were treated with antisense oligonucleotides selected from studies described above and evaluated for changes in the levels of various plasma chemistry markers.

The 9 antisense oligonucleotides identified in the examples, above, were tested in CD1 mice for tolerability. The mice were divided into groups of four mice per group, and were injected subcutaneously twice a week for six weeks with 50 mg/kg of antisense oligonucleotides (100 mg/kg/week dose). One group of male CD1 mice was injected subcutaneously twice a week for six weeks with PBS. Mice were euthanized 48 hours after the last dose, and organs and plasma were harvested for further analysis.

### Body and Organ Weights

Body weights of ASO-treated CD1 mice were measured weekly. On day 43, the mice were sacrificed and organs harvested and weighed. The body and organ weights in grams (g) at the end of the study are shown in Table 43.

TABLE 43
Body and organ weights (grams) of CD1
mice treated with nine lead ASOs

| ISIS NO | body day 41 | liver | kidney | spleen |
|---|---|---|---|---|
| PBS | 39.1 | 2.2 | 0.7 | 0.2 |
| 619998 | 42.5 | 2.5 | 0.6 | 0.3 |
| 620003 | 38.9 | 2.5 | 0.6 | 0.2 |
| 654451 | 31.6 | 1.8 | 0.5 | 0.1 |
| 654452 | 37.1 | 2.3 | 0.6 | 0.2 |
| 654472 | 37.2 | 2.3 | 0.6 | 0.1 |
| 654481 | 37.7 | 2.2 | 0.6 | 0.2 |
| 654483 | 35.1 | 2.3 | 0.6 | 0.2 |
| 654691 | 37.5 | 2.3 | 0.7 | 0.3 |
| 654999 | 35.9 | 2.2 | 0.5 | 0.5 |

### Plasma Chemistry Markers

To evaluate the effect of the oligonucleotides on liver and kidney function, plasma levels of ALT (alanine transaminase) and AST (aspartate transaminase), bilirubin, creatinine, and BUN were measured using an automated clinical chemistry analyzer (Hitachi Olympus AU400e, Melville, NY).

The results were averaged for each group, and a selection of these is presented in Table 44.

TABLE 44
Plasma chemistry markers in CD1 mice

| | | ALT | AST | BUN | Cre | T. Bilirubin |
|---|---|---|---|---|---|---|
| ISIS NO | Compound | (U/L) | (U/L) | (mg/dL) | (mg/dL) | (mg/dL) |
| PBS | N/A | 25 | 41 | 28 | 0.15 | 0.14 |
| 619998 | 5-10-5 MOE | 79 | 124 | 28 | 0.16 | 0.12 |
| 620003 | 5-10-5 MOE | 30 | 46 | 29 | 0.16 | 0.14 |
| 654451 | 5-10-5 MOE | 46 | 84 | 22 | 0.08 | 0.16 |
| 654452 | 5-10-5 MOE | 122 | 182 | 25 | 0.10 | 0.11 |
| 654472 | 5-10-5 MOE | 50 | 65 | 29 | 0.11 | 0.11 |
| 654481 | 5-10-5 MOE | 35 | 50 | 25 | 0.08 | 0.14 |
| 654483 | 5-10-5 MOE | 107 | 108 | 25 | 0.09 | 0.17 |
| 654691 | 3-10-4 MOE | 95 | 109 | 25 | 0.11 | 0.13 |
| 654999 | 5-8-7 MOE | 71 | 135 | 28 | 0.11 | 0.10 |

In a separate study antisense compounds ISIS 568637, 594622, 594624, 594625 and 594627 were also tested in CD1 mice, but exhibited some tolerability issues and the study was terminated early.

### Example 7: Tolerability of Nine Lead Antisense Oligonucleotides (ASOs) Targeting Human AGT in Sprague-Dawley Rats

Sprague-Dawley (SD) rats are a multipurpose model used for safety and efficacy evaluations. The SD rats were treated with 9 antisense oligonucleotides selected from the studies described in the Examples above and evaluated for changes in the levels of various plasma chemistry markers.

### Treatment

Male SD rats were maintained on a 12-hour light/dark cycle and fed ad libitum with Purina normal rat chow. The rats were divided into groups of four rats per group, and each group was injected subcutaneously with 100 mg/kg/week for six weeks. Forty eight hours after the last dose, rats were euthanized and organs and plasma were harvested for further analysis.

### Organ Weights

Liver, spleen and kidney weights of antisense oligonucleotide treated rats were measured at the end of the study. The body and organ weights are shown in grams in Table 45.

TABLE 45
Body and organ weights (grams) of Sprague-
Dawley rats treated with nine lead ASOs

| ISIS NO | body | kidney | liver | spleen |
|---|---|---|---|---|
| 619998 | 333 | 3.0 | 12.1 | 2.6 |
| 620003 | 361 | 2.9 | 11.7 | 1.4 |
| 654451 | 316 | 2.7 | 13.4 | 1.5 |
| 654452 | 320 | 2.5 | 11.6 | 0.9 |
| 654472 | 361 | 3.0 | 13.1 | 1.5 |
| 654481 | 370 | 3.2 | 11.4 | 1.3 |
| 654483 | 366 | 3.3 | 13.5 | 1.2 |
| 654691 | 288 | 3.1 | 14.3 | 2.1 |
| 654999 | 344 | 2.7 | 11.5 | 2.0 |

### Liver and Kidney Function

To evaluate the effect of the 9 antisense oligonucleotides on liver and kidney function, plasma levels of ALT (alanine transaminase) and AST (aspartate transaminase), albumin, BUN, creatinine and bilirubin were measured using an automated clinical chemistry analyzer (Hitachi Olympus AU400e, Melville, NY), and total urine protein and urine creatinine levels were measured, and the ratio of total urine protein to creatinine (P/C Ratio) was determined.

Results of each group were averaged, and a selection of these is presented in Table 46.

TABLE 46
Liver and kidney function markers in Sprague-Dawley rats

| | | | | | | | | urine | | |
|---|---|---|---|---|---|---|---|---|---|---|
| | | plasma | | | | | | | Total | Urine |
| | | ALT | AST | Albumin | BUN | Cre | T. bil | Cre | protein | P/C |
| ISIS NO | Compound | (U/L) | (U/L) | (g/dL) | (mg/dL) | (mg/dL) | (mg/dL) | (mg/dL) | (mg/dL) | Ratio |
| PBS | N/A | 28 | 72 | 3.2 | 19 | 0.28 | 0.08 | 86 | 88 | 1 |
| 619998 | 5-10-5 MOE | 57 | 125 | 2.8 | 28 | 0.31 | 0.10 | 76 | 251 | 3 |
| 620003 | 5-10-5 MOE | 54 | 106 | 3.1 | 25 | 0.30 | 0.09 | 81 | 356 | 4 |
| 654401 | 5-10-5 MOE | 69 | 136 | 3.3 | 25 | 0.36 | 0.12 | 64 | 343 | 6 |
| 654451 | 5-10-5 MOE | 62 | 149 | 2.8 | 28 | 0.28 | 0.10 | 37 | 209 | 6 |
| 654452 | 5-10-5 MOE | 159 | 196 | 3.0 | 30 | 0.34 | 0.11 | 44 | 356 | 8 |
| 654472 | 5-10-5 MOE | 44 | 98 | 3.1 | 28 | 0.36 | 0.09 | 69 | 413 | 6 |
| 654481 | 5-10-5 MOE | 43 | 101 | 3.2 | 26 | 0.37 | 0.09 | 56 | 323 | 6 |
| 654483 | 5-10-5 MOE | 42 | 87 | 3.0 | 28 | 0.30 | 0.08 | 54 | 360 | 6 |
| 654691 | 3-10-4 MOE | 41 | 94 | 2.7 | 31 | 0.31 | 0.08 | 40 | 237 | 6 |
| 654999 | 5-8-7 MOE | 40 | 120 | 2.8 | 28 | 0.30 | 0.09 | 47 | 335 | 7 |

### Histology

Liver and kidney from antisense oligonucleotide-treated rats were microscopically examined, and no remarkable treatment-related adverse finding was observed.

In a separate study, antisense compounds ISIS 568637, 594622, 594624, 594625 and 594627 were also tested in SD rats, but exhibited some tolerability issues and the study was terminated early.

### Example 8: Potency in Cynomolgus Monkey Hepatocytes of Nine Lead Antisense Oligonucleotides (ASOs) Targeting Human AGT

At the time this study was undertaken, the cynomolgus monkey genomic sequence was not available in the National Center for Biotechnology Information (NCBI) database; therefore, cross-reactivity with the cynomolgus monkey gene sequence could not be confirmed. Instead, the sequences of the antisense oligonucleotides used in the cynomolgus monkeys were compared to a rhesus monkey sequence for complementarity. It is expected that antisense oligonucleotides with complementarity to the rhesus monkey sequence are fully cross-reactive with the cynomolgus monkey sequence as well.

The human antisense oligonucleotides tested had at most 3 mismatches with the rhesus genomic sequence (GENBANK Accession NW_001109259.1 truncated from nucleotide 16090000 to 16106000, designated herein as SEQ ID NO: 7). The greater the complementarity between the human oligonucleotide and the rhesus monkey sequence, the more likely the human oligonucleotide can cross-react with the rhesus monkey sequence and the cynomolgus monkey sequence. The start and stop sites of each oligonucleotide to SEQ ID NO: 7 is presented in Table 47. “Start site” indicates the 5′-most nucleotide to which the gapmer is targeted in the rhesus monkey gene sequence.

Nine antisense oligonucleotides exhibiting significant inhibition of AGT mRNA and tolerability in previous studies were selected and tested at various doses in cryopreserved individual male cynomolgus monkey primary hepatocytes. These 9 lead antisense oligonucleotides are described in the table below.

TABLE 47
ASO complementarity to the rhesus AGT
genomic sequence (SEQ ID NO: 7)

| | Target | Target | | | # | SEQ |
|---|---|---|---|---|---|---|
| ISIS | Start | Stop | | | mismatches | ID |
| NO | Site | Site | Sequence | Chemistry | in Rhesus | NO |
| 619998 | 13777 | 13796 | TCGGTTGGAATTCTTTTTGG | 5-10-5 MOE | 0 | 1714 |
| 620003 | 13792 | 13811 | TCACAAACAAGCTGGTCGGT | 5-10-5 MOE | 0 | 1719 |
| 654451 | N/A | N/A | TGGAACAGTAGTCCCGCGCT | 5-10-5 MOE | 2 | 1893 |
| 654452 | N/A | N/A | TTGGAACAGTAGTCCCGCGC | 5-10-5 MOE | 2 | 1894 |
| 654472 | 13791 | 13810 | CACAAACAAGCTGGTCGGTT | 5-10-5 MOE | 0 | 1914 |
| 654481 | 13822 | 13841 | CTCAACTTGAAAAGGGAACA | 5-10-5 MOE | 0 | 1923 |
| 654483 | 13825 | 13844 | GTTCTCAACTTGAAAAGGGA | 5-10-5 MOE | 0 | 1925 |
| 654691 | N/A | N/A | TCGCTGATTTGTCCGGG | 3-10-4 MOE | 3 | 2003 |
| 654999 | N/A | N/A | ACACATCGCTGATTTGTCCG | 5-8-7 MOE | 3 | 238 |

Cynomolgus monkey primary hepatocytes were plated at a density of 35,000 cells per well and transfected using electroporation with 0.156 μM, 0.313 μM, 0.625 μM, 1.25 μM, 2.5 μM, 5.0 μM, 10.0 μM and 20.0 μM concentrations of antisense oligonucleotide, as specified in Table 48 below. After a treatment period of approximately 24 hours, the cells were washed and lysed, and RNA was isolated. Monkey AGT mRNA levels were measured by quantitative real-time PCR, using primer probe set RTS4039. AGT mRNA target levels were adjusted according to total RNA content, as measured by RIBOGREEN®. Results are presented as percent inhibition of AGT, relative to untreated control cells.

TABLE 48
Dose response in primary hepatocytes from cynomolgus monkeys

| | 0.156 | 0.313 | 0.625 | 1.25 | 2.5 | 5.0 | 10.0 | 20.0 | IC50 | SEQ |
|---|---|---|---|---|---|---|---|---|---|---|
| ISIS NO | μM | μM | μM | μM | μM | μM | μM | μM | (μM) | ID NO |
| 619998 | 3 | 1 | 0 | 13 | 20 | 31 | 36 | 64 | 13.9 | 1714 |
| 620003 | 9 | 7 | 15 | 27 | 30 | 62 | 76 | 80 | 3.0 | 1719 |
| 654451 | 13 | 24 | 20 | 30 | 38 | 42 | 47 | 29 | 10.6 | 1893 |
| 654452 | 13 | 13 | 25 | 47 | 44 | 41 | 62 | 35 | >20 | 1894 |
| 654472 | 12 | 24 | 22 | 37 | 39 | 55 | 74 | 78 | 3.4 | 1914 |
| 654481 | 0 | 14 | 27 | 26 | 43 | 48 | 53 | 45 | >20 | 1923 |
| 654483 | 25 | 24 | 39 | 46 | 61 | 50 | 56 | 61 | 3.2 | 1925 |
| 654691 | 0 | 12 | 18 | 0 | 23 | 18 | 19 | 24 | >20 | 2003 |
| 654999 | 0 | 19 | 0 | 0 | 9 | 17 | 37 | 42 | >20 | 238 |

Most monkey AGT mRNA levels were significantly reduced in a dose-dependent manner in antisense oligonucleotide treated cells.

### Example 9: Effect of Antisense Oligonucleotides Targeting Human AGT in Cynomolgus Monkeys

In a 12-week dose response study, cynomolgus monkeys were treated with the nine antisense oligonucleotides selected from studies described in the Examples above. Antisense oligonucleotide efficacy and tolerability, as well as their pharmacokinetic profile in the liver and kidney, were evaluated.

### Treatment

Prior to the study, the monkeys were kept in quarantine during which the animals were observed daily for general health. The monkeys were two to four years old and weighed 2-4 kg. Ten groups of five randomly assigned male cynomolgus monkeys each were injected subcutaneously with antisense oligonucleotide or PBS. The monkeys were dosed once a week for 12 weeks with 40 mg/kg/wk of antisense oligonucleotide for a total of 15 doses (monkeys received a loading treatment of two doses of 40 mg/kg in weeks 1 and 2). A control group of cynomolgus monkeys was injected with PBS in a similar manner and served as the control group.

During the study period, the monkeys were observed twice daily for signs of illness or distress. Any animal experiencing more than momentary or slight pain or distress due to the treatment, injury or illness was treated by the veterinary staff with approved analgesics or agents to relieve the pain after consultation with the Study Director. Any animal in poor health or in a possible moribund condition was identified for further monitoring and possible euthanasia. At the end of the 12-week study, the monkeys were sacrificed and organs removed. The protocols described in the Example were approved by the Institutional Animal Care and Use Committee (IACUC).

### Body and Organ Weights

Body weight was assessed weekly, and no remarkable effects of the antisense oligonucleotides on body weight were observed. Body weight at day 77 and organ weights at day 79 were measured and are presented in Table 49 below

TABLE 49
Body and organ weights (grams) of cynomolgus
monkeys treated with nine lead ASOs

| | Weight (g) | | | | |
|---|---|---|---|---|---|
| ISIS NO | body (day 77) | heart | kidney | liver | spleen |
| PBS | 2524 | 10.2 | 12.2 | 51.8 | 2.7 |
| 619998 | 2520 | 9.3 | 23.4 | 73.9 | 4.4 |
| 620003 | 2638 | 9.5 | 14.5 | 67.8 | 3.3 |
| 654451 | 2488 | 9.4 | 15.9 | 68.5 | 3.0 |
| 654452 | 2510 | 9.8 | 14.2 | 60.8 | 3.1 |
| 654472 | 2623 | 9.8 | 14.8 | 62.1 | 4.0 |
| 654481 | 2549 | 9.6 | 14.2 | 59.8 | 4.0 |
| 654483 | 2525 | 10.0 | 15.8 | 68.6 | 4.0 |
| 654691 | 2497 | 8.8 | 15.3 | 67.9 | 4.3 |
| 654999 | 2590 | 10.1 | 16.6 | 69.1 | 5.7 |

### Pharmacodynamics

Plasma, serum and urine were collected for analysis during the study. To evaluate the effect of the nine lead antisense oligonucleotides on liver and kidney function, on day 79, plasma levels of ALT (alanine transaminase) and AST (aspartate transaminase), BUN and bilirubin were measured using an automated clinical chemistry analyzer (Hitachi Olympus AU400e, Melville, NY). As shown in Table 50, no significant effects on ALT, AST, BUN and bilirubin were observed.

TABLE 50
Plasma chemistry markers in monkeys treated
with antisense oligonucleotides

| | ALT | AST | BUN | T. Bil |
|---|---|---|---|---|
| ISIS NO | (U/L) | (U/L) | (mg/dL) | (mg/dL) |
| PBS | 54 | 87 | 25.1 | 0.20 |
| 619998 | 74 | 98 | 36.6 | 0.16 |
| 620003 | 53 | 87 | 24.9 | 0.16 |
| 654451 | 61 | 74 | 30.1 | 0.13 |
| 654452 | 63 | 100 | 25.8 | 0.20 |
| 654472 | 62 | 77 | 27.1 | 0.16 |
| 654481 | 58 | 63 | 21.7 | 0.16 |
| 654483 | 70 | 78 | 25.0 | 0.14 |
| 654691 | 57 | 97 | 26.4 | 0.14 |
| 654999 | 62 | 111 | 23.2 | 0.14 |

In addition, no significant changes in ECG, blood pressure, plasma electrolytes, proteinuria, inflammatory response (e.g., CRP levels) or renal accumulation were observed. In general, the antisense oligonucleotides were well tolerated.

### RNA Analysis

At the end of the study, RNA was extracted from monkey livers and kidneys for real-time PCR analysis of measurement of mRNA expression of AGT. Primer probe set RTS4039 was used, and the results for each group were averaged and presented as percent inhibition of mRNA, relative to the PBS control, normalized with RIBOGREEN®. As shown in Table 51, treatment with antisense oligonucleotides resulted in variable effects on AGT mRNA levels.

TABLE 51
Percent inhibition of AGT mRNA in the cynomolgus
monkey liver relative to the PBS control

| ISIS NO | % inhibit | SEQ ID NO |
|---|---|---|
| 619998 | 75 | 1714 |
| 620003 | 40 | 1719 |
| 654451 | 33 | 1893 |
| 654452 | 0 | 1894 |
| 654472 | 9 | 1914 |
| 654481 | 1 | 1923 |
| 654483 | 38 | 1925 |
| 654691 | 36 | 2003 |
| 654999 | 3 | 238 |

### Example 10: Tolerability of GalNAc Conjugated Antisense Oligonucleotides in CD-1 Mice

A lead candidate (ISIS 654472) 5-10-5 full phosphorothioate MOE gapmer was chosen from studies above and used as the basis for design of six 5′-Trishexylamino-(THA)-C6 GalNAc₃ (a.k.a. “GalNAc”)-conjugated 5-10-5 MOE gapmers having the same nucleotide sequence but differences in the backbone structure, as described in Table 52 below. “s” is a phosphorothioate internucleoside linkage. “o” is a phosphodiester internucleoside linkage. “A” is an adenine nucleobase. “mC” is a 5′-methylcytosine nucleobase. “G” is a guanine nucleobase. “T” is a thymine nucleobase. “e” indicates a MOE modification. 10 “d” indicates deoxyribose.

TABLE 52
GalNAc-conjugated ASOs and unconjugated parent ASO

| ISIS | | | SEQ ID |
|---|---|---|---|
| NO | Chemistry notation | | NO |
| 654472 | mCes Aes mCes Aes Aes Ads mCds Ads Ads Gds | (PS) | 1914 |
| (parent) | mCds Tds Gds Gds Tds mCes Ges Ges Tes Te | | |
| 757456 | mCes Aes mCes Aes Aes Ads mCds Ads Ads Gds | (PS) GalNAc | 1914 |
| | mCds Tds Gds Gds Tds mCes Ges Ges Tes Te | | |
| 757457 | mCes Aeo mCeo Aeo Aeo Ads mCds Ads Ads Gds | (mixed backbone) | 1914 |
| | mCds Tds Gds Gds Tds mCeo Geo Ges Tes Te | GalNAc | |
| 775493 | mCes Aeo mCeo Aeo Aes Ads mCds Ads Ads Gds | (mixed backbone) | 1914 |
| | mCds Tds Gds Gds Tds mCeo Geo Ges Tes Te | GalNAc | |
| 775494 | mCes Aes mCeo Aeo Aes Ads mCds Ads Ads Gds | (mixed backbone) | 1914 |
| | mCds Tds Gds Gds Tds mCeo Geo Ges Tes Te | GalNAc | |
| 775495 | mCes Aeo mCes Aeo Aes Ads mCds Ads Ads Gds | (mixed backbone) | 1914 |
| | mCds Tds Gds Gds Tds mCeo Ges Geo Tes Te | GalNAc | |
| 775496 | mCes Aes mCeo Aes Aes Ads mCds Ads Ads Gds | (mixed backbone) | 1914 |
| | mCds Tds Gds Gds Tds mCes Geo Ges Tes Te | GalNAc | |

For a three-point dose response study, sixteen groups of four CD1 mice each were subcutaneously injected with 10 mg/kg/week of GalNAc-conjugated antisense oligonucleotide over the course of four weeks. One group of mice was injected subcutaneously twice a week for six weeks with PBS. Body weights of ASO-treated CD1 mice were measured weekly. Mice were euthanized 48 hours after the last dose, and organs and plasma were harvested for further analysis. Plasma and urine were collected and plasma levels of transaminases, bilirubin and BUN were measured using an automated clinical chemistry analyzer (Hitachi Olympus AU400e, Melville, NY) to evaluate the effect of antisense oligonucleotides on liver and kidney function. At the end of the experiment, the livers, kidneys and spleens were harvested and weighed.

The results were averaged for each group is presented in Table 53.

TABLE 53
Plasma chemistry markers in CD1 mice treated with GalNAc-conjugated ASOs

| | | Weight (g) | | | | | | | |
|---|---|---|---|---|---|---|---|---|---|
| | | body | | | | ALT | AST | BUN | T. Bil |
| ISIS NO | mg/kg | (day 29) | kidney | liver | spleen | (U/L) | (U/L) | (mg/dL) | (mg/dL) |
| PBS | n/a | 36.1 | 0.58 | 2.1 | 0.1 | 31 | 41 | 25.8 | 0.14 |
| 757456 | 20 | 39.3 | 0.64 | 2.5 | 0.1 | 28 | 31 | 24.2 | 0.18 |
| | 10 | 38.1 | 0.56 | 2.3 | 0.1 | 38 | 74 | 24.8 | 0.22 |
| | 5 | 36.1 | 0.54 | 2.1 | 0.1 | 39 | 59 | 27.7 | 0.21 |
| 757457 | 20 | 40.6 | 0.59 | 2.4 | 0.2 | 34 | 38 | 26.2 | 0.23 |
| | 10 | 38.7 | 0.60 | 2.2 | 0.1 | 24 | 29 | 23.1 | 0.26 |
| | 5 | 39.1 | 0.58 | 2.2 | 0.2 | 39 | 46 | 29.0 | 0.20 |
| 775493 | 20 | 36.3 | 0.59 | 2.0 | 0.1 | 36 | 51 | 28.3 | 0.21 |
| | 10 | 38.6 | 0.58 | 2.2 | 0.1 | 30 | 45 | 25.0 | 0.34 |
| | 5 | 37.1 | 0.58 | 2.3 | 0.1 | 23 | 32 | 26.5 | 0.15 |
| 775494 | 20 | 37.9 | 0.56 | 2.0 | 0.2 | 47 | 55 | 29.1 | 0.31 |
| | 10 | 36.4 | 0.59 | 2.1 | 0.3 | 25 | 34 | 25.4 | 0.20 |
| | 5 | 38.4 | 0.59 | 2.0 | 0.1 | 35 | 69 | 24.9 | 0.21 |
| 775495 | 20 | 39.3 | 0.67 | 2.3 | 0.2 | 42 | 86 | 23.7 | 0.19 |
| | 10 | 37.0 | 0.55 | 2.1 | 0.1 | 34 | 44 | 25.1 | 0.21 |
| | 5 | 38.1 | 0.62 | 2.2 | 0.1 | 20 | 28 | 22.5 | 0.28 |
| 775496 | 20 | 37.0 | 0.58 | 2.1 | 0.2 | 32 | 38 | 24.7 | 0.15 |
| | 10 | 36.4 | 0.59 | 1.9 | 0.2 | 38 | 42 | 25.0 | 0.24 |
| | 5 | 36.7 | 0.56 | 2.1 | 0.1 | 23 | 28 | 25.6 | 0.34 |

### Example 11: Tolerability of GalNAc Conjugated ASOs in SD Rats

Twenty-eight male SD rats were divided into seven groups, four rats per group. Rats were subcutaneously injected with PBS as an untreated control or 10 mg/kg/week of a GalNAc conjugated antisense oligonucleotide over the course of four weeks.

Plasma and urine were collected and analyzed using an automated clinical chemistry analyzer (Hitachi Olympus AU400e, Melville, NY) to evaluate the effect of antisense oligonucleotides on liver and kidney function. At the end of the experiment, the livers, kidneys and spleens were harvested and weighed.

Results are presented as average of 4 animals in each group and presented in Table 54.

TABLE 54
Tolerability of GalNAc-conjugated ASOs in SD rats

| | | | | | | | | Weight (g) | | | |
|---|---|---|---|---|---|---|---|---|---|---|---|
| | plasma | | | | urine | | | body | | | |
| Tx | ALT | AST | BUN | T. Bil | Cre | MTP | MTP/Cre | (day 30) | kidney | liver | spleen |
| PBS | 29 | 76 | 17 | 0.08 | 95.8 | 106.8 | 0.99 | 395 | 3.1 | 11.2 | 0.70 |
| 757456 | 42 | 103 | 19 | 0.12 | 55.5 | 65.3 | 1.24 | 387 | 3.0 | 11.1 | 0.84 |
| 757457 | 36 | 84 | 18 | 0.08 | 82.8 | 103.0 | 1.13 | 396 | 3.0 | 11.2 | 0.69 |
| 775493 | 42 | 102 | 16 | 0.14 | 66.0 | 85.3 | 1.29 | 407 | 3.3 | 12.3 | 0.78 |
| 775494 | 43 | 84 | 17 | 0.09 | 91.3 | 119.0 | 1.34 | 396 | 2.8 | 10.7 | 0.89 |
| 775495 | 37 | 92 | 17 | 0.10 | 65.0 | 70.3 | 1.01 | 387 | 3.0 | 10.4 | 0.81 |
| 775496 | 36 | 90 | 16 | 0.08 | 58.3 | 92.8 | 1.39 | 397 | 3.1 | 11.7 | 0.91 |

### Example 12: Dose Response Comparison of Unconjugated and GalNAc Conjugated Antisense Oligonucleotides in Male and Female huAGT Mice

As described in previous examples, huAGT mice are useful in testing the potency of antisense oligonucleotides. A dose response comparison of the parent 5-10-5 MOE gapmer (ISIS 654472) to a GalNAc conjugated compound with the same sequence (ISIS 757456) was performed. The GalNAc conjugated antisense oligonucleotide is 8-fold more potent than the unconjugated antisense oligonucleotide as shown in Table 55.

TABLE 55
Dose response of conjugated versus unconjugated ASO

| | | ED50 | | | % reduction |
|---|---|---|---|---|---|
| | | AGT | Liver | Kidney | plasma |
| | mg/kg | mRNA | % inhib | % inhib | AGT protein |
| Females | Saline | n/a | 0 | 0 | 0 |
| 654472 | 2.5 | 24 | 15 | 4 | 0 |
| (parent) | 8 | | 17 | 26 | 8 |
| | 25 | | 48 | 38 | 31 |
| | 80 | | 76 | 51 | 70 |
| 757456 | 0.3 | 3 | 10 | 0 | 0 |
| (GalNAc) | 1 | | 19 | 12 | 15 |
| | 3 | | 47 | 0 | 53 |
| | 10 | | 79 | 7 | 75 |
| Males | Saline | n/a | 0 | 0 | 2 |
| 654472 | 8 | 24 | 35 | 23 | 33 |
| (parent) | 25 | | 48 | 36 | 39 |
| | 80 | | 79 | 49 | 78 |
| 757456 | 1 | 3 | 19 | 1 | 17 |
| (GalNAc) | 3 | | 50 | 10 | 39 |
| | 10 | | 78 | 20 | 71 |

1. (canceled)

2. A compound comprising a modified oligonucleotide consisting of 16 to 20 linked nucleosides comprising the nucleobase sequence of SEQ ID NO: 129, wherein the modified oligonucleotide comprises:
a gap segment consisting of 8-10 linked deoxynucleosides;
5′ wing segment consisting of 3-7 linked nucleosides; and
a 3′ wing segment consisting of 3-7 linked nucleosides;
or a pharmaceutically acceptable salt thereof;
wherein the gap segment is positioned between the 5′ wing segment and the 3′ wing segment, wherein each nucleoside of each wing segment comprises a modified sugar, and wherein at least one internucleoside linkage in the gap segment is a phosphorothioate internucleoside linkage.

3. The compound of claim 2, wherein the compound comprises a modified oligonucleotide having the nucleobase sequence consisting of SEQ ID NO: 129, SEQ ID NO: 2003, SEQ ID NO: 2015, SEQ ID NO: 2035 or SEQ ID NO: 236.

4. The compound of claim 2, wherein each cytosine in the modified oligonucleotide is a 5-methylcytosine.

5. The compound of claim 2, wherein the compound is single-stranded.

6. The compound of claim 1, wherein the modified oligonucleotide comprises at least one modified sugar comprising a 2′-O-methoxyethyl or a constrained ethyl (cEt).

7. The compound of claim 1, wherein the modified oligonucleotide has a sugar motif eekddddddddddkke, kkkddddddddddkkk, eekkdddddddkkeee, eekkddddddddkkee, ekkdddddddddkkee, ekkddddddddddkke, eeeeeddddddddddeeeee, eeeeeeeddddddddeeeee, eeeeeeddddddddeeeeee, eeeeedddddddddeeeeee, eeeeeddddddddeeeeeee, eeeeeedddddddddeeeee, eeeedddddddddeeeeee, eeeeedddddddddeeeee, eeeeddddddddddeeeee, eeeeddddddddeeeeeee, eeeeeeddddddddeeeee, eeeeddddddddddeeee, eeeeeddddddddeeeee, or eeeddddddddddeeee, wherein:
e=a 2′-MOE sugar moiety,
d=a 2′-deoxyribosyl sugar moiety, and
k=a cEt sugar moiety.

8. The compound of claim 1, wherein the compound is in a pharmaceutically acceptable salt form, and the pharmaceutically acceptable salt is a sodium salt or a potassium salt.

9. A pharmaceutical composition comprising the compound of claim 1 and a pharmaceutically acceptable carrier or diluent.

10. The pharmaceutical composition of claim 8, wherein the pharmaceutically acceptable diluent is water, saline, or phosphate-buffered saline (PBS).

11. A method of treating a disease, disorder, or condition associated with the renin-angiotensin-aldosterone system (RAAS) in a subject in need thereof comprising administering to the subject the compound of claim 2 or a pharmaceutical composition of claim 9.

12. The method of claim 11, wherein the disease, disorder, or condition is hypertension.

13. The use of claim 12, wherein the hypertension is resistant hypertension.

14. The method of claim 11, wherein the disease, disorder, or condition is heart disease.

15. The method of claim 11, wherein the disease, disorder, or condition is stroke, pre-eclampsia, aneurysms of the blood vessels, abdominal aneurysm, peripheral artery disease, tissue fibrosis, pulmonary arterial hypertension, heart damage, liver damage, or kidney damage.

16. The method of claim 11, wherein the compound or pharmaceutical composition is administered systemically, subcutaneously, or intramuscularly.

17. A method for reducing angiotensinogen in a subject in need thereof comprising administering to the subject the compound of claim 2 or the pharmaceutical composition of claim 9.

18. The method of claim 17, wherein the level of AGT mRNA is reduced or the level of AGT protein is reduced.

19. The method of claim 11, wherein the compound or pharmaceutical composition is administered in combination with one or more additional therapeutic agents, wherein the therapeutic agent is an anti-fibrotic drug, an anti-hypertensive drug, a RAS or RAAS inhibitor, an endothelin receptor antagonist, a neprilysin inhibitor, a diuretic, a calcium channel blocker, an adrenergic receptor antagonist, an adrenergic agonist, or a vasodilator.

20. A kit for treating, ameliorating, or slowing progression of a disease, disorder, or condition associated with the RAAS pathway in a subject in need thereof, wherein the kit comprises:
(i) instructions for using the kit;
(ii) the compound of claim 2; and
(iii) optionally one or more additional therapeutic agents.
